(19) United States
(12) Patent Application Publication
(10) Pub. No.: US 2024/0124573 A1
(43) Pub. Date: Apr. 18, 2024

(54) Use of Anti-Il-6 Antibody, e.g., Clazakizumab for Desensitization of Solid Organ Transplant Recipients and/or for Preventing, Stabilizing or Reducing Antibody Mediated Rejection (ABMR)

(71) Applicants: VITAERIS, INC., Vancouver (CA); CEDARS-SINAI MEDICAL CENTER, Los Angeles, CA (US)

(72) Inventors: Kevin CHOW, Sammamish, WA (US); Edward CHONG, North Vancouver (CA); Nuala MOONEY, Paris (FR); Julien LION, Camon (FR); Stanley C. JORDAN, Manhattan Beach, CA (US)

(73) Assignees: VITAERIS, INC., Vancouver, BC (CA); CEDARS-SINAI MEDICAL CENTER, Los Angeles, CA (US)

(21) Appl. No.: 18/447,623

(22) Filed: Aug. 10, 2023

Related U.S. Application Data
(63) Continuation of application No. 16/959,923, filed on Jul. 2, 2020, now abandoned.
(60) Provisional application No. 62/773,630, filed on Nov. 30, 2018.
(60) Provisional application No. 62/736,205, filed on Sep. 25, 2018.
(60) Provisional application No. 62/684,870, filed on Jun. 14, 2018.
(60) Provisional application No. 62/613,447, filed on Jan. 4, 2018.

(51) Int. Cl. C07K 16/24; A61P 37/06

(52) U.S. Cl. CPC C07K 16/248; A61P 37/06; A61K 2039/505

(57) ABSTRACT

Novel therapeutic protocols are provided relating to the use of an anti-IL-6 antibody, e.g., Clazakizumab, in order to prevent, stabilize, reduce or arrest antibody mediated rejection responses in patients receiving solid organ transplants, e.g., patients receiving transplanted kidney, heart, liver, lungs, pancreas, intestines or combinations of any of the foregoing. Also novel therapeutic protocols are provided pertaining to the use of an anti-IL-6 antibody, e.g., Clazakizumab, as part of a desensitization protocol for treating highly sensitized subjects waiting for and/or after allograft transplants, e.g., patients who are to receive solid organ transplants, e.g., kidney, heart, liver, lungs, pancreas, intestines, skin or combinations of any of the foregoing. The foregoing treatments may be effected in combination with one or more other immunosuppressant regimens or other desensitization procedures.

### PRIORITY CLAIMS

This application is a continuation of U.S. application Ser. No. 16/959,923, filed Jul. 2, 2020, which is a national phase entry pursuant to 35 U.S.C. § 371 of International Application No. PCT/US2019/012372, filed Jan. 4, 2019, which claims priority to U.S. Provisional No. 62/613,447 filed on Jan. 4, 2018; U.S. Provisional No. 62/684,870 filed on Jun. 14, 2018; U.S. Provisional No. 62/736,205 filed on Sep. 25, 2018; and U.S. Provisional No. 62/773,630 filed on Nov. 30, 2018. The contents of each of these applications is incorporated by reference in its entirety herein.

### SEQUENCE LISTING

The present application contains a Sequence Listing which has been submitted electronically in XML format. Said XML copy, created on Aug. 10, 2023, is named “2023-08-10_01113-0017-01US-ST26.xml” and is 723,799 bytes in size. The information in the electronic format of the sequence listing is incorporated herein by reference in its entirety.

### FIELD

This invention pertains to the use of an anti-IL-6 antibody, e.g., Clazakizumab in order to prevent, stabilize or reduce antibody mediated rejection responses in patients receiving solid organ transplants, e.g., patients receiving transplanted kidney, heart, liver, lungs, pancreas, intestines, skin, or combinations of any of the foregoing.

This invention further pertains to the use of an anti-IL-6 antibody or anti-IL-6 antibody fragment, e.g., Clazakizumab as part of a desensitization protocol for treating highly sensitized subjects waiting for or after allograft transplants, e.g., patients who are to receive solid organ transplants, e.g., kidney, heart, liver, lungs, pancreas, intestines, skin, stomach, gall bladder or combinations of any of the foregoing. The foregoing treatments may be effected in combination with one or more other immunosuppressant regimens or other desensitization procedures.

### BACKGROUND

Despite significant improvements in pre- and post-transplant care, both short and long term graft survival rates are currently less than optimal. Many patients awaiting transplants are sensitized to antigens (e.g. human leukocyte antigen (HLA) antigens and non-HLA antigens) present in the donor organ and may remain on the waiting list for a prolonged period. These patients generally become sensitized because of a history of blood transfusions, pregnancies or previous transplants, and develop pre-formed donor specific antibodies (DSA) to the donor organ. Such patients are at high risk of acute and chronic rejection, allograft failure and death after transplantation. For a successful transplant to occur, these patients must undergo pre-transplant desensitization procedures to remove or reduce these DSA. However, these treatments are not always successful and many patients remain sensitized or have to undergo prolonged desensitization before they are transplanted.

Furthermore many patients, whether sensitized pre-transplant or not, develop antibody mediated rejection (ABMR) after transplantation. ABMR is now recognized as the largest single cause of post-transplant allograft failure. Indolent ABMR can start soon after transplantation but often leads to clinical signs of allograft dysfunction and eventually graft failure many months or even years after the transplant procedure. Moreover, ABMR is not amenable to treatment with the current standard-of-care immunosuppressive medications, despite the availability of laboratory tests to predict patients at risk of and to diagnose ABMR.

The underlying pathophysiology of ABMR indicates a primary role of B-cells and plasma cells producing DSA against HLA- and non-HLA antigens present in the donor organ. These antibodies damage the organ via complement and non-complement pathways. Recently developed diagnostic tests allow for the prediction and early diagnosis of ABMR: these tests include assays to detect pre-formed and de novo HLA DSA (especially those detecting complement binding DSA such as Clq) and assays for non-HLA antibodies associated with ABMR. Histological features of antibody damage include, in kidney allograft biopsies, evidence of microvascular inflammation, complement deposition (C4d) in the peritubular capillaries, peritubular capillaritis, glomerulitis and transplant glomerulopathy (double glomerular basement membrane contour). Similar histological features caused by ABMR are observed in other transplanted organs. Active antibody-mediated rejection (ABMR), especially chronic active antibody-mediated rejection (CABMR), is now recognized as the most common cause of allograft failure after a successful kidney transplant. Current standard of care anti-rejection treatments target cellular-mediated (i.e., T cell-mediated rejection (TCMR)) processes and do not affect this antibody-mediated process. Currently, there are no approved treatments for active ABMR, including CABMR.

### SUMMARY

This invention relates to the use of an anti-IL-6 monoclonal antibody (mAb), e.g., clazakizumab for the treatment of AMBR or CABMR in recipients of a transplant, e.g., kidney transplant recipients by inhibiting the production of DSA alloimmune responses. Clazakizumab comprises the heavy and light chain sequences set forth below:

(Heavy chain)
SEQ ID NO: 745
EVQLVESGGGLVQPGGSLRLSCAASGFSLSNYYVTWVRQAPGKGLEWVGI
IYGSDETAYATSAIGRFTISRDNSKNTLYLQMNSLRAEDTAVYYCARDDS
SDWDAKFNLWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPKP
KDTLMISRTPEVTCVVVDVSHEDPEVKFNW YVDGVEVHNAKTKPREEQY
ASTYRVVSVLTVLHQDWLNGKEYKCKVSNKALPAPIEKTIS KAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK
(Light chain)
SEQ ID NO: 746
AIQMTQSPSSLSASVGDRVTITCQASQSINNELSWYQQKPGKAPKLLIYR
ASTLASGVPSRFSGSGSGTDFTLTISSLQPDDFATYYCQQGYSLRNIDNA
FGGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEVT
HQGLSSPVTK SFNRGEC

It is an object of the invention to provide therapeutic protocols for treating or preventing ABMR or CAMBR in patients in need thereof, particularly those receiving solid organ transplants by the use of specific anti-IL-6 antibodies and anti-IL-6 antibody fragments.

It is another object of the invention to provide novel protocols for desensitization of highly sensitized subjects awaiting allograft transplant transplants and after an allograft transplant transplants by the use of specific anti-IL-6 antibodies and antibody fragments, e.g., Clazakizumab.

More specifically it is object of the invention to provide methods of preventing, stabilizing or reducing antibody mediated rejection (ABMR) in a subject who is or has received a solid organ transplant, comprising administering to said subject a prophylactically or therapeutically effective amount of an anti-human interleukin-6 (IL-6) antibody or anti-IL-6 antibody fragment, wherein the antibody or antibody fragment comprises: a variable light chain polypeptide comprising the CDRs of SEQ ID NOs:4, 5 and 6 and, and a variable heavy chain polypeptide comprising the CDRs of SEQ ID NOs:7, 8 or 120, and 9, e.g., wherein the antibody comprises a VH and VL polypeptide respectively at least 90, 95, 96, 97, 98 or 99% identical to the polypeptides of SEQ ID NO:657 and 709 and preferably wherein the antibody is clazakizumab. In exemplary embodiments the solid organ is selected from kidney, heart, liver, lungs, pancreas, skin, intestine, stomach, skin, gall bladder, bladder, or a combination of any of the foregoing or preferably is a kidney.

It is an object of the invention to treat or prevent ABMR in patients in need thereof, particularly those receiving solid organ transplants by the use of specific anti-IL-6 antibodies and antibody fragments, e.g., Clazakizumab, wherein the patient is evaluated to diagnose the onset of or progression of ABMR, e.g., wherein the evaluation comprises one or more of: detecting pre-formed and de novo HLA DSA (especially those detecting complement binding DSA such as Clq), detecting non-HLA antibodies associated with ABMR, or identifying at least one histological feature characteristic of antibody mediated organ damage.

It is an object of the invention to treat or prevent ABMR in patients in need thereof, particularly those receiving solid organ transplants by the use of specific anti-IL-6 antibodies and antibody fragments, e.g., Clazakizumab, wherein the patient is evaluated for a histological feature characteristic of antibody mediated organ damage is detected by obtaining a biopsy from the transplanted organ and optionally the histological feature characteristic of antibody mediated organ damage includes any of microvascular inflammation, complement deposition (C4d), and capillaritis.

It is another more specific object of the invention to treat or prevent ABMR in patients in need thereof, particularly those receiving solid organ transplants by the use of specific anti-IL-6 antibodies and antibody fragments, e.g., Clazakizumab, wherein the patient is evaluated to diagnose the onset of or progression of ABMR, e.g., wherein the evaluation comprises one or more of: detecting pre-formed and de novo HLA DSA (especially those detecting complement binding DSA such as Clq), detecting non-HLA antibodies associated with ABMR, or identifying at least one histological feature characteristic of antibody mediated organ damage wherein the transplanted organ is a kidney and the histological feature characteristic of antibody mediated organ damage includes any of microvascular inflammation, complement deposition (C4d) in the peritubular capillaries, peritubular capillaritis, glomerulitis and transplant glomerulopathy (double glomerular basement membrane contour).

It is another more specific object of the invention to treat or prevent ABMR in patients in need thereof, particularly those receiving solid organ transplants by the use of specific anti-IL-6 antibodies and antibody fragments, e.g., Clazakizumab, wherein the treatment further includes the administration of at least one other immunosuppressant, e.g., a standard of care pre- or post-transplant immunosuppressive medication, optionally any of thymoglobulin, basiliximab, mycophenolate mofetil, tacrolimus, an anti-CD20 mAb such as rituximab, and corticosteroids.

It is another more specific object of the invention to treat or prevent ABMR in patients in need thereof, particularly those receiving solid organ transplants by the use of specific anti-IL-6 antibodies and antibody fragments, e.g., Clazakizumab, wherein the antibody is administered intravenously or subcutaneously.

It is another more specific object of the invention to treat or prevent ABMR in patients in need thereof, particularly those receiving solid organ transplants by the use of specific anti-IL-6 antibodies and antibody fragments, e.g., Clazakizumab, wherein the anti-IL-6 antibody is administered at a dose ranging from about 0.01 mg-5000 mg, more typically from 0.1-1000 mg, and even more typically from 1-500 mg, preferably by intravenous or subcutaneous administration.

It is another more specific object of the invention to treat or prevent ABMR in patients in need thereof, particularly those receiving solid organ transplants by the use of specific anti-IL-6 antibodies and antibody fragments, e.g., Clazakizumab, wherein the antibody is administered intravenously at a dose ranging from about 5 mg-50 mg or subcutaneously at a dose ranging from about 10 mg-50 mg.

It is another more specific object of the invention to treat or prevent ABMR in patients in need thereof, particularly those receiving solid organ transplants by the use of specific anti-IL-6 antibodies and antibody fragments, e.g., Clazakizumab, wherein the antibody is administered about every 4 weeks, 8 weeks, 12 weeks, 16 weeks, 20 weeks, or 24 weeks.

It is another more specific object of the invention to treat or prevent ABMR in patients in need thereof, particularly those receiving solid organ transplants by the use of specific anti-IL-6 antibodies and antibody fragments, e.g., Clazakizumab, wherein the antibody is administered within about a month of detecting signs of ABMR.

It is another more specific object of the invention to treat or prevent ABMR in patients in need thereof, particularly those receiving solid organ transplants by the use of specific anti-IL-6 antibodies and antibody fragments, e.g., Clazakizumab, wherein the antibody is administered for several months prior to and months or even years after transplant in order to prevent or reduce antibody mediated damage to the transplanted organ.

It is another object of the invention to provide a method of preventing, stabilizing or reducing pre- or post-transplant sensitization in a subject who has or is to receive a solid organ transplant, comprising administering a prophylactically or therapeutically effective amount of an anti-human interleukin-6 (IL-6) antibody or antibody fragment, e.g., Clazakizumab, wherein the antibody or antibody fragment comprises: a variable light chain polypeptide comprising the CDRs of SEQ ID NOs:4, 5 and 6 and, and a variable heavy chain polypeptide comprising the CDRs of SEQ ID NOs:7, 8 or 120, and 9, e.g., wherein the antibody comprises a VH and VL polypeptide respectively at least 90, 95, 96, 97, 98 or 99% identical to the polypeptides of SEQ ID NO:657 and 709, and preferably is Clazakizumab.

It is another object of the invention to provide a method of preventing, stabilizing or reducing pre- or post-transplant sensitization in a subject who has or is to receive a solid organ transplant, comprising administering a prophylactically or therapeutically effective amount of an anti-human interleukin-6 (IL-6) antibody or antibody fragment, e.g., Clazakizumab, wherein the antibody or antibody fragment comprises: a variable light chain polypeptide comprising the CDRs of SEQ ID NOs:4, 5 and 6 and, and a variable heavy chain polypeptide comprising the CDRs of SEQ ID NOs:7, 8 or 120, and 9, wherein the solid organ is selected from kidney, heart, liver, lungs, pancreas, skin, intestine, stomach, or a combination of any of the foregoing or the solid organ is a kidney.

It is another object of the invention to provide a method of preventing, stabilizing or reducing pre- or post-transplant sensitization in a subject who has or is to receive a solid organ transplant, comprising administering a prophylactically or therapeutically effective amount of an anti-human interleukin-6 (IL-6) antibody or antibody fragment, e.g., Clazakizumab, as above described wherein the patient is at risk of or is sensitized because of a history of blood transfusions, pregnancies or a previous transplant.

It is another object of the invention to provide a method of preventing, stabilizing or reducing pre- or post-transplant sensitization in a subject who has or is to receive a solid organ transplant, comprising administering a prophylactically or therapeutically effective amount of an anti-human interleukin-6 (IL-6) antibody or antibody fragment, e.g., Clazakizumab, as above described wherein the patient develops pre-formed donor specific antibodies (DSA) to the donor organ.

It is another object of the invention to provide a method of preventing, stabilizing or reducing pre- or post-transplant sensitization in a subject who has or is to receive a solid organ transplant, comprising administering a prophylactically or therapeutically effective amount of an anti-human interleukin-6 (IL-6) antibody or antibody fragment, e.g., Clazakizumab, as above described wherein the patient which further includes a pre-transplant desensitization procedure to remove or reduce these alloantibodies (DSA).

It is another object of the invention to provide a method of preventing, stabilizing or reducing pre- or post-transplant sensitization in a subject who has or is to receive a solid organ transplant, comprising administering a prophylactically or therapeutically effective amount of an anti-human interleukin-6 (IL-6) antibody or antibody fragment, e.g., Clazakizumab, as above described wherein the patient wherein said desensitization treatments include plasmapheresis or plasma exchange optionally in combination with any one of intravenous immunoglobulin, anti-B cell agents such rituximab (an anti-CD20 mAb), and plasma cell inhibitors such as bortezomib (a proteosome inhibitor).

It is another object of the invention to provide a method of preventing, stabilizing or reducing pre- or post-transplant sensitization in a subject who has or is to receive a solid organ transplant, comprising administering a prophylactically or therapeutically effective amount of an anti-human interleukin-6 (IL-6) antibody or antibody fragment, e.g., Clazakizumab, as above described, wherein the patient wherein the antibody is administered intravenously or subcutaneously.

It is another object of the invention to provide a method of preventing, stabilizing or reducing pre- or post-transplant sensitization in a subject who has or is to receive a solid organ transplant, comprising administering a prophylactically or therapeutically effective amount of an anti-human interleukin-6 (IL-6) antibody or antibody fragment, e.g., Clazakizumab, as above described by the use of specific anti-IL-6 antibodies and antibody fragments, e.g., wherein the antibody is administered at a dose ranging from about 0.01 mg-5000 mg, more typically from 0.1-1000 mg, and even more typically from 1-500 mg, preferably by intravenous or subcutaneous administration.

It is another object of the invention to provide a method of preventing, stabilizing or reducing pre- or post-transplant sensitization in a subject who has or is to receive a solid organ transplant, comprising administering a prophylactically or therapeutically effective amount of an anti-human interleukin-6 (IL-6) antibody or antibody fragment, e.g., Clazakizumab, as above described wherein the patient wherein the antibody is administered intravenously at doses of 5 mg-50 mg or subcutaneously at doses of 10 mg-50 mg.

It is another object of the invention to provide a method of preventing, stabilizing or reducing pre- or post-transplant sensitization in a subject who has or is to receive a solid organ transplant, comprising administering a prophylactically or therapeutically effective amount of an anti-human interleukin-6 (IL-6) antibody or antibody fragment, e.g., Clazakizumab, as above described wherein the patient, wherein the antibody is administered about every 4 or 8 weeks, starting several months (e.g. 6 months) prior to transplantation.

It is another object of the invention to provide a method of preventing, stabilizing or reducing pre- or post-transplant sensitization in a subject who has or is to receive a solid organ transplant, comprising administering a prophylactically or therapeutically effective amount of an anti-human interleukin-6 (IL-6) antibody or antibody fragment, e.g., Clazakizumab, as above described wherein the patient wherein the patient is periodically assessed by various antibody detection methods (e.g. cytotoxic cross-match, flow cytometric cross match, LUMINEX® antibody testing) pre-desensitization to detect levels of DSA during the desensitization treatment process.

It is another object of the invention to provide a method of preventing, stabilizing or reducing pre- or post-transplant sensitization in a subject who has or is to receive a solid organ transplant, comprising administering a prophylactically or therapeutically effective amount of an anti-human interleukin-6 (IL-6) antibody or antibody fragment, e.g., Clazakizumab, as above described wherein a positive response (e.g. conversion of positive to negative cytotoxic cross-match) is used to determine that the patient is eligible for and may proceed to transplantation.

It is another object of the invention to provide a method of preventing, stabilizing or reducing pre- or post-transplant sensitization in a subject who has or is to receive a solid organ transplant, comprising administering a prophylactically or therapeutically effective amount of an anti-human interleukin-6 (IL-6) antibody or antibody fragment, e.g., Clazakizumab, as above described wherein the patient is treated with the antibody, preferably Clazakizumab pre- and/or post-transplant.

It is another object of the invention to provide a method of preventing, stabilizing or reducing pre- or post-transplant sensitization in a subject who has or is to receive a solid organ transplant, comprising administering a prophylactically or therapeutically effective amount of an anti-human interleukin-6 (IL-6) antibody or antibody fragment, e.g., Clazakizumab, as above described wherein said antibody administration is continued for several months or years post-transplant to prevent or treat early acute or late chronic rejections.

It is another object of the invention to provide a method of preventing, stabilizing or reducing pre- or post-transplant sensitization in a subject who has or is to receive a solid organ transplant, comprising administering a prophylactically or therapeutically effective amount of an anti-human interleukin-6 (IL-6) antibody or antibody fragment, e.g., Clazakizumab, as above described wherein the patient is monitored for clinical signs of rejection such as increases in serum creatinine and/or proteinuria, or decreases in eGFR in kidney transplants), or development of new DSA (de novo DSA).

It is another object of the invention to provide a method of preventing, stabilizing or reducing pre- or post-transplant sensitization in a subject who has or is to receive a solid organ transplant, comprising administering a prophylactically or therapeutically effective amount of an anti-human interleukin-6 (IL-6) antibody or antibody fragment, e.g., Clazakizumab, as above described wherein the patient is monitored for histological signs of organ rejection.

It is another object of the invention to provide a method of preventing, stabilizing or reducing pre- or post-transplant sensitization in a subject who has or is to receive a solid organ transplant, comprising administering a prophylactically or therapeutically effective amount of an anti-human interleukin-6 (IL-6) antibody or antibody fragment, e.g., Clazakizumab, as above described wherein ABMR organ damage is confirmed by biopsy evidence (e.g., microvascular inflammation, interstitial fibrosis, transplant glomerulopathy, CD4 deposition).

It is another object of the invention to use any of the afore-mentioned methods in combination with the standard of care immunosuppression regimens (e.g. thymoglobulin, basiliximab, mycophenolate mofetil, tacrolimus, and corticosteroids) that are normally administered to the patient pre- and post-transplant.

It is another object of the invention to use any of the afore-mentioned methods, wherein the anti-IL-6 antibody or antibody fragment contains an Fc region that has been modified to alter effector function, half-life, proteolysis, and/or glycosylation.

It is another object of the invention to use any of the afore-mentioned methods wherein the anti-IL-6 antibody is selected from a humanized, single chain, or chimeric antibody and the antibody fragment is selected from a Fab, Fab′, F(ab′)2, Fv, or scFv.

It is another object of the invention to use any of the afore-mentioned methods wherein the anti-IL-6 antibody dose is between about 0.001 and 100 mg/kg of body weight of recipient patient, more preferably from 0.01 to 20 mg/kg of body weight.

It is another object of the invention to use any of the afore-mentioned methods wherein the antibody or fragment inhibits the binding of IL-6 to gp130 and/or 11-6 binding to IL-6R1.

It is another object of the invention to use any of the afore-mentioned methods wherein the wherein the anti-IL-6 antibody or antibody fragment e.g., Clazakizumab, comprises a human constant region.

It is another object of the invention to use any of the afore-mentioned methods wherein the anti-II-6 antibody e.g., Clazakizumab, comprises a human constant region such as an IgG1, IgG2, IgG3 or IgG4 constant region or preferably comprises a human IgG1 constant region.

It is another object of the invention to provide a method of preventing, stabilizing or reducing antibody mediated rejection (ABMR) in a subject who is to receive, is receiving or has received a solid organ transplant, comprising administering to said subject a prophylactically or therapeutically effective amount of an anti-human interleukin-6 (IL-6) antibody or anti-human 11-6 antibody fragment, wherein the antibody or antibody fragment comprises: a variable light chain polypeptide comprising the CDRs of SEQ ID NOs:4, 5 and 6 and, and a variable heavy chain polypeptide comprising the CDRs of SEQ ID NOs:7, 8 or 120, and 9.

It is another object of the invention to provide a method of reversing, stabilizing and/or slowing the progression of active antibody mediated-rejection (AMBR) in a transplant recipient in need thereof comprising administering an effective amount of an anti-IL-6 antibody or antibody fragment, optionally wherein the antibody or antibody fragment comprises: a variable light chain polypeptide comprising the CDRs of SEQ ID NOs:4, 5 and 6 and, and a variable heavy chain polypeptide comprising the CDRs of SEQ ID NOs:7, 8 or 120, and 9.

It is another object of the invention to provide methods as above-identified, wherein the anti-human IL-6 antibody comprises the heavy chain polypeptide of SEQ ID NO: 704 or 745 and comprises the light chain polypeptide of SEQ ID NO: 702 or 746.

It is another object of the invention to provide methods as above-identified, wherein the anti-human IL-6 antibody, wherein the anti-human IL-6 antibody is administered for at least 1 year.

It is another object of the invention to provide methods as above-identified, wherein the anti-human IL-6 antibody wherein the anti-human IL-6 antibody is administered for at least 2 years.

It is another object of the invention to provide methods as above-identified, wherein the anti-human IL-6 antibody is administered for at least 3 years.

It is another object of the invention to provide methods as above-identified, wherein the anti-human IL-6 antibody is administered for at least 4 years.

It is another object of the invention to provide methods as above-identified, wherein the anti-human IL-6 antibody is administered for at least 5 years.

It is another object of the invention to provide methods as above-identified, wherein the anti-human IL-6 antibody is administered for more than 5 years.

It is another object of the invention to provide methods as above-identified, wherein the transplant recipient comprises active antibody mediated-rejection (AMBR) or chronic active antibody mediated-rejection (CABMR), optionally when treatment is started, optionally at least once within the time period spanning 1-6 months prior to treatment.

It is another object of the invention to provide methods as above-identified, wherein the transplant recipient has been diagnosed as having AMBR or CAMBR prior to anti-IL-6 antibody administration.

It is another object of the invention to provide methods as above-identified, wherein treatment with the anti-IL-6 antibody stabilizes or increases the estimated glomerular filtration rate (eGFR) during treatment, optionally throughout the entire treatment period.

It is another object of the invention to provide methods as above-identified, wherein treatment with the anti-IL-6 antibody stabilizes or increases the estimated glomerular filtration rate (eGFR) during treatment, optionally throughout the treatment period and further optionally wherein said stabilization or increase in eGFR is maintained for at least 3, 6, 9 or 12 months after treatment has ended.

It is another object of the invention to provide methods as above-identified, wherein the treated patient does not comprise neutropenia (less than 1,000 mm3) or thrombocytopenia (less than 50,000 mm3) when treatment is commenced and/or during the treatment regimen.

It is another object of the invention to provide methods as above-identified, wherein the treated patient does not receive intravenous immunoglobulin within the time period spanning 0-6 months prior to treatment.

It is another object of the invention to provide methods as above-identified, wherein the treated patient comprises human leukocyte antigen (HLA) DSAs prior to treatment, optionally wherein this has been confirmed by an assay which detects for human leukocyte antigen (HLA) DSAs within the time period spanning 0-6 months prior to treatment.

It is another object of the invention to provide methods as above-identified, wherein the treatment elicits one or more of the following:
(i) reduces the number of or eliminates donor specific antibodies (DSAs),
(ii) reduces CCL2 levels;
(iii) reduces complement activation and/or reduces the amount of detected C5b. C9 and/or C5b/C9 complexes;
(iv) reduces the number of plasma cells secreting DSAs;
(v) prevents allograft loss;
(vi) prevents return to dialysis,
(vii) prevents allograft nephrectomy, and/or
(viii) prevents the need for re-transplantation,
(ix) maintains or increases estimated glomerular filtration rate (eGFR) such that it is at least ≥mL/min/1.73 m².

It is another object of the invention to provide methods as above-identified, wherein the transplant comprises a solid organ.

It is another object of the invention to provide methods as above-identified, wherein the solid organ comprises kidney, heart, lung, bladder, pancreas, liver, gall bladder, thyroid, skin or any combination of the foregoing.

It is another object of the invention to provide methods as above-identified, wherein the solid organ comprises or consists of a kidney.

It is another object of the invention to provide methods as above-identified, wherein the transplant is from a living or deceased donor.

It is another object of the invention to provide methods as above-identified, wherein the treatment is effective an efficacy during or after treatment is evaluated at least in part by detecting eGFR values, optionally using the Modification of Diet in Renal Disease 4 (MDRD4) equation.

It is another object of the invention to provide methods as above-identified, wherein efficacy is evaluated at least in part by evaluating the histology of kidney biopsies according to the Banff 2015 lesion grading scores.

It is another object of the invention to provide methods as above-identified, wherein efficacy is evaluated at least in part by detecting DSA titers and/or mean fluorescence intensity (MFI) scores.

It is another object of the invention to provide methods as above-identified, wherein efficacy is evaluated at least in part by evaluating the incidence of acute rejection episodes (TCMR and ABMR).

It is another object of the invention to provide methods as above-identified, wherein efficacy is evaluated at least in part by evaluating the effects of treatment on albuminuria.

It is another object of the invention to provide methods as above-identified, wherein efficacy is evaluated at least in part by evaluating survival rates compared to controls and/or conventional AMBR or CAMBR treatments.

It is another object of the invention to provide methods as above-identified, wherein the anti-IL-6 antibody comprises human IgG1 constant regions e.g., wherein the human IgG1 constant regions comprise the constant light polypeptide of SEQ ID NO: 586 and the constant heavy polypeptide of SEQ ID NO: 588.

It is another object of the invention to provide methods as above-identified, wherein the anti-IL-6 antibody comprises the variable heavy chain polypeptide of SEQ ID NO: 657 and the variable light chain polypeptide of SEQ ID NO: 709.

It is another object of the invention to provide methods as above-identified, wherein the anti-IL-6 antibody comprises the heavy chain polypeptide of SEQ ID NO: 704 or 745 and the light chain polypeptide of SEQ ID NO: 702 or 746.

It is another object of the invention to provide methods as above-identified, wherein the anti-IL-6 antibody is dosed intravenously or subcutaneously every 4 weeks or monthly.

It is another object of the invention to provide methods as above-identified, wherein a 25 mg or 12.5 mg dose of the anti-IL-6 antibody is administered intravenously or subcutaneously every 4 weeks or monthly.

It is another object of the invention to provide methods as above-identified, wherein a 25 mg or 12.5 mg dose of Claza is administered subcutaneously every 4 weeks or monthly.

It is another object of the invention to provide methods as above-identified, wherein the treatment is effected for at least 1 year, 2 years, 3 years, 4 years or 5 years without an adverse event selected from return to dialysis, allograft nephrectomy, re-transplantation or eGFR 15 mL/min/1.73m2.

It is another object of the invention to provide methods as above-identified, wherein the transplant recipient optionally is further treated with any of the following:
(i) azathioprine (e.g., 1.0-2.0 mg/kg/day),
(ii) calcineurin inhibitors (CNIs),
(iii) mycophenolate mofetil (MMF) (e.g., 1.0-2.0 g/day)/mycophenolic acid (MPA) (e.g., 720-1440 mg/day),
(iv) mTOR inhibitors (e.g., tacrolimus, (e.g., target trough levels 5-8 ng/ml) everolimus, sirolimus),
(v) low dose corticosteroids (e.g., prednisone/prednisolone 10 mg/day),
(vi) antihypertensive agents (e.g., angiotensin converting enzyme inhibitors (ACEIs),
(vii) angiotensin II receptor blockers (ARBs),
(viii) cyclosporine, (e.g., target trough levels 50-150 ng/ml)
(ix) antidiabetogenic agents;
(x) or a combination of any of the foregoing.

It is another object of the invention to provide methods as above-identified, wherein the transplant recipient optionally is further treated with Pneumocystis jiroveci pneumonia (PJP) prophylaxis, e.g., trimethoprim (e.g., 80 mg daily pill), and/or sulfamethoxazole (e.g., 160 mg 3 times weekly pill), inhaled pentamidine or oral dapsone (optionally commenced within at least 1 week of treatment).

The method of any of the foregoing claims wherein if the transplant recipient experiences acute TCMR it is treated, e.g., with a pulse steroid such as oral prednisone, e.g., 200 mg/day).

It is another object of the invention to provide methods as above-identified, wherein during anti-IL-6 antibody treatment and optionally within the period spanning the 0, 1, 2, 3, 4, 5 or 6 months prior to starting treatment the transplant recipient is not treated with any of the following:
(i) rituximab,
(ii) eculizumab,
(iii) proteasome inhibitors,
(iv) intravenous immunoglobulin (IVIG), (except for treatment of hypogammaglobulinemia,
(v) plasma exchange (PLEX), belatacept,
(vi) anti-IL-6R antibody and/or
(vii) any combination of the foregoing.

It is another object of the invention to provide methods as above-identified, wherein the transplant recipient comprises any or all of the following:
(i) is 18-75 years old,
(ii) treatment started 6 months from time of transplant,
(iii) diagnosis of CABMR according to BANFF 2015 diagnostic criteria which include the following: Biopsy proven CABMR (i.e., chronic glomerulopathy (cg)>0) with/without C4d staining (repeat biopsy to be performed if previous biopsy is not within 6 months of screening),
(iv) if subject has received treatment for ABMR (including CABMR) or TCMR a repeat biopsy (to show continuing CABMR) are performed wherein subjects without evidence of chronic tissue injury on light microscopy but who have glomerular basement membrane double contours on electron microscopy (cgla) are eligible;
(v) presence of human leukocyte antigen (HLA) DSA (using single-antigen bead-based assays) post-transplant.

It is another object of the invention to provide methods as above-identified, wherein the transplant recipient does not comprise one or more of the following:
(i) has not had treatment for ABMR or CABMR or TCMR within the time period spanning 0-3 months or 0-6 months of IL-6 antibody treatment or screening;
(ii) is not receiving any T cell depleting agents, no treatment for ABMR (including CABMR) or TCMR within 3 months of screening or treatment;
(iii) has not received T cell depleting agents (e.g., alemtuzumab, anti-thymocyte globulin) within 3 months of screening or IL-6 antibody treatment;
(iv) no biopsy showing pure TCMR or advanced interstitial fibrosis (ci3),
(v) no advanced tubular atrophy (ct3);
(vi) no vascular fibrous intimal thickening (cv3) or other significant causes of renal dysfunction (e.g., polyoma BK virus (BKV) nephropathy, glomerulonephritis);
(vii) no impaired renal function due to disorders in the transplanted allograft (e.g., renal artery stenosis, hydronephrosis);
(viii) no eGFR <25 mL/min/1.73 m² or >65 mL/min/1.73 m² (MDRD4), (viii) no nephrotic range proteinuria defined as spot urine protein creatinine ratio (UPCR) 3,000 mg/g (300 mg/mmol) or spot urine albumin creatinine ratio (UACR) 2,200 mg/g (220 mg/mmol);
(ix) is not pregnant or breastfeeding;
(x) no history of anaphylaxis;
(xi) no abnormal liver function tests (LFTs) (alanine aminotransferase (ALT)/aspartate aminotransferase (AST)/bilirubin >1.5×upper limit of normal) or other significant liver disease;
(xii) no history of active tuberculosis (TB);
(xiii) no history of latent TB without history of active TB (e.g., positive QuantiFERON-TB® (QFT®) test) unless subject has completed a full course of prophylactic treatment,
(xiv) no history of human immunodeficiency virus (HIV) infection or positive for HIV;
(xv) is not seropositive for hepatitis B surface antigen (HBsAg);
(xvi) is not Hepatitis C virus (HCV) RNA positive;
(xvii) no known Epstein-Barr virus (EBV) mismatch: donor seropositive, recipient seronegative;
(xviii) no history of gastrointestinal perforation, diverticular disease or diverticulitis, or inflammatory bowel disease;
(xix) no neutropenia (<1,000/mm³) or thrombocytopenia (<50,000/mm³);
(xx) no active infections requiring systemic antimicrobial agents and unresolved prior to screening;
(xxi) no history of or current invasive fungal infection or other opportunistic infection, including (but not limited to) the following: a nontuberculous mycobacterial infection, aspergillosis, pneumocystosis, and toxoplasmosis;
(xxii) no active viral infections such as BKV, cytomegalovirus (CMV), or EBV based on polymerase chain reaction (PCR) testing;
(xxii) no current or recent (in the period spanning 0-3 or 0-6 months prior to treatment,
(xxiii) no administration of a live vaccine within 6 weeks of screening, including but not limited to the following: Adenovirus, measles, mumps, and rubella, oral polio, oral typhoid, rotavirus, varicella zoster, yellow fever, no history of alcohol or illicit substance (including marijuana) abuse;
(xxiv) no present or previous (within 3 years) malignancy except for basal cell carcinoma, fully excised squamous cell carcinoma of the skin, or non-recurrent (within 5 years) cervical carcinoma in-situ;
(xxv) no presence of a condition or abnormality (i.e., clinically significant endocrine, autoimmune, metabolic, neurological, psychiatric/psychological, renal, gastrointestinal, hepatic, and hematological or any other system abnormalities that are uncontrolled with standard treatment) that could compromise safety or life expectancy;
(xxvi) no history of intolerance to trimethoprim or and/or sulfamethoxazole, no previous treatment with anti-IL-6 antibody and/or
(xxvii) any combination of the foregoing.

It is another object of the invention to provide methods of preventing, stabilizing or reducing complement activity in a subject in need thereof comprising administering to said subject a prophylactically or therapeutically effective amount of an anti-human interleukin-6 (IL-6) antibody or antibody fragment, e.g., one wherein the antibody or antibody fragment comprises: a variable light chain polypeptide comprising the CDRs of SEQ ID NOs:4, 5 and 6 and, and a variable heavy chain polypeptide comprising the CDRs of SEQ ID NOs:7, 8 or 120, and 9.

It is another object of the invention to provide methods as above-identified, wherein complement activity is measured in the subject before, during or after treatment.

It is another object of the invention to provide methods as above-identified, wherein the antibody comprises a VH and VL polypeptide respectively at least 90, 95, 96, 97, 98 or 99% identical to the polypeptides of SEQ ID NO:657 and 709.

It is another object of the invention to provide methods as above-identified, wherein the antibody comprises a heavy chain and light polypeptide respectively at least 90, 95, 96, 97, 98 or 99% identical to the polypeptides of SEQ ID NO:704 or 745 and 702 or 746.

It is another object of the invention to provide methods as above-identified, wherein the antibody is clazakizumab.

It is another object of the invention to provide methods as above-identified, wherein the solid organ is selected from kidney, heart, liver, lungs, pancreas, gall bladder skin, intestine, stomach, or a combination of any of the foregoing.

It is another object of the invention to provide methods as above-identified, wherein the solid organ comprises or consists of a kidney.

It is another object of the invention to provide methods as above-identified, wherein the patient is evaluated and has been diagnosed as having ABMR or CAMBR prior to treatment, e.g., wherein the evaluation comprises one or more of: detecting pre-formed and de novo HLA DSA (especially those detecting complement binding DSA such as Clq), detecting non-HLA antibodies associated with ABMR, and/or identifying at least one histological feature characteristic of antibody mediated organ damage and/or the histological feature characteristic of antibody mediated organ damage is detected by obtaining a biopsy from the transplanted organ and/or the histological feature characteristic of antibody mediated organ damage includes any of microvascular inflammation, complement deposition (C4d), and capillaritis.

It is another object of the invention to provide methods as above-identified, wherein the patient has a transplanted organ which consists of a kidney and the histological feature characteristic of antibody mediated organ damage includes any of microvascular inflammation, complement deposition (C4d) in the peritubular capillaries, peritubular capillaritis, glomerulitis and transplant glomerulopathy (double glomerular basement membrane contour).

It is another object of the invention to provide methods as above-identified, wherein the treatment further includes the administration of at least one other immunosuppressant, e.g., wherein the at least one other immunosuppressant is a standard of care pre- or post-transplant immunosuppressive medication.

It is another object of the invention to provide methods as above-identified, wherein the treatment further includes the administration of at least one other immunosuppressant, e.g., the at least one other immunosuppressant comprises any of thymoglobulin, basiliximab, mycophenolate mofetil, tacrolimus, an anti-CD20 mAb such as rituximab, and corticosteroids.

It is another object of the invention to provide methods as above-identified, wherein the anti-IL-6 antibody is administered intravenously or subcutaneously.

It is another object of the invention to provide methods as above-identified, wherein the anti-IL-6 antibody is administered at doses ranging from 0.01-5000 mg.

It is another object of the invention to provide methods as above-identified, wherein the anti-IL-6 antibody is administered at doses ranging from 0.1-1000 mg.

It is another object of the invention to provide methods as above-identified, wherein the anti-IL-6 antibody is administered at doses ranging from 1-500 mg.

It is another object of the invention to provide methods as above-identified, wherein the anti-IL-6 antibody is administered intravenously at doses ranging from about of 5 mg-50 mg or subcutaneously at doses ranging from about 10 mg-50 mg.

It is another object of the invention to provide methods as above-identified, wherein the anti-IL-6 antibody is administered at a dose of about 25 mg which is dosed about every 2 weeks, 4 weeks, 6 weeks, 8 weeks, 12 weeks, 16 weeks, 20 weeks, 24 weeks, monthly, bimonthly, every 2 months, every 3 months, every 4 months, every 5 months, every 6 months, every year or less frequently.

It is another object of the invention to provide methods as above-identified, wherein the anti-IL-6 antibody is administered about every 4 weeks, 8 weeks, 12 weeks, 16 weeks, 20 weeks, or 24 weeks.

It is another object of the invention to provide methods as above-identified, wherein the anti-IL-6 antibody is administered subcutaneously at a dosage of 25 mg or 12.5 mg every 4 weeks or monthly.

It is another object of the invention to provide methods as above-identified, wherein the anti-IL-6 antibody is administered within about 1, 2 or 3 months of detecting signs of ABMR or CAMBR.

It is another object of the invention to provide methods as above-identified, wherein the anti-IL-6 antibody is administered for several months prior to and months or years after transplant in order to prevent, stabilize or reduce antibody mediated damage to the transplanted organ.

It is another object of the invention to provide methods which prevent, stabilize or reduce pre- or post-transplant sensitization in a subject who has or is to receive a solid organ transplant, comprising administering a prophylactically or therapeutically effective amount of an anti-human interleukin-6 (IL-6) antibody or antibody fragment, wherein the antibody or antibody fragment comprises: a variable light chain polypeptide comprising the CDRs of SEQ ID NOs:4, 5 and 6 and, and a variable heavy chain polypeptide comprising the CDRs of SEQ ID NOs:7, 8 or 120, and 9.

It is another object of the invention to provide methods as above-identified, wherein the anti-IL-6 antibody comprises a VH and VL polypeptide respectively at least 90, 95, 96, 97, 98 or 99% identical to the polypeptides of SEQ ID NO:657 and 709.

It is another object of the invention to provide methods as above-identified, wherein the antibody comprises a VH and VL polypeptide identical to the polypeptides of SEQ ID NO:657 and 709.

It is another object of the invention to provide methods as above-identified, wherein the antibody comprises a light and heavy chain polypeptide respectively identical to the polypeptides of SEQ ID NO: 702 or 746 and 704 or 745.

It is another object of the invention to provide methods as above-identified, wherein the patient has been transplanted with a solid organ is selected from kidney, heart, liver, lungs, pancreas, skin, intestine, stomach, or a combination of any of the foregoing.

It is another object of the invention to provide methods as above-identified, wherein the solid organ comprises or consists of a kidney.

It is another object of the invention to provide methods as above-identified, wherein the patient is at risk of or is sensitized because of a history of blood transfusions, pregnancies or a previous transplant.

It is another object of the invention to provide methods as above-identified, wherein the patient comprises pre-formed donor specific antibodies (DSA) to the donor organ prior to and/or during anti-IL-6 antibody treatment.

It is another object of the invention to provide methods as above-identified, which further includes a pre-transplant desensitization procedure to remove or reduce donor specific alloantibodies (DSAs), e.g., wherein said desensitization treatments include plasmapheresis or plasma exchange optionally in combination with any one of intravenous immunoglobulin, anti-B cell agents such rituximab (an anti-CD20 mAb), and plasma cell inhibitors such as bortezomib (a proteosome inhibitor).

It is another object of the invention to provide methods as above-identified, wherein, wherein the anti-IL-6 antibody is administered intravenously or subcutaneously.

It is another object of the invention to provide methods as above-identified, wherein the anti-IL-6 antibody is administered at doses ranging from 0.01-5000 mg.

It is another object of the invention to provide methods as above-identified, wherein the anti-IL-6 antibody is administered at doses ranging from 0.1-1000 mg.

It is another object of the invention to provide methods as above-identified, wherein the anti-IL-6 antibody is administered at doses ranging from 1-500 mg.

It is another object of the invention to provide methods as above-identified, wherein the anti-IL-6 antibody is administered at a dose of about 25 mg which is dosed about every 2 weeks, 4 weeks, 6 weeks, 8 weeks, 12 weeks, 16 weeks, 20 weeks, 24 weeks, monthly, bimonthly, every 2 months, every 3 months, every 4 months, every 5 months, every 6 months, every year or less frequently.

It is another object of the invention to provide methods as above-identified, wherein the anti-IL-6 antibody is administered about every 4 or 8 weeks, starting several months (e.g. within the period spanning 0-6 months prior to transplantation.

It is another object of the invention to provide methods as above-identified, wherein the patient is periodically assessed during treatment by one or more antibody detection methods (e.g. cytotoxic cross-match, flow cytometric cross match, LUMINEX® antibody testing) pre-desensitization to detect levels of DSA during the desensitization treatment process, e.g., wherein a positive response (e.g. conversion of positive to negative cytotoxic cross-match) is used to determine that the patient is eligible or still eligible for IL-6 antibody treatment and/or transplantation.

It is another object of the invention to provide methods as above-identified, wherein the anti-IL-6 antibody the patient is treated with the anti-IL-6 antibody, e.g., Clazakizumab, post-transplant.

It is another object of the invention to provide methods as above-identified, wherein the anti-IL-6 antibody said anti-IL-6 antibody administration is continued for several months or years post-transplant to prevent or treat early acute or late chronic rejections.

It is another object of the invention to provide methods as above-identified, wherein the anti-IL-6 antibody the patient is monitored for clinical signs of rejection such as increases in serum creatinine and/or proteinuria, or decreases in eGFR in kidney transplants), or the development of new DSA (de novo DSA).

It is another object of the invention to provide methods as above-identified, wherein the patient is monitored for histological signs of organ rejection.

It is another object of the invention to provide methods as above-identified, wherein prevention, stabilization or reduction of ABMR organ damage prior, during and/or after is confirmed by biopsy evidence (e.g., microvascular inflammation, interstitial fibrosis, transplant glomerulopathy, CD4 deposition).

It is another object of the invention to provide methods as above-identified, wherein Clazakizumab is used in combination with the standard of care immunosuppression regimens (e.g. thymoglobulin, basiliximab, mycophenolate mofetil, tacrolimus, corticosteroids) that are normally administered to the patient pre- and post-transplant.

It is another object of the invention to provide methods as above-identified, wherein the anti-IL-6 antibody or antibody fragment contains an Fc region that has been modified to alter effector function, half-life, proteolysis, and/or glycosylation.

The method of any of the previous claims wherein the anti-II-6 antibody is selected from a humanized, single chain, or chimeric antibody and the antibody fragment is selected from a Fab, Fab′, F(ab′)2, Fv, or scFv.

It is another object of the invention to provide methods as above-identified, wherein the antibody dose is between about 0.001 and 100 mg/kg of body weight of recipient patient.

It is another object of the invention to provide methods as above-identified, wherein the anti-IL-6 antibody dose is between about 0.1 and 20 mg/kg of body weight of recipient patient or comprises about 25 mg.

It is another object of the invention to provide methods as above-identified, wherein the anti-IL-6 antibody or fragment inhibits the binding of IL-6 to gp130 and/or to IL-6R1.

It is another object of the invention to provide methods as above-identified, wherein the anti-IL-6 antibody or antibody fragment comprises a human constant region, e.g., wherein said human constant region comprises an IgG1, IgG2, IgG3 or IgG4 constant region or said human constant region comprises an IgG1 constant region.

It is another object of the invention to provide methods as above-identified, wherein the anti-IL-6 antibody is clazakizumab.

It is another object of the invention to provide methods as above-identified, wherein the treated subject has late or advanced AMBR (Acute/active or chronic/active phenotype according to the Banff 2015 classification).

It is another object of the invention to provide methods as above-identified, wherein the administered anti-IL-6 antibody is clazakizumab and the treated subject has late or advanced AMBR (Acute/active or chronic/active phenotype according to the Banff 2015 classification).

It is another object of the invention to provide methods as above-identified, wherein the treated subject has a complement-related condition selected from age-related and degenerative diseases such as Age-related macular degeneration (AMD) (wet and dry), Alzheimer's Disease, glomerular diseases e.g., atypical hemolytic uremic syndrome (aHUS), hemolytic uremic syndrome caused by Shiga toxin-producing E. coli (STEC-HUS), thrombotic thrombocytopenic purpura (TTP), systemic lupus erythematosus (SLE), antiphospholipid antibody syndrome (APS), anti-neutrophil cytoplasmic antibody (ANCA)-induced vasculitis, inflammatory small-vessel disorders caused by autoantibodies against neutrophil constituents; antibody-dependent (i.e., in women with APS), pregnancy loss involving C5a-mediated impairment of placental angiogenesis; complement mediated hemolytic disorders such as paroxysmal nocturnal hemoglobinuria (PNH), aHUS and cold-agglutinin disease (CAD), Ischemia-reperfusion injury; stroke, myocardial infarction, e.g., caused by trauma, sepsis, shock and cardiopulmonary bypass (CPB) surgery, CPB cardiopulmonary bypass surgery, allergic asthma, periodontitis. bone-related disorders and bone injury associated with aberrant complement activation (e.g., via anaphylatoxin effects on osteoclast formation), acute-phase conditions, in which the host is confronted with a dramatic increase of damage- and/or pathogen-associated molecular patterns.

### BRIEF DESCRIPTION OF THE FIGURES

FIG. 1 contains experimental results showing the effect of clazakizumab on the transcription of HLA-DR, CD54, IL-6 and PDL-1.

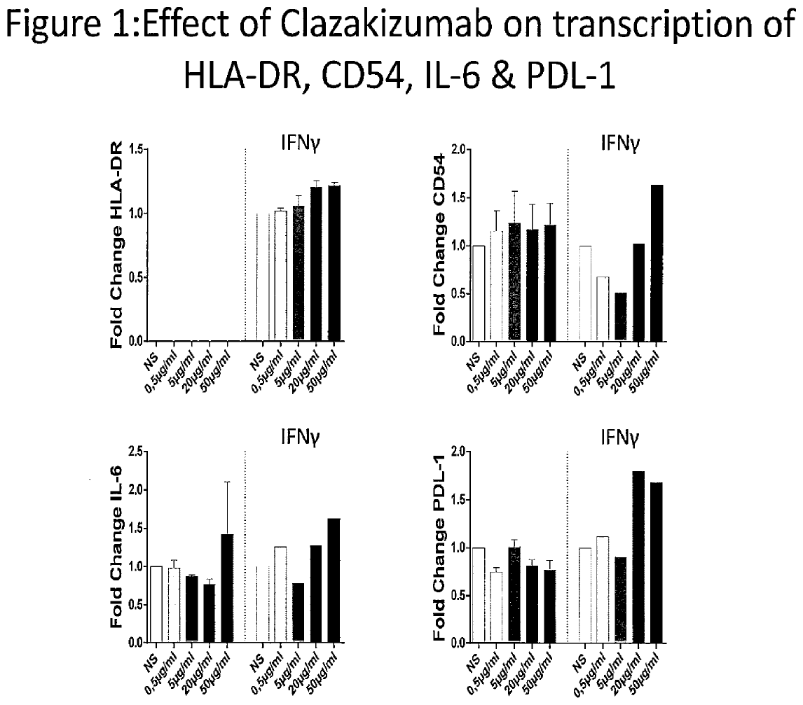

FIG. 2 schematically shows pre-treatment of epithelial cells (ECs) with Clazakizumab prior to co-culture with allogeneic PBMC's.

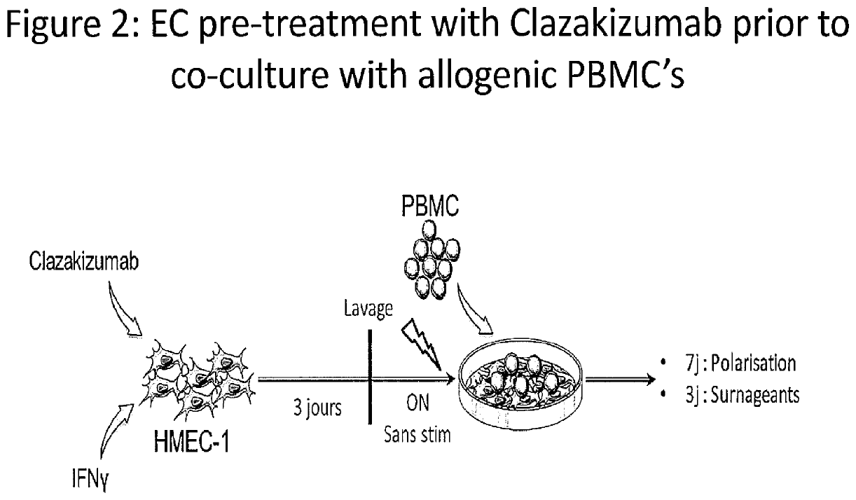

FIG. 3 contains experimental results showing IL-6 secretion in co-cultures with clazakizumab.

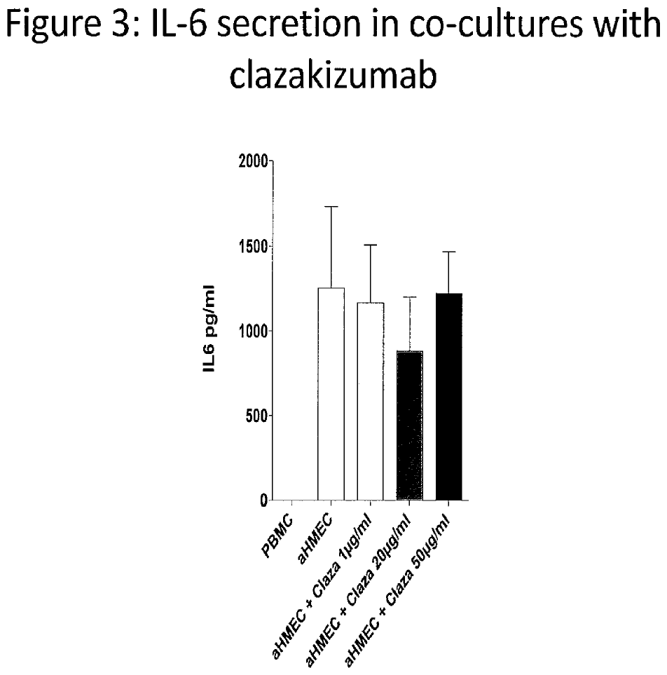

FIG. 4 contains experimental results showing the effect of direct addition of Clazakizumab into EC-allo PBMC co-cultures.

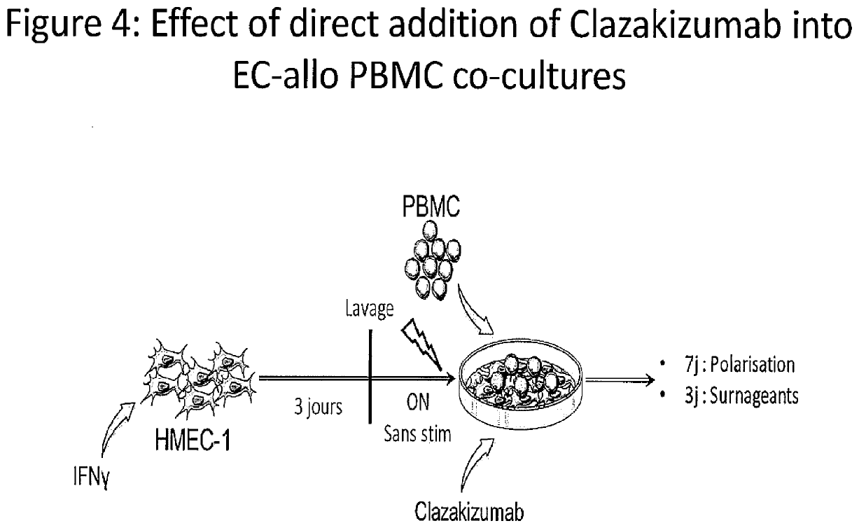

FIG. 5 contains experimental results showing the effect of Clazakizumab on levels of IL-6, MCP-1 & RANTES in EC-PBMC co-cultures.

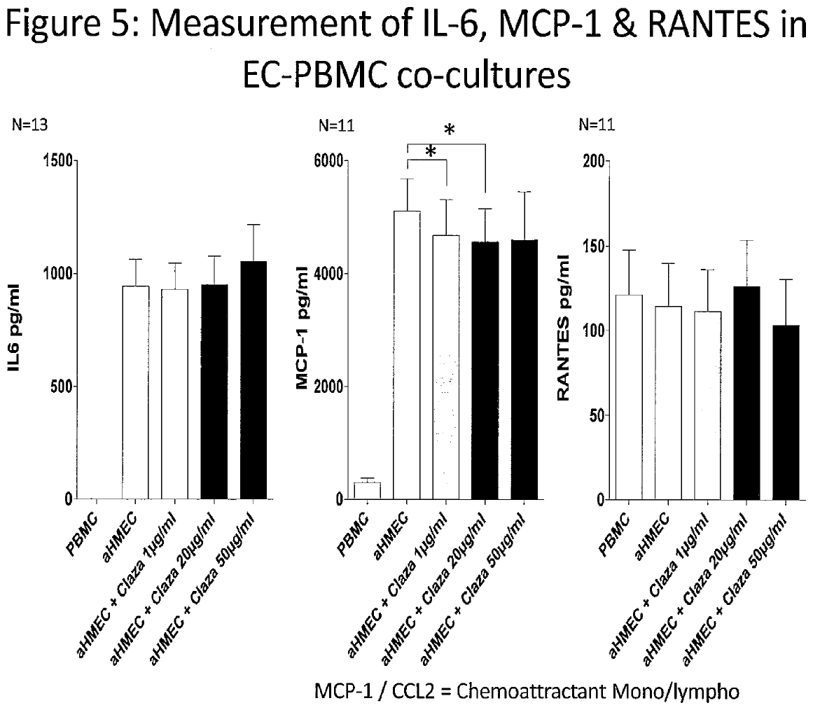

FIG. 6 contains experimental results showing the effect of Clazakizumab on the expansion of Tₘₑₘ and T_{reg} cells in EC co-cultures with allo-PBMC's.

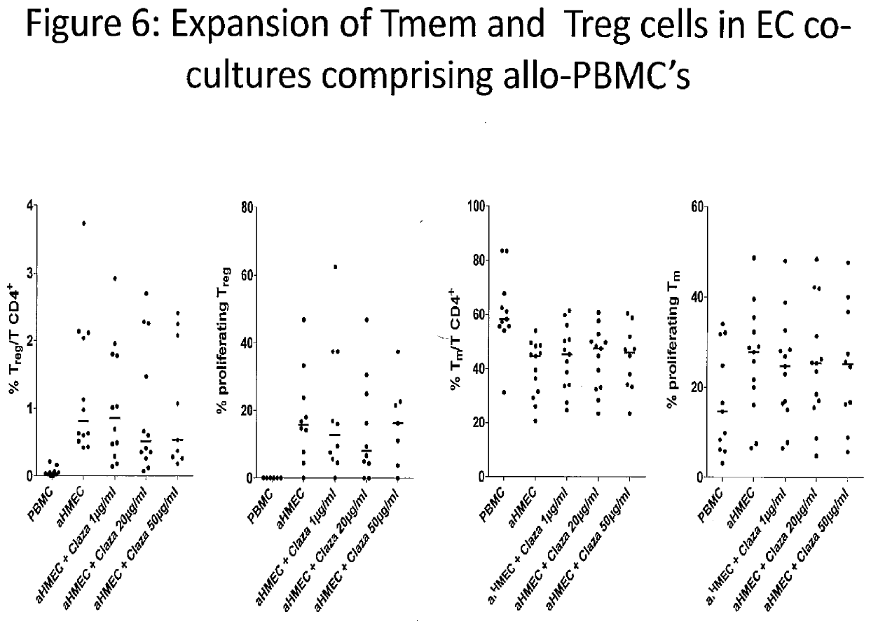

FIG. 7 contains experimental results showing the effect of Clazakizumab on the expansion of T17 and Th1 cells in EC-PBMC co-cultures.

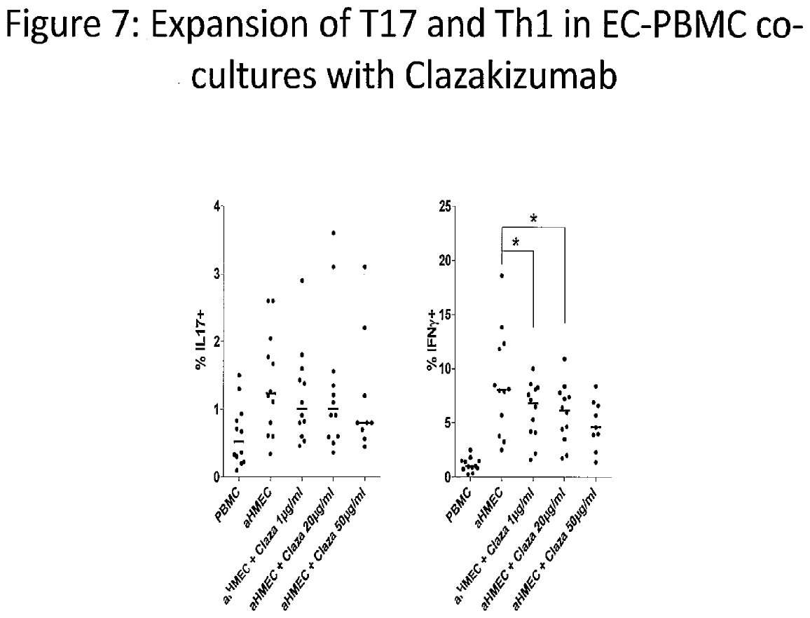

FIG. 8 contains experiments showing that IL-6R secretion is unchanged after EC stimulation.

FIG. 9 schematically depicts experiments showing the effect of Claza on EC proliferation and EC phenotype.

FIG. 10 shows experiments demonstrating that Claza does not alter EC proliferation.

FIG. 11 shows experiments demonstrating the effect of Claza on allogenicity mediators.

FIG. 12 schematically depicts experiments showing the effect of Claza on EC phenotype.

FIG. 13 schematically depicts experiments showing the effect of Claza on IL-6 ELISAs.

FIG. 14 depicts experiments showing the effect of Claza on IL-6 secretion by ECs.

FIG. 15 depicts experiments showing the effect of Claza on EC cocultures on EC allogenicity.

FIG. 16 depicts experiments showing that Claza reduces CCL-2 production in EC-PMBC cocultures.

FIG. 17 depicts experiments showing the effect of Claza on CD4+ T cell activation.

FIG. 18 depicts experiments showing the expansion of Th17 and Th1 cells in the presence of Claza.

FIG. 19 depicts experiments showing the reducing effect of Claza on Th1 responses of allogeneic CD4⁺ T cells.

FIG. 20 depicts experiments showing the expansion of Th1 cells in the presence of “low-dose” Claza.

FIG. 21 depicts experiments showing the effect of Claza on EC expression of complement regulatory proteins.

FIG. 22 depicts experiments showing the effect of Claza on complement activation.

FIG. 23 further depicts experiments showing the effect of Claza on complement activation.

### DETAILED DESCRIPTION

There is a need in the art for methods that improve the success of transplantation, including improving pre-transplant desensitization and post-transplant ABMR treatment and prevention. Interleukin-6 (IL-6) is a cytokine with powerful stimulatory effects on B cells and plasma cells and is responsible, in conjunction with other cytokines, for normal antibody production. IL-6 also has powerful stimulatory effects on T-cell mediated inflammatory processes. This invention relates to the use of specific anti-IL-6 antibodies or antibody fragments to treat recipients of organ transplant prior, concurrent or after organ transplant. In particular the invention pertains to methods of improving survival rates and/or quality of life in a transplant recipient in need thereof, in particular a sensitized pre-transplant patient, a patient who is at risk of becoming sensitized to a transplanted donor tissue or organ, e.g., because of a history of blood transfusions, pregnancies or a previous transplant; a pre-transplant patient or a post-transplant patient showing signs of ABMR or CAMBR, or any patient who may be at risk of developing ABMR or CAMBR.

In particular the invention provides novel therapeutic protocols for treating or preventing ABMR or CAMBR in patients in need thereof, particularly those receiving solid organ transplants by the use of specific anti-IL-6 antibodies and antibody fragments, e.g., Clazakizumab.

Also the invention provides novel therapeutic protocols for desensitization of for highly sensitized subjects awaiting and after an allograft transplant transplants by the use of specific anti-IL-6 antibodies and antibody fragments, e.g., Clazakizumab, and others having the sequences disclosed in U.S. Pat. No. 9,452,227, the contents of which including the sequence listing are incorporated by reference in their entirety.

More specifically the invention provides methods of preventing, stabilizing or reducing antibody mediated rejection (ABMR) or chronic antibody mediated rejection (CAMBR) in a subject who is or has received a solid organ transplant, comprising administering to said subject a prophylactically or therapeutically effective amount of an anti-human interleukin-6 (IL-6) antibody or antibody fragment, wherein the antibody or antibody fragment comprises: a variable light chain polypeptide comprising the CDRs of SEQ ID NOs:4, 5 and 6 and, and a variable heavy chain polypeptide comprising the CDRs of SEQ ID NOs:7, 8 or 120, and 9, e.g., wherein the antibody comprises a V_{H} and V_{L} polypeptide respectively at least 90, 95, 96, 97, 98 or 99% identical to the polypeptides of SEQ ID NO:657 and 709 or the antibody comprises a heavy chain and light chain polypeptide respectively at least 90, 95, 96, 97, 98 or 99% identical to the polypeptides of SEQ ID NO:704 and 702 and preferably wherein the antibody is clazakizumab. In exemplary embodiments the solid organ is selected from kidney, heart, liver, lungs, pancreas, skin, intestine, stomach, or a combination of any of the foregoing or preferably is a kidney.

Also the invention to provides methods of preventing, stabilizing or reducing pre- or post-transplant sensitization in a subject who has or is to receive a solid organ transplant, comprising administering a prophylactically or therapeutically effective amount of an anti-human interleukin-6 (IL-6) antibody or antibody fragment, wherein the antibody or antibody fragment comprises: a variable light chain polypeptide comprising the CDRs of SEQ ID NOs:4, 5 and 6 and, and a variable heavy chain polypeptide comprising the CDRs of SEQ ID NOs:7, 8 or 120, and 9, e.g., wherein the antibody comprises a VH and VL polypeptide respectively at least 90, 95, 96, 97, 98 or 99% identical to the polypeptides of SEQ ID NO:657 and 709, or the antibody comprises a heavy chain and light chain polypeptide respectively at least 90, 95, 96, 97, 98 or 99% identical to the polypeptides of SEQ ID NO:704 and 702 and preferably is Clazakizumab.

In some embodiments the anti-IL-6 antibodies contain specific CDRs, as described in U.S. Pat. No. 9,452,227, the disclosure of which is hereby incorporated by reference in its entirety. In preferred embodiments, an anti-IL-6 antibody is a humanized variant of Ab1 (see, e.g., column 46, line 8, to column 47, line, 12, of U.S. Pat. No. 9,452,227), e.g., Clazakizumab, or an antibody or antibody fragment that specifically binds to the same linear or conformational epitope(s) on an intact human IL-6 polypeptide fragment thereof as Clazakizumab or one comprising the same CDRs as this antibody.

Exemplary anti-IL-6 antibodies and antibody fragments comprise: a variable light chain polypeptide comprising the CDRs of SEQ ID NOs: 4, 5 and 6 and possessing at least 90% identity to the variable light chain polypeptide of SEQ ID NO: 709, and a variable heavy chain polypeptide comprising the CDRs of SEQ ID NOs: 7, 8 or 120, and 9 and possessing at least 90% identity to the variable heavy chain polypeptide of SEQ ID NO: 657, wherein the antibody or antibody fragment specifically binds to IL-6 and antagonizes one or more activities associated with IL-6 and specifically binds to the same epitope(s) on IL-6 as an anti-IL-6 antibody comprising the variable light chain polypeptide in of SEQ ID NO: 709 and the variable heavy chain polypeptide of SEQ ID NO: 657. (All of the sequences identified herein are described in U.S. Pat. No. 9,452,227).

In particularly preferred embodiments the anti-IL-6 antibody used in the inventive methods is Clazakizumab. Clazakizumab is a humanized monoclonal antibody that binds to and inhibits IL-6. This antibody potently inhibits or prevents ILI-6 from binding to IL-6R and to gp130. Clazakizumab has demonstrated efficacy in clinical and pre-clinical trials evaluating patients with rheumatoid arthritis, psoriatic arthritis, cancer and cachexia, and has potential applications for treating numerous diseases characterized by chronic inflammation.

This invention pertains to methods of treating patients pre-transplant, during transplant, post-transplant, or any combination thereof. The graft (transplant) can be any organ, tissue or cell(s) that will be/has been introduced into/onto the patient receiving the transplant (the recipient). In a preferred embodiment the graft organ, tissue or cell(s) are allogeneic such that the graft is an allograft. Also preferred are intestines (large and/or small) and solid organs (e.g. kidney, heart, liver, lungs, gall bladder, skin, stomach, and pancreas).

Treatment with the subject anti-IL-6 antibodies e.g., Clazakizumab, may improve the efficacy of desensitization procedures in patients pre-transplant. In particular, antibody treatment may improve the transplant rates in patients who have failed desensitization or shorten the time to transplant for these sensitized patients. Pre-transplant treatment with anti-IL-6 antibodies e.g., Clazakizumab, may also improve transplant success for patients who are not sensitized. Treatment with anti-IL-6 antibodies e.g., Clazakizumab, may also improve the efficacy of treatment in patients post-transplant, by preventing, reducing or ameliorating the damage caused by ABMR.

As used herein, “improved,” “improvement,” and other grammatical variants, includes any beneficial change resulting from a treatment. A beneficial change is any way in which a patient's condition is better than it would have been in the absence of the treatment. “Improved” includes prevention of an undesired condition, slowing the rate at which a condition worsens, delaying the development of an undesired condition, and increasing the rate at which a desired condition is reached. For example, improvement in a sensitized patient encompasses any decrease in sensitization as well as any increase in the amount or rate at which DSA are prevented, removed or reduced. For another example, improvement in a transplant recipient encompasses any prevention, decrease, delay or slowing in the rate or amount of antibody mediated damage or loss of function to the transplanted organ.

The anti-IL-6 antibodies e.g., Clazakizumab, can be administered to a patient pre-transplant with or without one or more additional standard desensitization treatments (e.g. plasmapheresis or plasma exchange intravenous immunoglobulin, anti-B cell agents such rituximab (an anti-CD20 mAb), and plasma cell inhibitors such as bortezomib (a proteosome inhibitor)). In some embodiments the anti-IL-6 antibodies e.g., Clazakizumab, are be administered intravenously (e.g., at doses ranging from 0.01-5000 mg, more typically from 0.1-1000 mg or 1-500 mg, and in exemplary embodiments from 5 mg-50 mg) or via subcutaneous injection (e.g., at doses ranging from 0.01-5000 mg, more typically from 0.1-1000 mg or 1-500 mg, and in exemplary embodiments at doses of 10 mg-50 mg) every 4 weeks, starting several months (e.g. 6 months) prior to transplant.

The treated patient can be assessed by various antibody detection methods (e.g. cytotoxic cross-match, flow cytometric cross match, LUMINEX® antibody testing) pre-desensitization and at regular intervals during the desensitization treatment process for their levels of DSA. A positive response (e.g. conversion of positive to negative cytotoxic cross-match) enables a patient to proceed to transplantation, with a reduced risk of antibody mediated rejection post-transplant.

Treatment with anti-IL-6 antibodies e.g., Clazakizumab, may be continued post-transplant for several months (e.g. one month to 36 months) to prevent or treat early acute or late chronic rejections. Early acute rejection episodes are usually T-cell mediated and late chronic rejection episodes are usually antibody mediated. Episodes of rejections are generally manifested by non-specific evidence (e.g., increases in serum creatinine and/or proteinuria, or decreases in eGFR in kidney transplants), and/or development of new DSA (de novo DSA) and can be confirmed by known diagnostic blood tests and biopsy (e.g. organ biopsy) evidence (e.g., microvascular inflammation, interstitial fibrosis, transplant glomerulopathy, CD4 deposition). Anti-IL-6 antibodies may be administered with or without one or more additional immunosuppression agents (e.g. thymoglobulin, basiliximab, mycophenolate mofetil, tacrolimus, anti-CD20 mAb such as rituximab and corticosteroids).

In addition, in post-transplant patients undergoing or at risk of antibody mediated rejection (ABMR) or chronic antibody mediated rejection (CABMR), plasma levels of IL-6 are significantly elevated and the levels decrease as the rejection subsides. Regardless of whether or not the patient was treated with anti-IL-6 antibodies pre-transplant, post-transplant administration of anti-IL-6 antibodies may therefore be useful to ameliorate or reduce the antibody mediated damage caused by HLA- and non-HLA DSA in ABMR patients.

Similar to above, in ABMR patients the anti-IL-6 antibodies e.g., Clazakizumab, can be administered with or without one or more additional immunosuppressive agents, and the antibodies can be administered intravenously ((e.g., at doses ranging from 0.01-5000 mg, more typically from 0.1-1000 mg or 1-500 mg, and in exemplary embodiments at doses of 5 mg-50 mg) or via subcutaneous injection ((e.g., at doses ranging from 0.01-5000 mg, more typically from 0.1-1000 mg or 1-500 mg, and in exemplary embodiments at doses of 10 mg-50 mg) every 4 weeks, starting before transplant, at the time of transplant or when evidence of rejection develops. Again the first signs of rejection commonly include non-specific evidence such as a rise in serum creatinine or the development of proteinuria, and confirmation of ABMR can be accomplished using known diagnostic blood tests and biopsies. Treatment with anti-IL-6 antibodies may be continued for several months (e.g. one month to several years) to prevent antibody mediated damage to the allograft and the resulting loss of function which can ultimately result in the total loss of the transplanted organ.

In some embodiments, the present invention provides a pharmaceutical composition suitable for preventing or treating ABMR or for treating or preventing sensitization of recipients of organ transplants. The pharmaceutical composition may include Clazakizumab and a pharmaceutically acceptable carrier or excipient and may optionally include one or more other immunosuppressants.

Pharmaceutical compositions for use in methods according to the invention can contain any pharmaceutically acceptable excipient. Examples of excipients include but are not limited to starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, antioxidants, plasticizers, gelling agents, thickeners, hardeners, setting agents, suspending agents, surfactants, humectants, carriers, stabilizers, and combinations thereof.

In various embodiments, the pharmaceutical compositions according to the invention may be formulated for delivery via any route of administration. This may include e.g., aerosol, nasal, oral, transmucosal, transdermal, parenteral or enteral.

“Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the parenteral route, the compositions may be in the form of solutions or suspensions for infusion or for injection. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Typically, the compositions are administered by injection. Methods for these administrations are known to one skilled in the art.

Pharmaceutical compositions according to the invention can contain any pharmaceutically acceptable carrier. For example, the carrier may be a liquid or solid filler, diluent, excipient, solvent, or encapsulating material, or a combination thereof.

In order to further describe the invention the following examples are provided.

### Example 1

Use of Clazakizumab as Part of Desensitization Protocol for Highly Sensitized Subjects Awaiting Transplant and after Allograft Transplants

A patient awaiting kidney transplant who has previously become sensitized or who is at risk of becoming sensitized present to the donor organ (e.g., because of a history of blood transfusions, pregnancies or a previous transplant), is therapeutically or prophylactically treated in order to reduce or eliminate or prevent sensitization to antigens (e.g. HLA antigens and non-HLA antigens) present in the donor organ. For example the patient is treated by one or more of plasmapheresis, plasma exchange optionally in combination with intravenous immunoglobulin and anti-B cell agents such rituximab or plasma cell inhibitors such as bortezomib (a proteosome inhibitor). These procedures are repeated as necessary and typically continued until organ transplant is effected and may be continued after organ transplant.

Additionally, in order to enhance the efficacy of the desensitization treatment regimen the patient is further therapeutically or prophylactically treated with an anti-IL-6 antibody, e.g., Clazakizumab. This anti-IL-6 antibody is administered intravenously at a dose ranging from 5 mg-50 mg or is administered subcutaneously at a dose ranging from 10 mg-50 mg. The antibody dosing is effected every 4 weeks or monthly, preferably commencing about a month or several months prior to transplantation, e.g., from about 1-6 months prior to transplant.

In addition pre-desensitization and at regular intervals throughout these desensitization procedures the patient is also assessed by one or more antibody detection methods (e.g. cytotoxic cross-match, flow cytometric cross match, LUMINEX® antibody testing) to assess the patient's levels of DSA.

If a positive response (e.g. conversion of positive to negative cytotoxic cross-match) the patient is then determined to be suitable for organ transplantation and the patient is then transplanted with the donor kidney by known procedures.

Concurrent or post-transplant, the patient is treated with Clazakizumab for several months (e.g. commencing at the time of transplant, or about one month after and is continued for months or years after transplant, e.g., 6, 12, 18, 24, 30, 36 months or even 5, 10, 20 years after transplant to prevent or treat early acute or late chronic rejections. The early acute rejection episodes are usually T-cell mediated and the late chronic rejection episodes are usually antibody mediated.

Rejection episodes if present in the transplant recipient may be manifested by one or more clinical signs (e.g., increases in serum creatinine and/or proteinuria, or decreases in eGFR in kidney transplants), development of new DSA (de novo DSA) which may be confirmed by biopsy evidence (e.g., microvascular inflammation, interstitial fibrosis, transplant glomerulopathy, CD4 deposition).

Additionally the patient may also be treated by the use of other standard of care immunosuppression regimens (e.g. thymoglobulin, basiliximab, mycophenolate mofetil, tacrolimus, and corticosteroids). These additional immunosuppression regimens are effected pre- and post-transplant, e.g., from about 1-6 months pre-transplant and continued for months or even years post-transplant. The patient is periodically assessed post-transplant for any clinical signs of a rejection response such as increases in serum creatinine and/or proteinuria, or decreases in eGFR in kidney transplants. If any such clinical responses are observed the patient may be more aggressively treated with immunosuppressants, e.g., the immunosuppressant dose may be increased or the patient treated more frequently with immunosuppressant and/or the patient may be treated with other immunosuppressants in order to stabilize or eliminate the rejection response.

### Example 2: Use of Clazakizumab in Highly-HLA Sensitized Patients Awaiting Renal Transplant

Patients who have had a previous allograft failure represent a major problem for transplant centers as they are highly-human leukocyte antigen (HLA) sensitized and unlikely to receive another transplant without significant desensitization. According to the invention transplant patients who qualify will generally receive up to 6 doses of clazakizumab 25 mg monthly pre-transplantation. If patients receive an HLAi transplant during treatment, the participants may continue to receive another 6 monthly doses of 25 mg of clazakizumab, followed by a 6 month protocol biopsy. Patients will receive another 6 doses over 6 months if improvements are seen after the 6th dose of clazakizumab. Patients who develop evidence of persistent allograft dysfunction may have non-protocol biopsies for cause. Patients who receive 12 doses of clazakizumab post-transplant generally will receive a 12M protocol biopsy.

Patients considered for treatment further may initially receive PLEX (5-7 sessions)+IVIG and then receive clazakizumab 25 mg SC one week post-IVIG. If no safety/tolerability/efficacy issues are observed after the initial dose, patients may receive 5 additional injections Q4W. If patients receive an HLAi transplant, clazakizumab are be continued for 6M post-transplant at 25 mg SC Q4W for 6 doses (starting at Day 5 post-transplant). A protocol biopsy may be performed at 6M post-transplant to assess the allograft for evidence of ABMR or CAMBR, including C4d staining and TG using Banff 2015 criteria. Patients will continue to receive another 6 doses over 6 months if improvements are seen after the 6th dose of clazakizumab. Patients who develop evidence of persistent allograft dysfunction may have non-protocol biopsies for cause. Patients who receive 12 doses of clazakizumab post-transplant may receive a 12M protocol biopsy. In the event a patient does not show improvement after receiving 6 doses of clazakizumab, generally no further treatment will be given.

The treated subjects generally will be followed to determine if the use of clazakizumab for desensitization in this high risk transplant population is safe and does not pose infectious risks. In addition, the effects of clazakizumab treatment on HLA antibodies will be evaluated. Renal biopsy assessments may be performed at 6M and again at 12M (e.g., for those who received 12 doses of therapy). The transplanted patients will then be assessed to determine the number who sustain a viable and functioning kidney allograft as well.

Generally patients are to receive clazakizumab monthly. Patients will generally receive up to 6 doses pre-transplantation. If patients are transplanted during IL-6 Ab treatment, they may then receive 6 doses of clazakizumab (monthly) and a 6 month protocol biopsy may be performed. Based on the biopsy results and clinical labs PI is determined to assess whether the patient should continue monthly doses for up to another 6 doses. Patients who receive 12 post-transplant doses of clazakizumab may then undergo a 12 month protocol biopsy.

### Example 3: Use of Clazakizumab in Treating Patients with Late AMBR

The safety, tolerability, pharmacokinetics, pharmacodynamics and efficacy (preliminary assessment) of humanized anti-IL-6 monoclonal antibody clazakizumab in kidney transplant recipients is assessed in patients with late antibody-mediated rejection (ABMR). The study is designed as a phase 2 trial and has two subsequent sub-parts, a randomized placebo-controlled trial (part A) of 12 weeks, where recipients are allocated to receive either anti-IL-6 antibody clazakizumab (n=10) or placebo (n=10), followed by an open-label prospective study, where all 20 study patients are receive clazakizumab for a period of 40 weeks. Study protocol biopsies are to be performed at the end of part A and part B.

Part A:

Patients positive for anti-HLA donor-specific antibodies (DSA) and with biopsy-proven late ABMR (Acute/active or chronic/active phenotype according to the Banff 2015 classification) are be identified and recruited at the kidney transplantation outpatient services of the two center sites. Participants are to be randomized to receive either clazakizumab or placebo subcutaneously (1:1 randomization stratified for ABMR type) for a period of 12 weeks (administration of clazakizumab/placebo at day 0, and after 4 and 8 weeks). After 12 weeks, patients generally will be subjected to a first follow-up biopsy. Primary goals of this part of the trial are to assess the safety, tolerability, pharmacokinetics and pharmacodynamics of a short course of treatment. Moreover, part A provides for a first preliminary assessment of the impact of clazakizumab on ABMR-associated inflammation detected in peripheral blood and in the rejecting organ allograft, on the pharmacokinetics of pantoprazole as a probe drug to investigate influence of IL-6 blockade on cytochrome P450 (CYP) dependent drug metabolism (potential effects on the half-life of CYP-metabolized drugs such as pantoprazole, and on the short-term course of DSA mean fluorescence intensity (MFI) and kidney allograft function (eGFR, urinary protein excretion).

Part B:

After completion of part A after 12 weeks, study patients may enter part B, an open-label part of the study. The subjects will generally receive subcutaneous clazakizumab in 4-weekly intervals until the end-of-study (EOS) visit after 52 weeks and are then to be subjected to a second protocol biopsy. Major goals of part B are to evaluate the safety and tolerability of a prolonged period of treatment with clazakizumab and the long-term impact of this antibody on the evolution of ABMR, rejection-associated biomarkers and kidney allograft function and survival over a period of 12 months.

### Example 4: Use of Clazakizumab as Treatment of Patients with Post-Transplant Antibody Mediated Rejection (ABMR)

A patient who has received a solid organ transplant (e.g., kidney, heart, liver, lungs, pancreas, skin, gall cladder, stomach, intestines or combinations of the foregoing) who shows signs of developing antibody mediated rejection (ABMR) or who exhibit ABMR is identified. As mentioned herein these patients are not amenable to treatment with the current standard-of-care immunosuppressive medications which is unfortunate as this is the largest single cause of post-transplant allograft failure.

In particular, the patient is monitored after transplant by diagnostic tests which allow for the prediction and early diagnosis of ABMR. For example the patient may be assessed by the use of one or more tests which detect pre-formed and de novo HLA DSA (especially those detecting complement binding DSA such as Clq) and/or the use of assays which detect the presence of non-HLA antibodies associated with ABMR.

Also, the transplanted organ may be examined for histological signs of ABMR-mediated damage which may be detected by the use of kidney allograft biopsies and screening of the biopsy sample for pathological symptoms characteristic of ABMR-mediated organ damage such as microvascular inflammation, complement deposition (C4d) in the peritubular capillaries, peritubular capillaritis, glomerulitis and transplant glomerulopathy (double glomerular basement membrane contour).

The identified patient, i.e., an individual who shows clinical or histological signs of developing antibody mediated rejection (ABMR) or chronic antibody mediated rejection (CABMR) or who exhibits ABMR or CAMBR is then prophylactically or therapeutically treated with Clazakizumab in order to prevent, stabilize or reverse the onset of ABMR. This treatment, i.e., the administration of an anti-IL-6 antibody should ameliorate or reduce the ABM damage caused by these HLA- and non-HLA DSAs.

The patient further may be treated with a combination of the standard of care post-transplant immunosuppressive medications (e.g. thymoglobulin, basiliximab, mycophenolate mofetil, tacrolimus, or anti-CD20 mAb such as rituximab, and corticosteroids), and Clazakizumab which is given either as an intravenous (at doses of 5 mg-50 mg) or as a subcutaneous injection (at doses of 10 mg-50 mg) typically every 4 weeks, starting at the time of transplant or when evidence of rejection develops. The first signs of rejection commonly include non-specific evidence such as a rise in serum creatinine or the development of proteinuria. Confirmation of ABMR or CAMBR is accomplished by the specific diagnostic blood tests and organ biopsies as described above.

Treatment with clazakizumab may be continued for several months (e.g. one month to several years) to prevent antibody mediated damage to the allograft and the resulting loss of function which can ultimately result in the total loss of the transplanted organ.

In order to further establish proof of concept, experiments were conducted to assess the effects of clazakizumab on co-cultures comprising allogeneic cells, on the proliferation of specific immune cells, and on the expression of cytokines involved in rejection responses.

In particular, experiments were conducted to assess the effects of clazakizumab on the transcription of HLA-DR, CD54, IL-6 and PDL-1. These experiments are described in the following examples.

### Example 5: Effect of Antagonist Anti-IL-6 Antibody (Clazakizumab) On EC Proliferation

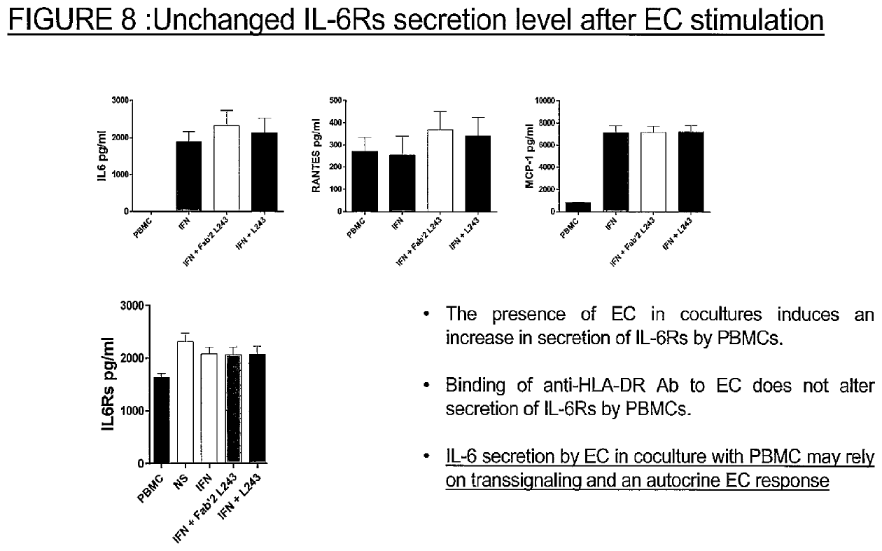

The presence of endothelial cells in co-cultures has been observed to induce an increase in the secretion of IL-6Rs by PBMCs (See FIG. 8). By contrast the binding of anti-HLA-DR Ab to endothelial cells does not alter secretion of IL-6Rs by PBMCs.

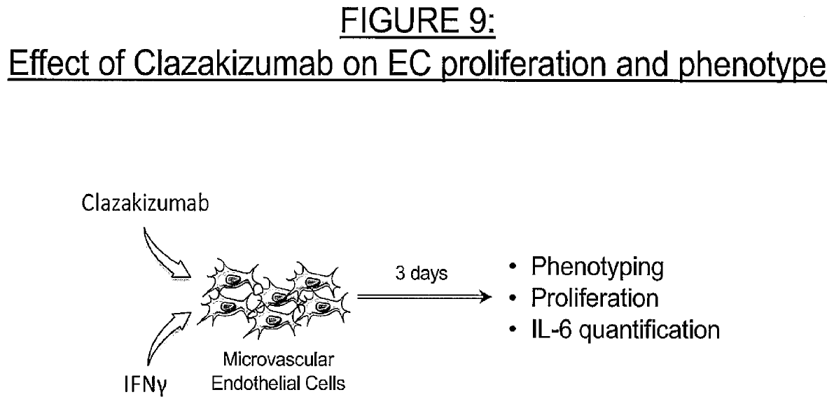

Based thereon it is theorized that IL-6 secretion by ECs in coculture with PBMC may rely on trans-signaling and an autocrine EC response. Based on the foregoing experiments were conducted to study the effect of different dosages of an antagonist anti-IL-6 antibody (Claza) on the proliferation of endothelial cells. As depicted schematically in FIG. 9 the effect of an antagonist anti-IL-6 antibody (Clazakizumab) on EC proliferation and phenotype was ascertained.

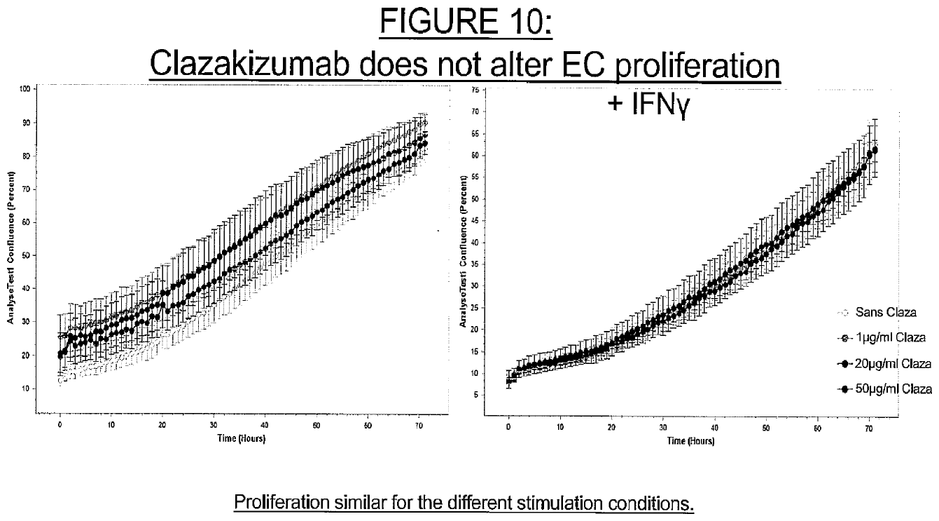

Particularly endothelial cell cultures were contacted with dosages of 1 μg/ml Claza, 20 μg/ml Claza and 50 μg/ml of Claza and no Claza. As shown in FIG. 10, irrespective of the IL-6 antagonist antibody dosage amount, there was no effect on endothelial cell proliferation.

### Example 7: Effect of Clazakizumab on Mediators of Allogenicity

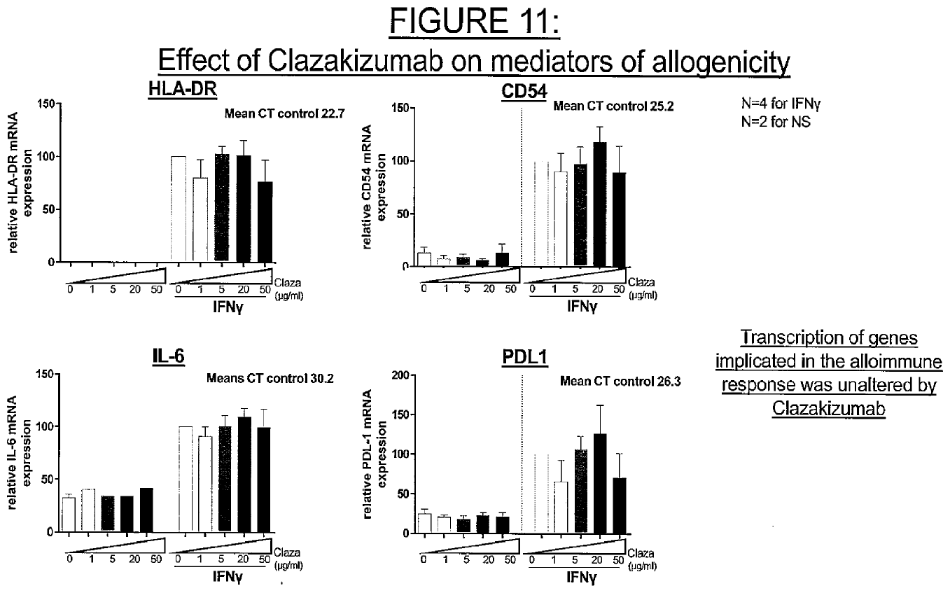

Experiments were conducted to assess the effects of an IL-6 antagonist antibody (Clazakizumab) on the transcription of genes implicated in alloimmune responses. As evidenced by the experiments shown in FIG. 11, the transcription of particular genes known to be involved in alloimmune responses was unaltered by Clazakizumab.

Specifically, in these experiments the levels of HLA-DR, CD54, IL-6, PDL-1 and Glyceraldehyde-3-phosphate dehydrogenase (GADPH) mRNAs were assayed using fluorescence-based real-time PCR after 3 days of treatment with different doses of Clazakizumab with or without IFNγ, and total RNA was isolated from Endothelial cells (ECs) using TRI REAGENT™(Ambion®, Applied Biosystems™, Thermo Fischer Scientific) protocol.

RNA was quantified using a spectrophotometer (ND-1000; NanoDrope), and converted to cDNA (1 μg RNA/reaction) by reverse transcription (RT) using the SuperScript™ III First-Strand Synthesis System for RT-PCR (Invitrogen Life Technologies). Real-time PCR was performed with ViiA™ 7 Real-Time PCR System (Applied Biosystems™, Thermo Fischer Scientific) and TaqMan™ gene Expression Assay (Applied Biosystems™, Thermo Fischer Scientific).

The primers and probe sets used for this study were: IL-6 (Hs00174131_m¹), CD54 (Hs00164932_m¹), HLA-DR (Hs00219575_m¹), PDL1 (Hs01125301_m¹) and GAPDH (Hs027558991_g1).

Threshold cycles (Ct) were determined as the mean of duplicate determinations. The differences in relative abundances of mRNA were calculated as ΔCt (Target gene—GAPDH ‘housekeeping’ gene) expressed as the percentage of the control condition (endothelial cells incubated with IFNγ). The mean±SEM values are shown in FIG. 11.

### Example 8: Effect of Clazakizumab on EC Phenotype

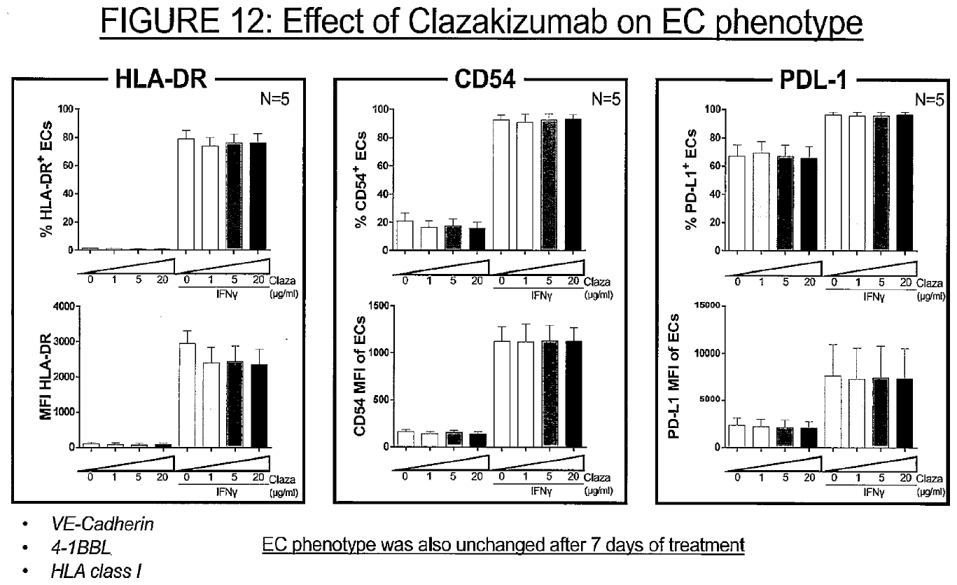

Experiments were conducted to assess the effects of an IL-6 antagonist antibody (Clazakizumab) on EC phenotype. As evidenced by the experiments shown in FIG. 12, EC phenotype was not impacted by the anti-IL-6 antibody after 7 days of treatment.

In these experiments endothelial cells were cultured with interferon γ (IFN-γ) at 20 ng/ml (Eurobio) in tissue culture flasks and incubated, as shown in FIG. 12 with different doses of clazakizumab for 3 days.

For phenotypic analysis of endothelial cells, the following antibodies were used: HLA-DR APC (Clone L243, BioLegend®), CD54 Pacific Blue™ (clone HCD54, BioLegend®), CD274 PC7 (Clone MIH1, BD Pharmingen™).

EC's were trypsinized with trypsin 0.05% EDTA (Gibco) before washing with 1 ml of cold Phosphate Buffered Saline (PBS) with 0.5% of Bovine Serum Albumin (BSA) and centrifuging at 4° C. Monoclonal antibodies were added and incubated 30 min on ice. Afterward cells were washed again using the same washing conditions and the cells were resuspended in PBS 0.5% BSA before analysis on a FACS Canto™ II clinical flow cytometry system (BD Biosciences).

As shown in FIG. 12 the results of these experiments are expressed as the percentage of cells expressing the relevant antigen with the mean±SEM values shown. It can be seen that EC phenotype was not impacted by the anti-IL-6 antibody after 7 days of treatment.

### Example 9: Effect of Clazakizumab on ELISA Detection of IL-6

Experiments were also conducted to assess the effects of an IL-6 antagonist antibody (Clazakizumab) on the amount of IL-6 detected in ELISA assays. Specifically it was assessed whether Clazakizumab interferes with the detection of IL-6 when IL6 was quantified with an enzyme-linked immunosorbent assay (ELISA) detection kit from BioLegend®, which was used according to the manufacturer's protocol.

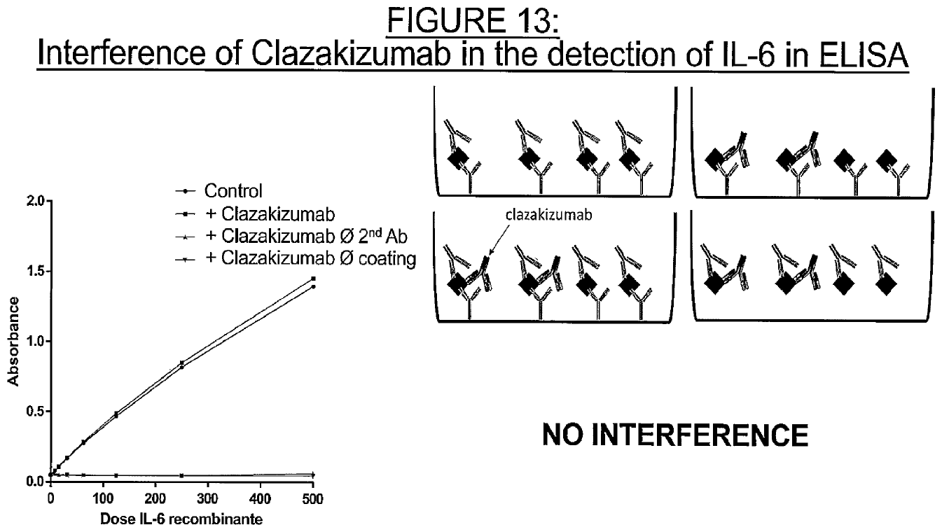

In these experiments IL6 was assayed in supernatants with a known concentration of IL-6 to which clazakizumab (20 μg/ml) was added or not added. Control conditions were included in which secondary antibody was not added or without coating the ELISA plates with the detection antibody. The schematic in FIG. 13 represents the different conditions tested. The results are expressed as absorbance units.

Based on the results in FIG. 13 Clazakizumab does not appear to interfere with the detection of IL-6 using an enzyme-linked immunosorbent assay (ELISA).

### Example 10: Effect of Clazakizumab on IL-6 Secretion

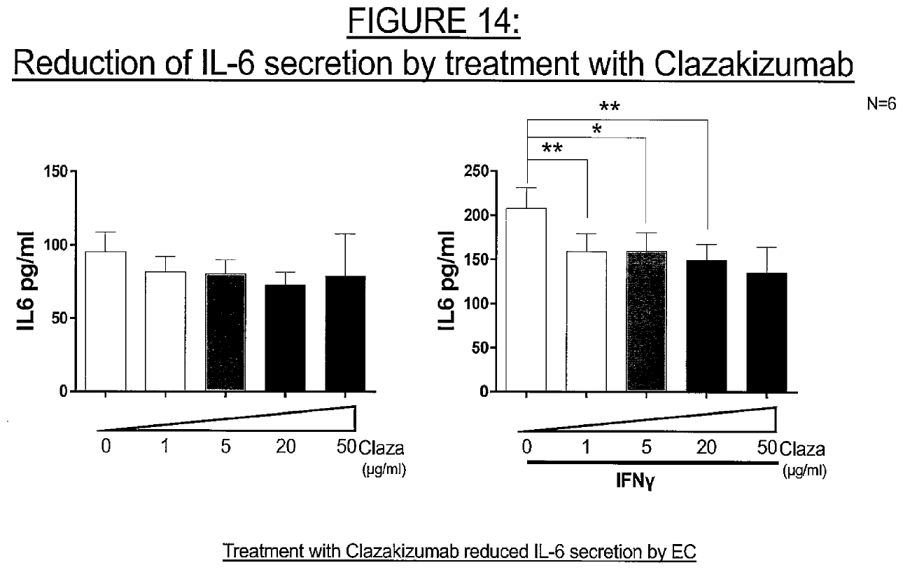

As shown in FIG. 14 experiments were also conducted to assess the effects of an IL-6 antagonist antibody (Clazakizumab) on the secretion of IL-6 by endothelial cells. In these experiments endothelial cells were cultured with or without interferon γ (IFN-γ) at 20 ng/ml (Eurobio) in tissue culture flasks and incubated, as shown in FIG. 14, with different doses of clazakizumab.

After 3 days, IL-6 was quantified in the supernatants of ECs using an enzyme-linked immunosorbent assay detection kit from BioLegend®, which was used according to the manufacturer's protocol. The results as shown in FIG. 14 are expressed as quantity of IL-6 secreted. In the Figure the mean±SEM values (*p<0.05 and **p<0.01, paired t-test) are shown.

The detected effects of the IL-6 antagonist antibody (Clazakizumab) at different dosage concentrations are shown in FIG. 14.

### Example 11: Effect of Clazakizumab on CCL-2 Production in EC-PBMC Co-Cultures

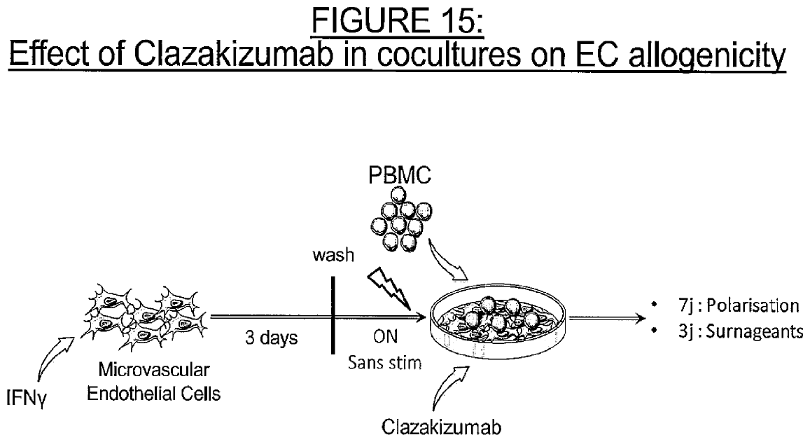

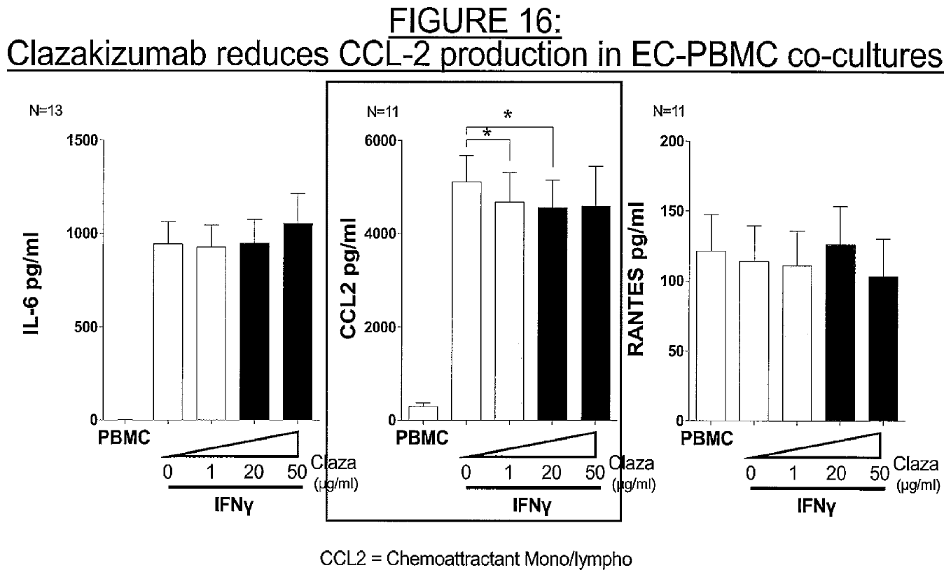

As shown schematically in FIG. 15 experiments were also conducted to assess the effects of an IL-6 antagonist antibody (Clazakizumab) on allogenicity observed in EC co-cultures. Specifically, as shown in FIG. 16 experiments were conducted to assess the effects of an IL-6 antagonist antibody (Clazakizumab) on CCL-2 production in EC-PBMC co-cultures. In these experiments ECs were activated by IFNγ (20 ng/ml (Eurobio)) for 3 days and then starved of IFNγ overnight before co-culture with non-HLA-matched PBMCs. ECs were washed and irradiated at 20 Gy.

As shown in FIG. 16 the irradiation step did not prevent cytokine secretion by ECs within the following 3 days. Carboxyfluorescein succinimidyl ester (CFSE)—labeled PBMCs (2.5 μM; Molecular Probes/Invitrogen) were stimulated with irradiated ECs (1:1) for 7 days in RPMI-10% human AB serum (EFS).

At T0, as shown in FIG. 16, different concentrations of clazakizumab were added to the EC/PBMC co-cultures. The supernatants of co-cultures were collected after 72 hours and assayed to detect amounts of IL-6, CCL2 and RANTES by enzyme-linked immunosorbent assays detection kits from BioLegend®, again performed according to the manufacturer's protocol. The results of these experiments which are shown in FIG. 16 are expressed as quantity of cytokines secreted. The mean±SEM values (*p<0.05, paired t-test) are shown. The results indicate that the IL-6 antagonist antibody (Clazakizumab) reduces CCL-2 production in EC-PBMC co-cultures.

### Example 12: Effect of IL-6 Antagonist Ab (Clazakizumab) on T-CD4+Activation bv ECs

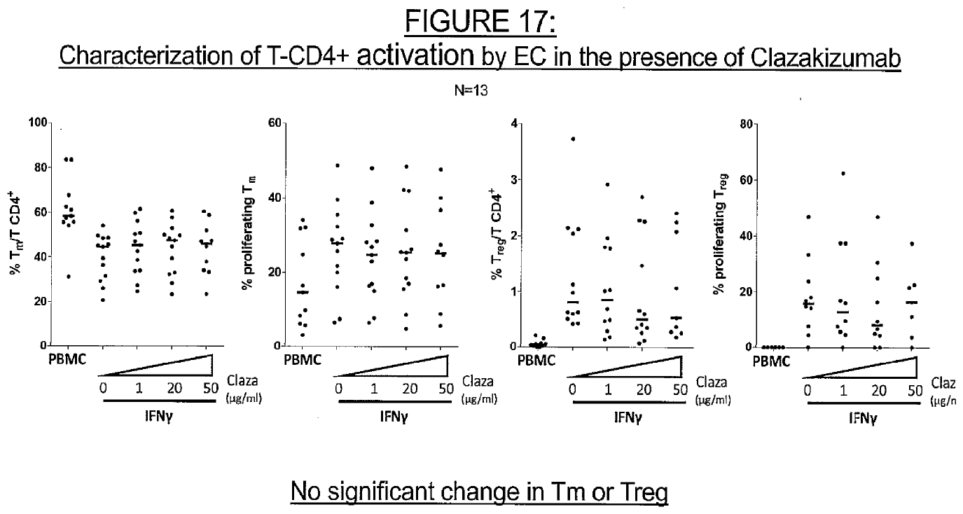

As shown in FIG. 17 experiments were also conducted to assess the effects of an IL-6 antagonist antibody (Clazakizumab) on T-CD4+activation by ECs. In these experiments EC/PBMC co-cultures were obtained as previously described. After seven days of coculture, carboxyfluorescein succinimidyl ester (CFSE)—labeled PBMCs were used for the study of the proliferation of Treg identified as (CD4⁺CD45^{RA-}CD25^{high}CD127^{low}mFoxP3^{bright}) and Tmem identified as (CD4⁺CD45^{RA-}FoxP3^{low}) subpopulations.

For flow cytometry, the following antibodies were used: CD4 PB (Clone RPA-T4), CD45RA PE/Cy7 (clone H100), CD25 PE (clone M-A251), CD127 PerCP/Cy5.5 (clone A019D5) (BioLegend®). Intracellular staining of FoxP3 was carried out with the anti-Human Foxp3 Staining Set APC (clone 236A/E7) (eBioscience™) Flow cytometry was carried out on a FACS Canto™ II clinical flow cytometry system (BD Biosciences).

The results are expressed as percentage of each T cells subset and the percentage of proliferating cells in these population. Median values (red line) are shown.

### Example 13: Effect of IL-6 Antagonist Ab (Clazakizumab) on Endothelial Expansion of Th17 and Th1 Cells

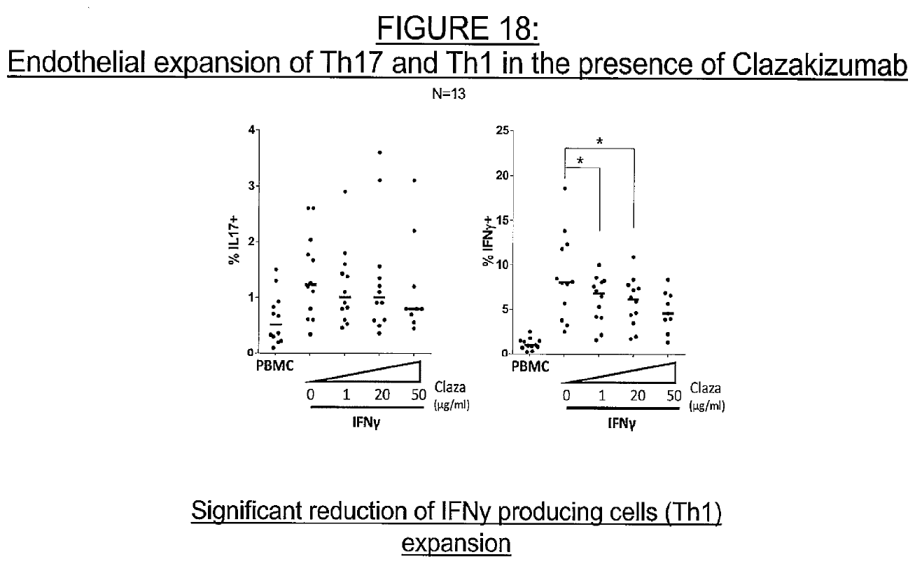

As shown in FIG. 18 experiments were also conducted to assess the effects of an IL-6 antagonist antibody (Clazakizumab) on endothelial expansion of Th17 and Th1 cells. In these experiments EC/PBMC cocultures were obtained as previously described. After seven days of coculture, PBMCs were stimulated by phorbol 12 myristatel3 acetate (PMA) 50 ng/mL and ionomycin 1 μM (Cell Signaling Technologye) with GolgiStop 1× (BD Biosciences) for 4 h, and Th17 (CD3⁺CD8⁻IL17⁺) and Th1 (CD3⁺CD8⁻IFNγ⁺) subpopulations were analyzed by flow cytometry to detect intracellular cytokines.

For flow cytometry, the following antibodies were used: IFN-γ FITC (Clone B27) (BD Pharmingen™ BD Biosciences), CD4 PE (Clone RPA-T4), CD3 PerCP (clone SK7), CD8 PB (Clone RPA-T8) (BioLegend®) and IL-17 efluor660 (eBioscience). Flow cytometry was carried out on a FACS Canto™ II clinical flow cytometry system (BD Biosciences). The results in FIG. 18 are expressed as percentage of each T cells subset. Median values (red line) are shown (*p<0.05, paired t-test).

Based on the results in FIG. 18 it can be seen that there was a significant reduction of IFNγ producing cells (Th1) expansion in the presence of the IL-6 antagonist antibody.

### Example 14: Effect of IL-6 Antagonist Ab (Clazakizumab) on Th1 Response of Allogeneic CD4⁺ T Cells

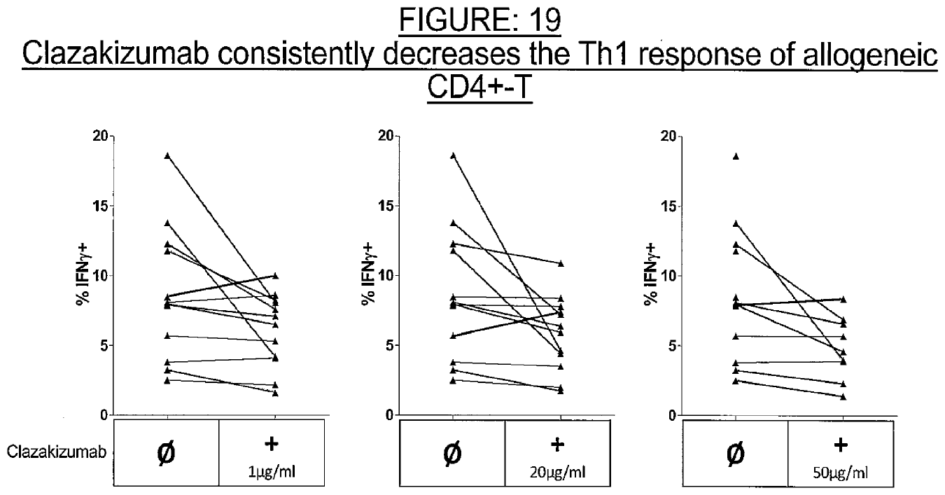

As shown in FIG. 19 experiments were also conducted to assess the effects of an IL-6 antagonist antibody (Clazakizumab) on Th1 responses of allogeneic CD4+T cells. This Figure represents the distribution of Th1 cells in different donors and compares control conditions and conditions using different doses of clazakizumab as indicated in the 7 days co-cultures. The analysis of Th1 populations was performed as previously described. These results show that Clazakizumab consistently decreased the Th1 response elicited by allogeneic CD4⁺T cells.

### Example 15: Effect of a Low Dose of IL-6 Antagonist Ab (Clazakizumab) on the Expansion of Th1 Cells

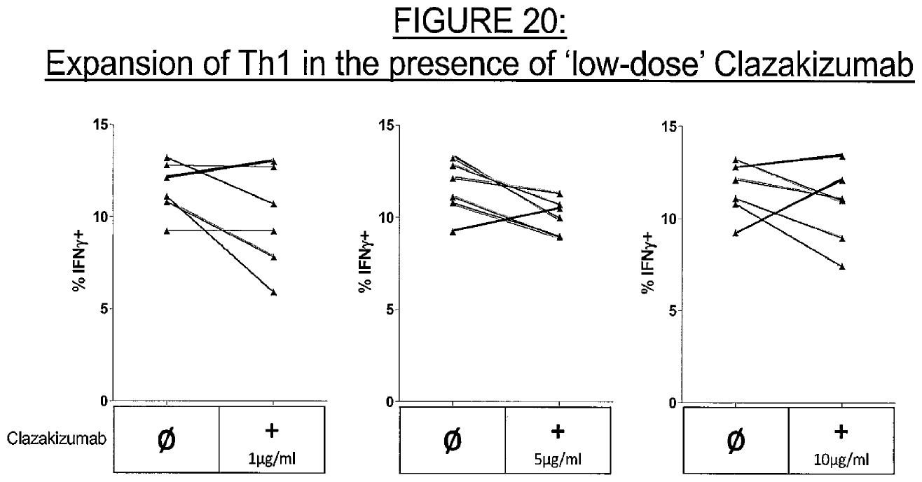

As shown in FIG. 20 experiments were also conducted to assess the effects of an IL-6 antagonist antibody (Clazakizumab) on the expansion of Th1 cells in the presence of ‘low-dose’ Clazakizumab. The results in FIG. 20 represent the distribution of the Th1 cells in different donors and compare the control condition with conditions involving the addition of different doses of clazakizumab as indicated in the 7 day co-cultures. The analysis of the Th1 population were performed as previously described.

The results indicate that the distribution of the Th1 cells was decreased even at low Claza antibody dosages.

### Example 16: Effect of IL-6 Antagonist Ab (Clazakizumab) on EC Expression of Complement Regulatory Proteins

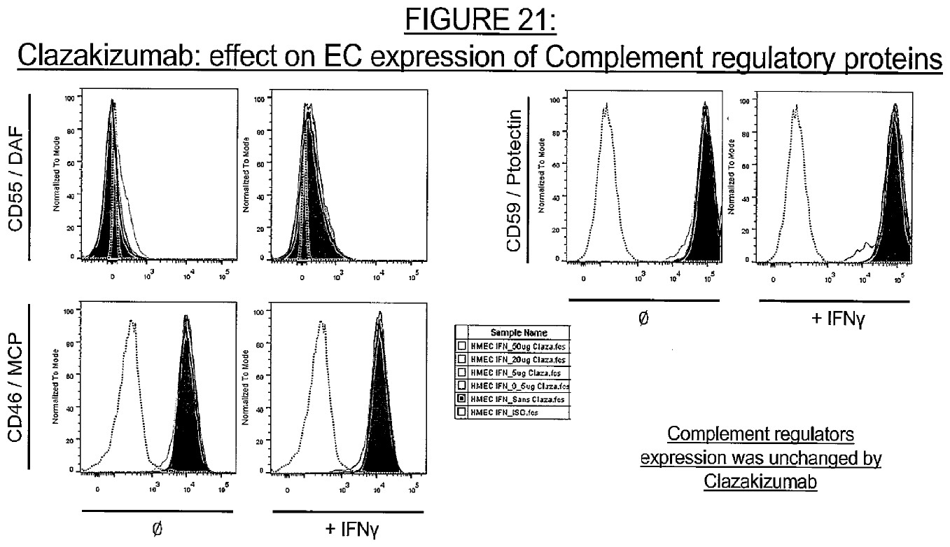

As shown in FIG. 21 experiments were also conducted to assess the effects of an IL-6 antagonist antibody (Clazakizumab) on t of IL-6 Antagonist Ab (Clazakizumab) on EC expression of Complement regulatory proteins. In these experiments endothelial cells were cultured with interferon γ (IFN-γ) at 20 ng/ml (Eurobio) in tissue culture flasks and incubated, where indicated, with different doses of clazakizumab (0, 5; 5; 20; 50 μg/ml for 3 days).

Phenotypic analysis of endothelial cells was carried out using the following antibodies: CD55 FITC (Clone JS11), CD46 PC7 (clone TRA-2-10) and CD59 PE (p282(H19)) (BioLegend®).

ECs were detached with Versene 1× (Gibco™) and washed in 1 ml of cold Phosphate Buffered Saline (PBS) with 0.5% of Bovine Serum Albumin (BSA) before centrifuging at 4° C. mAb were added and incubated 30 min on ice. Then cells were washed again as previously described and resuspended in PBS 0.5% BSA.

FIG. 21 shows the overlays of histograms of expression for each antigen at all concentrations of clazakizumab tested. Isotype controls are represented by the dotted line and the control without clazakizumab in grey.

### Example 17: Effect of IL-6 Antagonist Ab (Clazakizumab) on Complement Activation

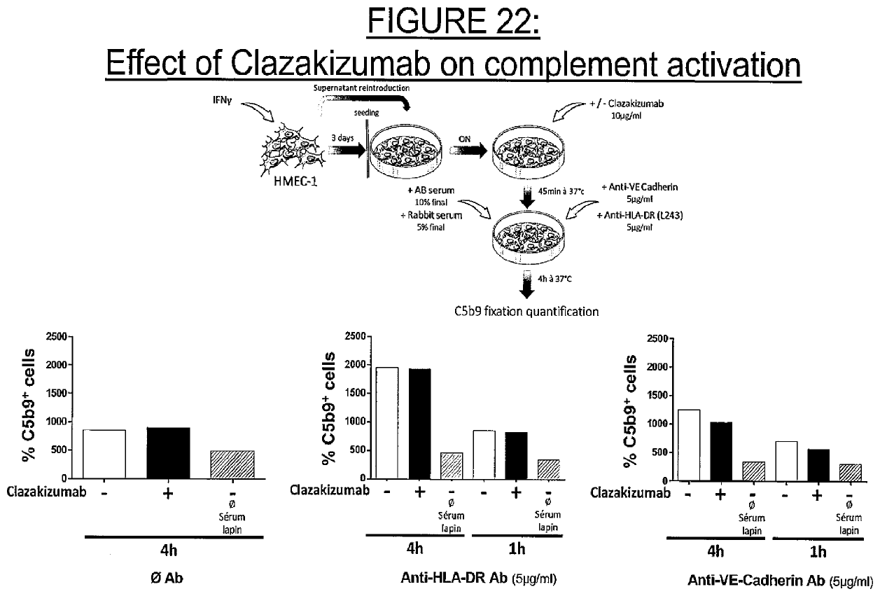

As shown in FIG. 22 experiments were also conducted to assess the effects of an IL-6 antagonist antibody (Clazakizumab) on complement activation. In these experiments endothelial cells were cultured with interferon γ (IFN-γ) at 20 ng/ml (Eurobio) in tissue culture flasks for 3 days. ECs were then detached with trypsin 0.05% EDTA (Gibco™) and the supernatants from the 3-day cultures were stored and reused on the reseeded cells. After 18h, 10 μg/ml of clazakizumab were added or not added to cultures for a further 45 min at 37° C.

Following the above of human AB serum was added to make 10% final of human AB serum and rabbit serum was added to make 5% final of rabbit serum and 5 μg/ml of mAb directed against HLA-DR or VE-cadherin were added. The antibodies were left for 4 hours at 37° C. in order to allow activation of the complement cascade.

In order to study complement activation, the fixation of C5b9 on EC was quantified by flow cytometry. The following antibodies were used: SC5b9 Biotinylated (Quidel, San Diego) and Streptavidin A647 (Invitrogen).

EC were detached with Versene 1X (Gibco™) and washed with 1 ml of cold Phosphate Buffered Saline (PBS) with 0.5% of Bovine Serum Albumin (BSA) and centrifuged at 4° C.

C5b9-biotinylated mAb was added and incubated 30 min on ice. Then cells were washed again as previously described and stained with Streptavidin A647 for 15 min at 4° C. Finally, ECs were washed twice with PBS 0.5% BSA before flow cytometry analysis.

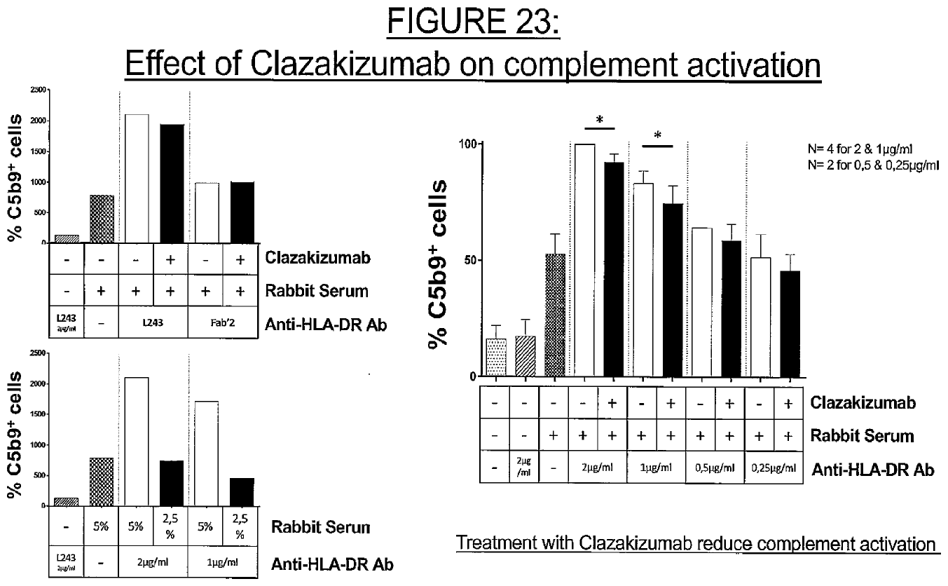

The results in FIG. 23 are expressed as the percentage of cells positive for the fixation of C5b9. These results show that an IL-6 antagonist antibody (Clazakizumab) significantly reduced complement activation and should be well suited for treating AMBR or CAMBR and other indications where complement activity is involved in disease pathology. Further the experimental results obtained in the EC-PBMC co-cultures demonstrated that clazakizumab by itself resulted in a decrease in Tregs and further reduced the expansion of Th1 pro-inflammatory lymphocytes.

### Example 18: Clazakizumab Acts On Endothelial Cells To Limit Antibody Mediated Damage

Human microvascular endothelial cell expression of HLA class II antigens is strongly increased, both in vitro and in vivo, under inflammatory conditions. HLA class II antibody binding to endothelial cells enhances IL-6 secretion and thereby increases the ability of the endothelial cell to activate and to differentiate pro-inflammatory Th17 CD4⁺ lymphocytes mediated by an IL-6 dependent activation of Stat-3 (Taflin, PNAS 2011, Lion Am J Trans. 2016). The Interleukin-6-specific antibody, Clazakizumab, was studied to determine its ability to act upon HLA II expressing endothelial cells.

Methods:

Endothelial cells were pre-incubated with Clazakizumab prior to and during co-culture with PBMC from non-related individuals. Additionally, binding of HLA-specific antibodies to endothelial cells results in complement activation and leads to C5b-C9 deposition. This was tested in the presence of Clazakizumab. CD4⁺ T cell sub-populations were identified by intracellular cytokine staining and C5b-C9 was detected by multicolor flow cytometry.

Results:

This study reports that pre-incubation of endothelial cells with Clazakizumab decreased IL-6 secretion by human endothelial cells. Clazakizumab also reduced levels of the chemoattractant CCL2 in endothelial cell co-cultures with allogeneic PBMCs. Moreover the endothelial cell mediated expansion of pro-inflammatory Th17 and Th1 populations was decreased. Deposition of C5b-C9 was determined after HLA-antibody binding to endothelial cells and was significantly reduced when Clazakizumab was present.

Conclusions:

Together these data support the idea that Clazakizumab acts directly on endothelial cells. The combined outcomes of reduced CCL2 production, reduced pro-inflammatory CD4⁺-T differentiation and decreased formation of the C5b-C9 complex, should result in an overall protective effect on the allograft endothelium in the context of chronic humoral rejection associated with HLA-specific alloantibodies.

These results therefore show that the tested anti-IL-6 antagonist antibody (Clazakizumab) significantly reduced complement activation and should be well suited for treating AMBR or CAMBR and other indications where complement activity is involved in disease pathology. Suh conditions include age-related and degenerative diseases such as Age-related macular degeneration (AMD) (wet and dry), Alzheimer's Disease, glomerular diseases e.g., atypical hemolytic uremic syndrome (aHUS), hemolytic uremic syndrome caused by Shiga toxin-producing E. coli (STEC-HUS), thrombotic thrombocytopenic purpura (TTP), systemic lupus erythematosus (SLE), antiphospholipid antibody syndrome (APS), anti-neutrophil cytoplasmic antibody (ANCA)-induced vasculitis, inflammatory small-vessel disorders caused by autoantibodies against neutrophil constituents; antibody-dependent (i.e., in women with APS), pregnancy loss involving C5a-mediated impairment of placental angiogenesis; complement mediated hemolytic disorders such as paroxysmal nocturnal hemoglobinuria (PNH), aHUS and cold-agglutinin disease (CAD), Ischemia-reperfusion injury; stroke, myocardial infarction e.g., caused by to trauma, sepsis, shock and cardiopulmonary bypass (CPB) surgery, et al. Also, complement mediated conditions which may be treated according to the invention include transplant-related complications, especially when organs are transplanted after circulatory arrest of the donor, which can lead to the induction of IRI. Both the production (via B cell-costimulation) and effect of alloantibodies (via CP/LP activation) are complement-driven events in antibody-mediated rejection (ABMR). In the case of Langerhans islet transplantation in diabetic patients, the occurrence of a thromboinflammatory response known as ‘instant blood-mediated inflammatory reaction’ is caused by rapid complement activation and limits transplantation efficiency due to islet destruction. A particularly interesting, yet still incompletely understood, phenomenon in the context of transplantation is accommodation, in which transplant cells become ‘resistant’ to complement-mediated destruction. Such incompatibility responses may influence the outcome of CPB cardiopulmonary bypass surgery, during which circuit materials, blood/air interfaces in the oxygenator, activated platelets, and protamine complexes (generated to neutralize soluble heparin at the end of the procedure) can activate complement and contribute to systemic inflammatory response syndrome.

Other inflammatory diseases with complement contribution include allergic asthma and periodontitis. The ties between complement and asthma have long been recognized, yet the involvement appears to be complex. Under asthmatic conditions, complement is not only activated through the CP via allergen-antibody complexes but C3 and C5 might also be cleaved by proteases derived from certain allergens (e.g., house dust mites). The resulting C3a and C5a act synergistically in creating a proallergenic immune environment, yet C5a may also protect from maladaptive Th2 immunity during allergen sensitization. An important yet complex role in asthma has also been attributed to C5L2. Whereas previous therapeutic attempts focused on C5aR, the scope has recently been expanded to include inhibitors at the levels of C5 and C3. Relatedly, C5a has also been implicated in the exacerbation of chronic obstructive pulmonary disease. Finally, complement-mediated processes have been recognized as critical for bone-related disorders and injury (e.g., via anaphylatoxin effects on osteoclast formation), thereby suggesting another potential indication area for complement therapeutics.

Perhaps the most severe effects of complement activation are seen in acute-phase conditions, often associated with systemic inflammatory response syndrome (SIRS), in which the host is confronted with a dramatic increase of damage- and/or pathogen-associated molecular patterns. In trauma, for example, the initial traumatic impact combined with posttraumatic IRI can trigger a devastating cascade of immuno-inflammatory reactions with complement contribution, which may sustain SIRS. As a complication of trauma, or as an independent incident, massive infection may overwhelm the protective functions of complement and other innate immunity components (e.g., TLR) and provoke sepsis immune cell activation, a cytokine storm and coagulopathy may result in SIRS and persist even after the pathogen is cleared; C5a-dependent signaling seems to be a major player in those devastating events.

These experimental results further corroborate that clazakizumab may be used to treat or prevent AMBR or CAMBR for prolonged duration in subjects in need thereof, i.e., patients who are to receive, have already received or are receiving transplanted allogeneic or xenogeneic cells, tissues or one or more organs, e.g., allogeneic or xenogeneic cells used in gene or cell therapy such as immune cells, fibroblasts, skin cells, neural cells, adult stem cells, or solid organs such as kidney, bladder, lung, heart, liver, skin, pancreas, stomach, intestine or any combination of the foregoing.

### Example 19: Clazakizumab Clinical Regimen For Treating AMBR or CAMBR

Subjects treated in the instant AMBR or CAMBR clinical regimen will in general comprise the following inclusion criteria:
1. Age 18-75 years;
2. Living donor/deceased donor kidney transplant recipients months from time of transplant;
3. Diagnosis of CABMR (according to Banff 2015 diagnostic criteria) to include all of the following:
   i. Biopsy proven CABMR (i.e., chronic glomerulopathy (cg)>0) with/without C4d staining
   ii. Presence of HLA DSA (using single-antigen bead-based assays) post-transplant.

Patients excluded from treatment in the subject clinical regimen include those who meet all of the following exclusion criteria:
1. Multi-organ transplant recipient.
2. Treatment for ABMR (including CABMR) or TCMR within 3 months of screening.
3. Received T cell depleting agents (e.g., alemtuzumab, anti-thymocyte globulin) within 3 months of screening.
4. Biopsy showing pure TCMR or advanced interstitial fibrosis (ci3), advanced tubular atrophy (ct3), vascular fibrous intimal thickening (cv3) or other significant causes of renal dysfunction (e.g., BKV nephropathy, glomerulonephritis).
5. Impaired renal function due to disorders in the transplanted allograft (e.g., renal artery stenosis, hydronephrosis).
6. eGFR <25 mL/min/1.73 m² or >65 mL/min/1.73 m².
7. Nephrotic range proteinuria defined as spot urine protein creatinine ratio (UPCR) ≥3,000 mg/g (≥300 mg/mmol) or spot urine albumin creatinine ratio (UACR) ≥2,200 mg/g (≥220 mg/mmol). If spot UPCR or UACR is above defined limits, repeat test on separate day (or collect 24-hour urine to confirm nephrotic range proteinuria (≥3.0 g/day)).
8. Pregnant, breastfeeding, or unwillingness to practice highly effective birth control during the study and for 5 months after last dose of Clazakizumab.
9. History of anaphylaxis.
10. Abnormal LFTs (alanine aminotransferase (ALT)/aspartate aminotransferase (AST)/bilirubin >1.5× upper limit of normal) or other significant liver disease.
11. History of active tuberculosis (TB).
12. History of latent TB without history of active TB (e.g., positive QuantiFERON-TB® (QFT®) test) unless subject has completed a full course of prophylactic treatment.
13. History of human immunodeficiency virus (HIV) infection or positive for HIV.
14. Seropositive for hepatitis B surface antigen (HBsAg).
15. Hepatitis C virus (HCV) RNA positive.
16. Known EBV mismatch: donor seropositive, recipient seronegative.
17. History of GI perforation, diverticular disease or diverticulitis, or inflammatory bowel disease.
18. Neutropenia (<1,000/mm³) or thrombocytopenia (<50,000/mm³).
19. Active infections requiring systemic antimicrobial agents and unresolved prior to Screening.
20. History of or current invasive fungal infection or other opportunistic infection, including (but not limited to) the following: a nontuberculous mycobacterial infection, aspergillosis, pneumocystosis, and toxoplasmosis.
21. Active viral infections such as BKV, CMV, or EBV based on polymerase chain reaction (PCR) testing.
22. Current or recent (within 3 months) participation in an Clazakizumab trial.
23. Administration of a live vaccine within 6 weeks of screening, including but not limited to the following:
   i) Adenovirus
   ii) Measles, mumps, and rubella
   iii) Oral polio
   iv) Oral typhoid
   v) Rotavirus
   vi) Varicella zoster
   vii) Yellow fever
24. History of alcohol or illicit substance (including marijuana) abuse.
25. Present or previous (within 3 years) malignancy except for basal cell carcinoma, fully excised squamous cell carcinoma of the skin, or non-recurrent (within 5 years) cervical carcinoma in-situ.
26. Presence of a condition or abnormality (i.e., clinically significant endocrine, autoimmune, metabolic, neurological, psychiatric/psychological, renal, GI, hepatic, and hematological or any other system abnormalities that are uncontrolled with standard treatment) that in the opinion of the Investigator would compromise the safety or life expectancy of the patient or the quality of the data.
27. History of intolerance to trimethoprim and/or sulfamethoxazole. This criterion does not apply if subject is already taking inhaled pentamidine or oral dapsone for Pneumocystis jirovecipneumonia (PJP) prophylaxis, or if subject is willing to begin taking either of these drugs at least 1 week prior to the Day 1 Baseline visit (Visit 2).
28. Prior exposure to clazakizumab.

Subjects may be permanently discontinued from anti-IL-6 antibody administration upon the appearance of an unacceptable adverse event (AE) selected from the following:
1. AST or ALT >5.0× upper limit of normal (ULN)
2. Total bilirubin >3.0× ULN
3. AST or ALT >3.0 to 5.0× ULN and total bilirubin ≥2.0× ULN (or international normalized ratio (INR)>1.5)

Subjects may be permanently discontinued from anti-IL-6 antibody administration Due to Neutropenia and/or Thrombocytopenia. In particular, subjects who meet any of the following conditions during treatment may have anti-IL-6 antibody administration treatment stopped:
1. Neutrophil count <1,000 cells per mm³
2. Platelets <50,000 per mm³

Subjects may be permanently discontinued from anti-IL-6 antibody administration due to BKV, CMV, or EBV Viral Infection. For example subjects who meet any of the following conditions at any time during treatment may have Clazakizumab treatment stopped:
1. BKV ≥10,000 copies/mL (by PCR) or biopsy proven BKV nephropathy
2. CMV end-organ disease (e.g., hepatitis, colitis, pneumonitis, retinitis)
3. EBV ≥10,000 copies/mL (by PCR) or post-transplant lymphoproliferative disorder or primary EBV infection in seronegative recipient

### Claza Treatment Regimen

Clazakizumab is generally provided as 25 mg/mL and 12.5 mg/mL dosage formulations. The excipients comprise L-histidine, L-histidine monohydrochloride, sorbitol, polysorbate-80, and water for injection. The dosage form comprises single-dose vials (25 mg/mL and 12.5 mg/mL) suitable for injection.

The antibody is stored at −20±5° C. (−4±9° F.) or colder with protection from light.

### Concomitant Trimethoprim/Sulfamethoxazole Prophylactic Therapy

Oral trimethoprim/sulfamethoxazole in the form of a single-strength pill (80 mg as trimethoprim) daily or double-strength pill (160 mg as trimethoprim) 3 times per week are to be prescribed for PJP prophylaxis at investigational sites. Trimethoprim/sulfamethoxazole is generally started for at least 1 week before the Day 1 Baseline visit (Visit 2) (for subjects who were not already taking trimethoprim/sulfamethoxazole prior to entry in the study and who are not already receiving inhaled pentamidine or oral dapsone).

### Concomitant Inhaled Pentamidine and Oral Dapsone Prophylactic Therapy

Subjects who are already receiving inhaled pentamidine or oral dapsone for PJP prophylaxis at screening, generally will remain on these drugs and not start trimethoprim/sulfamethoxazole. Subjects who are intolerant to trimethoprim/sulfamethoxazole and who are not already receiving inhaled pentamidine or oral dapsone generally will be started on either one of these drugs at least 1 week before the Day 1 Baseline visit (Visit 2).

### Dosage and Administration of Clazakizumab

Clazakizumab is administered at a target dose of 25 mg every 4 weeks (Q4W) by SC injection or at a reduced dose of 12.5 mg Q4W by SC injection to support potential dose-reductions directed by protocol-defined safety parameters. Each 25 mg/12.5 mg dose is administered as a 1 mL injection of clazakizumab (25 mg/mL/12.5 mg/mL).

Clazakizumab is generally prepared and dispensed in identical filled, colored syringes. Each colored syringe generally contains a label with details including protocol number, subject ID, visit number, and date dispensed. The pharmacist generally will record the kit/vial number dispensed for each subject, including the date and time of dispensing on an accountability log. Prepared syringes may be stored for up to 24 hours in a refrigerator, 2° C. to 8° C. (36° F. to 46° F.), and up to 4 hours of the 24 hours may be at room temperature, 15° C. to 25° C. (59° F. to 77° F.). The prepared syringes should be protected from light. Prior to administration, the prepared syringe must reach room temperature by removing from refrigeration for 30 to 60 minutes before use.

To ensure patient safety, the most recent LFT and CBC analyses and viral monitoring results from a prior visit (scheduled or otherwise) generally are reviewed prior to dosing. All assessments per protocol for a given visit generally are completed prior to dosing with Clazakizumab.

### Packaging

Clazakizumab generally is supplied as single-dose vials. Vials are 2 mL flint glass, containing a minimum of 1.1 mL (25 mg/mL or 12.5 mg/mL) clazakizumab to deliver 1 mL (25 mg or 12.5 mg).

### Storage

Clazakizumab preferably is stored at −20±5° C. or colder, with protection from light.

### Clazakizumab Dose Modification

During Clazakizumab treatment subjects generally are monitored for abnormal LFTs, neutrophil and platelet counts, and viral infection with BKV, CMV and EBV. Based on the results of these assessments, the dose of Clazakizumab may be reduced to 12.5 mg SC Q4W, temporarily withheld, or permanently discontinued.

In general, Clazakizumab termination or dose-reduction described for abnormal LFTs is effected at the discretion of the treating clinician for any laboratory abnormality depending on the Common Toxicity Criteria for Adverse Events (CTCAE) severity (CTCAE Grade 1 (mild), Grade 2 (moderate), Grade 3 (severe or medically significant)) and corrective actions taken. In the case of neutropenia or thrombocytopenia, guidelines for modification of mycophenolate mofetil (MMF)/mycophenolic acid (MPA)/azathioprine (AZA) may be effected.

Also, Clazakizumab termination or dose-reduction may be effected for any other clinically significant infection. Once the infection has been treated and resolved, Clazakizumab potentially can be restarted at a reduced dose or the dose may be increased back to 25 mg SC Q4W at the discretion of the clinician. If Clazakizumab is withheld for doses because of an AE, the clinician generally may consider stopping Clazakizumab permanently.

Modification of Dose of Clazakizumab and/or Background Immunosuppression Based on Abnormal LFTs, Neutropenia, or Thrombocytopenia

During the clinical regimen, monitoring for LFT abnormalities, neutropenia and thrombocytopenia is performed at the start of treatment and every 4 to 12 weeks thereafter. Clazakizumab discontinuation or dose-reduction (to 12.5 mg SC Q4W) depending on CTCAE severity grading may be effected in the event of abnormal LFTs (i.e., AST/ALT), neutrophil, or platelet counts. Clazakizumab may be discontinued for any LFT abnormalities, neutrophil or platelet counts that meet CTCAE Grade ≥3.

Table 1 below provides further guidelines for dose adjustment of Clazakizumab and/or background immunosuppression according to CTCAE severity grade. Decisions regarding dose modification should be made in consultation with the clinician.

TABLE 1
Modification of Dose of Clazakizumab and/or
Background Immunosuppression Based on Abnormal
LFTs, Neutropenia, or Thrombocytopenia

| | |
|---|---|
| | Modification of Dose of Clazakizumab |
| Parameter | and/or Background Immunosuppression |

LFTs (AST/ALT)

| | |
|---|---|
| ≤3.0 × ULN | No change to Clazakizumab dose. |
| (CTCAE Grade 1) | |
| >3.0-5.0 × ULN | In addition, if total bilirubin is ≥2.0 × |
| (CTCAE Grade 2) | ULN (or INR >1.5), stop Clazakizumab. |
| | If total bilirubin is <2.0 × ULN, reduce |
| | dose of Clazakizumab to 12.5 mg SC Q4W. |
| | Perform investigations to exclude other |
| | causes of abnormal LFTs (e.g., other |
| | hepatotoxic drugs, alcohol, viral infections, |
| | autoimmune hepatitis, hemochromatosis, |
| | etc.). |
| | Increase dose of Clazakizumab back up to |
| | 25 mg SC Q4W if circumstances allow or |
| | continue 12.5 mg SC Q4W |
| >5.0 × ULN | Stop Clazakizumab. |
| (CTCAE Grade ≥3) | |

Neutrophil Count (cells per mm³)

| | |
|---|---|
| >1,500-LLN | Reduce dose of MMF/MPA/AZA by 50% |
| (CTCAE Grade 1) | No change to Clazakizumab dose. |
| <1,500-1,000 | Reduce does of MMF/MPA/AZA by 50% |
| (CTCAE Grade 2) | Reduce dose of Clazakizumab to 12.5 mg |
| | SC Q4W |
| | Increase dose of Clazakizumab back up to |
| | 25 mg SC Q4W if circumstances allow or |
| | continue 12.5 mg SC Q4W. |
| <1,000 | Stop Clazakizumab. |
| (CTCAE Grade ≥3) | |

Platelets (cells per mm³)

| | |
|---|---|
| ≥75,000-LLN | Reduce dose of MMF/MPA/AZA by 50% |
| (CTCAE Grade 1) | No change to Clazakizumab dose. |
| <75,000-50,000 | Reduce dose of MMF/MPA/AZA by 50%. |
| (CTCAE Grade 2) | Reduce dose of Clazakizumab to 12.5 mg |
| | SC Q4W. |
| | Increase dose of Clazakizumab back up to |
| | 25 mg SC Q4W if circumstances allow or |
| | continue |
| <50,000 | Stop Clazakizumab. |
| (CTCAE Grade ≥3) | |

Notes:
ALT = Alanine aminotransferase; AST = Aspartate aminotransferase; AZA = Azathioprine; CTCAE = Common Terminology Criteria for Adverse Events; INR = International normalized ratio; LFT = Liver function test; LLN = Lower limit of normal; MMF = Mycophenolate mofetil; MPA = Mycophenolic acid; Q4W = Once every 4 weeks; SC = Subcutaneous; ULN = Upper limit of normal.

Also monitoring of CNI levels is conducted throughout the clinical regimen. Also, CNIs are monitored every 2 weeks following a change in dose of Clazakizumab/discontinuation of Clazakizumab (or change in CNI dose) until target CNI trough levels are achieved.

### Monitoring for BKV, CMV, and EBV Infection

During treatment, monitoring for BKV, CMV, and EBV infection is performed by PCR test at Screening and every 8 to 12 weeks thereafter. If PCR DNA test becomes positive (i.e., exceeds the lower limit of quantitation) or viral load increases, Clazakizumab discontinuation or dose-reduction (to 12.5 mg SC Q4W) may be effected. Clazakizumab may be discontinued for BKV, CMV, or EBV infections that meet the criteria (see Table 2). Table 2 provides further guidelines for dose adjustment of Clazakizumab and/or background immunosuppression according to the viral load as detected by the PCR test. Decisions regarding dose modification are made in consultation with the treating clinician.

TABLE 2
Modification of Dose of Clazakizumab and/or Background
Immunosuppression Based on Results of Monitoring
for BKV, CMV, and EBV Infection

| | |
|---|---|
| | Modification of Dose of Clazakizumab |
| Parameter | and/or Background Immunosuppression |

BKV

| | |
|---|---|
| BKV >LLOQ to | Reduce dose of MMF/MPA/AZA by 50% or |
| <1,000 copies/mL | reduce CNI target trough levels (i.e., |
| | cyclosporine: 25-75 ng/mL; tacrolimus: |
| | 4-6 ng/mL). |
| | No change to Clazakizumab drug dose. |
| | Repeat PCR test every 2 weeks. |
| BKV ≥1,000 to | Reduce dose of MMF/MPA/AZA by 50% |
| <10,000 copies/mL | and/or reduce CNI target trough levels |
| | (i.e., cyclosporine: 25-75 ng/mL; tacrolimus: |
| | 4-6 ng/mL). Reduce Clazakizumab to 12.5 mg |
| | SC Q4W or consider stopping Clazakizumab |
| | depending on severity of infection. |
| | Repeat PCR test every 2 weeks. |
| | Increase Clazakizumab dose back up to 25 |
| | mg SC Q4W if circumstances allow. |
| BKV ≥10,000 | Stop Clazakizumab. (Adjustment of MMF/ |
| copies/mL or | MPA/AZA dose and CNI levels at |
| biopsy-proven BKV | Investigator's discretion.) |
| nephropathy | |

CMV

| | |
|---|---|
| CMV >LLOQ to <1,000 | No change to Clazakizumab dose. Repeat PCR |
| IU/mL | test weekly. |
| CMV ≥1,000 IU/mL | Treat with oral valganciclovir or IV |
| to <5,000 IU/mL | ganciclovir. Repeat PCR test weekly. |
| CMV ≥5,000 IU/mL | Reduce dose of MMF/MPA/AZA by 50% |
| | and/or reduce CNI target trough levels |
| | (i.e., cyclosporine: 25-75 ng/mL; tacrolimus: |
| | 4-6 ng/mL). Treat with oral valganciclovir |
| | or IV ganciclovir. |
| | Repeat PCR test weekly. |
| | Reduce Clazakizumab to 12.5 mg SC Q4W or |
| | consider stopping Clazakizumab depending |
| | on severity of infection. |
| | Increase Clazakizumab dose back up to 25 |
| | mg SC Q4W if circumstances allow. |
| CMV end-organ | Stop Clazakizumab. (Adjustment of MMF/ |
| disease (e.g., | MPA/AZA dose and CNI levels at |
| hepatitis, colitis, | Investigator's discretion.) |
| pneumonitis, | |
| retinitis) | |

EBV

| | |
|---|---|
| EBV >LLOQ | Reduce dose of MMF/MPA/AZA by 50% |
| to <10,000 | and/or reduce CNI target trough levels |
| copies/mL | (i.e., cyclosporine: 25 75 ng/mL; tacrolimus: |
| | 4-6 ng/mL). Repeat PCR test every 2 weeks. |
| EBV ≥10,000 | Stop Clazakizumab. (Adjustment of MMF/ |
| copies/mL or | MPA/AZA dose and CNI levels at |
| post-transplant | Investigator's discretion.) |
| lymphoproliferative | |
| disorder or primary | |
| EBV infection in | |
| seronegative | |
| recipient | |

Notes:
AZA = Azathioprine; BKV = Polyoma BK virus; CMV = Cytomegalovirus; CNI = Calcineurin inhibitor; EBV = Epstein-Barr virus; IU = International units; IV = Intravenous; LLOQ = Lower limit of quantitation; MMF = Mycophenolate mofetil; MPA = Mycophenolic acid; PCR = Polymerase chain reaction; Q4W = Once every 4 weeks; SC = Subcutaneous.

In general, in cases where Clazakizumab is reduced to 12.5 mg SC Q4W, it should be continued at the reduced dose for 1 or 2 doses and PCR test monitoring performed before increasing Clazakizumab dose back to 25 mg SC Q4W. Restoring the Clazakizumab dose back to 25 mg SC Q4W is effected first, before restarting/increasing MMF/MPA/AZA or increasing CNI levels. Also monitoring of CNI levels is conducted throughout treatment. In addition, CNIs are monitored every 2 weeks following a change in CNI dose or change in dose of Clazakizumab/discontinuation of Clazakizumab, until target CNI trough levels are achieved.

### Prohibited Therapy and Concomitant Treatments

No other treatments for ABMR (including CABMR) and TCMR are effected during treatment. For patients who received these treatments at any time before the 3-month pre-screening period these subjects must have a renal biopsy performed after halting treatment in order to confirm eligibility per the inclusion criteria below.

The following substances are generally excluded during treatment:
1. Rituximab
2. Eculizumab
3. Proteasome inhibitors
4. IVIG, except for treatment of hypogammaglobulinemia
5. PLEX
6. Belatacept
7. Other Clazakizumabs/treatments
8. Anti-IL-6/IL-6R receptor mAbs (both approved and investigational)

### Allowed Concomitant Medications

The following concomitant medications are permitted during 11-6 Ab treatment:
1. AZA Recommended AZA dose: 1.0-2.0 mg/kg/day (however in case of neutropenia/thrombocytopenia or viral infection, the dose of AZA may be reduced as indicated in Table 1 and Table 2).
2. CNIs
3. Recommended target tacrolimus plasma trough levels: 5-8 ng/mL
4. Recommended target cyclosporine plasma trough levels: 50-150 ng/mL
5. In case of viral infection, the CNI target level may be modified as indicated in Table 2.
6. CNI trough levels are monitored at Day 1, and at 1 week and 4 weeks after the first dose of Clazakizumab; and then every 4 weeks up to Week 12; and then every 8 weeks thereafter during treatment. CNIs are also to be monitored every 2 weeks following a change in CNI dose or change in dose of Clazakizumab/discontinuation of Clazakizumab, until target CNI levels achieved.
7. MMF/MPA (Recommended MMF dose: 1.0-2.0 g/day); Recommended MPA dose: 720-1,440 mg/day; however in case of neutropenia/thrombocytopenia or viral infection, the dose of MMF/MPA may be reduced.
8. mTOR inhibitors (everolimus, sirolimus).
9. Low dose corticosteroids (prednisone/prednisolone 0 mg/day).
10. Anti-hypertensive agents (e.g., angiotensin converting enzyme inhibitors (ACEIs), angiotensin II receptor blockers (ARBs)) (ACEIs and ARBs should be started and dose stabilized for at least 2 months prior to screening visit).
11. Antidiabetic agents.
12. Treatment for acute TCMR allowed: pulse steroid (e.g., oral prednisone 200 mg/day) and taper to baseline level over 2 weeks.
13. Treatment with oral valganciclovir or IV ganciclovir for CMV infection.
14. Trimethoprim/sulfamethoxazole or inhaled pentamidine or oral dapsone
15. In general, the use of herbal and homeopathic medicines (e.g., St. John's Wort, echinacea, goldenseal, Schisandra sphenanthera extracts) is discouraged.

### Prophylactic Therapy

Subjects generally will take prophylactic treatment for PJP. Oral trimethoprim/sulfamethoxazole generally will be prescribed. If the subject is already on trimethoprim/sulfamethoxazole prior to treatment, the dose should be stabilized for at least 1 week prior to the screening visit. If a subject is not on trimethoprim/sulfamethoxazole prior to treatment (and is not already receiving inhaled pentamidine or oral dapsone), trimethoprim/sulfamethoxazole generally is started at least 1 week before the Day 1 Baseline visit (Visit 2).

If subject is already receiving inhaled pentamidine or oral dapsone for PJP prophylaxis, subject should remain on these drugs and not start trimethoprim/sulfamethoxazole. Subjects who are intolerant to trimethoprim/sulfamethoxazole and not already receiving inhaled pentamidine or oral dapsone is generally started on either one of these drugs at least 1 week before treatment is commenced.

### Infection

Clazakizumab may reduce immune response to infections, therefore clazakizumab generally should not be administered to subjects with active bacterial, viral, or fungal infections, or subjects who meet certain laboratory criteria that could predispose subjects to infections (e.g., low absolute neutrophil count). Accordingly clinicians during treatment should look for any signs or symptoms of infection. Infections should be monitored and treated according to standard of care; for serious and opportunistic infections, Investigators should consider withholding and/or discontinuing treatment with clazakizumab and/or reducing background immunosuppression. Decisions regarding dose modification should be made in consultation with the treating clinician.

### Viral Monitoring

During treatment, routine monitoring for BKV, CMV, and EBV infection generally will be performed by PCR tests at initial Screening and every 8-12 weeks thereafter. In the event of positive results, modification of the dose of Clazakizumab and/or background immunosuppression may be considered. These guidelines may be followed for any other clinically significant infection.

### Liver Function

Treatment with clazakizumab may elevate transaminases. Accordingly subjects with evidence of significant liver disease and significant alcohol or illegal drug use are generally excluded from Claza treatment. During treatment liver function tests and hepatobiliary AEs are closely monitored. Also during treatment, routine monitoring of LFTs is performed at Screening and every 4-12 weeks thereafter. In the case of mild to moderate LFT abnormalities, the dose of clazakizumab may be modified, and in the case of severe LFT abnormalities (CTCAE Grade treatment with clazakizumab is generally discontinued. To ensure subject safety, the most recent LFTs generally are reviewed prior to Clazakizumab dosing.

### Hematology Parameters

Treatment with clazakizumab has been associated with decreased numbers of platelets and neutrophils, accordingly platelet and neutrophil numbers are monitored during treatment. During treatment a CBC is performed when treatment is started and every 4-12 weeks thereafter. In the case of mild to moderate neutropenia or thrombocytopenia, the dose of clazakizumab and/or background immunosuppression may be modified, and in the case of severe neutropenia or thrombocytopenia (CTCAE Grade treatment with clazakizumab may be discontinued (see Table 1). To ensure subject safety, the most recent neutrophil and platelet results generally are reviewed prior to Claza dosing.

### Dyslipidemia

Treatment with clazakizumab has been associated with dyslipidemia. Accordingly, routine monitoring of lipid levels is generally performed for the subjects being treated with clazakizumab.

### Gastrointestinal Perforation

Three cases of GI perforation were seen in a study of subjects with Crohn's Disease who were given high doses of clazakizumab (i.e., 150 mg IV, 300 mg IV/100 mg SC, and 600 mg IV). Based thereon, transplant recipients patients with inflammatory bowel disease, diverticular disease or history of GI perforation will generally not be treated with clazakizumab.

### Malignancies

Malignancies are known risks associated with prolonged immunosuppression. Malignancies are identified as a potential risk for therapies that modulate the immune system and generally should be monitored during clazakizumab treatment.

### Autoimmunity

Development of certain autoimmune disorders has been associated with some biologic therapies for RA. Accordingly signs of autoimmunity generally will be monitored during the subject clazakizumab treatment.

### Immunogenicity

The development of anti-drug antibodies (ADAs) is associated with many therapeutic Abs. Such antibodies can lead to reduced efficacy or have safety consequences. To date, ADAs have not been detected in healthy volunteers treated with clazakizumab. By contrast ADAs have been detected in some subjects with RA and PsA treated with Claza. Accordingly the presence of anti-clazakizumab antibodies generally will be monitored during the instant Claza treatment.

### Drug Interactions

No formal clinical drug interaction studies of clazakizumab have been performed. However, in vitro studies have shown that clazakizumab has a similar effect to TCZ in reversing the IL-6 effect on the down-regulation of mRNA levels of multiple CYP enzymes. Therefore, treatment with clazakizumab may restore CYP enzyme-mediated drug clearance, resulting in a potential lowering of systemic exposure of drugs metabolized by CYP enzymes, as has been observed with TCZ. This effect could be particularly important for CYP enzyme substrate drugs that have a narrow therapeutic index where the dose is individually adjusted. Accordingly, caution may be exercised when co-administering clazakizumab with CYP3A4 substrate drugs where a decrease in effectiveness is undesirable (e.g., oral contraceptives, 3-hydroxy-3-methyl-glutaryl-co-enzyme A reductase inhibitors).

Also given the potential for drug-drug interactions between clazakizumab and CNIs, CNI trough levels generally are monitored; e.g., at Day 1, and at 1 week and 4 weeks after the first dose of Clazakizumab; and then every 4 weeks up to Week 12; and then every 8 weeks thereafter for the remainder of the study. CNIs also may be monitored every 2 weeks following a change in CNI dose or change in dose of Clazakizumab/discontinuation of Clazakizumab, until target CNI trough levels are achieved.

### Overdose

There are no specific antidotes or measures to take in the event of an overdose of clazakizumab injection. Subjects should be treated with the appropriate supportive care.

### Injection Site Events and Infusion Related (Allergic) Reactions

Injection site reactions (ISRs) have been reported with SC administration, and most frequently reported as erythema. Reactions have been mild or moderate and have resolved without treatment. To date, no infusion reactions have been associated with clazakizumab administered by IV infusion.

As with any protein therapeutic, there is a risk of a serious allergic reaction (infusion reaction). Clazakizumab generally should not be administered to subjects who have had any previous allergic reactions to mAbs. Both allergic reactions and ISRs should be treated with standard of care. Subjects who have developed significant allergic reaction to Clazakizumabs generally should not be rechallenged.

### Clinical Laboratory Assessments

Patient blood samples generally are analyzed using standard validated methods. Blood and urine samples for the following efficacy and safety assessments generally will be drawn in each of Years 1-5 and later if applicable. Blood and urine samples generally will be collected prior to dosing at the clinic visit. A summary of such laboratory assessments is provided in Table 3.

TABLE 3
Summary of Laboratory Assessments

| Test Type | Test Parameters | Collection |
|---|---|---|
| Clinical | BUN | At Visit 1 (Screening), Visits |
| Chemistry | Chloride | 2, 4, 5, 6, 8, 10, 12, 14, 16, |
| (scrum) | CO₂ | 19, 22, 25, 28, 31, 34, 37, 40, |
| | Creatinine | 43, 46, 49, 52, 55, 58, 61, 64, |
| | Liver function tests | 67, and 68 (EOS). |
| | (AST, ALT, alkaline | |
| | phosphatase, GGT, | |
| | total bilirubin, | |
| | direct bilirubin, INR) | |
| | hsCRP | |
| | Potassium | |
| | Sodium | |
| | Fasting glucose⁽¹⁾ | At Visits 2, 4, 5, 6, 8, 10, 12, |
| | | 14, 16, 19, 22, 25, 28, 31, 34, |
| | | 37, 40, 43, 46, 49, 52, 55, 58, |
| | | 61, 64, 67, and 68 (EOS). |
| Liquid | Cholesterol | At Visits 2, 4, 5, 6, 8, 10, 12, |
| (serum) | (total, HDL, and | 14, 16, 19, 22, 25, 28, 31, 34, |
| | LDL), fasting | 37, 40, 43, 46, 49, 52, 55, 58, |
| | Triglycerides, | 61, 64, 67, and 68 (EOS). |
| | fasting | |
| Hematology | CBC | At Visit 1 (Screening), Visits |
| | Hb | 2, 4, 5, 6, 8, 10, 12, 14, 16, |
| | Hematocrit | 19, 22, 25, 28, 31, 34, 37, 40, |
| | Red blood cell | 43, 46, 49, 52, 55, 58, 61, 64, |
| | count | 67, and 68 (EOS). |
| | White blood cell | |
| | count | |
| | White blood cell | |
| | differential | |
| | (absolute and %) | |
| | Platelet count | |
| Serology | HIV virus | At Visit 1 (Screening) (unless |
| | HBsAg | known seropositive history). |
| Viral | Hepatitis B DNA | At Visit 1 (Screening). |
| monitoring | Hepatitis C RNA | |
| (PCR) | BKV, CMV, EBV, | At Visit 1 (Screening), Visits |
| | DNA | 6, 8, 10, 12, 14, 16, 19, 22, 25, |
| | | 28, 31, 34, 37, 40, 43, 46, 49, |
| | | 52, 55, 58, 61, 64, 67, and 68 |
| | | (EOS). |
| Urinalysis | Dipstick chemical | Screening |
| | profile with optional | |
| | microscopic profile | |
| | if dipstick is abnormal | |
| | UACR spot urine | At Visit 1(Screening), Visits |
| | test (UPCR may be | 6, 9, 15, 21, 27, 33, 39, 45, 51, |
| | substituted at Visit 1) | 57, 63 and 68 (EOS). |
| | 24-hour urine | At Visit 1 (Screening), if required |
| Pregnancy | POCT urine | At all visits except Visit 3 |
| test | pregnancy test for | (Visits 1, 2 and 4 through 68). |
| | WOCBP (any positive | |
| | results are be | |
| | confirmed by a | |
| | serum test at the | |
| | central laboratory)⁽²⁾ | |
| Special test | eGFR (MDRD4) | At all visits except Visit 3 |
| | (See Section 9.1.1.1) | (Visits 1, 2 and 4 through 68). |
| | DSA MFI scores | At Visit 1 (Screening), Visits |
| | (for anti-HLA | 2 (Baseline), 6, 10, 16, 28, 40, |
| | antibodies) | 52, 64 and 68 (EOS). |
| | (See Section 9.1.1.2) | |
| | DSA titers (for | At Visit 1 (Screening), Visits |
| | anti-HLA antibodies) | 2 (Baseline), 6, 10, 16, 28, 40, |
| | (See Section 9.1.1.2) | 52, 64 and 68 (EOS). |
| | Plasma IL-6 (See | At Visits 2 (Baseline), 6, 9, 15, |
| | Section 9.1.1.3) | 21, 27, 33, 39, 45, 51, 57, 63, |
| | | and 68 (EOS). |
| | Plasma | At Visits 2 (Baseline), 6, 9, 15, |
| | clazakizumab (See | 21, 27, 33, 39, 45, 51, 57, 63, |
| | Section 9.1.1.4) | and 68 (EOS). |
| | Anti-clazakizumab | At Visits 2 (Baseline), 6, 9, 15, |
| | antibodies (See | 21, 27, 33, 39, 45, 51, 57, 63, |
| | Section 9.1.1.5) | and 68 (EOS). |
| | MPA levels (See | At Visits 2 (Baseline), 4, 5, 6, |
| | Section 9.1.1.6) | 8, 10, 12, 14, 16, 19, 22, 25, |
| | | 28, 31, 34, 37, 40, 43, 46, 49, |
| | | 52, 55, 58, 61, 64, 67, and 68 |
| | | (EOS). |
| | CNI levels (See | At Visits 2 (Baseline), 3 to 6, |
| | Section 9.1.1.7) | 8, 10, 12, 14, 16, 18, 20, 22, |
| | | 24, 26, 28, 30, 32, 34, 36, 38, |
| | | 40, 42, 44, 46, 48, 50, 52, 54, |
| | | 56, 58, 60, 62, 64, 66, and 68 |
| | | (EOS). |
| Serology | HIV virus | At Visit 1 (Screening) (unless |
| | HBsAg | known seropositive history). |
| Biomarkers | See Section 9.1.1.8 | At Visits 2 (Baseline), 6, 9, 15, |
| | | 21, 27, 33, 39, 45, 51, 59, 63, |
| | | and 68 (EOS). |

⁽¹⁾At least 10 hours fasting required. If the Subject failed to fast, samples should still be collected and processed with failure to fast recorded on the lab requisition and as a protocol deviation.
⁽²⁾POCT urinary pregnancy test prior to every dose of Clazakizumab. For subjects who discontinue treatment with clazakizumab/placebo, this assessment may be conducted for an additional 5 months following the last dose of Clazakizumab.
Notes:
ALT = Alanine aminotransferase; AST = Aspartate aminotransferase; BKV = Polyoma BK virus; BUN = Blood urea nitrogen; CBC = Complete blood count; CMV = Cytomegalovirus; CNI = Calcineurin inhibitor; DNA = Deoxyribonucleic acid; DSA = Donor- specific antibodies; EBV = Epstein Barr virus; eGFR = Estimated glomerular filtration rate; EOS = End of study; GGT = Gamma-glutamyl transferase; Hb = Hemoglobin; HBsAg = Hepatitis B surface antigen; HDL = High density lipoprotein; HIV = Human immunodeficiency virus; HLA = Human leukocyte antigen; hsCRP = High-sensitivity C-reactive protein; INR = International normalized ratio; IL-6 = Interleukin 6; LDL = Low density lipoprotein; MDRD4 = Modification of Diet in Renal Disase-4; MFI = Mean fluorescence intensity; MPA = Mycophenolic acid; PCR = Polymerase chain reaction; POCT = Point of care test; RNA = Ribonucleic acid; UACR = Urine albumin creatinine ratio; UPCR = Urine protein creatinine ratio; WOCBP = Women of childbearing potential.

eGFR

Estimated glomerular filtration rate generally will be determined using the MDRD4 equation:
eGFR=175×(serum creatinine [mg/dL])−1.154×(Age)−0.203×(0.742 if female; 1 otherwise)×(1.212 if black; 1 otherwise)

The eGFR generally is determined substantially every visit (Q4W) throughout treatment 3.

### DSA Titers and MFI Scores

DSAs generally will be determined using single-antigen bead-based assays.
1. MFI scores for HLA DSA generally are determined at Visit 1 (Screening), Visits 2 (Baseline), 6, 10, 16, 22, 28, 34, 40, 46, 52, 58, 64, and 68.
2. DSA titers generally are determined at Visit 1 (Screening), Visits 2 (Baseline), 6, 10, 16, 28, 40, 52, 64 and 68 (EOS).

At Screening, these results may be used for determination of DSA eligibility criteria. If presence of HLA DSA is confirmed within 6 months of screening, the test does not need to be repeated for eligibility.

### Plasma IL-6

Total IL-6 (ligand bound/unbound to soluble IL-6 receptor and bound/unbound to clazakizumab) and free IL-6 (ligand unbound to soluble IL-6 receptor and unbound to clazakizumab) levels generally may be measured using a validated SIMOA® single molecule array assay.

Plasma IL-6 levels (total and free) generally are measured at Visits 2 (Baseline), 6, 9, 15, 21, 27, 33, 39, 45, 51, 57, 63, and 68 (EOS).

### Plasma Clazakizumab

A validated enzyme-linked immunosorbent assay method generally is used to measure concentrations of clazakizumab in serum. Plasma clazakizumab levels generally is measured at Visits 2 (Baseline), 6, 9, 15, 21, 27, 33, 39, 45, 51, 57, 63, and 68 (EOS).

### Anti-clazakizumab Antibodies

A validated electrochemiluminescence immunoassay method generally is used to measure titers of clazakizumab antibodies in serum. Plasma anti-clazakizumab antibody levels generally may be measured at Visits 2 (Baseline), 6, 9, 15, 21, 27, 33, 39, 45, 51, 57, 63, and 68 (EOS).

### MPA Levels

MPA levels in serum/plasma may be measured, e.g., by a validated quantitative liquid chromatography-tandem mass spectrometry (LC-MS/MS) method. In general MPA levels may be measured at Visits 2 (Baseline), 4, 5, 6, 8, 10, 12, 14, 16, 19, 22, 25, 28, 31, 34, 37, 40, 43, 46, 49, 52, 55, 58, 61, 64, 67, and 68 (EOS). At these visits, prophylactic treatment with MMF/MPA generally is withheld until determination of MPA levels.

### CNI Levels

CNI (tacrolimus and cyclosporine) trough levels in serum/plasma may be measured, e.g., by a validated quantitative LC-MS/MS method. CNI trough levels e.g., may be measured at Visits 2 (Baseline), 3 to 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, and 68 (EOS). At these visits, prophylactic treatment with CNIs generally is withheld until determination of CNI levels.

CNIs also may be monitored every 2 weeks following a change in CNI dose or change in dose of Clazakizumab/discontinuation of Clazakizumab, e.g., until target CNI trough levels are achieved.

### Physical Examinations

Typically at Visit 1 (Screening), a complete physical examination as per standard of care generally is conducted by a physician. Additional abbreviated physical examinations further may be conducted; e.g., at Visit 2 (Baseline) and at each visit from Visit 4 (Week 4) to Visit 68 (Week 260). Generally the subject weight will be recorded at each physical examination.

### Vital Signs

Vital signs generally are measured at about every visit (Q4W) throughout the treatment. Generally these assessments are taken after the subject has been in a sitting position after 5 minutes of rest. To avoid variability, the same method of obtaining body temperature generally is used throughout treatment.

The following vital signs generally are measured:
1. Blood pressure (systolic and diastolic)
2. Heart rate
3. Body temperature (° C. or ° F.), axillary or tympanic
4. Respirations

### Standard 12-Lead Electrocardiogram

An electrocardiogram (ECG) which comprises a standard 12-lead tracing generally is performed which results are generally assessed by a qualified physician, and generally retained as a source document. Results including any abnormalities are to be recorded in the eCRF (electronic case report form).

Electrocardiograms generally will be recorded digitally after the subject has been in a resting, supine position for at least 5 minutes. Significant abnormalities, including findings that may prompt discontinuation of Clazakizumab should be evaluated. Typically electrocardiograms are performed at Visit 1 (Screening), and any or all of Visits 6, 9, 15, 21, 27, 33, 39, 45, 51, 57, 63, and 68 (EOS).

### Tuberculosis Screening

Screening for active and latent TB generally is required for assessment of subject eligibility for Claza treatment. The following procedures generally are required:
1. A complete physical exam and medical history to evaluate exposure to TB and whether subjects with a history of latent TB (without active TB) completed a full course of prophylactic treatment
2. Chest x-ray
3. QuantiFERON-TB® (QFT®) Gold interferon-γ release assay

Positive results for the interferon-γ release assay generally is not repeated. An indeterminate result may be repeated 1 time. If the second test is positive or indeterminate, the result generally is considered positive for that subject. A third test generally is not performed. Subjects who have newly diagnosed TB generally should have Clazakizumab discontinued or managed according to the appropriate standard of care.

### Renal Biopsy

Biopsy proven CABMR (according to Banff 2015 diagnostic criteria within 6 months of screening is generally required for subject eligibility for treatment. A repeat biopsy generally is performed if the previous biopsy is not within 6 months of screening. If the subject has received treatment for ABMR (including CABMR) or TCMR, a repeat biopsy (to show continuing CABMR) generally is performed.

Biopsy eligibility for entry into Claza treatment generally is based on the pathologist diagnosis and Banff scoring. Repeat biopsies per protocol may be performed at Visit 16 (Week 52). Unscheduled biopsies may be performed at any time if clinically indicated. If a for-cause biopsy has been performed within 2 months of Week 52, a repeat biopsy at Week 52 generally is not required.

### Screening Visit (Assessment Visit 1)

The screening visit (Visit 1) generally may take place within 28 days prior to Visit 2 (Baseline, Day 1). The initial screening assessment may include provision of informed consent; review of inclusion/exclusion criteria; complete physical examination; vital signs measurements, including weight and height; medical history (including historical serology for viral infections); a urine pregnancy test (for WOCBP); TB screening; 12-lead ECG; blood and urine sample collection for central laboratory assessments per SOE, eGFR, standard urinalysis; spot urine collection (for determination of UPCR/UACR); 24-hour urine collection (if necessary); serology for HIV and HBsAg if seronegative or history unknown; PCR monitoring for BKV, CMV, and EBV DNA and for HCV RNA; and review of prior and concomitant medications and entry criteria.

A minimum of 10 hours fasting is generally required for determination of fasting glucose and lipids/triglycerides. Any AEs which occur after informed consent but before Visit 2 generally are recorded as medical history.

Biopsy diagnosis to determine eligibility for entry into the treatment generally is based on the pathologist diagnosis and Banff scoring. At Screening, laboratory results may be used for determination of DSA eligibility criteria. If presence of HLA DSA is confirmed within 6 months of screening, the test generally does not need to be repeated. A subject determined to be a screen failure generally may be reevaluated once.

### Treatment Procedures

Throughout the treatment period (Visits 2 through 68), subjects are in general administered 25 mg of Claza subcutaneously Q4W. Prior to dosing, the following assessments generally are conducted at Visit 2 and Visits 4 through 67:
1. AEs and concomitant medications (are to be collected and recorded prior to the conduct of any other study assessments).
2. Abbreviated physical exam (including vital signs).
3. Pregnancy test for WOCBP.
4. Blood collection for central laboratory analysis of eGFR.
5. Review of the most recent LFT and CBC analyses and viral monitoring results from a prior visit (scheduled or otherwise) to confirm if the subject is eligible to receive Clazakizumab according to the safety limits for these criteria or if a modification of the dose of Clazakizumab and/or background immunosuppression is required/recommended (see Section 7.5).

Additional assessments may be conducted prior to dosing every 4 to 12 weeks, as detailed in the SOE, and may include the following:
1. A check of all inclusion/exclusion criteria (Visit 2 (Baseline, Day 1) only).
2. Blood and urine sample collection for additional central laboratory analyses as detailed in Table 7 and in the SOE. A minimum of 10 hours fasting are required for determination of fasting glucose and lipids/triglycerides. At relevant visits per the SOE, CNIs are to be withheld until after collection of the blood sample for determination of CNI trough levels.
3. Health-Related Quality of Life (HRQoL) questionnaires. At applicable visits, these questionnaires should be completed prior to any other assessments.
4. Renal biopsy (Visit 16; may be performed at an earlier visit if clinically indicated).

At Visit 3, typically the only assessment conducted comprises a blood sample collection for monitoring of CNI trough levels. As noted above, CNIs generally are to be withheld until after collection of the blood sample for determination of CNI trough levels.

### End of Treatment Procedures

On completion of treatment the following assessments may be effected:
1. AEs and concomitant medications (recorded prior to the conduct of other study assessments).
2. Abbreviated physical exam (including vital signs).
3. Pregnancy test for WOCBP.
4. Blood and urine sample collection for central laboratory analyses. A minimum of 10 hours fasting are required for determination of fasting glucose and lipids/triglycerides. CNIs are to be withheld until after collection of the blood sample for determination of CNI trough levels.
5. Renal biopsy (if subject withdrawal prior to Week 52).

### Follow-up Procedures

All subjects who are treated in general will be evaluated monthly to detect any new AEs, SAEs, or pregnancies. Subjects who discontinue Clazakizumab due to graft loss may undergo EOS assessments (Visit 68) and be followed with monthly TCs for 5 months after the last dose of Clazakizumab.

All withdrawn patients generally will complete the EOS assessments (Visit 68) and be followed with monthly TCs for 5 months after the last dose of Clazakizumab, if possible and may be called in for a clinic visit at the discretion of a physician.

### Unscheduled Visits

Unscheduled visits may be performed during the course of treatment for safety reasons. Also subjects who discontinue Clazakizumab may be seen in the clinic for an unscheduled visit.

### Definition of Adverse Event

An AE is defined as any untoward medical occurrence or worsening of a pre-existing medical condition in a clinical investigation subject administered Clazakizumab and that does not necessarily have a causal relationship with this treatment. An AE can therefore be any unfavorable and unintended sign (such as an abnormal laboratory finding), symptom, or disease temporally associated with the use of Clazakizumab, whether or not considered related to the Clazakizumab.

A treatment-emergent AE (TEAE) is defined as any event not present prior to exposure to Clazakizumab or any event already present that worsens in either intensity or frequency following exposure to Clazakizumab.

### Definition of Adverse Drug Reaction

In the pre-approval clinical experience with a new medicinal product or its new usages, particularly as the therapeutic dose(s) may not be established, all noxious and unintended responses to a medicinal product related to any dose generally may be considered adverse drug reactions (ADRs). The phrase “responses to a medicinal product” means that a causal relationship between a medicinal product and an adverse event is at least a reasonable possibility, i.e., the relationship cannot be ruled out.

### Definition of Unexpected Adverse Events/Adverse Drug Reactions

An AE/ADR may be considered unexpected if the nature, severity, or frequency of the event is not consistent with the risk information previously described for the study agent. Identified and potential risks for clazakizumab are described herein.

### Definition of Serious Adverse Event

An SAE is defined as any AE or suspected adverse reaction that in general, results in any of the following outcomes:
1. Death.
2. A life-threatening AE. (Note: the term life-threatening definition of an SAE refers to an event in which the subject was at risk of death at the time of the event; it does not refer to an event which hypothetically might have caused death if it was more severe.)
3. Inpatient hospitalization or prolongation of existing hospitalization.
4. A persistent or significant incapacity or substantial disruption of the ability to conduct normal life functions.
5. Congenital anomaly/birth defect.
6. Important medical events (see below).

Important medical events that may not be life-threatening, nor require hospitalization, nor result in death may be considered serious when, based upon appropriate medical judgment, they may jeopardize the subject and may require medical or surgical intervention to prevent one of the outcomes listed in this definition. Examples of such medical events include allergic bronchospasm requiring intensive treatment in an emergency room or at home, blood dyscrasias or convulsions that do not result in inpatient hospitalization, or the development of drug dependency or drug abuse.

The following hospitalizations generally are not considered SAEs herein:
1. A visit to the emergency room or other hospital department for <24 hours, that does not result in admission (unless considered an important medical or life-threatening event).
2. Elective surgery, planned prior to signing informed consent for this study.
3. Admissions as per protocol for a planned medical/surgical procedure (e.g., renal biopsy).
4. Routine health assessment requiring admission for baseline/trending of health status (e.g., routine colonoscopy).
5. Medical/surgical admission other than to remedy ill health and planned prior to entry into the study. Appropriate documentation is required in these cases.
6. Admission encountered for another life circumstance that carries no bearing on health status and requires no medical/surgical intervention (e.g., lack of housing, economic inadequacy, caregiver respite, family circumstances, administrative reason).

### Definition of Suspected Unexpected Serious Adverse Reaction

A SUSAR is defined as any ADR that is both serious and unexpected, and that is considered to have a reasonable suspected causal relationship to Clazakizumab.

### Definition of Adverse Events of Special Interest (AESI)

Adverse events of special interest (AESIs) are AEs of scientific or medical concern for which ongoing monitoring and rapid communication is important. These may include events that are either specific to the Clazakizumab or events that, in general, may be of clinical significance to the treatment. As such, an AESI may or may not be related to Clazakizumab. For clazakizumab, the following AESIs have been defined: LFT abnormalities, neutropenia, thrombocytopenia, hyperlipidemia, GI perforations, hypersensitivity and anaphylaxis, malignancy, opportunistic infections, and pregnancy. Each of these AESIs is discussed herein.

### LFT Abnormalities, Neutropenia, Thrombocytopenia, and Hyperlipidemia

Throughout Claza treatment, subjects generally will undergo regular hematology and biochemical laboratory tests to monitor for abnormal LFTs, neutropenia, thrombocytopenia, and hyperlipidemia. In general, these and any other abnormal test results that are Grade 3 (severe) or higher should be considered an AESI and stopping Clazakizumab treatment potentially should be considered.

Specifically, Clazakizumab treatment may be stopped for subjects who meet any of the following criteria which generally are considered AESIs:
1. AST/ALT >5×ULN
2. Total bilirubin >3×ULN
3. AST/ALT >3 to ≤5×ULN and total bilirubin ≥2×ULN (or INR>1.5)
4. Neutrophil count <1,000 cells per mm³
5. Platelets <50,000 cells per mm³

Also the following total cholesterol and triglyceride levels are considered AESIs:
1. Total cholesterol >400 mg/dL or >10.34 mmol/L, irrespective of baseline level
2. Triglyceride >500 mg/dL or >5.7 mmol/L, irrespective of baseline level

### Gastrointestinal Perforation

Gastrointestinal perforations are identified risks of treatment with anti-IL-6 antibodies and are reported as an AESI.

### Hypersensitivity and Anaphylaxis

Hypersensitivity reactions and anaphylaxis reactions, e.g., those meeting the definition of the Joint NIAID/FAAN Second Symposium on Anaphylaxis generally are considered AESI:
1. Acute onset of an illness (minutes to several hours) with involvement of the skin, mucosal tissue, or both (e.g., generalized hives, pruritus or flushing, swollen lips-tongue-uvula) and at least one of the following
2. Respiratory compromise (e.g., dyspnea, wheeze-bronchospasm, stridor, reduced peak expiratory flow, hypoxemia)
3. Reduced blood pressure or associated symptoms of end-organ dysfunction (e.g., hypotonia (collapse), syncope, incontinence)

### Malignancy

Any new malignancy or progression of pre-existing malignancy (excluding non-melanoma skin cancers (squamous cell or basal cell carcinoma)) typically are considered AESIs.

### Opportunistic Infections

Throughout Claza treatment, monitoring for potential infections typically will be effected according to the recommendations of the American Society of Transplantation and/or the KDIGO guideline. Recognized viruses that may cause significant morbidity in kidney transplant recipients include BKV, CMV and EBV and the presence thereof typically will be monitored by PCR, e.g., at regular 8- to 12-week intervals.

The following infections are considered AESIs:
1. Any bacterial pneumonia or bronchitis
2. Any gram-negative bacteria GI infections (including Salmonella (enterica serotypes, Typhimurium and Enteritidis), Shigella, Campylobacter, Escherichia coli, and Clostridium difficile)
3. BKV nephropathy
4. CMV infections/disease
5. Cryptosporidiosis with Cryptosporidium
6. Invasive candidiasis
7. Invasive mycosis which includes cryptococcosis, histoplasmosis, aspergillosis and coccidioidomycosis
8. JC virus infection (progressive multifocal leukoencephalopathy)
9. Hepatitis B virus (HBV) and HCV infections
10. Human papillomavirus (HPV) disease
11. HIV infection
12. Pneumocystis pneumonia with Pneumocystis jiroveci
13. Mycobacterium tuberculosis infections and other mycobacterium infections (e.g., Mycobacterium kansasii, Mycobacterium avium)
14. Non-CMV disease including herpes simplex virus Type 1 (HSV-1) and Type 2 (HSV-2) disease, varicella-zoster virus disease, human herpesvirus-8 (HHV-8) disease
15. Toxoplasmosis infections with Toxoplasma gondii

The above list is not meant to be complete and other infections which are not commonly observed in the kidney transplant population may also be monitored.

Clazakizumab treatment may be stopped for subjects who meet any of the following criteria and these abnormalities are considered AESIs:
1. BKV 0,000 copies/mL or biopsy-proven BKV nephropathy
2. CMV end-organ disease (e.g., hepatitis, colitis, pneumonitis, retinitis)
3. EBV 0,000 copies/mL or post-transplant lymphoproliferative disorder or primary EBV infection in seronegative recipient

### Pregnancy

Any pregnancy occurring in a female subject or female partner of a male subject during treatment or for 5 months after the last dose of Clazakizumab should be considered an AESI and recorded/reported on the special pregnancy form. In the event of a pregnancy, the subject generally should discontinue Clazakizumab treatment.

### Classification of Adverse Events

### Intensity/Severity Classification

The severity of all AEs typically is assessed and graded according to the National Cancer Institute's CTCAE Version 5.0 (see Table 4).

TABLE 4
AE Severity Grading

| Grade (Severity) | Description |
|---|---|
| Grade 1 (mild) | Asymptomatic or mild symptoms; clinical |
| | or diagnostic observations only; |
| | intervention not indicated. |
| Grade 2 (moderate) | Minimal, local or non-invasive |
| | intervention indicated; limiting age- |
| | appropriate instrumental activities of |
| | daily living (e.g., preparing meals, |
| | shopping for groceries or clothes, using |
| | the telephone, managing money, etc.). |
| Grade 3 (severe) | Severe or medically significant but not |
| | immediately life-threatening; |
| | hospitalization or prolongation of |
| | hospitalization indicated; disabling; |
| | limiting self-care activities of daily |
| | living (e.g., bathing, dressing and |
| | undressing, feeding self, using the |
| | toilet, taking medications, and not |
| | bedridden). |
| Grade 4 (life- | Life-threatening consequences; urgent |
| threatening) | intervention indicated. |
| Grade 5 (death) | Death related to AE. |

Note:
AE = Adverse event.
Source: National Cancer Institute's Common Terminology Criteria for Adverse Events (CTCAE) Version 5.0 [42].

The term severe is often used to describe the intensity (severity) of a specific event; however, the event itself may be of relatively minor medical significance (e.g., a severe headache). This is not the same as “serious”, which is based on the subject/event outcome or action criteria.

### Relationship to Clazakizumab

For all collected AEs, the clinician who examines and evaluates the patient will generally determine the AE's causality based on temporal relationship and his/her clinical judgment. The degree of certainty about causality generally are graded using 2 categories (related/unrelated) as shown in Table 5.

TABLE 5
AE Relationship to Clazakizumab

| Term | Relationship | Description |
|---|---|---|
| Related | Yes | The temporal relationship of the |
| | | clinical event to Clazakizumab |
| | | administration indicates a causal |
| | | relationship, and other drugs, |
| | | therapeutic interventions or |
| | | underlying conditions do not |
| | | provide a sufficient explanation |
| | | for the observed event. |
| Unrelated | No | The temporal relationship of the |
| | | clinical event to Clazakizumab |
| | | administration does not indicate |
| | | a causal relationship, or other |
| | | drugs, therapeutic interventions |
| | | or underlying conditions provide |
| | | a sufficient explanation for the |
| | | observed event. |

Note:
AE = Adverse Event.

### Outcome Categorization

Outcome of AEs in general should be classified as follows: recovered/resolved (i.e., without sequelae); recovered/resolved with sequelae; recovering/resolving; not recovered/not resolved; fatal; or unknown (if follow-up is not possible).

### Pre-Existing Medical Condition

A pre-existing medical condition is one that is present prior to treatment (unless the event is an SAE). A pre-existing medical condition should be recorded as an AE only if the frequency, severity, or character of the condition worsens during the study.

### Symptoms of the Disease Under Study

Signs and symptoms of the disease under study (CAMBR or AMBR) generally are not classified as AEs as long as they are within the normal day-to-day fluctuation of the disease. Worsening of disease symptoms, however, may be classified as an AE.

### Clinical Laboratory Evaluations

A change in the value of a safety laboratory investigation may be reported as an AE if the change is considered clinically relevant, or if during treatment with Claza, a shift in a laboratory parameter from a normal to a pathological value is observed, or a further worsening of an already pathological value is observed.

### Physical Examinations and Vital Signs

Worsening of any physical examination findings or vital signs, or any new physical examination findings may be reported as an AE if the change is considered clinically relevant, or in the case of vital signs, if a shift from a normal to a pathological value is observed.

### Reporting of Adverse Events

If any AE is reported, the date of onset, relationship to Clazakizumab, any action taken, date of resolution (or the fact that it is still continuing or has become chronic), outcome, intensity (worst at any point during the event) and whether the AE was serious or not at any time during the event may be recorded. In order to establish the duration of any SAE, the dates of hospitalization and discharge or dates of meeting other SAE criteria may be recorded.

The AE reporting period generally will begin at the time the informed consent form (ICF) is signed by the subject and continues until the end of treatment or until the follow-up period 5 months after the last dose of Clazakizumab. If the subject reports an AE, in general the clinician will acquire sufficient information in order to assess causality. This may require additional laboratory testing, physical examinations, telephone contacts, etc.

In general SAEs are to be followed until satisfactory resolution or until the site clinician deems the event to be chronic or stable, or the subject is lost to follow-up. The onset date of the SAE is generally defined as the date the signs and symptoms/diagnosis became serious. The resolution date of the SAE is defined as when the symptoms resolve, or the event is considered chronic or stable, and/or if the seriousness criteria are no longer applicable.

### Reporting of Pregnancy

Any pregnancy occurring in a female subject, or in the female partner of a male subject during the study or for 5 months after the last dose of Clazakizumab, should be reported to the clinician. The clinician should counsel the subject (or in the case of a male subject, the subject's partner) and discuss the risks of continuing with the pregnancy and any possible effects on the fetus. Monitoring of the pregnancy in a female subject should continue until conclusion of the pregnancy. Women who have a confirmed positive pregnancy test during treatment generally should be permanently discontinued from Clazakizumab.

Pregnancy in and of itself is not an SAE. However, complications of pregnancy such as abortion (spontaneous or induced), premature birth, or congenital abnormality are considered SAEs and generally should be documented.

### Data Analysis

Table 6 and Table 7 below provide sample size estimates and predicted data analysis.

TABLE 6
Sample Size Estimates for All-Cause Graft Failure Based on eGFR
Data Restricted to 52 Weeks Post-Diagnosis of Active ABMR

| | All-Cause | | |
|---|---|---|---|
| | Graft | | Total |
| | Survival at | | Number of |
| | 5-Years | | Events/Total |
| | Post-Diagnosis | | Number of |
| | of Active | Hazard | Subjects |
| Scenario | ABMR | Ratio, λ | Power = 80% |
| Mean eGFR | 0.233 | 1.000 | — |
| 50% improvement | 0.368 | 0.686 | 221/316 |
| in slope⁽¹⁾ | | | |

2 Relative to the average slope change for the mean eGFR scenario, slope = −0.753, based on eGFR data restricted to first 12 months post-diagnosis of ABMR.
Notes:
ABMR = Antibody-mediated rejection; eGFR = Estimated glomerular filtration rate.
Source: Modeling Report, Section 10, Table 9-2-1b [36].

The interim efficacy analysis may be performed when approximately 200 (100 per group) subjects have been randomized and received at least 52 weeks of treatment with Clazakizumab to evaluate the difference between the treatment groups. As shown in Table 6, a fixed sample size of 180 subjects (90 per group) will have 90% power (two-sided alpha of 0.05) to detect a minimum difference in the 52-week eGFR of 4.515 mL/min/1.73 m² between the treatment groups (assuming eGFR declines at a rate of 0.75 mL/min/1.73 m²/month in the placebo treated group and that clazakizumab reduces eGFR decline by 50%). The sample size determination for the fixed design is based on a two-sided alpha of 0.05 and a common standard deviation of 9.252 mL/min/1.73 m² for the mean eGFR change from Baseline to Week 52 (effect size=4.515/9.252=0.488). The planned sample size has been increased to a minimum of 200 subjects to allow 10% for subjects lost to follow-up or withdrawals.

TABLE 7
Sample Size Estimates for Change in eGFR (mL/min/1.73
m²) at 52 Weeks Post-Diagnosis of Active ABMR

| | | | % | Slope | |
|---|---|---|---|---|---|
| | | eGFR | Change | (eGFR | |
| | eGFR | 52 | from | Change/ | Power = |
| Scenario | Baseline | Weeks | Baseline | Month) | 90%⁽¹⁾ |
| Mean eGFR | 45.577 | 36.547 | −19.8% | −0.753 | |
| 50% improvement | 45.577 | 41.062 | −9.9% | −0.376 | 180 |
| in slope(2) | | | | | |

⁽¹⁾Standard deviation of the difference in eGFR from baseline at 12 months post-diagnosis of ABMR = 9.252, when restricted to 0-12 month eGFR data.
(2)Relative to the average slope change for the mean eGFR scenario, slope = −0.753, based on eGFR data restricted to first 12 months post-diagnosis of ABMR.
Notes:
ABMR = Antibody-mediated rejection; eGFR = Estimated glomerular filtration rate.
Source: Modeling Report, Section 10, Table 9-2-1a [36].

Once at least 100 subjects (50 per group) have received Clazakizumab for at least 52 weeks, re-estimation of the planned sample size of 200 subjects may be conducted using the inverse normal method with pre-specified information rates (0.5556, 1) to control the Type I error rate. The sample size re-estimation ensures a power of 95.9%, when the assumed eGFR effect size is 0.488. The average sample size under these assumptions is 202 evaluable subjects (corresponding to approximately 224 enrolled subjects, assuming 10% loss to follow-up or withdrawals). When the assumed eGFR effect size is 0.368, then the power is 79.6% and the average sample size is 218 evaluable subjects (approximately 242 enrolled subjects). The sample size for the interim efficacy analysis surrogate endpoint generally will not exceed a total of 250 evaluable subjects (approximately 280 enrolled subjects).

### Therapeutic Efficacy Endpoint

The primary efficacy endpoint herein generally comprises the composite clinical endpoint of time to all-cause allograft loss, defined as return to dialysis, allograft nephrectomy, re-transplantation, eGFR <15 mL/min/1.73 m² or death from any cause (including death with functioning allograft). (Temporary (60 days) return to dialysis due to acute kidney injury (AKI) generally is excluded).

An eGFR <15 mL/min/1.73 m² generally is confirmed by a repeat measurement taken between 14 to 30 days later in order to meet the primary endpoint definition of graft loss. Temporary (60 days) eGFR decline to <15 mL/min/1.73 m² due to AKI is excluded.

Return to dialysis or confirmed eGFR decline (to <15 mL/min/1.73 m²)>60 days in duration is considered permanent and fulfills the endpoint of allograft loss.

AKI are be identified as AE(s) leading to acute worsening of graft function (including but not limited to acute glomerulonephritis, acute thrombotic event, dehydration, drug toxicity or exposure to known nephrotoxic agents, interstitial nephritis, sepsis, urinary tract obstruction, urosepsis, worsening of diabetes, and worsening of heart failure) accompanied by the presence of one or more of the following:
1. An increase in serum creatinine by 0.3 mg/dl within 48 hours of the start date of the associated AE
2. An increase in serum creatinine by 1.5 fold, known or presumed to have occurred within 7 days prior to the start date of the associated AE
3. Histologically confirmed acute rejection or any other acute condition confirmed by graft biopsy

A stratified log rank test are be used to compare the median time-to-event between each treatment arm. Incidence rates and hazard ratios are also to be presented.

To assess the robustness of the primary efficacy analysis, the primary efficacy variable may be repeated in sensitivity analyses using the PP set. An additional sensitivity analysis optionally may be conducted are address the nature of all-cause allograft loss as a recurrent event.

### Secondary Endpoints

The following secondary efficacy endpoints may also be analyzed:
1. Incidence and time to death-censored allograft loss (defined as return to dialysis, allograft nephrectomy, re-transplantation or eGFR <15 mL/min/1.73 m² but excluding death from any cause)
2. Change in mean eGFR from Baseline to EO
3. Change in spot UACR from Baseline to EOS
4. Change in DSA titers and MFI intensity scores from Baseline to EOS
5. Incidence of acute rejection episodes (TCMR and ABMR) from Baseline to EOS
6. Change in Banff lesion grading score (2015 criteria) of pre-treatment to post-treatment (Week 52) kidney biopsies
7. Overall patient survival

In addition, secondary endpoints related to healthcare utilization and patient reported outcomes may be examined such as the following:
1. Healthcare utilization associated with the treatment of ABMR to Week 52 as well as to EOS
2. Change in patient reported outcomes, including HRQoL, from Baseline to Week 52 as well as to EOS

### Additional Analyses

IL-6 (free and total) levels, presence of anti-clazakizumab antibodies, and other evaluations/assessments may be presented.

CNI and MPA levels generally are measured throughout treatment. An analysis may be conducted to analyze the concentrations of these drugs. A comparison of these concentrations between the clazakizumab and control groups may be used to determine whether or not there have been any meaningful drug-drug PK interactions after initiation of Clazakizumab. The analysis are also to investigate and account for any significant differences in the doses of these drugs during the trial between the clazakizumab and control groups.

### Safety Evaluations

The following safety endpoints may further be evaluated and analyzed:
1. TEAE, serious TEAEs, and AESI
2. Viral infection monitoring for BKV, CMV, and EBV by PCR
3. Laboratory tests including LFTs, CBC, plasma lipids, high-sensitivity CRP
4. Vital signs, ECGs and physical examination

### Interim Analyses

Once approximately 100 subjects have been randomized and received Clazakizumab, an interim analysis for safety may be conducted. Further safety interim analyses may also be determined.

Two formal interim efficacy analyses may also be conducted.
1. Sample size re-estimation: After at least 100 subjects have been randomized and received Clazakizumab for at least 52 weeks, a formal interim analysis may be conducted by an independent statistician to assess the adequacy of the sample size for the interim efficacy analysis of the 52-week eGFR endpoint.
2. An interim efficacy analysis of the 52-week eGFR endpoint (i.e., change in mean eGFR from Baseline to Week 52) may be effected when approximately 200 subjects (100 per group) have been randomized and received at least 52 weeks of treatment with Clazakizumab.

The interim efficacy endpoint are be analyzed using a mixed model repeated measures approach. The model may include terms for treatment, stratification factors, baseline eGFR and other pre-defined covariates.

Sensitivity analyses may include the following:
1. Missing values imputed using the mean of the observed values at that time point within the same treatment group.
2. For subjects who are missing values after having experienced an AE, the missing values are to be imputed by the worst (lowest) eGFR value that is observed in the control group at the given time point. For subjects who are missing values for reasons other than an AE, values are to be imputed by the mean of the observed values at that time point within the same treatment group.
3. The delta adjustment method are be used to estimate the tipping point beyond which the active treatment would have an unfavorable effect.
4. Nonparametric rank-based method where subjects are first to be ranked on the time point that they last provided data, and then by the value of eGFR at that visit. A Wilcoxon rank sum test may then be applied to compare treatment groups using the ranks.

These and other changes can be made to the invention in light of the above detailed description. In general, in the following claims, the terms used should not be construed to limit the invention to the specific embodiments disclosed in the specification and the claims. Accordingly, the invention is not limited by the disclosure, but instead the scope of the invention is to be determined entirely by the following claims.

### REFERENCES

1. Keller E T, Wanagat J, Ershler W B. Molecular and cellular biology of interleukin-6 and its receptor. Front Biosci. 1996; 1: d340-57.
2. Hirano T, Taga T, Nakano N, Yasukawa K, Kashiwamura S, Shimizu K, et al. Purification to homogeneity and characterization of human B-cell differentiation factor (BCDF or BSFp-2). Proc Natl Acad Sci USA. 1985 August; 82(16):5490-4.
3. Kawano M M, Mihara K, Huang N, Tsujimoto T, Kuramoto A. Differentiation of early plasma cells on bone marrow stromal cells requires interleukin-6 for escaping from apoptosis. Blood. 1995 Jan. 15; 85(2):487-94.
4. Muraguchi A, Hirano T, Tang B, Matsuda T, Horii Y, Nakajima K, et al. The essential role of B cell stimulatory factor 2 (BSF-2/IL-6) for the terminal differentiation of B cells. J Exp Med. 1988 Feb. 1; 167:332-44.
5. Lotz M, Jirik F, Kabouridis P, Tsoukas C, Hirano T, Kishimoto T, et al. B cell stimulating factor 2/interleukin 6 is a costimulant for human thymocytes and T lymphocytes. J Exp Med. 1988 Mar. 1; 167(3):1253-8.
6. Okada M, Kitahara M, Kishimoto S, Matsuda T, Hirano T, Kishimoto T. IL-6/BSF-2 functions as a killer helper factor in the in vitro induction of cytotoxic T cells. J Immunol. 1988 Sep. 1; 141(5):1543-9.
7. Jordan S C, Choi J, Kim I, Wu G, Toyoda M, Shin B, et al. Interleukin-6, a cytokine critical to mediation of inflammation, autoimmunity and allograft rejection: therapeutic implications of IL-6 receptor blockade. Transplantation. 2017 January; 101(1):32-44.
8. Gaston R S, Cecka J M, Kasiske B L, Fieberg A M, Leduc R, Cosio F C, et al. Evidence for antibody-mediated injury as a major determinant of late kidney allograft failure. Transplantation. 2010 Jul. 15; 90(1):68-74.
9. Thomas K A, Valenzuela N M, Reed E F. The perfect storm: HLA antibodies, complement, FcγRs, and endothelium in transplant rejection. Trends Mol Med. 2015 May; 21(5):319-29.
10. Wiebe C, Gibson I W, Blydt-Hansen T D, Karpinksi M, Ho J, Storsley L J, et al. Evolution and clinical pathologic correlations of de novo donor-specific HLA antibody post kidney transplant. Am J Transplant. 2012 May; 12(5):1157-67.
11. Wiebe C, Gibson I W, Blydt-Hansen T D, Pochinco D, Birk P E, Ho J, et al. Rates and determinants of progression to graft failure in kidney allograft recipients with de novo donor-specific antibody. Am J Transplant. 2015 November; 15(11):2921-30.
12. Hariharan S, McBride M A, Cherikh W S, Tolleris C B, Bresnahan B A, Johnson C P. Post-transplant renal function in the first year predicts long-term kidney transplant survival. Kidney Int. 2002 July; 62(1):311-8.
13. Kasiske B L, Israni A K, Snyder J J, Skeans M A. The relationship between kidney function and long-term graft survival after kidney transplant. Am J Kidney Dis. 2011 March; 57(3):466-75.
14. Clayton P A, Lim W H, Wong G, Chadban S J. Relationship between eGFR decline and hard outcomes after kidney transplants. J Am Soc Nephrol. 2016; 27:3440-6.
15. Eskandary F, Bond G, Schwaiger E, Kikic Z, Winzer C, Wahrmann M, et al. Bortezomib in late antibody-mediated kidney transplant rejection (BORTEJECT Study): study protocol for a randomized controlled trial. Trials. 2014 Apr. 3; 15:107. p. 1-9.
16. Stegall M D, Gaston R S, Cosio F G, Matas A. Through a glass darkly: seeking clarity in preventing late kidney transplant failure. J Am Soc Nephrol. 2015 Jan.; 26(1):20-9. Epub 2014 Aug. 5.
17. Kidney Disease: Improving Global Outcomes (KDIGO) Transplant Work Group. KDIGO clinical practice guideline for the care of kidney transplant recipients. Am J Transplant. 2009 November; 9 Suppl 3:S1-155.
18. Roberts D M, Jiang S H, Chadban S J. The treatment of acute antibody-mediated rejection in kidney transplant recipients-a systematic review. Transplantation. 2012 October; 94(8):775-83.
19. Wan S S, Ying T D, Wyburn K, Roberts D M, Wyld M, Chadban S J. The treatment of antibody-mediated rejection in kidney transplantation: an updated systematic review and meta-analysis. Transplantation. 2018 Apr.; 102(4): 557-68.
20. Bailly E, Blancho G, Ville S, Morelon E, Bamoulid J, Caillard S, et al. Five-year outcomes after randomization treatment by rituximab in early acute antibody-mediated rejection in renal transplantation: long term outcomes of the RITUX ERAH Study. Am J Transplant. 2017; 17(Suppl 3).
21. Sautenet B, Blancho G, BUchler M, Morelon E, Toupance O, Barrou B, et al. One-year results of the effects of rituximab on acute antibody-mediated rejection in renal transplantation: RITUX ERAH, a multicenter double-blind randomized placebo-controlled trial. Transplantation. 2016 February; 100(2):391-9.
22. Kulkarni S, Kirkiles-Smith N C, Deng Y H, Formica R N, Moeckel G, Broecker V, et al. Eculizumab therapy for chronic antibody-mediated injury in kidney transplant recipients: a pilot randomized controlled trial. Am J Transplant. 2017 Mar.; 17(3):682-91.
23. Eskandary F, Regele H, Baumann L, Bond G, Kozakowski N, Wahrmann M, et al. A randomized trial of bortezomib in late antibody-mediated kidney transplant rejection. J Am Soc Nephrol. 2018; 29:591-605.
24. Moreso F, Crespo M, Ruiz J C, Torres A, Gutierrez-Dalmau A, Osuna A, et al. Treatment of chronic antibody mediated rejection with intravenous immunoglobulins and rituximab: a multicenter, prospective, randomized, double-blind clinical trial. Am J Transplant. 2018; 18:927-935.
25. Pineiro G, Rovira J, De Sousa-Amorim E, Villarreal J, Sole M, Revuelta I, et al. Treatment of active chronic antibody-mediated rejection with rituximab, IVIG and plasma exchange Am J Transplant. 2017; 17(Suppl 3).
26. Choi J, Aubert O, Vo A, Loupy A, Haas M, Puliyanda D, et al. Assessment of tocilizumab (anti-interleukin-6 receptor monoclonal) as a potential treatment for chronic antibody-mediated rejection and transplant glomerulopathy in HLA-sensitized renal allograft recipients. Am J Transplant. 2017; 17:2381-9.
27. Choi J, Aubert O, Louie S, Ammerman N, Vo A, Peng A, et al. Extended experience using tocilizumab (anti-IL6R, TCZ) for the treatment of chronic antibody mediated rejection (CABMR) Am J Transplant. 2017; 17(Suppl 3).
28. Puliyanda D, Kim I, Choi J, Haas M, Zhang X, Vo A, et al. Safety and efficacy of tocilizumab (anti-IL6R, TCZ) therapy in the treatment of chronic antibody mediated rejection (cABMR) in pediatric renal transplant recipients Am J Transplant. 2017; 17(Suppl 3).
29. Venkatachalam K, Reem D, Andrew M, Delos Santos R, Alhamad T. Efficacy and safety of tocilizumab in treatment of acute antibody mediated rejection. Am J Transplant. 2017; 17(Suppl 3).
30. Chandran S, Leung J, Laszik Z, Tang Q, Hu C, Sarwal M, et al. IL-6 inhibition with tocilizumab to promote tregs and control renal graft inflammation: a prospective randomized controlled trial. Am J Transplant. 2017; 17(Suppl 3).
31. Patel S, Mohan S, Fernandez H, Batal I, Ratner L, Crew J. Tocilizumab stabilizes renal function in kidney transplant recipients with chronic active antibody mediated rejection (CAAMR) Am J Transplant. 2017; 17(Suppl 3).
32. Vo A A, Choi J, Kim I, Louie S, Cisneros K, Kahwaji J, et al. A phase I/II trial of the interleukin 6 receptor specific humanized monoclonal (tocilizumab)+intravenous immunoglobulin in difficult to desensitize patients. Transplantation. 2015; 99(11):2356-63.
33. Investigator's Brochure Clazakizumab. Version 4.0. Final report. Victoria (BC): Vitaeris Inc.; 2018 May 1.
34. Actemra® (tocilizumab) injection, for intravenous or subcutaneous use. Prescribing Information. Genentech, Inc. USA. 2017 Aug.
35. RoActemra® 20 mg/ml concentrate for solution for infusion. Summary of Product Characteristics. Roche Products Limited, Garden City, Herfordshire, UK. 2018 Apr 12.
36. Final report. Quantitative assessment of the relationship between change in estimated glomerular filtration rate and risk of allograft failure in patients diagnosed with active antibody-mediated rejection following kidney transplantation. Vancouver (BC): Vitaeris Inc.; 2018 Jun. 1.
37. Loupy A, Haas M, Solez K, Racusen L, Glotz D, Seron D, et al. The Banff 2015 Kidney Meeting Report: current challenges in rejection classification and prospects for adopting molecular pathology. Am J Transplant. 2017; 17(1):28-41.
38. Clinical study report IM133001. Phase IIB, randomized, multi-center, double-blind, dose-ranging, placebo/active controlled study to evaluate the efficacy and safety of BMS-95429 subcutaneous injection with or without methotrexate in subjects with moderate to severe rheumatoid arthritis (RA) with inadequate response to methotrexate. Final report. Bristol-Myers Squibb Company; 2014 Sep. 12. Report No.: IM133001.
39. Hoffmann-La Roche Inc. Tocilizumab Arthritis Advisory Committee. 2008 Jul 29.
40. Clinical trial results summary IM133066. A Phase IIb, randomized, multi-Center, double-blind, dose-Ranging Study to evaluate the efficacy and safety of clazakizumab in subjects with moderate to severe active rheumatoid arthritis who have experienced an inadequate response to TNF inhibitors. Report in preparation. Preliminary results available upon request. Report No.: IM133066.
41. Zhao Q, Pang J, Shuster D, Hung C, Baglino S, Dodge R, et al. Anti-IL-6 antibody clazakizumab is more potent than tocilizumab in blocking in vitro and in vivo IL-6-induced functions. ACR/ARNP Annual Meeting; 2013 Oct 25-30; San Diego, CA. Poster 2385.
42. National Cancer Institute. Common Terminology Criteria for Adverse Events (CTCAE). Version 5.0. 2017 Nov. 27.
43. Bohmig G A, Budde K. Safety, tolerability and efficacy of anti-IL-6 antibody clazakizumab in late antibody-mediated rejection after kidney transplantation-a pilot trial. Medical University of Vienna; 2018.
44. Kevzara (sarilumab) injection. Prescribing Information. Regeneron Pharmaceuticals, Inc./Sanofi-Aventis U.S. LLC, USA. 2017 May.
45. Levey A S, Coresh J, Greene T, Stevens L A, Yaping Z, Hendriksen S, et al. Using standardized serum creatinine values in the modification of diet in renal disease study equation for estimating glomerular filtration rate. Ann Intern Med. 2006 Aug. 15; 145(4):247-54.
46. Sampson H A, Munoz-Furlong A, Campbell R L, Adkinson N F, Bock S A, Branum A, et al. Second symposium on the definition and management of anaphylaxis: summary report—Second National Institute of Allergy and Infectious Disease/Food Allergy and Anaphylaxis Network Symposium. Ann Emerg Med. 2006 April; 47(4):373-80.
47. Humar A, Michaels M. American Society of Transplantation recommendations for screening, monitoring and reporting of infectious complications in immunosuppression trials in recipients of organ transplantation. Am J Transplant. 2006; 6:262-74.
48. Tong A, Sautenet B, Poggio E D, Lentine K L, Oberbauer R, Mannon R, et al. Establishing a core outcome measure for graft health: a standardized outcomes in Nephrology-Kidney Transplantation (SONG-Tx) Consensus Workshop Report. Transplantation. 2018 August; 102(8):1358-66.
49. Kidney International Supplements. Section 2: AKI Definition. KDIGO Clinical practice guideline for acute kidney injury. Kidney Int Suppl. 2012; 2:19-36.
50. World Medical Association. World Medical Association Declaration of Helsinki. Ethical principles for medical research involving human subjects. JAMA. 2013 November; 310(20):2191-4.
51. ICH Harmonised Tripartite Guideline. Guideline for good clinical practice. E6 (R1) 1996 Jun. 10.
52. Pharmaceutical Research and Manufacturers of America. Principles on conduct of clinical trials. Communication of Clinical Trial Results. Washington (DC); 2014 Dec. Available from: http://phrma-docs.phrma.org/sites/default/files/pdf/042009_clinical_trial_principles_final_0.pdf.
53. International Committee of Medical Journal Editors Criteria. Defining the role of authors and contributors. C2018 [accessed 2018 Jul 27]. Available from: http://www.icmje.org/recommendations/browse/roles-and-responsibilities/defining-the-role-of-authors-and-contributors.html.

1. A method of preventing, stabilizing or reducing antibody mediated rejection (ABMR) in a subject who is to receive, is receiving or has received a solid organ transplant, or a method of reversing, stabilizing and/or slowing the progression of active antibody mediated-rejection (AMBR) in a transplant recipient in need thereof, comprising administering to said subject a prophylactically or therapeutically effective amount of an anti-human interleukin-6 (IL-6) antibody or anti-human 11-6 antibody fragment, wherein the antibody or antibody fragment comprises: a variable light chain polypeptide comprising the CDRs of SEQ ID NOs:4, 5 and 6 and, and a variable heavy chain polypeptide comprising the CDRs of SEQ ID NOs:7, 8 or 120, and 9, and wherein the anti-human IL-6 antibody is administered for more than 5 years.

2. (canceled)

3. The method of claim 1, wherein the anti-human IL-6 antibody comprises the heavy chain polypeptide of SEQ ID NO: 704 or 745 and comprises the light chain polypeptide of SEQ ID NO: 702 or 746.

4.-10. (canceled)

11. The method of claim 1, wherein:
(i) the transplant recipient comprises active antibody mediated-rejection (AMBR) or chronic active antibody mediated-rejection (CABMR) when treatment is started, optionally at least once within the time period spanning 1-6 months prior to treatment;
(ii) the transplant recipient has been diagnosed as having AMBR or CAMBR prior to anti-IL-6 antibody administration;
(iii) treatment with the anti-IL-6 antibody stabilizes or increases the estimated glomerular filtration rate (eGFR) during treatment, optionally throughout the entire treatment period;
(iv) treatment with the anti-IL-6 antibody stabilizes or increases the estimated glomerular filtration rate (eGFR) during treatment, optionally throughout the entire treatment period and further optionally wherein said stabilization or increase in eGFR is maintained for at least 3, 6, 9 or 12 months after treatment has ended:
(v) the transplant recipient does not comprise neutropenia (less than 1,000 mm³) or thrombocytopenia (less than 50,000 mm³) when treatment is commenced and/or during the treatment regimen;
(vi) the transplant recipient does not receive intravenous immunoglobulin within the time period spanning 0-6 months prior to treatment;
(vii) the transplant recipient comprises human leukocyte antigen (HLA) DSAs prior to treatment, optionally wherein this has been confirmed by an assay which detects for human leukocyte antigen (HLA) DSAs within the time period spanning 0-6 months prior to treatment;
(viii) the anti-IL-6 antibody is administered in the period spanning 0-3, 1-3, 1-4, 1-5 or 1-6 months prior to transplant;
(ix) the treatment elicits one or more of the following:
(a) reduces the number of or eliminates donor specific antibodies (DSAs);
(b) reduces CCL2 levels;
(c) reduces complement activation and/or reduces the amount of detected C5b, C9 and/or C5b/C9 complexes;
(d) reduces the number of plasma cells secreting DSAs;
(e) prevents allograft loss;
(f) prevents return to dialysis;
(g) prevents allograft nephrectomy;
(h) prevents the need for re-transplantation, or
(i) maintains or increases estimated glomerular filtration rate (eGFR) such that it is at least ≥15 mL/min/1.73 m²; or
(j) a combination of the foregoing;
(x) the transplanted solid organ is selected from a kidney, heart, lung, bladder, pancreas, liver, gall bladder, thyroid, skin or any combination of the foregoing;
(xi) the transplanted solid organ comprises or consists of a kidney;
(xii) the transplanted organ is from a living or deceased donor;
(xiii) efficacy during or after treatment is evaluated at least in part by detecting eGFR values, optionally using the Modification of Diet in Renal Disease 4 (MDRD4) equation;
(xiv) efficacy is evaluated at least in part by evaluating the histology of kidney biopsies according to the Banff 2015 lesion grading scores;
(xv) efficacy is evaluated at least in part by detecting DSA titers and/or mean fluorescence intensity (MFI) scores;
(xvi) efficacy is evaluated at least in part by evaluating the incidence of acute rejection episodes (TCMR and ABMR) periodically during treatment;
(xvii) efficacy is evaluated at least in part by evaluating the effects of treatment on albuminuria;
(xviii) efficacy is evaluated at least in part by evaluating survival rates compared to controls and/or conventional AMBR or CAMBR treatments;
(xix) the anti-IL-6 antibody comprises human IgG1 constant regions;
(xx) efficacy the anti-IL-6 antibody comprises human IgG1 constant regions that comprise the constant light polypeptide of SEQ ID NO: 586 and the constant heavy polypeptide of SEQ ID NO: 588;
(xxi) the anti-IL-6 antibody comprises the variable heavy chain polypeptide of SEQ ID NO: 657 and the variable light chain polypeptide of SEQ ID NO: 709;
(xxii) the anti-IL-6 antibody comprises the heavy chain polypeptide of SEQ ID NO: 704 or 745 and the light chain polypeptide of SEQ ID NO: 702 or 746;
(xxiii) the anti-IL-6 antibody is dosed intravenously or subcutaneously every 4 weeks or monthly;
(xxiv) a 25 mg or 12.5 mg dose of the anti-IL-6 antibody is administered intravenously or subcutaneously every 4 weeks or monthly;
(xxv) a 25 mg or 12.5 mg dose of Claza is administered subcutaneously every 4 weeks or monthly;
(xxvi) the treatment is effected for at least 1 year, 2 years, 3 years, 4 years or 5 years without an adverse event selected from return to dialysis, allograft nephrectomy, re-transplantation or eGFR ≤15 mL/min/1.73 m²;
(xxvii) the transplant recipient optionally is further treated with any of the following:
(a) azathioprine (e.g., 1.0-2.0 mg/kg/day);
(b) calcineurin inhibitors (CNIs);
(c) mycophenolate mofetil (MMF)/mycophenolic acid (MPA);
(d) mTOR inhibitors;
(e) low dose corticosteroids;
(f) antihypertensive agents;
(g) angiotensin II receptor blockers (ARBs);
(h) cyclosporine;
(i) antidiabetogenic agents; or
(j) or a combination of any of the foregoing;
(xxviii) the transplant recipient is further treated with pneumocystis jiroveci pneumonia (PJP) prophylaxis;
(xxix) the transplant recipient if the transplant recipient subject experiences acute TCMR it is treated;
(xxx) during anti-IL-6 antibody treatment and optionally within the period spanning the 0, 1, 2, 3, 4, 5 or 6 months prior to starting treatment the transplant recipient subject is not treated with any of the following:
(a) rituximab,
(b) eculizumab,
(c) proteasome inhibitors,
(d) intravenous immunoglobulin (IVIG), except for treatment of hypogammaglobulinemia,
(e) plasma exchange (PLEX), belatacept,
(f) anti-IL-6R antibody and/or
(g) any combination of the foregoing;
(xxxi) the transplant recipient subject comprises any or all of the following:
(a) is 18-75 years old,
(b) treatment started ≥6 months from time of transplant,
(c) has received a diagnosis of CABMR according to BANFF 2015 diagnostic criteria which include the following: Biopsy proven CABMR (i.e., chronic glomerulopathy (cg)>0) with or without C4d staining, wherein repeat biopsy is performed if previous biopsy is not within 6 months of screening,
(d) if subject has received treatment for ABMR, CABMR, or TCMR, a repeat biopsy is performed wherein the subject is eligible if the subject lacks evidence of chronic tissue injury on light microscopy but has glomerular basement membrane double contours on electron microscopy (cgla);
(e) presence of HLA DSA is detected post-transplant;
(xxxii) the transplant recipient subject:
(a) has not had treatment for ABMR, CABMR, or TCMR within the time period spanning 0-3 months or 0-6 months of IL-6 antibody treatment or screening;
(b) is not receiving any T cell depleting agents, and has not received treatment for ABMR, CABMR, or TCMR within 3 months of screening or treatment;
(c) has not received T cell depleting agents within 3 months of screening or IL-6 antibody treatment;
(d) does not have a biopsy showing pure TCMR or advanced interstitial fibrosis (ci3);
(e) does not have advanced tubular atrophy (ct3);
(f) no vascular fibrous intimal thickening (cv3) or other significant causes of renal dysfunction;
(g) does not have impaired renal function due to disorders in the transplanted allograft;
(h) does not have eGFR <25 mL/min/1.73 m² or >65 mL/min/1.73 m² (MDRD4), (viii) no nephrotic range proteinuria defined as spot urine protein creatinine ratio (UPCR)≥3,000 mg/g (≥300 mg/mmol) or spot urine albumin creatinine ratio (UACR)≥2,200 mg/g (≥220 mg/mmol);
(i) is not pregnant or breastfeeding;
(j) does not have a history of anaphylaxis;
(k) does not have abnormal liver function tests (LFTs) (alanine aminotransferase (ALT)/aspartate aminotransferase (AST)/bilirubin >1.5× upper limit of normal) or other significant liver disease;
(l) does not have a history of active tuberculosis (TB);
(m) does not have a history of latent TB without history of active TB unless subject has completed a full course of prophylactic treatment;
(n) does not have a history of human immunodeficiency virus (HIV) infection or positive for HIV;
(o) is not seropositive for hepatitis B surface antigen (HBsAg);
(p) is not Hepatitis C virus (HCV) RNA positive;
(q) does not have a known Epstein-Barr virus (EBV) mismatch: donor seropositive, recipient seronegative;
(r) does not have a history of gastrointestinal perforation, diverticular disease or diverticulitis, or inflammatory bowel disease;
(s) does not have neutropenia (<1,000/mm³) or thrombocytopenia (<50,000/mm³);
(t) does not have active infections requiring systemic antimicrobial agents and unresolved prior to screening;
(u) does not have a history of or current invasive fungal infection or other opportunistic infection;
(v) does not have active viral infections;
(w) has not received a live vaccine within 6 weeks of screening, and/or has no history of alcohol or illicit substance abuse;
(x) has no present or previous (within 3 years) malignancy except for basal cell carcinoma, fully excised squamous cell carcinoma of the skin, or non-recurrent (within 5 years) cervical carcinoma in-situ;
(y) has no presence of a condition or abnormality that could compromise safety or life expectancy;
(z) has no history of intolerance to trimethoprim or and/or sulfamethoxazole, no previous treatment with anti-IL-6 antibody;
(aa) has complement activity measured in the subject before, during or after treatment or any combination of the foregoing; or
(xxxiii) any combination of (i) to (xxxii).

12.-42. (canceled)

43. A method of preventing, stabilizing or reducing complement activity in a subject in need thereof comprising administering to said subject a prophylactically or therapeutically effective amount of an anti-human interleukin-6 (IL-6) antibody or antibody fragment, e.g., one wherein the antibody or antibody fragment comprises: a variable light chain polypeptide comprising the CDRs of SEQ ID NOs: 4, 5 and 6 and, and a variable heavy chain polypeptide comprising the CDRs of SEQ ID NOs: 7, 8 or 120, and 9, and wherein the anti-human IL-6 antibody is administered for more than 5 years.

44. The method of claim 43, wherein:
(i) complement activity is measured in the subject before, during or after treatment;
(ii) the antibody comprises a V_{H} and V_{L} polypeptide respectively at least 90, 95, 96, 97, 98 or 99% identical to the polypeptides of SEQ ID NO:657 and 709;
(iii) the antibody comprises a heavy chain and light polypeptide respectively at least 90, 95, 96, 97, 98 or 99% identical to the polypeptides of SEQ ID NO:704 or 745 and 702 or 746;
(iv) the antibody is clazakizumab;
(v) the solid organ is selected from kidney, heart, liver, lungs, pancreas, gall bladder skin, intestine, stomach, or a combination of any of the foregoing;
(vi) the solid organ comprises or consists of a kidney;
(vii) the subject is evaluated and has been diagnosed as having ABMR or CAMBR prior to treatment, optionally wherein the evaluation comprises one or more of: detecting pre-formed and de novo HLA DSA (especially those detecting complement binding DSA such as Clq), detecting non-HLA antibodies associated with ABMR, and/or identifying at least one histological feature characteristic of antibody mediated organ damage;
(viii) the subject is evaluated and wherein the histological feature characteristic of antibody mediated organ damage is detected by obtaining a biopsy from the transplanted organ;
(ix) the histological feature characteristic of antibody mediated organ damage includes any of microvascular inflammation, complement deposition (C4d), and capillaritis;
(x) the transplanted organ is a kidney and the histological feature characteristic of antibody mediated organ damage includes any of microvascular inflammation, complement deposition (C4d) in the peritubular capillaries, peritubular capillaritis, glomerulitis and transplant glomerulopathy (double glomerular basement membrane contour);
(xi) the treatment further includes the administration of at least one other immunosuppressant;
(xii) the at least one other immunosuppressant is a standard of care pre- or post-transplant immunosuppressive medication;
(xiii) the at least one other immunosuppressant comprises any of thymoglobulin, basiliximab, mycophenolate mofetil, tacrolimus, an anti-CD20 mAb, and corticosteroids;
(xiv) the anti-IL-6 antibody is administered intravenously or subcutaneously;
(xv) the anti-IL-6 antibody is administered at doses ranging from 0.01-5000 mg;
(xvi) the anti-IL-6 antibody is administered at doses ranging from 0.1-1000 mg;
(xvii) the anti-IL-6 antibody is administered at doses ranging from 1-500 mg;
(xviii) the anti-IL-6 antibody is administered intravenously at doses ranging from about of 5 mg-50 mg or subcutaneously at doses ranging from about 10 mg-50 mg;
(xix) the anti-IL-6 antibody is administered at a dose of about 25 mg which is dosed about every 2 weeks, 4 weeks, 6 weeks, 8 weeks, 12 weeks, 16 weeks, 20 weeks, 24 weeks, monthly, bimonthly, every 2 months, every 3 months, every 4 months, every 5 months, every 6 months, every year or less frequently;
(xx) the anti-IL-6 antibody is administered about every 4 weeks, 8 weeks, 12 weeks, 16 weeks, 20 weeks, or 24 weeks;
(xxi) the anti-IL-6 antibody is administered subcutaneously at a dosage of 25 mg or 12.5 mg every 4 weeks or monthly;
(xxii) the anti-IL-6 antibody is administered within about 1, 2 or 3 months of detecting signs of ABMR or CAMBR;
(xxiii) the anti-IL-6 antibody is administered for several months prior to and months or years after transplant in order to prevent, stabilize or reduce antibody mediated damage to the transplanted organ; or
(xxiv) any combination of the foregoing.

45.-67. (canceled)

68. A method of preventing, stabilizing or reducing pre- or post-transplant sensitization in a subject who has or is to receive a solid organ transplant, comprising administering a prophylactically or therapeutically effective amount of an anti-human interleukin-6 (IL-6) antibody or antibody fragment, wherein the antibody or antibody fragment comprises: a variable light chain polypeptide comprising the CDRs of SEQ ID NOs: 4, 5 and 6 and, and a variable heavy chain polypeptide comprising the CDRs of SEQ ID NOs: 7, 8 or 120, and 9, wherein the anti-human IL-6 antibody is administered for more than 5 years.

69. The method of claim 68, wherein:
(i) the antibody comprises a VH and VL polypeptide respectively at least 90, 95, 96, 97, 98 or 99% identical to the polypeptides of SEQ ID NO:657 and 709,
(ii) the antibody respectively comprises the VH and VL polypeptides having the amino acid sequences of SEQ ID NO:657 and 709;
(iii) the antibody comprises a light and heavy chain polypeptide respectively identical to the polypeptides of SEQ ID NO: 702 or 746 and 704 or 745;
(iv) the subject has been transplanted with a solid organ is selected from kidney, heart, liver, lungs, pancreas, skin, intestine, stomach, or a combination of any of the foregoing;
(v) the solid organ comprises or consists of a kidney;
(vi) the subject is at risk of or is sensitized because of a history of blood transfusions, pregnancies or a previous transplant;
(vii) the subject comprises pre-formed donor specific antibodies (DSA) to the donor organ prior to and/or during anti-IL-6 antibody treatment;
(viii) the treatment further includes a pre-transplant desensitization procedure to remove or reduce donor specific alloantibodies (DSAS);
(ix) the treatment further includes a pre-transplant desensitization procedure which includes plasmapheresis or plasma exchange optionally in combination with any one of intravenous immunoglobulin, anti-B cell agents such rituximab (an anti-CD20 mAb), and plasma cell inhibitors;
(x) the antibody is administered intravenously or subcutaneously;
(xi) the antibody is administered at doses ranging from 0.01-5000 mg;
(xii) the antibody is administered at doses ranging from 0.1-1000 mg;
(xiii) the antibody is administered at doses ranging from 1-500 mg;
(xiv) the antibody is administered at a dose of about 25 mg which is dosed about every 2 weeks, 4 weeks, 6 weeks, 8 weeks, 12 weeks, 16 weeks, 20 weeks, 24 weeks, monthly, bimonthly, every 2 months, every 3 months, every 4 months, every 5 months, every 6 months, every year or less frequently;
(xv) the antibody is administered about every 4 or 8 weeks, starting several months (e.g. within the period spanning 0-6 months prior to transplantation;
(xvi) the subject is periodically assessed during treatment by one or more antibody detection methods (e.g. cytotoxic cross-match, flow cytometric cross match, Luminex antibody testing) pre-desensitization to detect levels of DSA during the desensitization treatment process;
(xvii) a positive response (e.g. conversion of positive to negative cytotoxic cross-match) is used to determine that the subject is eligible for IL-6 antibody treatment and/or transplantation;
(xviii) the subject is an organ transplant recipient and is treated with Clazakizumab post-transplant, optionally several months or years post-transplant to prevent or treat early acute or late chronic rejection events;
(xix) the subject is monitored for clinical signs of rejection, or the development of new DSA (de novo DSA);
(xx) the subject is monitored for histological signs of organ rejection;
(xxi) the subject is determined to have ABMR organ damage by biopsy evidence (e.g., microvascular inflammation, interstitial fibrosis, transplant glomerulopathy, CD4 deposition);
(xxii) the subject is treated with clazakizumab in combination with thymoglobulin, basiliximab, mycophenolate mofetil, tacrolimus, and/or corticosteroids pre- and post-transplant;
(xxiii) the administered anti-IL-6 antibody or antibody fragment contains an Fc region that has been modified to alter effector function, half-life, proteolysis, and/or glycosylation;
(xxiv) the anti-IL-6 antibody is selected from a humanized, single chain, or chimeric antibody and the antibody fragment is selected from a Fab, Fab′, F(ab′)2, Fv, or scFv:
(xxv) the IL-6 antibody dose is between about 0.001 and 100 mg/kg of body weight of recipient subject;
(xxvi) the IL-6 antibody dose is between about 0.1 and 20 mg/kg of body weight of recipient subject or comprises about 25 mg;
(xxvii) the IL-6 antibody comprises a human IgG1, IgG2, IgG3 or IgG4 constant region;
(xxviii) the IL-6 antibody comprises a human a human IgG1 constant region;
(xxix) the IL-6 antibody comprises a human the anti-IL-6 antibody is clazakizumab;
(xxx) the treated subject has late or advanced AMBR (Acute/active or chronic/active phenotype according to the Banff 2015 classification);
(xxxi) the administered anti-IL-6 antibody is clazakizumab and the treated subject has late or advanced AMBR (Acute/active or chronic/active phenotype according to the Banff 2015 classification);
(xxxii) the treated subject has a complement-related condition selected from age-related and degenerative diseases, Alzheimer's Disease, glomerular diseases, hemolytic uremic syndrome caused by Shiga toxin-producing E. coli (STEC-HUS), thrombotic thrombocytopenic purpura (TTP), systemic lupus erythematosus (SLE), antiphospholipid antibody syndrome (APS), anti-neutrophil cytoplasmic antibody (ANCA)-induced vasculitis, inflammatory small-vessel disorders caused by autoantibodies against neutrophil constituents; antibody-dependent, pregnancy loss involving C5a-mediated impairment of placental angiogenesis, complement mediated hemolytic disorders, ischemia-reperfusion injury; stroke, myocardial infarction, cardiopulmonary bypass surgery (CPB), allergic asthma, periodontitis, bone-related disorders and bone injury associated with aberrant complement activation, acute-phase conditions, in which the host is confronted with a dramatic increase of damage- and/or pathogen-associated molecular patterns; or
(xxxiii) any combination of the foregoing.

70.-103. (canceled)


SEQUENCE LISTING (746 sequences)

SEQ ID NO: 1 - protein, 183 aa, Homo sapiens
VPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 2 - protein, 163 aa, Oryctolagus cuniculus
MDTRAPTQLLGLLLLWLPGARCAYDMTQTPASVSAAVGGTVTIKCQASQSINNELSWYQQKPGQRPKLLIYRASTLASGVSSRFKGSGSGTEFTLTISDLECADAATYYCQQGYSLRNIDNAFGGGTEVVVKRTVAAPSVFIFPPSDEQLKSGTASVVCLLNN

SEQ ID NO: 3 - protein, 166 aa, Oryctolagus cuniculus
METGLRWLLLVAVLKGVQCQSLEESGGRLVTPGTPLTLTCTASGFSLSNYYVTWVRQAPGKGLEWIGIIYGSDETAYATWAIGRFTISKTSTTVDLKMTSLTAADTATYFCARDDSSDWDAKFNLWGQGTLVTVSSASTKGPSVFPLAPSSKSTSGGTAALGCLVK

SEQ ID NO: 4 - protein, 11 aa, Oryctolagus cuniculus
QASQSINNELS

SEQ ID NO: 5 - protein, 7 aa, Oryctolagus cuniculus
RASTLAS

SEQ ID NO: 6 - protein, 12 aa, Oryctolagus cuniculus
QQGYSLRNIDNA

SEQ ID NO: 7 - protein, 5 aa, Oryctolagus cuniculus
NYYVT

SEQ ID NO: 8 - protein, 16 aa, Oryctolagus cuniculus
IIYGSDETAYATWAIG

SEQ ID NO: 9 - protein, 12 aa, Oryctolagus cuniculus
DDSSDWDAKFNL

SEQ ID NO: 10 - DNA, 491 bp, Oryctolagus cuniculus
  source 1..491: mol_type = genomic DNA
atggacacgagggcccccactcagctgctggggctcctgctgctctggctcccaggtgccagatgtgcctatgatatgacccagactccagcctcggtgtctgcagctgtgggaggcacagtcaccatcaagtgccaggccagtcagagcattaacaatgaattatcctggtatcagcagaaaccagggcagcgtcccaagctcctgatctatagggcatccactctggcatctggggtctcatcgcggttcaaaggcagtggatctgggacagagttcactctcaccatcagcgacctggagtgtgccgatgctgccacttactactgtcaacagggttatagtctgaggaatattgataatgctttcggcggagggaccgaggtggtggtcaaacgtacggtagcggccccatctgtcttcatcttcccgccatctgatgagcagttgaaatctggaactgcctctgttgtgtgcctgctgaataactt

SEQ ID NO: 11 - DNA, 499 bp, Oryctolagus cuniculus
  source 1..499: mol_type = genomic DNA
atggagactgggctgcgctggcttctcctggtcgctgtgctcaaaggtgtccagtgtcagtcgctggaggagtccgggggtcgcctggtcacgcctgggacacccctgacactcacctgcacagcctctggattctccctcagtaactactacgtgacctgggtccgccaggctccagggaaggggctggaatggatcggaatcatttatggtagtgatgaaacggcctacgcgacctgggcgataggccgattcaccatctccaaaacctcgaccacggtggatctgaaaatgaccagtctgacagccgcggacacggccacctatttctgtgccagagatgatagtagtgactgggatgcaaaatttaacttgtggggccaaggcaccctggtcaccgtctcgagcgcctccaccaagggcccatcggtcttccccctggcaccctcctccaagagcacctctgggggcacagcggccctgggctgcctggtcaagg

SEQ ID NO: 12 - DNA, 33 bp, Oryctolagus cuniculus
  source 1..33: mol_type = genomic DNA
caggccagtcagagcattaacaatgaattatcc

SEQ ID NO: 13 - DNA, 21 bp, Oryctolagus cuniculus
  source 1..21: mol_type = genomic DNA
agggcatccactctggcatct

SEQ ID NO: 14 - DNA, 36 bp, Oryctolagus cuniculus
  source 1..36: mol_type = genomic DNA
caacagggttatagtctgaggaatattgataatgct

SEQ ID NO: 15 - DNA, 15 bp, Oryctolagus cuniculus
  source 1..15: mol_type = genomic DNA
aactactacgtgacc

SEQ ID NO: 16 - DNA, 48 bp, Oryctolagus cuniculus
  source 1..48: mol_type = genomic DNA
atcatttatggtagtgatgaaacggcctacgcgacctgggcgataggc

SEQ ID NO: 17 - DNA, 36 bp, Oryctolagus cuniculus
  source 1..36: mol_type = genomic DNA
gatgatagtagtgactgggatgcaaaatttaacttg

SEQ ID NO: 18 - protein, 109 aa, Oryctolagus cuniculus
EVQLVESGGGLVQPGGSLRLSCAASGFSLSNYYVTWVRQAPGKGLEWVGIIYGSDETAYATWAIGRFTISRDNSKNTLYLQMNSLRAEDTAVYYCARDDSSDWDAKFNL

SEQ ID NO: 19 - protein, 109 aa, Oryctolagus cuniculus
EVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNL

SEQ ID NO: 20 - protein, 99 aa, Oryctolagus cuniculus
IQMTQSPSSLSASVGDRVTITCQASQSINNELSWYQQKPGKAPKLLIYRASTLASGVPSRFSGSGSGTDFTLTISSLQPDDFATYYCQQGYSLRNIDNA

SEQ ID NO: 21 - protein, 170 aa, Oryctolagus cuniculus
MDTRAPTQLLGLLLLWLPGARCAYDMTQTPASVEVAVGGTVTINCQASETIYSWLSWYQQKPGQPPKLLIYQASDLASGVPSRFSGSGAGTEYTLTISGVQCDDAATYYCQQGYSGSNVDNVFGGGTEVVVKRTVAAPSVFIFPPSDEQLKSGTASVVCLLNNFYPREAK

SEQ ID NO: 22 - protein, 167 aa, Oryctolagus cuniculus
METGLRWLLLVAVLKGVQCQEQLKESGGRLVTPGTPLTLTCTASGFSLNDHAMGWVRQAPGKGLEYIGFINSGGSARYASWAEGRFTISRTSTTVDLKMTSLTTEDTATYFCVRGGAVWSIHSFDPWGPGTLVTVSSASTKGPSVFPLAPSSKSTSGGTAALGCLVK

SEQ ID NO: 23 - protein, 11 aa, Oryctolagus cuniculus
QASETIYSWLS

SEQ ID NO: 24 - protein, 7 aa, Oryctolagus cuniculus
QASDLAS

SEQ ID NO: 25 - protein, 12 aa, Oryctolagus cuniculus
QQGYSGSNVDNV

SEQ ID NO: 26 - protein, 5 aa, Oryctolagus cuniculus
DHAMG

SEQ ID NO: 27 - protein, 16 aa, Oryctolagus cuniculus
FINSGGSARYASWAEG

SEQ ID NO: 28 - protein, 12 aa, Oryctolagus cuniculus
GGAVWSIHSFDP

SEQ ID NO: 29 - DNA, 511 bp, Oryctolagus cuniculus
  source 1..511: mol_type = genomic DNA
atggacacgagggcccccactcagctgctggggctcctgctgctctggctcccaggtgccagatgtgcctatgatatgacccagactccagcctctgtggaggtagctgtgggaggcacagtcaccatcaattgccaggccagtgagaccatttacagttggttatcctggtatcagcagaagccagggcagcctcccaagctcctgatctaccaggcatccgatctggcatctggggtcccatcgcgattcagcggcagtggggctgggacagagtacactctcaccatcagcggcgtgcagtgtgacgatgctgccacttactactgtcaacagggttatagtggtagtaatgttgataatgttttcggcggagggaccgaggtggtggtcaaacgtacggtagcggccccatctgtcttcatcttcccgccatctgatgagcagttgaaatctggaactgcctctgttgtgtgcctgctgaataacttctatcccagagaggccaaag

SEQ ID NO: 30 - DNA, 501 bp, Oryctolagus cuniculus
  source 1..501: mol_type = genomic DNA
atggagactgggctgcgctggcttctcctggtcgctgtgctcaaaggtgtccagtgtcaggagcagctgaaggagtccgggggtcgcctggtcacgcctgggacacccctgacacttacctgcacagcctctggattctccctcaatgaccatgcaatgggctgggtccgccaggctccagggaaggggctggaatacatcggattcattaatagtggtggtagcgcacgctacgcgagctgggcagaaggccgattcaccatctccagaacctcgaccacggtggatctgaaaatgaccagtctgacaaccgaggacacggccacctatttctgtgtcagagggggtgctgtttggagtattcatagttttgatccctggggcccagggaccctggtcaccgtctcgagcgcctccaccaagggcccatcggtcttccccctggcaccctcctccaagagcacctctgggggcacagcggccctgggctgcctggtcaag

SEQ ID NO: 31 - DNA, 33 bp, Oryctolagus cuniculus
  source 1..33: mol_type = genomic DNA
caggccagtgagaccatttacagttggttatcc

SEQ ID NO: 32 - DNA, 21 bp, Oryctolagus cuniculus
  source 1..21: mol_type = genomic DNA
caggcatccgatctggcatct

SEQ ID NO: 33 - DNA, 36 bp, Oryctolagus cuniculus
  source 1..36: mol_type = genomic DNA
caacagggttatagtggtagtaatgttgataatgtt

SEQ ID NO: 34 - DNA, 15 bp, Oryctolagus cuniculus
  source 1..15: mol_type = genomic DNA
gaccatgcaatgggc

SEQ ID NO: 35 - DNA, 48 bp, Oryctolagus cuniculus
  source 1..48: mol_type = genomic DNA
ttcattaatagtggtggtagcgcacgctacgcgagctgggcagaaggc

SEQ ID NO: 36 - DNA, 36 bp, Oryctolagus cuniculus
  source 1..36: mol_type = genomic DNA
gggggtgctgtttggagtattcatagttttgatccc

SEQ ID NO: 37 - protein, 165 aa, Oryctolagus cuniculus
MDTRAPTQLLGLLLLWLPGATFAAVLTQTPSPVSAAVGGTVSISCQASQSVYDNNYLSWFQQKPGQPPKLLIYGASTLASGVPSRFVGSGSGTQFTLTITDVQCDDAATYYCAGVYDDDSDNAFGGGTEVVVKRTVAAPSVFIFPPSDEQLKSGTASVVCLLNNF

SEQ ID NO: 38 - protein, 166 aa, Oryctolagus cuniculus
METGLRWLLLVAVLKGVQCQSLEESGGRLVTPGTPLTLTCTASGFSLSVYYMNWVRQAPGKGLEWIGFITMSDNINYASWAKGRFTISKTSTTVDLKMTSPTTEDTATYFCARSRGWGTMGRLDLWGPGTLVTVSSASTKGPSVFPLAPSSKSTSGGTAALGCLVK

SEQ ID NO: 39 - protein, 13 aa, Oryctolagus cuniculus
QASQSVYDNNYLS

SEQ ID NO: 40 - protein, 7 aa, Oryctolagus cuniculus
GASTLAS

SEQ ID NO: 41 - protein, 11 aa, Oryctolagus cuniculus
AGVYDDDSDNA

SEQ ID NO: 42 - protein, 5 aa, Oryctolagus cuniculus
VYYMN

SEQ ID NO: 43 - protein, 16 aa, Oryctolagus cuniculus
FITMSDNINYASWAKG

SEQ ID NO: 44 - protein, 12 aa, Oryctolagus cuniculus
SRGWGTMGRLDL

SEQ ID NO: 45 - DNA, 496 bp, Oryctolagus cuniculus
  source 1..496: mol_type = genomic DNA
atggacacgagggcccccactcagctgctggggctcctgctgctctggctcccaggtgccacatttgccgccgtgctgacccagactccatctcccgtgtctgcagctgtgggaggcacagtcagcatcagttgccaggccagtcagagtgtttatgacaacaactacttatcctggtttcagcagaaaccagggcagcctcccaagctcctgatctatggtgcatccactctggcatctggggtcccatcgcggttcgtgggcagtggatctgggacacagttcactctcaccatcacagacgtgcagtgtgacgatgctgccacttactattgtgcaggcgtttatgatgatgatagtgataatgccttcggcggagggaccgaggtggtggtcaaacgtacggtagcggccccatctgtcttcatcttcccgccatctgatgagcagttgaaatctggaactgcctctgttgtgtgcctgctgaataacttct

SEQ ID NO: 46 - DNA, 499 bp, Oryctolagus cuniculus
  source 1..499: mol_type = genomic DNA
atggagactgggctgcgctggcttctcctggtggctgtgctcaaaggtgtccagtgtcagtcgctggaggagtccgggggtcgcctggtcacccctgggacacccctgacactcacctgcacagcctctggattctccctcagtgtctactacatgaactgggtccgccaggctccagggaaggggctggaatggatcggattcattacaatgagtgataatataaattacgcgagctgggcgaaaggccgattcaccatctccaaaacctcgaccacggtggatctgaaaatgaccagtccgacaaccgaggacacggccacctatttctgtgccaggagtcgtggctggggtacaatgggtcggttggatctctggggcccaggcaccctcgtcaccgtctcgagcgcctccaccaagggcccatcggtcttccccctggcaccctcctccaagagcacctctgggggcacagcggccctgggctgcctggtcaagg

SEQ ID NO: 47 - DNA, 39 bp, Oryctolagus cuniculus
  source 1..39: mol_type = genomic DNA
caggccagtcagagtgtttatgacaacaactacttatcc

SEQ ID NO: 48 - DNA, 21 bp, Oryctolagus cuniculus
  source 1..21: mol_type = genomic DNA
ggtgcatccactctggcatct

SEQ ID NO: 49 - DNA, 33 bp, Oryctolagus cuniculus
  source 1..33: mol_type = genomic DNA
gcaggcgtttatgatgatgatagtgataatgcc

SEQ ID NO: 50 - DNA, 15 bp, Oryctolagus cuniculus
  source 1..15: mol_type = genomic DNA
gtctactacatgaac

SEQ ID NO: 51 - DNA, 48 bp, Oryctolagus cuniculus
  source 1..48: mol_type = genomic DNA
ttcattacaatgagtgataatataaattacgcgagctgggcgaaaggc

SEQ ID NO: 52 - DNA, 36 bp, Oryctolagus cuniculus
  source 1..36: mol_type = genomic DNA
agtcgtggctggggtacaatgggtcggttggatctc

SEQ ID NO: 53 - protein, 164 aa, Oryctolagus cuniculus
MDTRAPTQLLGLLLLWLPGAICDPVLTQTPSPVSAPVGGTVSISCQASQSVYENNYLSWFQQKPGQPPKLLIYGASTLDSGVPSRFKGSGSGTQFTLTITDVQCDDAATYYCAGVYDDDSDDAFGGGTEVVVKRTVAAPSVFIFPPSDEQLKSGTASVVCLLNN

SEQ ID NO: 54 - protein, 167 aa, Oryctolagus cuniculus
METGLRWLLLVAVLKGVQCQEQLKESGGGLVTPGGTLTLTCTASGFSLNAYYMNWVRQAPGKGLEWIGFITLNNNVAYANWAKGRFTFSKTSTTVDLKMTSPTPEDTATYFCARSRGWGAMGRLDLWGHGTLVTVSSASTKGPSVFPLAPSSKSTSGGTAALGCLVK

SEQ ID NO: 55 - protein, 13 aa, Oryctolagus cuniculus
QASQSVYENNYLS

SEQ ID NO: 56 - protein, 7 aa, Oryctolagus cuniculus
GASTLDS

SEQ ID NO: 57 - protein, 11 aa, Oryctolagus cuniculus
AGVYDDDSDDA

SEQ ID NO: 58 - protein, 5 aa, Oryctolagus cuniculus
AYYMN

SEQ ID NO: 59 - protein, 16 aa, Oryctolagus cuniculus
FITLNNNVAYANWAKG

SEQ ID NO: 60 - protein, 12 aa, Oryctolagus cuniculus
SRGWGAMGRLDL

SEQ ID NO: 61 - DNA, 494 bp, Oryctolagus cuniculus
  source 1..494: mol_type = genomic DNA
atggacacgagggcccccactcagctgctggggctcctgctgctctggctcccaggtgccatatgtgaccctgtgctgacccagactccatctcccgtatctgcacctgtgggaggcacagtcagcatcagttgccaggccagtcagagtgtttatgagaacaactatttatcctggtttcagcagaaaccagggcagcctcccaagctcctgatctatggtgcatccactctggattctggggtcccatcgcggttcaaaggcagtggatctgggacacagttcactctcaccattacagacgtgcagtgtgacgatgctgccacttactattgtgcaggcgtttatgatgatgatagtgatgatgccttcggcggagggaccgaggtggtggtcaaacgtacggtagcggccccatctgtcttcatcttcccgccatctgatgagcagttgaaatctggaactgcctctgttgtgtgcctgctgaataactt

SEQ ID NO: 62 - DNA, 502 bp, Oryctolagus cuniculus
  source 1..502: mol_type = genomic DNA
atggagactgggctgcgctggcttctcctggtggctgtgctcaaaggtgtccagtgtcaggagcagctgaaggagtccggaggaggcctggtaacgcctggaggaaccctgacactcacctgcacagcctctggattctccctcaatgcctactacatgaactgggtccgccaggctccagggaaggggctggaatggatcggattcattactctgaataataatgtagcttacgcgaactgggcgaaaggccgattcaccttctccaaaacctcgaccacggtggatctgaaaatgaccagtccgacacccgaggacacggccacctatttctgtgccaggagtcgtggctggggtgcaatgggtcggttggatctctggggccatggcaccctggtcaccgtctcgagcgcctccaccaagggcccatcggtcttccccctggcaccctcctccaagagcacctctgggggcacagcggccctgggctgcctggtcaagg

SEQ ID NO: 63 - DNA, 39 bp, Oryctolagus cuniculus
  source 1..39: mol_type = genomic DNA
caggccagtcagagtgtttatgagaacaactatttatcc

SEQ ID NO: 64 - DNA, 21 bp, Oryctolagus cuniculus
  source 1..21: mol_type = genomic DNA
ggtgcatccactctggattct

SEQ ID NO: 65 - DNA, 33 bp, Oryctolagus cuniculus
  source 1..33: mol_type = genomic DNA
gcaggcgtttatgatgatgatagtgatgatgcc

SEQ ID NO: 66 - DNA, 15 bp, Oryctolagus cuniculus
  source 1..15: mol_type = genomic DNA
gcctactacatgaac

SEQ ID NO: 67 - DNA, 48 bp, Oryctolagus cuniculus
  source 1..48: mol_type = genomic DNA
ttcattactctgaataataatgtagcttacgcgaactgggcgaaaggc

SEQ ID NO: 68 - DNA, 36 bp, Oryctolagus cuniculus
  source 1..36: mol_type = genomic DNA
agtcgtggctggggtgcaatgggtcggttggatctc

SEQ ID NO: 69 - protein, 164 aa, Oryctolagus cuniculus
MDTRAPTQLLGLLLLWLPGATFAQVLTQTPSPVSAAVGGTVTINCQASQSVDDNNWLGWYQQKRGQPPKYLIYSASTLASGVPSRFKGSGSGTQFTLTISDLECDDAATYYCAGGFSGNIFAFGGGTEVVVKRTVAAPSVFIFPPSDEQLKSGTASVVCLLNNF

SEQ ID NO: 70 - protein, 164 aa, Oryctolagus cuniculus
METGLRWLLLVAVLKGVQCQSVEESGGRLVTPGTPLTLTCTVSGFSLSSYAMSWVRQAPGKGLEWIGIIGGFGTTYYATWAKGRFTISKTSTTVDLRITSPTTEDTATYFCARGGPGNGGDIWGQGTLVTVSSASTKGPSVFPLAPSSKSTSGGTAALGCLVKD

SEQ ID NO: 71 - protein, 13 aa, Oryctolagus cuniculus
QASQSVDDNNWLG

SEQ ID NO: 72 - protein, 7 aa, Oryctolagus cuniculus
SASTLAS

SEQ ID NO: 73 - protein, 10 aa, Oryctolagus cuniculus
AGGFSGNIFA

SEQ ID NO: 74 - protein, 5 aa, Oryctolagus cuniculus
SYAMS

SEQ ID NO: 75 - protein, 16 aa, Oryctolagus cuniculus
IIGGFGTTYYATWAKG

SEQ ID NO: 76 - protein, 9 aa, Oryctolagus cuniculus
GGPGNGGDI

SEQ ID NO: 77 - DNA, 493 bp, Oryctolagus cuniculus
  source 1..493: mol_type = genomic DNA
atggacacgagggcccccactcagctgctggggctcctgctgctctggctcccaggtgccacatttgcccaagtgctgacccagactccatcgcctgtgtctgcagctgtgggaggcacagtcaccatcaactgccaggccagtcagagtgttgatgataacaactggttaggctggtatcagcagaaacgagggcagcctcccaagtacctgatctattctgcatccactctggcatctggggtcccatcgcggttcaaaggcagtggatctgggacacagttcactctcaccatcagcgacctggagtgtgacgatgctgccacttactactgtgcaggcggttttagtggtaatatctttgctttcggcggagggaccgaggtggtggtcaaacgtacggtagcggccccatctgtcttcatcttcccgccatctgatgagcagttgaaatctggaactgcctctgttgtgtgcctgctgaataacttct

SEQ ID NO: 78 - DNA, 493 bp, Oryctolagus cuniculus
  source 1..493: mol_type = genomic DNA
atggagactgggctgcgctggcttctcctggtcgctgtgctcaaaggtgtccagtgtcagtcggtggaggagtccgggggtcgcctggtcacgcctgggacacccctgacactcacctgcacagtctctggcttctccctcagtagctatgcaatgagctgggtccgccaggctccaggaaaggggctggagtggatcggaatcattggtggttttggtaccacatactacgcgacctgggcgaaaggccgattcaccatctccaaaacctcgaccacggtggatctgagaatcaccagtccgacaaccgaggacacggccacctatttctgtgccagaggtggtcctggtaatggtggtgacatctggggccaagggaccctggtcaccgtctcgagcgcctccaccaagggcccatcggtcttccccctggcaccctcctccaagagcacctctgggggcacagcggccctgggctgcctggtcaaggact

SEQ ID NO: 79 - DNA, 39 bp, Oryctolagus cuniculus
  source 1..39: mol_type = genomic DNA
caggccagtcagagtgttgatgataacaactggttaggc

SEQ ID NO: 80 - DNA, 21 bp, Oryctolagus cuniculus
  source 1..21: mol_type = genomic DNA
tctgcatccactctggcatct

SEQ ID NO: 81 - DNA, 30 bp, Oryctolagus cuniculus
  source 1..30: mol_type = genomic DNA
gcaggcggttttagtggtaatatctttgct

SEQ ID NO: 82 - DNA, 15 bp, Oryctolagus cuniculus
  source 1..15: mol_type = genomic DNA
agctatgcaatgagc

SEQ ID NO: 83 - DNA, 48 bp, Oryctolagus cuniculus
  source 1..48: mol_type = genomic DNA
atcattggtggttttggtaccacatactacgcgacctgggcgaaaggc

SEQ ID NO: 84 - DNA, 27 bp, Oryctolagus cuniculus
  source 1..27: mol_type = genomic DNA
ggtggtcctggtaatggtggtgacatc

SEQ ID NO: 85 - protein, 164 aa, Oryctolagus cuniculus
MDTRAPTQLLGLLLLWLPGATFAAVLTQTPSPVSVPVGGTVTIKCQSSQSVYNNFLSWYQQKPGQPPKLLIYQASKLASGVPDRFSGSGSGTQFTLTISGVQCDDAATYYCLGGYDDDADNAFGGGTEVVVKRTVAAPSVFIFPPSDEQLKSGTASVVCLLNNF

SEQ ID NO: 86 - protein, 170 aa, Oryctolagus cuniculus
METGLRWLLLVAVLKGVQCQSVEESGGRLVTPGTPLTLTCTVSGIDLSDYAMSWVRQAPGKGLEWIGIIYAGSGSTWYASWAKGRFTISKTSTTVDLKITSPTTEDTATYFCARDGYDDYGDFDRLDLWGPGTLVTVSSASTKGPSVFPLAPSSKSTSGGTAALGCLVKD

SEQ ID NO: 87 - protein, 12 aa, Oryctolagus cuniculus
QSSQSVYNNFLS

SEQ ID NO: 88 - protein, 7 aa, Oryctolagus cuniculus
QASKLAS

SEQ ID NO: 89 - protein, 11 aa, Oryctolagus cuniculus
LGGYDDDADNA

SEQ ID NO: 90 - protein, 5 aa, Oryctolagus cuniculus
DYAMS

SEQ ID NO: 91 - protein, 17 aa, Oryctolagus cuniculus
IIYAGSGSTWYASWAKG

SEQ ID NO: 92 - protein, 14 aa, Oryctolagus cuniculus
DGYDDYGDFDRLDL

SEQ ID NO: 93 - DNA, 492 bp, Oryctolagus cuniculus
  source 1..492: mol_type = genomic DNA
atggacacgagggcccccactcagctgctggggctcctgctgctctggctcccaggtgccacatttgcagccgtgctgacccagacaccatcgcccgtgtctgtacctgtgggaggcacagtcaccatcaagtgccagtccagtcagagtgtttataataatttcttatcgtggtatcagcagaaaccagggcagcctcccaagctcctgatctaccaggcatccaaactggcatctggggtcccagataggttcagcggcagtggatctgggacacagttcactctcaccatcagcggcgtgcagtgtgacgatgctgccacttactactgtctaggcggttatgatgatgatgctgataatgctttcggcggagggaccgaggtggtggtcaaacgtacggtagcggccccatctgtcttcatcttcccgccatctgatgagcagttgaaatctggaactgcctctgttgtgtgcctgctgaataacttc

SEQ ID NO: 94 - DNA, 511 bp, Oryctolagus cuniculus
  source 1..511: mol_type = genomic DNA
atggagactgggctgcgctggcttctcctggtcgctgtgctcaaaggtgtccagtgtcagtcggtggaggagtccgggggtcgcctggtcacgcctgggacacccctgacgctcacctgcacagtctctggaatcgacctcagtgactatgcaatgagctgggtccgccaggctccagggaaggggctggaatggatcggaatcatttatgctggtagtggtagcacatggtacgcgagctgggcgaaaggccgattcaccatctccaaaacctcgaccacggtggatctgaaaatcaccagtccgacaaccgaggacacggccacctatttctgtgccagagatggatacgatgactatggtgatttcgatcgattggatctctggggcccaggcaccctcgtcaccgtctcgagcgcctccaccaagggcccatcggtcttccccctggcaccctcctccaagagcacctctgggggcacagcggccctgggctgcctggtcaaggact

SEQ ID NO: 95 - DNA, 36 bp, Oryctolagus cuniculus
  source 1..36: mol_type = genomic DNA
cagtccagtcagagtgtttataataatttcttatcg

SEQ ID NO: 96 - DNA, 21 bp, Oryctolagus cuniculus
  source 1..21: mol_type = genomic DNA
caggcatccaaactggcatct

SEQ ID NO: 97 - DNA, 33 bp, Oryctolagus cuniculus
  source 1..33: mol_type = genomic DNA
ctaggcggttatgatgatgatgctgataatgct

SEQ ID NO: 98 - DNA, 15 bp, Oryctolagus cuniculus
  source 1..15: mol_type = genomic DNA
gactatgcaatgagc

SEQ ID NO: 99 - DNA, 51 bp, Oryctolagus cuniculus
  source 1..51: mol_type = genomic DNA
atcatttatgctggtagtggtagcacatggtacgcgagctgggcgaaaggc

SEQ ID NO: 100 - DNA, 42 bp, Oryctolagus cuniculus
  source 1..42: mol_type = genomic DNA
gatggatacgatgactatggtgatttcgatcgattggatctc

SEQ ID NO: 101 - protein, 164 aa, Oryctolagus cuniculus
MDTRAPTQLLGLLLLWLPGARCAYDMTQTPASVSAAVGGTVTIKCQASQSINNELSWYQQKSGQRPKLLIYRASTLASGVSSRFKGSGSGTEFTLTISDLECADAATYYCQQGYSLRNIDNAFGGGTEVVVKRTVAAPSVFIFPPSDEQLKSGTASVVCLLNNF

SEQ ID NO: 102 - protein, 166 aa, Oryctolagus cuniculus
METGLRWLLLVAVLSGVQCQSLEESGGRLVTPGTPLTLTCTASGFSLSNYYMTWVRQAPGKGLEWIGMIYGSDETAYANWAIGRFTISKTSTTVDLKMTSLTAADTATYFCARDDSSDWDAKFNLWGQGTLVTVSSASTKGPSVFPLAPSSKSTSGGTAALGCLVK

SEQ ID NO: 103 - protein, 11 aa, Oryctolagus cuniculus
QASQSINNELS

SEQ ID NO: 104 - protein, 7 aa, Oryctolagus cuniculus
RASTLAS

SEQ ID NO: 105 - protein, 12 aa, Oryctolagus cuniculus
QQGYSLRNIDNA

SEQ ID NO: 106 - protein, 5 aa, Oryctolagus cuniculus
NYYMT

SEQ ID NO: 107 - protein, 16 aa, Oryctolagus cuniculus
MIYGSDETAYANWAIG

SEQ ID NO: 108 - protein, 12 aa, Oryctolagus cuniculus
DDSSDWDAKFNL

SEQ ID NO: 109 - DNA, 492 bp, Oryctolagus cuniculus
  source 1..492: mol_type = genomic DNA
atggacacgagggcccccactcagctgctggggctcctgctgctctggctcccaggtgccagatgtgcctatgatatgacccagactccagcctcggtgtctgcagctgtgggaggcacagtcaccatcaaatgccaggccagtcagagcattaacaatgaattatcctggtatcagcagaaatcagggcagcgtcccaagctcctgatctatagggcatccactctggcatctggggtctcatcgcggttcaaaggcagtggatctgggacagagttcactctcaccatcagcgacctggagtgtgccgatgctgccacttactactgtcaacagggttatagtctgaggaatattgataatgctttcggcggagggaccgaggtggtggtcaaacgtacggtagcggccccatctgtcttcatcttcccgccatctgatgagcagttgaaatctggaactgcctctgttgtgtgcctgctgaataacttc

SEQ ID NO: 110 - DNA, 499 bp, Oryctolagus cuniculus
  source 1..499: mol_type = genomic DNA
atggagactgggctgcgctggcttctcctggtcgctgtgctctcaggtgtccagtgtcagtcgctggaggagtccgggggtcgcctggtcacgcctgggacacccctgacactcacctgcacagcctctggattctccctcagtaactactacatgacctgggtccgccaggctccagggaaggggctggaatggatcggaatgatttatggtagtgatgaaacagcctacgcgaactgggcgataggccgattcaccatctccaaaacctcgaccacggtggatctgaaaatgaccagtctgacagccgcggacacggccacctatttctgtgccagagatgatagtagtgactgggatgcaaaatttaacttgtggggccaagggaccctcgtcaccgtctcgagcgcctccaccaagggcccatcggtcttccccctggcaccctcctccaagagcacctctgggggcacagcggccctgggctgcctggtcaagg

SEQ ID NO: 111 - DNA, 33 bp, Oryctolagus cuniculus; identical to SEQ ID NO: 12
  source 1..33: mol_type = genomic DNA

SEQ ID NO: 112 - DNA, 21 bp, Oryctolagus cuniculus
  source 1..21: mol_type = genomic DNA
agggcatccactctggcatct

SEQ ID NO: 113 - DNA, 36 bp, Oryctolagus cuniculus; identical to SEQ ID NO: 14
  source 1..36: mol_type = genomic DNA

SEQ ID NO: 114 - DNA, 15 bp, Oryctolagus cuniculus
  source 1..15: mol_type = genomic DNA
aactactacatgacc

SEQ ID NO: 115 - DNA, 48 bp, Oryctolagus cuniculus
  source 1..48: mol_type = genomic DNA
atgatttatggtagtgatgaaacagcctacgcgaactgggcgataggc

SEQ ID NO: 116 - DNA, 36 bp, Oryctolagus cuniculus; identical to SEQ ID NO: 17
  source 1..36: mol_type = genomic DNA

SEQ ID NO: 117 - protein, 109 aa, Oryctolagus cuniculus
EVQLVESGGGLVQPGGSLRLSCAASGFSLSNYYMTWVRQAPGKGLEWVGMIYGSDETAYANWAIGRFTISRDNSKNTLYLQMNSLRAEDTAVYYCARDDSSDWDAKFNL

SEQ ID NO: 118 - protein, 109 aa, Oryctolagus cuniculus
EVQLVESGGGLVQPGGSLRLSCAASGFSLSNYYMTWVRQAPGKGLEWVGMIYGSDETAYANSAIGRFTISRDNSKNTLYLQMNSLRAEDTAVYYCARDDSSDWDAKFNL

SEQ ID NO: 119 - protein, 100 aa, Oryctolagus cuniculus
DIQMTQSPSTLSASVGDRVTITCQASQSINNELSWYQQKPGKAPKLLIYRASTLASGVPSRFSGSGSGTEFTLTISSLQPDDFATYYCQQGYSLRNIDNA

SEQ ID NO: 120 - protein, 16 aa, Oryctolagus cuniculus
IIYGSDETAYATSAIG

SEQ ID NO: 121 - protein, 16 aa, Oryctolagus cuniculus
MIYGSDETAYANSAIG

SEQ ID NO: 122 - protein, 123 aa, Oryctolagus cuniculus
MDTRAPTQLLGLLLLWLPGATFAAVLTQTPSPVSAAVGGTVTISCQSSQSVGNNQDLSWFQQRPGQPPKLLIYEISKLESGVPSRFSGSGSGTHFTLTISGVQCDDAATYYCLGGYDDDADNA

SEQ ID NO: 123 - protein, 128 aa, Oryctolagus cuniculus
METGLRWLLLVAVLKGVQCHSVEESGGRLVTPGTPLTLTCTVSGFSLSSRTMSWVRQAPGKGLEWIGYIWSGGSTYYATWAKGRFTISKTSTTVDLKITSPTTEDTATYFCARLGDTGGHAYATRLNL

SEQ ID NO: 124 - protein, 13 aa, Oryctolagus cuniculus
QSSQSVGNNQDLS

SEQ ID NO: 125 - protein, 7 aa, Oryctolagus cuniculus
EISKLES

SEQ ID NO: 126 - protein, 11 aa, Oryctolagus cuniculus
LGGYDDDADNA

SEQ ID NO: 127 - protein, 5 aa, Oryctolagus cuniculus
SRTMS

SEQ ID NO: 128 - protein, 16 aa, Oryctolagus cuniculus
YIWSGGSTYYATWAKG

SEQ ID NO: 129 - protein, 15 aa, Oryctolagus cuniculus
LGDTGGHAYATRLNL

SEQ ID NO: 130 - DNA, 369 bp, Oryctolagus cuniculus
  source 1..369: mol_type = genomic DNA
atggacacgagggcccccactcagctgctggggctcctgctgctctggctcccaggtgccacatttgcagccgtgctgacccagacaccatcacccgtgtctgcagctgtgggaggcacagtcaccatcagttgccagtccagtcagagtgttggtaataaccaggacttatcctggtttcagcagagaccagggcagcctcccaagctcctgatctacgaaatatccaaactggaatctggggtcccatcgcggttcagcggcagtggatctgggacacacttcactctcaccatcagcggcgtacagtgtgacgatgctgccacttactactgtctaggcggttatgatgatgatgctgataatgct

SEQ ID NO: 131 - DNA, 384 bp, Oryctolagus cuniculus
  source 1..384: mol_type = genomic DNA
atggagactgggctgcgctggcttctcctggtcgctgtgctcaaaggtgtccagtgtcactcggtggaggagtccgggggtcgcctggtcacgcctgggacacccctgacactcacctgcacagtctctggattctccctcagtagtcgtacaatgtcctgggtccgccaggctccagggaaggggctggagtggatcggatacatttggagtggtggtagcacatactacgcgacctgggcgaaaggccgattcaccatctccaaaacctcgaccacggtggatctgaaaatcaccagtccgacaaccgaggacacggccacctatttctgtgccagattgggcgatactggtggtcacgcttatgctactcgcttaaatctc

SEQ ID NO: 132 - DNA, 39 bp, Oryctolagus cuniculus
  source 1..39: mol_type = genomic DNA
cagtccagtcagagtgttggtaataaccaggacttatcc

SEQ ID NO: 133 - DNA, 21 bp, Oryctolagus cuniculus
  source 1..21: mol_type = genomic DNA
gaaatatccaaactggaatct

SEQ ID NO: 134 - DNA, 33 bp, Oryctolagus cuniculus; identical to SEQ ID NO: 97
  source 1..33: mol_type = genomic DNA

SEQ ID NO: 135 - DNA, 15 bp, Oryctolagus cuniculus
  source 1..15: mol_type = genomic DNA
agtcgtacaatgtcc

SEQ ID NO: 136 - DNA, 48 bp, Oryctolagus cuniculus
  source 1..48: mol_type = genomic DNA
tacatttggagtggtggtagcacatactacgcgacctgggcgaaaggc

SEQ ID NO: 137 - DNA, 45 bp, Oryctolagus cuniculus
  source 1..45: mol_type = genomic DNA
ttgggcgatactggtggtcacgcttatgctactcgcttaaatctc

SEQ ID NO: 138 - protein, 123 aa, Oryctolagus cuniculus
MDTRAPTQLLGLLLLWLPGATFAAVLTQTPSSVSAAVGGTVSISCQSSQSVYSNKYLAWYQQKPGQPPKLLIYWTSKLASGAPSRFSGSGSGTQFTLTISGVQCDDAATYYCLGAYDDDADNA

SEQ ID NO: 139 - protein, 126 aa, Oryctolagus cuniculus
METGLRWLLLVAVLKGVQCQSVEESGGRLVKPDETLTLTCTASGFSLEGGYMTWVRQAPGKGLEWIGISYDSGSTYYASWAKGRFTISKTSSTTVDLKMTSLTTEDTATYFCVRSLKYPTVTSDDL

SEQ ID NO: 140 - protein, 13 aa, Oryctolagus cuniculus
QSSQSVYSNKYLA

SEQ ID NO: 141 - protein, 7 aa, Oryctolagus cuniculus
WTSKLAS

SEQ ID NO: 142 - protein, 11 aa, Oryctolagus cuniculus
LGAYDDDADNA

SEQ ID NO: 143 - protein, 5 aa, Oryctolagus cuniculus
GGYMT

SEQ ID NO: 144 - protein, 16 aa, Oryctolagus cuniculus
ISYDSGSTYYASWAKG

SEQ ID NO: 145 - protein, 12 aa, Oryctolagus cuniculus
SLKYPTVTSDDL

SEQ ID NO: 146 - DNA, 369 bp, Oryctolagus cuniculus
  source 1..369: mol_type = genomic DNA
atggacacgagggcccccactcagctgctggggctcctgctgctctggctcccaggtgccacatttgcagccgtgctgacccagacaccatcgtccgtgtctgcagctgtgggaggcacagtcagcatcagttgccagtccagtcagagtgtttatagtaataagtacctagcctggtatcagcagaaaccagggcagcctcccaagctcctgatctactggacatccaaactggcatctggggccccatcacggttcagcggcagtggatctgggacacaattcactctcaccatcagcggcgtgcagtgtgacgatgctgccacttactactgtctaggcgcttatgatgatgatgctgataatgct

SEQ ID NO: 147 - DNA, 378 bp, Oryctolagus cuniculus
  source 1..378: mol_type = genomic DNA
atggagactgggctgcgctggcttctcctggtcgctgtgctcaaaggtgtccagtgtcagtcggtggaagagtccgggggtcgcctggtcaagcctgacgaaaccctgacactcacctgcacagcctctggattctccctggagggcggctacatgacctgggtccgccaggctccagggaaggggctggaatggatcggaatcagttatgatagtggtagcacatactacgcgagctgggcgaaaggccgattcaccatctccaagacctcgtcgaccacggtggatctgaaaatgaccagtctgacaaccgaggacacggccacctatttctgcgtcagatcactaaaatatcctactgttacttctgatgacttg

SEQ ID NO: 148 - DNA, 39 bp, Oryctolagus cuniculus
  source 1..39: mol_type = genomic DNA
cagtccagtcagagtgtttatagtaataagtacctagcc

SEQ ID NO: 149 - DNA, 21 bp, Oryctolagus cuniculus
  source 1..21: mol_type = genomic DNA
tggacatccaaactggcatct

SEQ ID NO: 150 - DNA, 33 bp, Oryctolagus cuniculus
  source 1..33: mol_type = genomic DNA
ctaggcgcttatgatgatgatgctgataatgct

SEQ ID NO: 151 - DNA, 15 bp, Oryctolagus cuniculus
  source 1..15: mol_type = genomic DNA
ggcggctacatgacc

SEQ ID NO: 152 - DNA, 48 bp, Oryctolagus cuniculus
  source 1..48: mol_type = genomic DNA
atcagttatgatagtggtagcacatactacgcgagctgggcgaaaggc

SEQ ID NO: 153 - DNA, 36 bp, Oryctolagus cuniculus
  source 1..36: mol_type = genomic DNA
tcactaaaatatcctactgttacttctgatgacttg

SEQ ID NO: 154 - protein, 123 aa, Oryctolagus cuniculus
MDTRAPTQLLGLLLLWLPGATFAAVLTQTPSPVSAAVGGTVTISCQSSQSVYNNNDLAWYQQKPGQPPKLLIYYASTLASGVPSRFKGSGSGTQFTLTISGVQCDDAAAYYCLGGYDDDADNA

SEQ ID NO: 155 - protein, 129 aa, Oryctolagus cuniculus
METGLRWLLLVAVLKGVQCQSVEESGGRLVTPGTPLTLTCTVSGLSLSSNTINWVRQAPGKGLEWIGYIWSGGSTYYASWVNGRFTISKTSTTVDLKITSPTTEDTATYFCARGGYASGGYPYATRLDL

SEQ ID NO: 156 - protein, 13 aa, Oryctolagus cuniculus
QSSQSVYNNNDLA

SEQ ID NO: 157 - protein, 7 aa, Oryctolagus cuniculus
YASTLAS

SEQ ID NO: 158 - protein, 11 aa, Oryctolagus cuniculus
LGGYDDDADNA

SEQ ID NO: 159 - protein, 5 aa, Oryctolagus cuniculus
SNTIN

SEQ ID NO: 160 - protein, 16 aa, Oryctolagus cuniculus
YIWSGGSTYYASWVNG

SEQ ID NO: 161 - protein, 16 aa, Oryctolagus cuniculus
GGYASGGYPYATRLDL

SEQ ID NO: 162 - DNA, 369 bp, Oryctolagus cuniculus
  source 1..369: mol_type = genomic DNA
atggacacgagggcccccactcagctgctggggctcctgctgctctggctcccaggtgccacatttgcagccgtgctgacccagacaccatcacccgtgtctgcagctgtgggaggcacagtcaccatcagttgccagtccagtcagagtgtttataataataacgacttagcctggtatcagcagaaaccagggcagcctcctaaactcctgatctattatgcatccactctggcatctggggtcccatcgcggttcaaaggcagtggatctgggacacagttcactctcaccatcagcggcgtgcagtgtgacgatgctgccgcttactactgtctaggcggttatgatgatgatgctgataatgct

SEQ ID NO: 163 - DNA, 387 bp, Oryctolagus cuniculus
  source 1..387: mol_type = genomic DNA
atggagactgggctgcgctggcttctcctggtcgctgtgctcaaaggtgtccagtgtcagtcggtggaggagtccgggggtcgcctggtcacgcctgggacacccctgacactcacctgcacagtatctggattatccctcagtagcaatacaataaactgggtccgccaggctccagggaaggggctggagtggatcggatacatttggagtggtggtagtacatactacgcgagctgggtgaatggtcgattcaccatctccaaaacctcgaccacggtggatctgaaaatcaccagtccgacaaccgaggacacggccacctatttctgtgccagagggggttacgctagtggtggttatccttatgccactcggttggatctc

SEQ ID NO: 164 - DNA, 39 bp, Oryctolagus cuniculus
  source 1..39: mol_type = genomic DNA
cagtccagtcagagtgtttataataataacgacttagcc

SEQ ID NO: 165 - DNA, 21 bp, Oryctolagus cuniculus
  source 1..21: mol_type = genomic DNA
tatgcatccactctggcatct

SEQ ID NO: 166 - DNA, 33 bp, Oryctolagus cuniculus; identical to SEQ ID NO: 97
  source 1..33: mol_type = genomic DNA

SEQ ID NO: 167 - DNA, 15 bp, Oryctolagus cuniculus
  source 1..15: mol_type = genomic DNA
agcaatacaataaac

SEQ ID NO: 168 - DNA, 48 bp, Oryctolagus cuniculus
  source 1..48: mol_type = genomic DNA
tacatttggagtggtggtagtacatactacgcgagctgggtgaatggt

SEQ ID NO: 169 - DNA, 48 bp, Oryctolagus cuniculus
  source 1..48: mol_type = genomic DNA
gggggttacgctagtggtggttatccttatgccactcggttggatctc

SEQ ID NO: 170 - protein, 123 aa, Oryctolagus cuniculus
MDTRAPTQLLGLLLLWLPGATFAAVLTQTPSSVSAAVGGTVTINCQSSQSVYNNDYLSWYQQRPGQRPKLLIYGASKLASGVPSRFKGSGSGKQFTLTISGVQCDDAATYYCLGDYDDDADNT

SEQ ID NO: 171 - protein, 123 aa, Oryctolagus cuniculus
METGLRWLLLVAVLKGVQCQSLEESGGRLVTPGTPLTLTCTVSGFTLSTNYYLSWVRQAPGKGLEWIGIIYPSGNTYCAKWAKGRFTISKTSSTTVDLKMTSPTTEDTATYFCARNYGGDESL

SEQ ID NO: 172 - protein, 13 aa, Oryctolagus cuniculus
QSSQSVYNNDYLS

SEQ ID NO: 173 - protein, 7 aa, Oryctolagus cuniculus
GASKLAS

SEQ ID NO: 174 - protein, 11 aa, Oryctolagus cuniculus
LGDYDDDADNT

SEQ ID NO: 175 - protein, 6 aa, Oryctolagus cuniculus
TNYYLS

SEQ ID NO: 176 - protein, 16 aa, Oryctolagus cuniculus
IIYPSGNTYCAKWAKG

SEQ ID NO: 177 - protein, 8 aa, Oryctolagus cuniculus
NYGGDESL

SEQ ID NO: 178 - DNA, 369 bp, Oryctolagus cuniculus
  source 1..369: mol_type = genomic DNA
atggacacgagggcccccactcagctgctggggctcctgctgctctggctcccaggtgccacatttgcagccgtgctgacccagacaccatcctccgtgtctgcagctgtgggaggcacagtcaccatcaattgccagtccagtcagagtgtttataataacgactacttatcctggtatcaacagaggccagggcaacgtcccaagctcctaatctatggtgcttccaaactggcatctggggtcccgtcacggttcaaaggcagtggatctgggaaacagtttactctcaccatcagcggcgtgcagtgtgacgatgctgccacttactactgtctgggcgattatgatgatgatgctgataatact

SEQ ID NO: 179 - DNA, 369 bp, Oryctolagus cuniculus
  source 1..369: mol_type = genomic DNA
atggagactgggctgcgctggcttctcctggtcgctgtgctcaaaggtgtccagtgtcagtcgctggaggagtccgggggtcgcctggtcacgcctgggacacccctgacactcacttgcacagtctctggattcaccctcagtaccaactactacctgagctgggtccgccaggctccagggaaggggctagaatggatcggaatcatttatcctagtggtaacacatattgcgcgaagtgggcgaaaggccgattcaccatctccaaaacctcgtcgaccacggtggatctgaaaatgaccagtccgacaaccgaggacacagccacgtatttctgtgccagaaattatggtggtgatgaaagtttg

SEQ ID NO: 180 - DNA, 39 bp, Oryctolagus cuniculus
  source 1..39: mol_type = genomic DNA
cagtccagtcagagtgtttataataacgactacttatcc

SEQ ID NO: 181 - DNA, 21 bp, Oryctolagus cuniculus
  source 1..21: mol_type = genomic DNA
ggtgcttccaaactggcatct

SEQ ID NO: 182 - DNA, 33 bp, Oryctolagus cuniculus
  source 1..33: mol_type = genomic DNA
ctgggcgattatgatgatgatgctgataatact

SEQ ID NO: 183 - DNA, 18 bp, Oryctolagus cuniculus
  source 1..18: mol_type = genomic DNA
accaactactacctgagc

SEQ ID NO: 184 - DNA, 48 bp, Oryctolagus cuniculus
  source 1..48: mol_type = genomic DNA
atcatttatcctagtggtaacacatattgcgcgaagtgggcgaaaggc

SEQ ID NO: 185 - DNA, 24 bp, Oryctolagus cuniculus
  source 1..24: mol_type = genomic DNA
aattatggtggtgatgaaagtttg

SEQ ID NO: 186 - protein, 119 aa, Oryctolagus cuniculus
MDTRAPTQLLGLLLLWLPGARCDVVMTQTPASVEAAVGGTVTIKCQASETIGNALAWYQQKSGQPPKLLIYKASKLASGVPSRFKGSGSGTEYTLTISDLECADAATYYCQWCYFGDSV

SEQ ID NO: 187 - protein, 128 aa, Oryctolagus cuniculus
METGLRWLLLVTVLKGVQCQEQLVESGGGLVQPEGSLTLTCTASGFDFSSGYYMCWVRQAPGKGLEWIACIFTITTNTYYASWAKGRFTISKTSSTTVTLQMTSLTAADTATYLCARGIYSDNNYYAL

SEQ ID NO: 188 - protein, 11 aa, Oryctolagus cuniculus
QASETIGNALA

SEQ ID NO: 189 - protein, 7 aa, Oryctolagus cuniculus
KASKLAS

SEQ ID NO: 190 - protein, 9 aa, Oryctolagus cuniculus
QWCYFGDSV

SEQ ID NO: 191 - protein, 6 aa, Oryctolagus cuniculus
SGYYMC

SEQ ID NO: 192 - protein, 17 aa, Oryctolagus cuniculus
CIFTITTNTYYASWAKG

SEQ ID NO: 193 - protein, 11 aa, Oryctolagus cuniculus
GIYSDNNYYAL

SEQ ID NO: 194 - DNA, 357 bp, Oryctolagus cuniculus
  source 1..357: mol_type = genomic DNA
atggacacgagggcccccactcagctgctggggctcctgctgctctggctcccaggtgccagatgtgatgttgtgatgacccagactccagcctccgtggaggcagctgtgggaggcacagtcaccatcaagtgccaggccagtgagaccattggcaatgcattagcctggtatcagcagaaatcagggcagcctcccaagctcctgatctacaaggcatccaaactggcatctggggtcccatcgcggttcaaaggcagtggatctgggacagagtacactctcaccatcagcgacctggagtgtgccgatgctgccacttactactgtcaatggtgttattttggtgatagtgtt

SEQ ID NO: 195 - DNA, 384 bp, Oryctolagus cuniculus
  source 1..384: mol_type = genomic DNA
atggagactgggctgcgctggcttctcctggtcactgtgctcaaaggtgtccagtgtcaggagcagctggtggagtccgggggaggcctggtccagcctgagggatccctgacactcacctgcacagcctctggattcgacttcagtagcggctactacatgtgctgggtccgccaggctccagggaaggggctggagtggatcgcgtgtattttcactattactactaacacttactacgcgagctgggcgaaaggccgattcaccatctccaagacctcgtcgaccacggtgactctgcaaatgaccagtctgacagccgcggacacggccacctatctctgtgcgagagggatttattctgataataattattatgccttg

SEQ ID NO: 196 - DNA, 33 bp, Oryctolagus cuniculus
  source 1..33: mol_type = genomic DNA
caggccagtgagaccattggcaatgcattagcc

SEQ ID NO: 197 - DNA, 21 bp, Oryctolagus cuniculus
  source 1..21: mol_type = genomic DNA
aaggcatccaaactggcatct

SEQ ID NO: 198 - DNA, 27 bp, Oryctolagus cuniculus
  source 1..27: mol_type = genomic DNA
caatggtgttattttggtgatagtgtt

SEQ ID NO: 199 - DNA, 18 bp, Oryctolagus cuniculus
  source 1..18: mol_type = genomic DNA
agcggctactacatgtgc

SEQ ID NO: 200 - DNA, 51 bp, Oryctolagus cuniculus
  source 1..51: mol_type = genomic DNA
tgtattttcactattactactaacacttactacgcgagctgggcgaaaggc

SEQ ID NO: 201 - DNA, 33 bp, Oryctolagus cuniculus
  source 1..33: mol_type = genomic DNA
gggatttattctgataataattattatgccttg

SEQ ID NO: 202 - protein, 119 aa, Oryctolagus cuniculus
MDTRAPTQLLGLLLLWLPGARCDVVMTQTPASVEAAVGGTVTIKCQASESIGNALAWYQQKPGQPPKLLIYKASTLASGVPSRFSGSGSGTEFTLTISGVQCADAAAYYCQWCYFGDSV

SEQ ID NO: 203 - protein, 128 aa, Oryctolagus cuniculus
METGLRWLLLVAVLKGVQCQQQLVESGGGLVKPGASLTLTCKASGFSFSSGYYMCWVRQAPGKGLESIACIFTITDNTYYANWAKGRFTISKPSSPTVTLQMTSLTAADTATYFCARGIYSTDNYYAL

SEQ ID NO: 204 - protein, 11 aa, Oryctolagus cuniculus
QASESIGNALA

SEQ ID NO: 205 - protein, 7 aa, Oryctolagus cuniculus
KASTLAS

SEQ ID NO: 206 - protein, 9 aa, Oryctolagus cuniculus
QWCYFGDSV

SEQ ID NO: 207 - protein, 6 aa, Oryctolagus cuniculus
SGYYMC

SEQ ID NO: 208 - protein, 17 aa, Oryctolagus cuniculus
CIFTITDNTYYANWAKG

SEQ ID NO: 209 - protein, 11 aa, Oryctolagus cuniculus
GIYSTDNYYAL

SEQ ID NO: 210 - DNA, 357 bp, Oryctolagus cuniculus
  source 1..357: mol_type = genomic DNA
atggacacgagggcccccactcagctgctggggctcctgctgctctggctcccaggtgccagatgtgatgttgtgatgacccagactccagcctccgtggaggcagctgtgggaggcacagtcaccatcaagtgccaggccagtgagagcattggcaatgcattagcctggtatcagcagaaaccagggcagcctcccaagctcctgatctacaaggcatccactctggcatctggggtcccatcgcggttcagcggcagtggatctgggacagagttcactctcaccatcagcggcgtgcagtgtgccgatgctgccgcttactactgtcaatggtgttattttggtgatagtgtt

SEQ ID NO: 211 - DNA, 384 bp, Oryctolagus cuniculus
  source 1..384: mol_type = genomic DNA
atggagactgggctgcgctggcttctcctggtcgctgtgctcaaaggtgtccagtgtcagcagcagctggtggagtccgggggaggcctggtcaagccgggggcatccctgacactcacctgcaaagcctctggattctccttcagtagcggctactacatgtgctgggtccgccaggctccagggaaggggctggagtcgatcgcatgcatttttactattactgataacacttactacgcgaactgggcgaaaggccgattcaccatctccaagccctcgtcgcccacggtgactctgcaaatgaccagtctgacagccgcggacacggccacctatttctgtgcgagggggatttattctactgataattattatgccttg

SEQ ID NO: 212 - DNA, 33 bp, Oryctolagus cuniculus
  source 1..33: mol_type = genomic DNA
caggccagtgagagcattggcaatgcattagcc

SEQ ID NO: 213 - DNA, 21 bp, Oryctolagus cuniculus
  source 1..21: mol_type = genomic DNA
aaggcatccactctggcatct

SEQ ID NO: 214 - DNA, 27 bp, Oryctolagus cuniculus
  source 1..27: mol_type = genomic DNA
caatggtgttattttggtgatagtgtt

SEQ ID NO: 215 - DNA, 18 bp, Oryctolagus cuniculus
  source 1..18: mol_type = genomic DNA
agcggctactacatgtgc

SEQ ID NO: 216 - DNA, 51 bp, Oryctolagus cuniculus
  source 1..51: mol_type = genomic DNA
tgcatttttactattactgataacacttactacgcgaactgggcgaaaggc

SEQ ID NO: 217 - DNA, 33 bp, Oryctolagus cuniculus
  source 1..33: mol_type = genomic DNA
gggatttattctactgataattattatgccttg

SEQ ID NO: 218 - protein, 123 aa, Oryctolagus cuniculus
MDTRAPTQLLGLLLLWLPGARCDVVMTQTPASVEAAVGGTVTIKCQASQSVSSYLNWYQQKPGQPPKLLIYRASTLESGVPSRFKGSGSGTEFTLTISDLECADAATYYCQCTYGTSSSYGAA

SEQ ID NO: 219 - protein, 133 aa, Oryctolagus cuniculus
METGLRWLLLVAVLKGVQCQSVEESGGRLVTPGTPLTLTCTVSGISLSSNAISWVRQAPGKGLEWIGIISYSGTTYYASWAKGRFTISKTSSTTVDLKITSPTTEDTATYFCARDDPTTVMVMLIPFGAGMDL

SEQ ID NO: 220 - protein, 11 aa, Oryctolagus cuniculus
QASQSVSSYLN

SEQ ID NO: 221 - protein, 7 aa, Oryctolagus cuniculus
RASTLES

SEQ ID NO: 222 - protein, 13 aa, Oryctolagus cuniculus
QCTYGTSSSYGAA

SEQ ID NO: 223 - protein, 5 aa, Oryctolagus cuniculus
SNAIS

SEQ ID NO: 224 - protein, 16 aa, Oryctolagus cuniculus
IISYSGTTYYASWAKG

SEQ ID NO: 225 - protein, 19 aa, Oryctolagus cuniculus
DDPTTVMVMLIPFGAGMDL

SEQ ID NO: 226 - DNA, 369 bp, Oryctolagus cuniculus
  source 1..369: mol_type = genomic DNA
atggacacgagggcccccactcagctgctggggctcctgctgctctggctcccaggtgccagatgtgatgttgtgatgacccagactccagcctccgtggaggcagctgtgggaggcacagtcaccatcaagtgccaggccagtcagagcgttagtagctacttaaactggtatcagcagaaaccagggcagcctcccaagctcctgatctacagggcatccactctggaatctggggtcccatcgcggttcaaaggcagtggatctgggacagagttcactctcaccatcagcgacctggagtgtgccgatgctgccacttactactgtcaatgtacttatggtactagtagtagttatggtgctgct

SEQ ID NO: 227 - DNA, 399 bp, Oryctolagus cuniculus
  source 1..399: mol_type = genomic DNA
atggagactgggctgcgctggcttctcctggtcgctgtgctcaaaggtgtccagtgtcagtcggtggaggagtccgggggtcgcctggtcacgcctgggacacccctgacactcacctgcaccgtctctggtatctccctcagtagcaatgcaataagctgggtccgccaggctccagggaaggggctggaatggatcggaatcattagttatagtggtaccacatactacgcgagctgggcgaaaggccgattcaccatctccaaaacctcgtcgaccacggtggatctgaaaatcactagtccgacaaccgaggacacggccacctacttctgtgccagagatgaccctacgacagttatggttatgttgataccttttggagccggcatggacctc

SEQ ID NO: 228 - DNA, 33 bp, Oryctolagus cuniculus
  source 1..33: mol_type = genomic DNA
caggccagtcagagcgttagtagctacttaaac

SEQ ID NO: 229 - DNA, 21 bp, Oryctolagus cuniculus
  source 1..21: mol_type = genomic DNA
agggcatccactctggaatct

SEQ ID NO: 230 - DNA, 39 bp, Oryctolagus cuniculus
  source 1..39: mol_type = genomic DNA
caatgtacttatggtactagtagtagttatggtgctgct

SEQ ID NO: 231 - DNA, 15 bp, Oryctolagus cuniculus
  source 1..15: mol_type = genomic DNA
agcaatgcaataagc

SEQ ID NO: 232 - DNA, 48 bp, Oryctolagus cuniculus
  source 1..48: mol_type = genomic DNA
atcattagttatagtggtaccacatactacgcgagctgggcgaaaggc

SEQ ID NO: 233 - DNA, 57 bp, Oryctolagus cuniculus
  source 1..57: mol_type = genomic DNA
gatgaccctacgacagttatggttatgttgataccttttggagccggcatggacctc

SEQ ID NO: 234 - protein, 125 aa, Oryctolagus cuniculus
MDTRAPTQLLGLLLLWLPGATFAQVLTQTASPVSAAVGGTVTINCQASQSVYKNNYLSWYQQKPGQPPKGLIYSASTLDSGVPLRFSGSGSGTQFTLTISDVQCDDAATYYCLGSYDCSSGDCYA

SEQ ID NO: 235 - protein, 119 aa, Oryctolagus cuniculus
METGLRWLLLVAVLKGVQCQSLEESGGDLVKPEGSLTLTCTASGFSFSSYWMCWVRQAPGKGLEWIACIVTGNGNTYYANWAKGRFTISKTSSTTVTLQMTSLTAADTATYFCAKAYDL

SEQ ID NO: 236 - protein, 13 aa, Oryctolagus cuniculus
QASQSVYKNNYLS

SEQ ID NO: 237 - protein, 7 aa, Oryctolagus cuniculus
SASTLDS

SEQ ID NO: 238 - protein, 13 aa, Oryctolagus cuniculus
LGSYDCSSGDCYA

SEQ ID NO: 239 - protein, 5 aa, Oryctolagus cuniculus
SYWMC

SEQ ID NO: 240 - protein, 17 aa, Oryctolagus cuniculus
CIVTGNGNTYYANWAKG

SEQ ID NO: 241 - protein, 4 aa, Oryctolagus cuniculus
AYDL

SEQ ID NO: 242 - DNA, 375 bp, Oryctolagus cuniculus
  source 1..375: mol_type = genomic DNA
atggacacgagggcccccactcagctgctggggctcctgctgctctggctcccaggtgccacatttgcccaagtgctgacccagactgcatcgcccgtgtctgcagctgtgggaggcacagtcaccatcaactgccaggccagtcagagtgtttataagaacaactacttatcctggtatcagcagaaaccagggcagcctcccaaaggcctgatctattctgcatcgactctagattctggggtcccattgcggttcagcggcagtggatctgggacacagttcactctcaccatcagcgacgtgcagtgtgacgatgctgccacttactactgtctaggcagttatgattgtagtagtggtgattgttatgct

SEQ ID NO: 243 - DNA, 357 bp, Oryctolagus cuniculus
  source 1..357: mol_type = genomic DNA
atggagactgggctgcgctggcttctcctggtcgctgtgctcaaaggtgtccagtgtcagtcgttggaggagtccgggggagacctggtcaagcctgagggatccctgacactcacctgcacagcctctggattctccttcagtagctactggatgtgctgggtccgccaggctccagggaaggggctggagtggatcgcatgcattgttactggtaatggtaacacttactacgcgaactgggcgaaaggccgattcaccatctccaaaacctcgtcgaccacggtgactctgcaaatgaccagtctgacagccgcggacacggccacctatttttgtgcgaaagcctatgacttg

SEQ ID NO: 244 - DNA, 39 bp, Oryctolagus cuniculus
  source 1..39: mol_type = genomic DNA
caggccagtcagagtgtttataagaacaactacttatcc

SEQ ID NO: 245 - DNA, 21 bp, Oryctolagus cuniculus
  source 1..21: mol_type = genomic DNA
tctgcatcgactctagattct

SEQ ID NO: 246 - DNA, 39 bp, Oryctolagus cuniculus
  source 1..39: mol_type = genomic DNA
ctaggcagttatgattgtagtagtggtgattgttatgct

SEQ ID NO: 247 - DNA, 15 bp, Oryctolagus cuniculus
  source 1..15: mol_type = genomic DNA
agctactggatgtgc

SEQ ID NO: 248 - DNA, 51 bp, Oryctolagus cuniculus
  source 1..51: mol_type = genomic DNA
tgcattgttactggtaatggtaacacttactacgcgaactgggcgaaaggc

SEQ ID NO: 249 - DNA, 12 bp, Oryctolagus cuniculus
  source 1..12: mol_type = genomic DNA
gcctatgacttg

SEQ ID NO: 250 - protein, 123 aa, Oryctolagus cuniculus
MDTRAPTQLLGLLLLWLPGSTFAAVLTQTPSPVSAAVGGTVSISCQASQSVYDNNYLSWYQQKPGQPPKLLIYGASTLASGVPSRFKGTGSGTQFTLTITDVQCDDAATYYCAGVFNDDSDDA

SEQ ID NO: 251 - protein, 125 aa, Oryctolagus cuniculus
METGLRWLLLVAVPKGVQCQSLEESGGRLVTPGTPLTLTCTLSGFSLSAYYMSWVRQAPGKGLEWIGFITLSDHISYARWAKGRFTISKTSTTVDLKMTSPTTEDTATYFCARSRGWGAMGRLDL

SEQ ID NO: 252 - protein, 13 aa, Oryctolagus cuniculus
QASQSVYDNNYLS

SEQ ID NO: 253 - protein, 7 aa, Oryctolagus cuniculus
GASTLAS

SEQ ID NO: 254 - protein, 11 aa, Oryctolagus cuniculus
AGVFNDDSDDA

SEQ ID NO: 255 - protein, 5 aa, Oryctolagus cuniculus
AYYMS

SEQ ID NO: 256 - protein, 16 aa, Oryctolagus cuniculus
FITLSDHISYARWAKG

SEQ ID NO: 257 - protein, 12 aa, Oryctolagus cuniculus
SRGWGAMGRLDL

SEQ ID NO: 258 - DNA, 369 bp, Oryctolagus cuniculus
  source 1..369: mol_type = genomic DNA
atggacacgagggcccccactcagctgctggggctcctgctgctctggctcccaggttccacatttgccgccgtgctgacccagactccatctcccgtgtctgcagctgtgggaggcacagtcagcatcagttgccaggccagtcagagtgtttatgacaacaactatttatcctggtatcagcagaaaccaggacagcctcccaagctcctgatctatggtgcatccactctggcatctggggtcccatcgcggttcaaaggcacgggatctgggacacagttcactctcaccatcacagacgtgcagtgtgacgatgctgccacttactattgtgcaggcgtttttaatgatgatagtgatgatgcc

SEQ ID NO: 259 - DNA, 375 bp, Oryctolagus cuniculus
  source 1..375: mol_type = genomic DNA
atggagactgggctgcgctggcttctcctggtcgctgtgcccaaaggtgtccagtgtcagtcgctggaggagtccgggggtcgcctggtcacgcctgggacacccctgacactcacctgcacactctctggattctccctcagtgcatactatatgagctgggtccgccaggctccagggaaggggctggaatggatcggattcattactctgagtgatcatatatcttacgcgaggtgggcgaaaggccgattcaccatctccaaaacctcgaccacggtggatctgaaaatgaccagtccgacaaccgaggacacggccacctatttctgtgccaggagtcgtggctggggtgcaatgggtcggttggatctc

SEQ ID NO: 260 - DNA, 39 bp, Oryctolagus cuniculus
  source 1..39: mol_type = genomic DNA
caggccagtcagagtgtttatgacaacaactatttatcc

SEQ ID NO: 261 - DNA, 21 bp, Oryctolagus cuniculus
  source 1..21: mol_type = genomic DNA
ggtgcatccactctggcatct

SEQ ID NO: 262 - DNA, 33 bp, Oryctolagus cuniculus
  source 1..33: mol_type = genomic DNA
gcaggcgtttttaatgatgatagtgatgatgcc

SEQ ID NO: 263 - DNA, 15 bp, Oryctolagus cuniculus
  source 1..15: mol_type = genomic DNA
gcatactatatgagc

SEQ ID NO: 264 - DNA, 48 bp, Oryctolagus cuniculus
  source 1..48: mol_type = genomic DNA
ttcattactctgagtgatcatatatcttacgcgaggtgggcgaaaggc

SEQ ID NO: 265 - DNA, 36 bp, Oryctolagus cuniculus; identical to SEQ ID NO: 68
  source 1..36: mol_type = genomic DNA

SEQ ID NO: 266 - protein, 123 aa, Oryctolagus cuniculus
MDTRAPTQLLGLLLLWLPGATFAAVLTQTPSPVSAAVGGTVTISCQASQSVYNNKNLAWYQQKSGQPPKLLIYWASTLASGVSSRFSGSGSGTQFTLTVSGVQCDDAATYYCLGVFDDDADNA

SEQ ID NO: 267 - protein, 121 aa, Oryctolagus cuniculus
METGLRWLLLVAVLKGVQCQSVEESGGRLVTPGTPLTLTCTASGFSLSSYSMTWVRQAPGKGLEYIGVIGTSGSTYYATWAKGRFTISRTSTTVALKITSPTTEDTATYFCVRSLSSITFL

SEQ ID NO: 268 - protein, 13 aa, Oryctolagus cuniculus
QASQSVYNNKNLA

SEQ ID NO: 269 - protein, 7 aa, Oryctolagus cuniculus
WASTLAS

SEQ ID NO: 270 - protein, 11 aa, Oryctolagus cuniculus
LGVFDDDADNA

SEQ ID NO: 271 - protein, 5 aa, Oryctolagus cuniculus
SYSMT

SEQ ID NO: 272 - protein, 16 aa, Oryctolagus cuniculus
VIGTSGSTYYATWAKG

SEQ ID NO: 273 - protein, 8 aa, Oryctolagus cuniculus
SLSSITFL

SEQ ID NO: 274 - DNA, 369 bp, Oryctolagus cuniculus
  source 1..369: mol_type = genomic DNA
atggacacgagggcccccactcagctgctggggctcctgctgctctggctcccaggtgccacattcgcagccgtgctgacccagacaccatcgcccgtgtctgcggctgtgggaggcacagtcaccatcagttgccaggccagtcagagtgtttataacaacaaaaatttagcctggtatcagcagaaatcagggcagcctcccaagctcctgatctactgggcatccactctggcatctggggtctcatcgcggttcagcggcagtggatctgggacacagttcactctcaccgtcagcggcgtgcagtgtgacgatgctgccacttactactgtctaggcgtttttgatgatgatgctgataatgct

SEQ ID NO: 275 - DNA, 363 bp, Oryctolagus cuniculus
  source 1..363: mol_type = genomic DNA
atggagactgggctgcgctggcttctcctggtcgctgtgctcaaaggtgtccaatgtcagtcggtggaggagtccgggggtcgcctggtcacgcctgggacacccctgacactcacctgcacagcctctggattctccctcagtagctactccatgacctgggtccgccaggctccagggaaggggctggaatatatcggagtcattggtactagtggtagcacatactacgcgacctgggcgaaaggccgattcaccatctccagaacctcgaccacggtggctctgaaaatcaccagtccgacaaccgaggacacggccacctatttctgtgtcaggagtctttcttctattactttcttg

SEQ ID NO: 276 - DNA, 39 bp, Oryctolagus cuniculus
  source 1..39: mol_type = genomic DNA
caggccagtcagagtgtttataacaacaaaaatttagcc

SEQ ID NO: 277 - DNA, 21 bp, Oryctolagus cuniculus
  source 1..21: mol_type = genomic DNA
tgggcatccactctggcatct

SEQ ID NO: 278 - DNA, 33 bp, Oryctolagus cuniculus
  source 1..33: mol_type = genomic DNA
ctaggcgtttttgatgatgatgctgataatgct

SEQ ID NO: 279 - DNA, 15 bp, Oryctolagus cuniculus
  source 1..15: mol_type = genomic DNA
agctactccatgacc

SEQ ID NO: 280 - DNA, 48 bp, Oryctolagus cuniculus
  source 1..48: mol_type = genomic DNA
gtcattggtactagtggtagcacatactacgcgacctgggcgaaaggc

SEQ ID NO: 281 - DNA, 24 bp, Oryctolagus cuniculus
  source 1..24: mol_type = genomic DNA
agtctttcttctattactttcttg

SEQ ID NO: 282 - protein, 120 aa, Oryctolagus cuniculus
MDTRAPTQLLGLLLLWLPGARCAFELTQTPASVEAAVGGTVTINCQASQNIYRYLAWYQQKPGQPPKFLIYLASTLASGVPSRFKGSGSGTEFTLTISDLECADAATYYCQSYYSSNSVA

SEQ ID NO: 283 - protein, 128 aa, Oryctolagus cuniculus
METGLRWLLLVAVLKGVQCQEQLVESGGDLVQPEGSLTLTCTASELDFSSGYWICWVRQVPGKGLEWIGCIYTGSSGSTFYASWAKGRFTISKTSSTTVTLQMTSLTAADTATYFCARGYSGFGYFKL

SEQ ID NO: 284 - protein, 11 aa, Oryctolagus cuniculus
QASQNIYRYLA

SEQ ID NO: 285 - protein, 7 aa, Oryctolagus cuniculus
LASTLAS

SEQ ID NO: 286 - protein, 10 aa, Oryctolagus cuniculus
QSYYSSNSVA

SEQ ID NO: 287 - protein, 6 aa, Oryctolagus cuniculus
SGYWIC

SEQ ID NO: 288 - protein, 18 aa, Oryctolagus cuniculus
CIYTGSSGSTFYASWAKG

SEQ ID NO: 289 - protein, 10 aa, Oryctolagus cuniculus
GYSGFGYFKL

SEQ ID NO: 290 - DNA, 360 bp, Oryctolagus cuniculus
  source 1..360: mol_type = genomic DNA
atggacacgagggcccccactcagctgctggggctcctgctgctctggctcccaggtgccagatgtgcattcgaattgacccagactccagcctccgtggaggcagctgtgggaggcacagtcaccatcaattgccaggccagtcagaacatttatagatacttagcctggtatcagcagaaaccagggcagcctcccaagttcctgatctatctggcatctactctggcatctggggtcccatcgcggtttaaaggcagtggatctgggacagagttcactctcaccatcagcgacctggagtgtgccgatgctgccacttactactgtcaaagttattatagtagtaatagtgtcgct

SEQ ID NO: 291 - DNA, 384 bp, Oryctolagus cuniculus
  source 1..384: mol_type = genomic DNA
atggagactgggctgcgctggcttctcctggtcgctgtgctcaaaggtgtccagtgtcaggagcagctggtggagtccgggggagacctggtccagcctgagggatccctgacactcacctgcacagcttctgagttagacttcagtagcggctactggatatgctgggtccgccaggttccagggaaggggctggagtggatcggatgcatttatactggtagtagtggtagcactttttacgcgagttgggcgaaaggccgattcaccatctccaaaacctcgtcgaccacggtgactctgcaaatgaccagtctgacagccgcggacacggccacctatttctgtgcgagaggttatagtggctttggttactttaagttg

SEQ ID NO: 292 - DNA, 33 bp, Oryctolagus cuniculus
  source 1..33: mol_type = genomic DNA
caggccagtcagaacatttatagatacttagcc

SEQ ID NO: 293 - DNA, 21 bp, Oryctolagus cuniculus
  source 1..21: mol_type = genomic DNA
ctggcatctactctggcatct

SEQ ID NO: 294 - DNA, 30 bp, Oryctolagus cuniculus
  source 1..30: mol_type = genomic DNA
caaagttattatagtagtaatagtgtcgct

SEQ ID NO: 295 - DNA, 18 bp, Oryctolagus cuniculus
  source 1..18: mol_type = genomic DNA
agcggctactggatatgc

SEQ ID NO: 296 - DNA, 54 bp, Oryctolagus cuniculus
  source 1..54: mol_type = genomic DNA
tgcatttatactggtagtagtggtagcactttttacgcgagttgggcgaaaggc

SEQ ID NO: 297 - DNA, 30 bp, Oryctolagus cuniculus
  source 1..30: mol_type = genomic DNA
ggttatagtggctttggttactttaagttg

SEQ ID NO: 298 - protein, 122 aa, Oryctolagus cuniculus
MDTRAPTQLLGLLLLWLPGARCAYDMTQTPASVEVAVGGTVTIKCQASEDIYRLLAWYQQKPGQPPKLLIYDSSDLASGVPSRFKGSGSGTEFTLAISGVQCDDAATYYCQQAWSYSDIDNA

SEQ ID NO: 299 - protein, 123 aa, Oryctolagus cuniculus
METGLRWLLLVAVLKGVQCQSVEESGGRLVTPGTPLTLTCTASGFSLSSYYMSWVRQAPGKGLEWIGIITTSGNTFYASWAKGRLTISRTSTTVDLKITSPTTEDTATYFCARTSDIFYYRNL

SEQ ID NO: 300 - protein, 11 aa, Oryctolagus cuniculus
QASEDIYRLLA

SEQ ID NO: 301 - protein, 7 aa, Oryctolagus cuniculus
DSSDLAS

SEQ ID NO: 302 - protein, 12 aa, Oryctolagus cuniculus
QQAWSYSDIDNA

SEQ ID NO: 303 - protein, 5 aa, Oryctolagus cuniculus
SYYMS

SEQ ID NO: 304 - protein, 16 aa, Oryctolagus cuniculus
IITTSGNTFYASWAKG

SEQ ID NO: 305 - protein, 10 aa, Oryctolagus cuniculus
TSDIFYYRNL

SEQ ID NO: 306 - DNA, 366 bp, Oryctolagus cuniculus
  source 1..366: mol_type = genomic DNA
atggacacgagggcccccactcagctgctggggctcctgctgctctggctcccaggtgccagatgtgcctatgatatgacccagactccagcctctgtggaggtagctgtgggaggcacagtcaccatcaagtgccaggccagtgaggacatttataggttattggcctggtatcaacagaaaccagggcagcctcccaagctcctgatctatgattcatccgatctggcatctggggtcccatcgcggttcaaaggcagtggatctgggacagagttcactctcgccatcagcggtgtgcagtgtgacgatgctgccacttactactgtcaacaggcttggagttatagtgatattgataatgct

SEQ ID NO: 307 - DNA, 369 bp, Oryctolagus cuniculus
  source 1..369: mol_type = genomic DNA
atggagactgggctgcgctggcttctcctggtcgctgtgctcaaaggtgtccagtgtcagtcggtggaggagtccgggggtcgcctggtcacgccggggacacccctgacactcacctgcacagcctctggattctccctcagtagctactacatgagctgggtccgccaggctccagggaaggggctggaatggatcggaatcattactactagtggtaatacattttacgcgagctgggcgaaaggccggctcaccatctccagaacctcgaccacggtggatctgaaaatcaccagtccgacaaccgaggacacggccacctatttctgtgccagaacttctgatattttttattatcgtaacttg

SEQ ID NO: 308 - DNA, 33 bp, Oryctolagus cuniculus
  source 1..33: mol_type = genomic DNA
caggccagtgaggacatttataggttattggcc

SEQ ID NO: 309 - DNA, 21 bp, Oryctolagus cuniculus
  source 1..21: mol_type = genomic DNA
gattcatccgatctggcatct

SEQ ID NO: 310 - DNA, 36 bp, Oryctolagus cuniculus
  source 1..36: mol_type = genomic DNA
caacaggcttggagttatagtgatattgataatgct

SEQ ID NO: 311 - DNA, 15 bp, Oryctolagus cuniculus
  source 1..15: mol_type = genomic DNA
agctactacatgagc

SEQ ID NO: 312 - DNA, 48 bp, Oryctolagus cuniculus
  source 1..48: mol_type = genomic DNA
atcattactactagtggtaatacattttacgcgagctgggcgaaaggc

SEQ ID NO: 313 - DNA, 30 bp, Oryctolagus cuniculus
  source 1..30: mol_type = genomic DNA
acttctgatattttttattatcgtaacttg

SEQ ID NO: 314 - protein, 123 aa, Oryctolagus cuniculus
MDTRAPTQLLGLLLLWLPGATFAAVLTQTASPVSAAVGATVTINCQSSQSVYNDMDLAWFQQKPGQPPKLLIYSASTLASGVPSRFSGSGSGTEFTLTISGVQCDDAATYYCLGAFDDDADNT

SEQ ID NO: 315 - protein, 129 aa, Oryctolagus cuniculus
METGLRWLLLVAVLKGVQCQSVEESGGRLVTPGTPLTLTCTVSGFSLTRHAITWVRQAPGKGLEWIGCIWSGGSTYYATWAKGRFTISKTSTTVDLRITSPTTEDTATYFCARVIGDTAGYAYFTGLDL

SEQ ID NO: 316 - protein, 13 aa, Oryctolagus cuniculus
QSSQSVYNDMDLA

SEQ ID NO: 317 - protein, 7 aa, Oryctolagus cuniculus
SASTLAS

SEQ ID NO: 318 - protein, 11 aa, Oryctolagus cuniculus
LGAFDDDADNT

SEQ ID NO: 319 - protein, 5 aa, Oryctolagus cuniculus
RHAIT

SEQ ID NO: 320 - protein, 16 aa, Oryctolagus cuniculus
CIWSGGSTYYATWAKG

SEQ ID NO: 321 - protein, 16 aa, Oryctolagus cuniculus
VIGDTAGYAYFTGLDL

SEQ ID NO: 322 - DNA, 369 bp, Oryctolagus cuniculus
  source 1..369: mol_type = genomic DNA
atggacacgagggcccccactcagctgctggggctcctgctgctctggctcccaggtgccacgtttgcagccgtgctgacccagactgcatcacccgtgtctgccgctgtgggagccacagtcaccatcaactgccagtccagtcagagtgtttataatgacatggacttagcctggtttcagcagaaaccagggcagcctcccaagctcctgatctattctgcatccactctggcatctggggtcccatcgcggttcagcggcagtggatctgggacagagttcactctcaccatcagcggcgtgcagtgtgacgatgctgccacttactactgtctaggcgcttttgatgatgatgctgataatact

SEQ ID NO: 323 - DNA, 387 bp, Oryctolagus cuniculus
  source 1..387: mol_type = genomic DNA
atggagactgggctgcgctggcttctcctggtcgctgtgctcaaaggtgtccagtgtcagtcggtggaggagtccgggggtcgcctggtcacgcctgggacacccctgacactcacctgcacagtctctggattctccctcactaggcatgcaataacctgggtccgccaggctccagggaaggggctggaatggatcggatgcatttggagtggtggtagcacatactacgcgacctgggcgaaaggccgattcaccatctccaaaacctcgaccacggtggatctcagaatcaccagtccgacaaccgaggacacggccacctacttctgtgccagagtcattggcgatactgctggttatgcttattttacggggcttgacttg

SEQ ID NO: 324 - DNA, 39 bp, Oryctolagus cuniculus
  source 1..39: mol_type = genomic DNA
cagtccagtcagagtgtttataatgacatggacttagcc

SEQ ID NO: 325 - DNA, 21 bp, Oryctolagus cuniculus
  source 1..21: mol_type = genomic DNA
tctgcatccactctggcatct

SEQ ID NO: 326 - DNA, 33 bp, Oryctolagus cuniculus
  source 1..33: mol_type = genomic DNA
ctaggcgcttttgatgatgatgctgataatact

SEQ ID NO: 327 - DNA, 15 bp, Oryctolagus cuniculus
  source 1..15: mol_type = genomic DNA
aggcatgcaataacc

SEQ ID NO: 328 - DNA, 48 bp, Oryctolagus cuniculus
  source 1..48: mol_type = genomic DNA
tgcatttggagtggtggtagcacatactacgcgacctgggcgaaaggc

SEQ ID NO: 329 - DNA, 48 bp, Oryctolagus cuniculus
  source 1..48: mol_type = genomic DNA
gtcattggcgatactgctggttatgcttattttacggggcttgacttg

SEQ ID NO: 330 - protein, 121 aa, Oryctolagus cuniculus
MDTRAPTQLLGLLLLWLPGARCAYDMTQTPASVEVAVGGTVTIKCQASQSVYNWLSWYQQKPGQPPKLLIYTASSLASGVPSRFSGSGSGTEFTLTISGVECADAATYYCQQGYTSDVDNV

SEQ ID NO: 331 - protein, 130 aa, Oryctolagus cuniculus
METGLRWLLLVAVLKGVQCQSLEEAGGRLVTPGTPLTLTCTVSGIDLSSYAMGWVRQAPGKGLEYIGIISSSGSTYYATWAKGRFTISQASSTTVDLKITSPTTEDSATYFCARGGAGSGGVWLLDGFDP

SEQ ID NO: 332 - protein, 11 aa, Oryctolagus cuniculus
QASQSVYNWLS

SEQ ID NO: 333 - protein, 7 aa, Oryctolagus cuniculus
TASSLAS

SEQ ID NO: 334 - protein, 11 aa, Oryctolagus cuniculus
QQGYTSDVDNV

SEQ ID NO: 335 - protein, 5 aa, Oryctolagus cuniculus
SYAMG

SEQ ID NO: 336 - protein, 16 aa, Oryctolagus cuniculus
IISSSGSTYYATWAKG

SEQ ID NO: 337 - protein, 16 aa, Oryctolagus cuniculus
GGAGSGGVWLLDGFDP

SEQ ID NO: 338 - DNA, 363 bp, Oryctolagus cuniculus
  source 1..363: mol_type = genomic DNA
atggacacgagggcccccactcagctgctggggctcctgctgctctggctcccaggtgccagatgtgcctatgatatgacccagactccagcctctgtggaggtagctgtgggaggcacagtcaccatcaagtgccaggccagtcagagtgtttataattggttatcctggtatcagcagaaaccagggcagcctcccaagctcctgatctatactgcatccagtctggcatctggggtcccatcgcggttcagtggcagtggatctgggacagagttcactctcaccatcagcggcgtggagtgtgccgatgctgccacttactactgtcaacagggttatactagtgatgttgataatgtt

SEQ ID NO: 339 - DNA, 390 bp, Oryctolagus cuniculus
  source 1..390: mol_type = genomic DNA
atggagactgggctgcgctggcttctcctggtcgctgtgctcaaaggtgtccagtgtcagtcgctggaggaggccgggggtcgcctggtcacgcctgggacacccctgacactcacctgcacagtctctggaatcgacctcagtagctatgcaatgggctgggtccgccaggctccagggaaggggctggaatacatcggaatcattagtagtagtggtagcacatactacgcgacctgggcgaaaggccgattcaccatctcacaagcctcgtcgaccacggtggatctgaaaattaccagtccgacaaccgaggactcggccacatatttctgtgccagagggggtgctggtagtggtggtgtttggctgcttgatggttttgatccc

SEQ ID NO: 340 - DNA, 33 bp, Oryctolagus cuniculus
  source 1..33: mol_type = genomic DNA
caggccagtcagagtgtttataattggttatcc

SEQ ID NO: 341 - DNA, 21 bp, Oryctolagus cuniculus
  source 1..21: mol_type = genomic DNA
actgcatccagtctggcatct

SEQ ID NO: 342 - DNA, 33 bp, Oryctolagus cuniculus
  source 1..33: mol_type = genomic DNA
caacagggttatactagtgatgttgataatgtt

SEQ ID NO: 343 - DNA, 15 bp, Oryctolagus cuniculus
  source 1..15: mol_type = genomic DNA
agctatgcaatgggc

SEQ ID NO: 344 - DNA, 48 bp, Oryctolagus cuniculus
  source 1..48: mol_type = genomic DNA
atcattagtagtagtggtagcacatactacgcgacctgggcgaaaggc

SEQ ID NO: 345 - DNA, 48 bp, Oryctolagus cuniculus
  source 1..48: mol_type = genomic DNA
gggggtgctggtagtggtggtgtttggctgcttgatggttttgatccc

SEQ ID NO: 346 - protein, 123 aa, Oryctolagus cuniculus
MDTRAPTQLLGLLLLWLPGAKCADVVMTQTPASVSAAVGGTVTINCQASENIYNWLAWYQQKPGQPPKLLIYTVGDLASGVSSRFKGSGSGTEFTLTISDLECADAATYYCQQGYSSSYVDNV

SEQ ID NO: 347 - protein, 130 aa, Oryctolagus cuniculus
METGLRWLLLVAVLKGVQCQEQLKESGGRLVTPGTPLTLTCTVSGFSLNDYAVGWFRQAPGKGLEWIGYIRSSGTTAYATWAKGRFTISATSTTVDLKITSPTTEDTATYFCARGGAGSSGVWILDGFAP

SEQ ID NO: 348 - protein, 11 aa, Oryctolagus cuniculus
QASENIYNWLA

SEQ ID NO: 349 - protein, 7 aa, Oryctolagus cuniculus
TVGDLAS

SEQ ID NO: 350 - protein, 12 aa, Oryctolagus cuniculus
QQGYSSSYVDNV

SEQ ID NO: 351 - protein, 5 aa, Oryctolagus cuniculus
DYAVG

SEQ ID NO: 352 - protein, 16 aa, Oryctolagus cuniculus
YIRSSGTTAYATWAKG

SEQ ID NO: 353 - protein, 16 aa, Oryctolagus cuniculus
GGAGSSGVWILDGFAP

SEQ ID NO: 354 - DNA, 369 bp, Oryctolagus cuniculus
  source 1..369: mol_type = genomic DNA
atggacacgagggcccccactcagctgctggggctcctgctgctctggctcccaggtgccaaatgtgccgatgttgtgatgacccagactccagcctccgtgtctgcagctgtgggaggcacagtcaccatcaattgccaggccagtgagaacatttataattggttagcctggtatcagcagaaaccagggcagcctcccaagctcctgatctatactgtaggcgatctggcatctggggtctcatcgcggttcaaaggcagtggatctgggacagagttcactctcaccatcagcgacctggagtgtgccgatgctgccacttactattgtcaacagggttatagtagtagttatgttgataatgtt

SEQ ID NO: 355 - DNA, 390 bp, Oryctolagus cuniculus
  source 1..390: mol_type = genomic DNA
atggagactgggctgcgctggcttctcctggtcgctgtgctcaaaggtgtccagtgtcaggagcagctgaaggagtccgggggtcgcctggtcacgcctgggacacccctgacactcacctgcacagtctctggattctccctcaatgactatgcagtgggctggttccgccaggctccagggaaggggctggaatggatcggatacattcgtagtagtggtaccacagcctacgcgacctgggcgaaaggccgattcaccatctccgctacctcgaccacggtggatctgaaaatcaccagtccgacaaccgaggacacggccacctatttctgtgccagagggggtgctggtagtagtggtgtgtggatccttgatggttttgctccc

SEQ ID NO: 356 - DNA, 33 bp, Oryctolagus cuniculus
  source 1..33: mol_type = genomic DNA
caggccagtgagaacatttataattggttagcc

SEQ ID NO: 357 - DNA, 21 bp, Oryctolagus cuniculus
  source 1..21: mol_type = genomic DNA
actgtaggcgatctggcatct

SEQ ID NO: 358 - DNA, 36 bp, Oryctolagus cuniculus
  source 1..36: mol_type = genomic DNA
caacagggttatagtagtagttatgttgataatgtt

SEQ ID NO: 359 - DNA, 15 bp, Oryctolagus cuniculus
  source 1..15: mol_type = genomic DNA
gactatgcagtgggc

SEQ ID NO: 360 - DNA, 48 bp, Oryctolagus cuniculus
  source 1..48: mol_type = genomic DNA
tacattcgtagtagtggtaccacagcctacgcgacctgggcgaaaggc

SEQ ID NO: 361 - DNA, 48 bp, Oryctolagus cuniculus
  source 1..48: mol_type = genomic DNA
gggggtgctggtagtagtggtgtgtggatccttgatggttttgctccc

SEQ ID NO: 362 - protein, 121 aa, Oryctolagus cuniculus
MDTRAPTQLLGLLLLWLPGATFAQVLTQTPSSVSAAVGGTVTINCQASQSVYQNNYLSWFQQKPGQPPKLLIYGAATLASGVPSRFKGSGSGTQFTLTISDLECDDAATYYCAGAYRDVDS

SEQ ID NO: 363 - protein, 130 aa, Oryctolagus cuniculus
METGLRWLLLVAVLKGVQCQSLEESGGDLVKPGASLTLTCTASGFSFTSTYYIYWVRQAPGKGLEWIACIDAGSSGSTYYATWVNGRFTISKTSSTTVTLQMTSLTAADTATYFCAKWDYGGNVGWGYDL

SEQ ID NO: 364 - protein, 13 aa, Oryctolagus cuniculus
QASQSVYQNNYLS

SEQ ID NO: 365 - protein, 7 aa, Oryctolagus cuniculus
GAATLAS

SEQ ID NO: 366 - protein, 9 aa, Oryctolagus cuniculus
AGAYRDVDS

SEQ ID NO: 367 - protein, 6 aa, Oryctolagus cuniculus
STYYIY

SEQ ID NO: 368 - protein, 18 aa, Oryctolagus cuniculus
CIDAGSSGSTYYATWVNG

SEQ ID NO: 369 - protein, 13 aa, Oryctolagus cuniculus
WDYGGNVGWGYDL

SEQ ID NO: 370 - DNA, 363 bp, Oryctolagus cuniculus
  source 1..363: mol_type = genomic DNA
atggacacgagggcccccactcagctgctggggctcctgctgctctggctcccaggtgccacatttgctcaagtgctgacccagactccatcctccgtgtctgcagctgtgggaggcacagtcaccatcaattgccaggccagtcagagtgtttatcagaacaactacttatcctggtttcagcagaaaccagggcagcctcccaagctcctgatctatggtgcggccactctggcatctggggtcccatcgcggttcaaaggcagtggatctgggacacagttcactctcaccatcagcgacctggagtgtgacgatgctgccacttactactgtgcaggcgcttatagggatgtggattct

SEQ ID NO: 371 - DNA, 390 bp, Oryctolagus cuniculus
  source 1..390: mol_type = genomic DNA
atggagactgggctgcgctggcttctcctggtcgctgtgctcaaaggtgtccagtgtcagtcgttggaggagtccgggggagacctggtcaagcctggggcatccctgacactcacctgcacagcctctggattctcctttactagtacctactacatctactgggtccgccaggctccagggaaggggctggagtggatcgcatgtattgatgctggtagtagtggtagcacttactacgcgacctgggtgaatggccgattcaccatctccaaaacctcgtcgaccacggtgactctgcaaatgaccagtctgacagccgcggacacggccacctatttctgtgcgaaatgggattatggtggtaatgttggttggggttatgacttg

SEQ ID NO: 372 - DNA, 39 bp, Oryctolagus cuniculus
  source 1..39: mol_type = genomic DNA
caggccagtcagagtgtttatcagaacaactacttatcc

SEQ ID NO: 373 - DNA, 21 bp, Oryctolagus cuniculus
  source 1..21: mol_type = genomic DNA
ggtgcggccactctggcatct

SEQ ID NO: 374 - DNA, 27 bp, Oryctolagus cuniculus
  source 1..27: mol_type = genomic DNA
gcaggcgcttatagggatgtggattct

SEQ ID NO: 375 - DNA, 18 bp, Oryctolagus cuniculus
  source 1..18: mol_type = genomic DNA
agtacctactacatctac

SEQ ID NO: 376 - DNA, 54 bp, Oryctolagus cuniculus
  source 1..54: mol_type = genomic DNA
tgtattgatgctggtagtagtggtagcacttactacgcgacctgggtgaatggc

SEQ ID NO: 377 - DNA, 39 bp, Oryctolagus cuniculus
  source 1..39: mol_type = genomic DNA
tgggattatggtggtaatgttggttggggttatgacttg

SEQ ID NO: 378 - protein, 120 aa, Oryctolagus cuniculus
MDTRAPTQLLGLLLLWLPGARCAFELTQTPSSVEAAVGGTVTIKCQASQSISSYLAWYQQKPGQPPKFLIYRASTLASGVPSRFKGSGSGTEFTLTISDLECADAATYYCQSYYDSVSNP

SEQ ID NO: 379 - protein, 127 aa, Oryctolagus cuniculus
METGLRWLLLVAVLKGVQCQSLEESGGDLVKPEGSLTLTCKASGLDLGTYWFMCWVRQAPGKGLEWIACIYTGSSGSTFYASWVNGRFTISKTSSTTVTLQMTSLTAADTATYFCARGYSGYGYFKL

SEQ ID NO: 380 - protein, 11 aa, Oryctolagus cuniculus
QASQSISSYLA

SEQ ID NO: 381 - protein, 7 aa, Oryctolagus cuniculus
RASTLAS

SEQ ID NO: 382 - protein, 10 aa, Oryctolagus cuniculus
QSYYDSVSNP

SEQ ID NO: 383 - protein, 6 aa, Oryctolagus cuniculus
TYWFMC

SEQ ID NO: 384 - protein, 18 aa, Oryctolagus cuniculus
CIYTGSSGSTFYASWVNG

SEQ ID NO: 385 - protein, 10 aa, Oryctolagus cuniculus
GYSGYGYFKL

SEQ ID NO: 386 - DNA, 360 bp, Oryctolagus cuniculus
  source 1..360: mol_type = genomic DNA
atggacacgagggcccccactcagctgctggggctcctgctgctctggctcccaggtgccagatgtgcattcgaattgacccagactccatcctccgtggaggcagctgtgggaggcacagtcaccatcaagtgccaggccagtcagagcattagtagttacttagcctggtatcagcagaaaccagggcagcctcccaagttcctgatctacagggcgtccactctggcatctggggtcccatcgcgattcaaaggcagtggatctgggacagagttcactctcaccatcagcgacctggagtgtgccgatgctgccacttactactgtcaaagctattatgatagtgtttcaaatcct

SEQ ID NO: 387 - DNA, 381 bp, Oryctolagus cuniculus
  source 1..381: mol_type = genomic DNA
atggagactgggctgcgctggcttctcctggtcgctgtgctcaaaggtgtccagtgtcagtcgttggaggagtccgggggagacctggtcaagcctgagggatccctgacactcacctgcaaagcctctggactcgacctcggtacctactggttcatgtgctgggtccgccaggctccagggaaggggctggagtggatcgcttgtatttatactggtagtagtggttccactttctacgcgagctgggtgaatggccgattcaccatctccaaaacctcgtcgaccacggtgactctgcaaatgaccagtctgacagccgcggacacggccacttatttttgtgcgagaggttatagtggttatggttattttaagttg

SEQ ID NO: 388 - DNA, 33 bp, Oryctolagus cuniculus
  source 1..33: mol_type = genomic DNA
caggccagtcagagcattagtagttacttagcc

SEQ ID NO: 389 - DNA, 21 bp, Oryctolagus cuniculus
  source 1..21: mol_type = genomic DNA
agggcgtccactctggcatct

SEQ ID NO: 390 - DNA, 30 bp, Oryctolagus cuniculus
  source 1..30: mol_type = genomic DNA
caaagctattatgatagtgtttcaaatcct

SEQ ID NO: 391 - DNA, 18 bp, Oryctolagus cuniculus
  source 1..18: mol_type = genomic DNA
acctactggttcatgtgc

SEQ ID NO: 392 - DNA, 54 bp, Oryctolagus cuniculus
  source 1..54: mol_type = genomic DNA
tgtatttatactggtagtagtggttccactttctacgcgagctgggtgaatggc

SEQ ID NO: 393 - DNA, 30 bp, Oryctolagus cuniculus
  source 1..30: mol_type = genomic DNA
ggttatagtggttatggttattttaagttg

SEQ ID NO: 394 - protein, 124 aa, Oryctolagus cuniculus
MDTRAPTQLLGLLLLWLPGVTFAIEMTQSPFSVSAAVGGTVSISCQASQSVYKNNQLSWYQQKSGQPPKLLIYGASALASGVPSRFKGSGSGTEFTLTISDVQCDDAATYYCAGAITGSIDTDG

SEQ ID NO: 395 - protein, 130 aa, Oryctolagus cuniculus
METGLRWLLLVAVLKGVQCQSLEESGGDLVKPGASLTLTCTTSGFSFSSSYFICWVRQAPGKGLEWIACIYGGDGSTYYASWAKGRFTISKTSSTTVTLQMTSLTAADTATYFCAREWAYSQGYFGAFDL

SEQ ID NO: 396 - protein, 13 aa, Oryctolagus cuniculus
QASQSVYKNNQLS

SEQ ID NO: 397 - protein, 7 aa, Oryctolagus cuniculus
GASALAS

SEQ ID NO: 398 - protein, 12 aa, Oryctolagus cuniculus
AGAITGSIDTDG

SEQ ID NO: 399 - protein, 6 aa, Oryctolagus cuniculus
SSYFIC

SEQ ID NO: 400 - protein, 17 aa, Oryctolagus cuniculus
CIYGGDGSTYYASWAKG

SEQ ID NO: 401 - protein, 14 aa, Oryctolagus cuniculus
EWAYSQGYFGAFDL

SEQ ID NO: 402 - DNA, 372 bp, Oryctolagus cuniculus
  source 1..372: mol_type = genomic DNA
atggacacgagggcccccactcagctgctggggctcctgctgctctggctcccaggtgtcacatttgccatcgaaatgacccagagtccattctccgtgtctgcagctgtgggaggcacagtcagcatcagttgccaggccagtcagagtgtttataagaacaaccaattatcctggtatcagcagaaatcagggcagcctcccaagctcctgatctatggtgcatcggctctggcatctggggtcccatcgcggttcaaaggcagtggatctgggacagagttcactctcaccatcagcgacgtgcagtgtgacgatgctgccacttactactgtgcaggcgctattactggtagtattgatacggatggt

SEQ ID NO: 403 - DNA, 390 bp, Oryctolagus cuniculus
  source 1..390: mol_type = genomic DNA
atggagactgggctgcgctggcttctcctggtcgctgtgctcaaaggtgtccagtgtcagtcgttggaggagtccgggggagacctggtcaagcctggggcatccctgacactcacctgcacaacttctggattctccttcagtagcagctacttcatttgctgggtccgccaggctccagggaaggggctggagtggatcgcatgcatttatggtggtgatggcagcacatactacgcgagctgggcgaaaggccgattcaccatctccaaaacctcgtcgaccacggtgacgctgcaaatgaccagtctgacagccgcggacacggccacctatttctgtgcgagagaatgggcatatagtcaaggttattttggtgcttttgatctc

SEQ ID NO: 404 - DNA, 39 bp, Oryctolagus cuniculus
  source 1..39: mol_type = genomic DNA
caggccagtcagagtgtttataagaacaaccaattatcc

SEQ ID NO: 405 - DNA, 21 bp, Oryctolagus cuniculus
  source 1..21: mol_type = genomic DNA
ggtgcatcggctctggcatct

SEQ ID NO: 406 - DNA, 36 bp, Oryctolagus cuniculus
  source 1..36: mol_type = genomic DNA
gcaggcgctattactggtagtattgatacggatggt

SEQ ID NO: 407 - DNA, 18 bp, Oryctolagus cuniculus
  source 1..18: mol_type = genomic DNA
agcagctacttcatttgc

SEQ ID NO: 408 - DNA, 51 bp, Oryctolagus cuniculus
  source 1..51: mol_type = genomic DNA
tgcatttatggtggtgatggcagcacatactacgcgagctgggcgaaaggc

SEQ ID NO: 409 - DNA, 42 bp, Oryctolagus cuniculus
  source 1..42: mol_type = genomic DNA
gaatgggcatatagtcaaggttattttggtgcttttgatctc

SEQ ID NO: 410 - protein, 124 aa, Oryctolagus cuniculus
MDTRAPTQLLGLLLLWLPGARCDVVMTQTPASVEAAVGGTVTIKCQASEDISSYLAWYQQKPGQPPKLLIYAASNLESGVSSRFKGSGSGTEYTLTISDLECADAATYYCQCTYGTISISDGNA

SEQ ID NO: 411 - protein, 124 aa, Oryctolagus cuniculus
METGLRWLLLVAVLKGVQCQSVEESGGRLVTPGTPLTLTCTVSGFSLSSYFMTWVRQAPGEGLEYIGFINPGGSAYYASWVKGRFTISKSSTTVDLKITSPTTEDTATYFCARVLIVSYGAFTI

SEQ ID NO: 412 - protein, 11 aa, Oryctolagus cuniculus
QASEDISSYLA

SEQ ID NO: 413 - protein, 7 aa, Oryctolagus cuniculus
AASNLES

SEQ ID NO: 414 - protein, 14 aa, Oryctolagus cuniculus
QCTYGTISISDGNA

SEQ ID NO: 415 - protein, 5 aa, Oryctolagus cuniculus
SYFMT

SEQ ID NO: 416 - protein, 16 aa, Oryctolagus cuniculus
FINPGGSAYYASWVKG

SEQ ID NO: 417 - protein, 11 aa, Oryctolagus cuniculus
VLIVSYGAFTI

SEQ ID NO: 418 - DNA, 372 bp, Oryctolagus cuniculus
  source 1..372: mol_type = genomic DNA
atggacacgagggcccccactcagctgctggggctcctgctgctctggctcccaggtgccagatgtgatgttgtgatgacccagactccagcctccgtggaggcagctgtgggaggcacagtcaccatcaagtgccaggccagtgaggatattagtagctacttagcctggtatcagcagaaaccagggcagcctcccaagctcctgatctatgctgcatccaatctggaatctggggtctcatcgcgattcaaaggcagtggatctgggacagagtacactctcaccatcagcgacctggagtgtgccgatgctgccacctattactgtcaatgtacttatggtactatttctattagtgatggtaatgct

SEQ ID NO: 419 - DNA, 372 bp, Oryctolagus cuniculus
  source 1..372: mol_type = genomic DNA
atggagactgggctgcgctggcttctcctggtcgctgtgctcaaaggtgtccaatgtcagtcggtggaggagtccgggggtcgcctggtcacgcctgggacacccctgacactcacctgcacagtctctggattctccctcagtagctacttcatgacctgggtccgccaggctccaggggaggggctggaatacatcggattcattaatcctggtggtagcgcttactacgcgagctgggtgaaaggccgattcaccatctccaagtcctcgaccacggtagatctgaaaatcaccagtccgacaaccgaggacacggccacctatttctgtgccagggttctgattgtttcttatggagcctttaccatc

SEQ ID NO: 420 - DNA, 33 bp, Oryctolagus cuniculus
  source 1..33: mol_type = genomic DNA
caggccagtgaggatattagtagctacttagcc

SEQ ID NO: 421 - DNA, 21 bp, Oryctolagus cuniculus
  source 1..21: mol_type = genomic DNA
gctgcatccaatctggaatct

SEQ ID NO: 422 - DNA, 42 bp, Oryctolagus cuniculus
  source 1..42: mol_type = genomic DNA
caatgtacttatggtactatttctattagtgatggtaatgct

SEQ ID NO: 423 - DNA, 15 bp, Oryctolagus cuniculus
  source 1..15: mol_type = genomic DNA
agctacttcatgacc

SEQ ID NO: 424 - DNA, 48 bp, Oryctolagus cuniculus
  source 1..48: mol_type = genomic DNA
ttcattaatcctggtggtagcgcttactacgcgagctgggtgaaaggc

SEQ ID NO: 425 - DNA, 33 bp, Oryctolagus cuniculus
  source 1..33: mol_type = genomic DNA
gttctgattgtttcttatggagcctttaccatc

SEQ ID NO: 426 - protein, 124 aa, Oryctolagus cuniculus
MDTRAPTQLLGLLLLWLPGARCDVVMTQTPASVSAAVGGTVTIKCQASEDIESYLAWYQQKPGQPPKLLIYGASNLESGVSSRFKGSGSGTEFTLTISDLECADAATYYCQCTYGIISISDGNA

SEQ ID NO: 427 - protein, 124 aa, Oryctolagus cuniculus
METGLRWLLLVAVLKGVQCQSVEESGGRLVTPGTPLTLTCTVSGFSLSSYFMTWVRQAPGEGLEYIGFMNTGDNAYYASWAKGRFTISKTSTTVDLKITSPTTEDTATYFCARVLVVAYGAFNI

SEQ ID NO: 428 - protein, 11 aa, Oryctolagus cuniculus
QASEDIESYLA

SEQ ID NO: 429 - protein, 7 aa, Oryctolagus cuniculus
GASNLES

SEQ ID NO: 430 - protein, 14 aa, Oryctolagus cuniculus
QCTYGIISISDGNA

SEQ ID NO: 431 - protein, 5 aa, Oryctolagus cuniculus
SYFMT

SEQ ID NO: 432 - protein, 16 aa, Oryctolagus cuniculus
FMNTGDNAYYASWAKG

SEQ ID NO: 433 - protein, 11 aa, Oryctolagus cuniculus
VLVVAYGAFNI

SEQ ID NO: 434 - DNA, 372 bp, Oryctolagus cuniculus
  source 1..372: mol_type = genomic DNA
atggacacgagggcccccactcagctgctggggctcctgctgctctggctcccaggtgccagatgtgatgttgtgatgacccagactccagcctccgtgtctgcagctgtgggaggcacagtcaccatcaagtgccaggccagtgaggacattgaaagctatctagcctggtatcagcagaaaccagggcagcctcccaagctcctgatctatggtgcatccaatctggaatctggggtctcatcgcggttcaaaggcagtggatctgggacagagttcactctcaccatcagcgacctggagtgtgccgatgctgccacttactattgtcaatgcacttatggtattattagtattagtgatggtaatgct

SEQ ID NO: 435 - DNA, 372 bp, Oryctolagus cuniculus
  source 1..372: mol_type = genomic DNA
atggagactgggctgcgctggcttctcctggtcgctgtgctcaaaggtgtccagtgtcagtcggtggaggagtccgggggtcgcctggtcacgcctgggacacccctgacactcacctgcacagtgtctggattctccctcagtagctacttcatgacctgggtccgccaggctccaggggaggggctggaatacatcggattcatgaatactggtgataacgcatactacgcgagctgggcgaaaggccgattcaccatctccaaaacctcgaccacggtggatctgaaaatcaccagtccgacaaccgaggacacggccacctatttctgtgccagggttcttgttgttgcttatggagcctttaacatc

SEQ ID NO: 436 - DNA, 33 bp, Oryctolagus cuniculus
  source 1..33: mol_type = genomic DNA
caggccagtgaggacattgaaagctatctagcc

SEQ ID NO: 437 - DNA, 21 bp, Oryctolagus cuniculus
  source 1..21: mol_type = genomic DNA
ggtgcatccaatctggaatct

SEQ ID NO: 438 - DNA, 42 bp, Oryctolagus cuniculus
  source 1..42: mol_type = genomic DNA
caatgcacttatggtattattagtattagtgatggtaatgct

SEQ ID NO: 439 - DNA, 15 bp, Oryctolagus cuniculus
  source 1..15: mol_type = genomic DNA
agctacttcatgacc

SEQ ID NO: 440 - DNA, 48 bp, Oryctolagus cuniculus
  source 1..48: mol_type = genomic DNA
ttcatgaatactggtgataacgcatactacgcgagctgggcgaaaggc

SEQ ID NO: 441 - DNA, 33 bp, Oryctolagus cuniculus
  source 1..33: mol_type = genomic DNA
gttcttgttgttgcttatggagcctttaacatc

SEQ ID NO: 442 - protein, 124 aa, Oryctolagus cuniculus
MDTRAPTQLLGLLLLWLPGATFAAVLTQTPSPVSEPVGGTVSISCQSSKSVMNNNYLAWYQQKPGQPPKLLIYGASNLASGVPSRFSGSGSGTQFTLTISDVQCDDAATYYCQGGYTGYSDHGT

SEQ ID NO: 443 - protein, 127 aa, Oryctolagus cuniculus
METGLRWLLLVAVLKGVQCQSVEESGGRLVKPDETLTLTCTVSGIDLSSYPMNWVRQAPGKGLEWIGFINTGGTIVYASWAKGRFTISKTSTTVDLKMTSPTTEDTATYFCARGSYVSSGYAYYFNV

SEQ ID NO: 444 - protein, 13 aa, Oryctolagus cuniculus
QSSKSVMNNNYLA

SEQ ID NO: 445 - protein, 7 aa, Oryctolagus cuniculus
GASNLAS

SEQ ID NO: 446 - protein, 12 aa, Oryctolagus cuniculus
QGGYTGYSDHGT

SEQ ID NO: 447 - protein, 5 aa, Oryctolagus cuniculus
SYPMN

SEQ ID NO: 448 - protein, 16 aa, Oryctolagus cuniculus
FINTGGTIVYASWAKG

SEQ ID NO: 449 - protein, 14 aa, Oryctolagus cuniculus
GSYVSSGYAYYFNV

SEQ ID NO: 450 - DNA, 372 bp, Oryctolagus cuniculus
  source 1..372: mol_type = genomic DNA
atggacacgagggcccccactcagctgctggggctcctgctgctctggctcccaggtgccacatttgccgccgtgctgacccagactccatctcccgtgtctgaacctgtgggaggcacagtcagcatcagttgccagtccagtaagagtgttatgaataacaactacttagcctggtatcagcagaaaccagggcagcctcccaagctcctgatctatggtgcatccaatctggcatctggggtcccatcacggttcagcggcagtggatctgggacacagttcactctcaccatcagcgacgtgcagtgtgacgatgctgccacttactactgtcaaggcggttatactggttatagtgatcatgggact

SEQ ID NO: 451 - DNA, 381 bp, Oryctolagus cuniculus
  source 1..381: mol_type = genomic DNA
atggagactgggctgcgctggcttctcctggtcgctgtgctcaaaggtgtccagtgtcagtcggtggaggagtccgggggtcgcctggtcaagcctgacgaaaccctgacactcacctgcacagtctctggaatcgacctcagtagctatccaatgaactgggtccgccaggctccagggaaggggctggaatggatcggattcattaatactggtggtaccatagtctacgcgagctgggcaaaaggccgattcaccatctccaaaacctcgaccacggtggatctgaaaatgaccagtccgacaaccgaggacacggccacctatttctgtgccagaggcagttatgtttcatctggttatgcctactattttaatgtc

SEQ ID NO: 452 - DNA, 39 bp, Oryctolagus cuniculus
  source 1..39: mol_type = genomic DNA
cagtccagtaagagtgttatgaataacaactacttagcc

SEQ ID NO: 453 - DNA, 21 bp, Oryctolagus cuniculus
  source 1..21: mol_type = genomic DNA
ggtgcatccaatctggcatct

SEQ ID NO: 454 - DNA, 36 bp, Oryctolagus cuniculus
  source 1..36: mol_type = genomic DNA
caaggcggttatactggttatagtgatcatgggact

SEQ ID NO: 455 - DNA, 15 bp, Oryctolagus cuniculus
  source 1..15: mol_type = genomic DNA
agctatccaatgaac

SEQ ID NO: 456 - DNA, 48 bp, Oryctolagus cuniculus
  source 1..48: mol_type = genomic DNA
ttcattaatactggtggtaccatagtctacgcgagctgggcaaaaggc

SEQ ID NO: 457 - DNA, 42 bp, Oryctolagus cuniculus
  source 1..42: mol_type = genomic DNA
ggcagttatgtttcatctggttatgcctactattttaatgtc

SEQ ID NO: 458 - protein, 121 aa, Oryctolagus cuniculus
MDTRAPTQLLGLLLLWLPGATFAAVLTQTPSPVSAAVGGTVSISCQSSQSVYNNNWLSWFQQKPGQPPKLLIYKASTLASGVPSRFKGSGSGTQFTLTISDVQCDDVATYYCAGGYLDSVI

SEQ ID NO: 459 - protein, 126 aa, Oryctolagus cuniculus
METGLRWLLLVAVLKGVQCQSVEESGGRLVTPGTPLTLTCTVSGFSLSTYSINWVRQAPGKGLEWIGIIANSGTTFYANWAKGRFTVSKTSTTVDLKITSPTTEDTATYFCARESGMYNEYGKFNI

SEQ ID NO: 460 - protein, 13 aa, Oryctolagus cuniculus
QSSQSVYNNNWLS

SEQ ID NO: 461 - protein, 7 aa, Oryctolagus cuniculus
KASTLAS

SEQ ID NO: 462 - protein, 9 aa, Oryctolagus cuniculus
AGGYLDSVI

SEQ ID NO: 463 - protein, 5 aa, Oryctolagus cuniculus
TYSIN

SEQ ID NO: 464 - protein, 16 aa, Oryctolagus cuniculus
IIANSGTTFYANWAKG

SEQ ID NO: 465 - protein, 13 aa, Oryctolagus cuniculus
ESGMYNEYGKFNI

SEQ ID NO: 466 - DNA, 363 bp, Oryctolagus cuniculus
  source 1..363: mol_type = genomic DNA
atggacacgagggcccccactcagctgctggggctcctgctgctctggctcccaggtgccacatttgccgccgtgctgacccagactccatctcccgtgtctgcagctgtgggaggcacagtcagcatcagttgccagtccagtcagagtgtttataataacaactggttatcctggtttcagcagaaaccagggcagcctcccaagctcctgatctacaaggcatccactctggcatctggggtcccatcgcggttcaaaggcagtggatctgggacacagttcactctcaccatcagcgacgtgcagtgtgacgatgttgccacttactactgtgcgggcggttatcttgatagtgttatt

SEQ ID NO: 467 - DNA, 378 bp, Oryctolagus cuniculus
  source 1..378: mol_type = genomic DNA
atggagactgggctgcgctggcttctcctggtcgctgtgctcaaaggtgtccagtgtcagtcggtggaggagtccgggggtcgcctggtcacgcctgggacacccctgacactcacctgcacagtctctggattctccctcagtacctattcaataaactgggtccgccaggctccagggaagggcctggaatggatcggaatcattgctaatagtggtaccacattctacgcgaactgggcgaaaggccgattcaccgtctccaaaacctcgaccacggtggatctgaaaatcaccagtccgacaaccgaggacacggccacctatttctgtgccagagagagtggaatgtacaatgaatatggtaaatttaacatc

SEQ ID NO: 468 - DNA, 39 bp, Oryctolagus cuniculus
  source 1..39: mol_type = genomic DNA
cagtccagtcagagtgtttataataacaactggttatcc

SEQ ID NO: 469 - DNA, 21 bp, Oryctolagus cuniculus
  source 1..21: mol_type = genomic DNA
aaggcatccactctggcatct

SEQ ID NO: 470 - DNA, 27 bp, Oryctolagus cuniculus
  source 1..27: mol_type = genomic DNA
gcgggcggttatcttgatagtgttatt

SEQ ID NO: 471 - DNA, 15 bp, Oryctolagus cuniculus
  source 1..15: mol_type = genomic DNA
acctattcaataaac

SEQ ID NO: 472 - DNA, 48 bp, Oryctolagus cuniculus
  source 1..48: mol_type = genomic DNA
atcattgctaatagtggtaccacattctacgcgaactgggcgaaaggc

SEQ ID NO: 473 - DNA, 39 bp, Oryctolagus cuniculus
  source 1..39: mol_type = genomic DNA
gagagtggaatgtacaatgaatatggtaaatttaacatc

SEQ ID NO: 474 - protein, 122 aa, Oryctolagus cuniculus
MDTRAPTQLLGLLLLWLPGARCASDMTQTPSSVSAAVGGTVTINCQASENIYSFLAWYQQKPGQPPKLLIFKASTLASGVSSRFKGSGSGTQFTLTISDLECDDAATYYCQQGATVYDIDNN

SEQ ID NO: 475 - protein, 128 aa, Oryctolagus cuniculus
METGLRWLLLVAVLKGVQCQSLEESGGRLVTPGTPLTLTCTVSGIDLSAYAMIWVRQAPGEGLEWITIIYPNGITYYANWAKGRFTVSKTSTAMDLKITSPTTEDTATYFCARDAESSKNAYWGYFNV

SEQ ID NO: 476 - protein, 11 aa, Oryctolagus cuniculus
QASENIYSFLA

SEQ ID NO: 477 - protein, 7 aa, Oryctolagus cuniculus
KASTLAS

SEQ ID NO: 478 - protein, 12 aa, Oryctolagus cuniculus
QQGATVYDIDNN

SEQ ID NO: 479 - protein, 5 aa, Oryctolagus cuniculus
AYAMI

SEQ ID NO: 480 - protein, 16 aa, Oryctolagus cuniculus
IIYPNGITYYANWAKG

SEQ ID NO: 481 - protein, 15 aa, Oryctolagus cuniculus
DAESSKNAYWGYFNV

SEQ ID NO: 482 - DNA, 366 bp, Oryctolagus cuniculus
  source 1..366: mol_type = genomic DNA
atggacacgagggcccccactcagctgctggggctcctgctgctctggctcccaggtgccagatgtgcctctgatatgacccagactccatcctccgtgtctgcagctgtgggaggcacagtcaccatcaattgccaggccagtgagaacatttatagctttttggcctggtatcagcagaaaccagggcagcctcccaagctcctgatcttcaaggcttccactctggcatctggggtctcatcgcggttcaaaggcagtggatctgggacacagttcactctcaccatcagcgacctggagtgtgacgatgctgccacttactactgtcaacagggtgctactgtgtatgatattgataataat

SEQ ID NO: 483 - DNA, 384 bp, Oryctolagus cuniculus
  source 1..384: mol_type = genomic DNA
atggagactgggctgcgctggcttctcctggtcgctgtgctcaaaggtgtccagtgtcagtcgctggaggagtccgggggtcgcctggtcacgcctgggacacccctgacactcacctgcacagtttctggaatcgacctcagtgcctatgcaatgatctgggtccgccaggctccaggggaggggctggaatggatcacaatcatttatcctaatggtatcacatactacgcgaactgggcgaaaggccgattcaccgtctccaaaacctcgaccgcgatggatctgaaaatcaccagtccgacaaccgaggacacggccacctatttctgtgccagagatgcagaaagtagtaagaatgcttattggggctactttaacgtc

SEQ ID NO: 484 - DNA, 33 bp, Oryctolagus cuniculus
  source 1..33: mol_type = genomic DNA
caggccagtgagaacatttatagctttttggcc

SEQ ID NO: 485 - DNA, 21 bp, Oryctolagus cuniculus
  source 1..21: mol_type = genomic DNA
aaggcttccactctggcatct

SEQ ID NO: 486 - DNA, 36 bp, Oryctolagus cuniculus
  source 1..36: mol_type = genomic DNA
caacagggtgctactgtgtatgatattgataataat

SEQ ID NO: 487 - DNA, 15 bp, Oryctolagus cuniculus
  source 1..15: mol_type = genomic DNA
gcctatgcaatgatc

SEQ ID NO: 488 - DNA, 48 bp, Oryctolagus cuniculus
  source 1..48: mol_type = genomic DNA
atcatttatcctaatggtatcacatactacgcgaactgggcgaaaggc

SEQ ID NO: 489 - DNA, 45 bp, Oryctolagus cuniculus
  source 1..45: mol_type = genomic DNA
gatgcagaaagtagtaagaatgcttattggggctactttaacgtc

SEQ ID NO: 490 - protein, 122 aa, Oryctolagus cuniculus
MDTRAPTQLLGLLLLWLPGARCASDMTQTPSSVSAAVGGTVTINCQASENIYSFLAWYQQKPGQPPKLLIFRASTLASGVSSRFKGSGSGTQFTLTISDLECDDAATYYCQQGATVYDIDNN

SEQ ID NO: 491 - protein, 128 aa, Oryctolagus cuniculus; identical to SEQ ID NO: 475

SEQ ID NO: 492 - protein, 11 aa, Oryctolagus cuniculus
QASENIYSFLA

SEQ ID NO: 493 - protein, 7 aa, Oryctolagus cuniculus
RASTLAS

SEQ ID NO: 494 - protein, 12 aa, Oryctolagus cuniculus
QQGATVYDIDNN

SEQ ID NO: 495 - protein, 5 aa, Oryctolagus cuniculus
AYAMI

SEQ ID NO: 496 - protein, 16 aa, Oryctolagus cuniculus
IIYPNGITYYANWAKG

SEQ ID NO: 497 - protein, 15 aa, Oryctolagus cuniculus
DAESSKNAYWGYFNV

SEQ ID NO: 498 - DNA, 366 bp, Oryctolagus cuniculus
  source 1..366: mol_type = genomic DNA
atggacacgagggcccccactcagctgctggggctcctgctgctctggctcccaggtgccagatgtgcctctgatatgacccagactccatcctccgtgtctgcagctgtgggaggcacagtcaccatcaattgccaggccagtgagaacatttatagctttttggcctggtatcagcagaaaccagggcagcctcccaagctcctgatcttcagggcttccactctggcatctggggtctcatcgcggttcaaaggcagtggatctgggacacagttcactctcaccatcagcgacctggagtgtgacgatgctgccacttactactgtcaacagggtgctactgtgtatgatattgataataat

SEQ ID NO: 499 - DNA, 384 bp, Oryctolagus cuniculus; identical to SEQ ID NO: 483
  source 1..384: mol_type = genomic DNA

SEQ ID NO: 500 - DNA, 33 bp, Oryctolagus cuniculus; identical to SEQ ID NO: 484
  source 1..33: mol_type = genomic DNA

SEQ ID NO: 501 - DNA, 21 bp, Oryctolagus cuniculus
  source 1..21: mol_type = genomic DNA
agggcttccactctggcatct

SEQ ID NO: 502 - DNA, 36 bp, Oryctolagus cuniculus; identical to SEQ ID NO: 486
  source 1..36: mol_type = genomic DNA

SEQ ID NO: 503 - DNA, 15 bp, Oryctolagus cuniculus
  source 1..15: mol_type = genomic DNA
gcctatgcaatgatc

SEQ ID NO: 504 - DNA, 48 bp, Oryctolagus cuniculus; identical to SEQ ID NO: 488
  source 1..48: mol_type = genomic DNA

SEQ ID NO: 505 - DNA, 45 bp, Oryctolagus cuniculus; identical to SEQ ID NO: 489
  source 1..45: mol_type = genomic DNA

SEQ ID NO: 506 - protein, 124 aa, Oryctolagus cuniculus
MDTRAPTQLLGLLLLWLPGATFAIEMTQTPSPVSAAVGGTVTINCQASESVFNNMLSWYQQKPGHSPKLLIYDASDLASGVPSRFKGSGSGTQFTLTISGVECDDAATYYCAGYKSDSNDGDNV

SEQ ID NO: 507 - protein, 123 aa, Oryctolagus cuniculus
METGLRWLLLVAVLKGVQCQSLEESGGRLVTPGTPLTLTCTVSGFSLNRNSITWVRQAPGEGLEWIGIITGSGRTYYANWAKGRFTISKTSTTVDLKMTSPTTEDTATYFCARGHPGLGSGNI

SEQ ID NO: 508 - protein, 12 aa, Oryctolagus cuniculus
QASESVFNNMLS

SEQ ID NO: 509 - protein, 7 aa, Oryctolagus cuniculus
DASDLAS

SEQ ID NO: 510 - protein, 13 aa, Oryctolagus cuniculus
AGYKSDSNDGDNV

SEQ ID NO: 511 - protein, 5 aa, Oryctolagus cuniculus
RNSIT

SEQ ID NO: 512 - protein, 16 aa, Oryctolagus cuniculus
IITGSGRTYYANWAKG

SEQ ID NO: 513 - protein, 10 aa, Oryctolagus cuniculus
GHPGLGSGNI

SEQ ID NO: 514 - DNA, 372 bp, Oryctolagus cuniculus
  source 1..372: mol_type = genomic DNA
atggacacgagggcccccactcagctgctggggctcctgctgctctggctcccaggtgccacatttgccattgaaatgacccagactccatcccccgtgtctgccgctgtgggaggcacagtcaccatcaattgccaggccagtgagagtgtttttaataatatgttatcctggtatcagcagaaaccagggcactctcctaagctcctgatctatgatgcatccgatctggcatctggggtcccatcgcggttcaaaggcagtggatctgggacacagttcactctcaccatcagtggcgtggagtgtgacgatgctgccacttactattgtgcagggtataaaagtgatagtaatgatggcgataatgtt

SEQ ID NO: 515 - DNA, 369 bp, Oryctolagus cuniculus
  source 1..369: mol_type = genomic DNA
atggagactgggctgcgctggcttctcctggtcgctgtgctcaaaggtgtccagtgtcagtcgctggaggagtccgggggtcgcctggtcacgcctgggacacccctgacactcacctgcacagtctctggattctccctcaacaggaattcaataacctgggtccgccaggctccaggggaggggctggaatggatcggaatcattactggtagtggtagaacgtactacgcgaactgggcaaaaggccgattcaccatctccaaaacctcgaccacggtggatctgaaaatgaccagtccgacaaccgaggacacggccacctatttctgtgccagaggccatcctggtcttggtagtggtaacatc

SEQ ID NO: 516 - DNA, 36 bp, Oryctolagus cuniculus
  source 1..36: mol_type = genomic DNA
caggccagtgagagtgtttttaataatatgttatcc

SEQ ID NO: 517 - DNA, 21 bp, Oryctolagus cuniculus
  source 1..21: mol_type = genomic DNA
gatgcatccgatctggcatct

SEQ ID NO: 518 - DNA, 39 bp, Oryctolagus cuniculus
  source 1..39: mol_type = genomic DNA
gcagggtataaaagtgatagtaatgatggcgataatgtt

SEQ ID NO: 519 - DNA, 15 bp, Oryctolagus cuniculus
  source 1..15: mol_type = genomic DNA
aggaattcaataacc

SEQ ID NO: 520 - DNA, 48 bp, Oryctolagus cuniculus
  source 1..48: mol_type = genomic DNA
atcattactggtagtggtagaacgtactacgcgaactgggcaaaaggc

SEQ ID NO: 521 - DNA, 30 bp, Oryctolagus cuniculus
  source 1..30: mol_type = genomic DNA
ggccatcctggtcttggtagtggtaacatc

SEQ ID NO: 522 - protein, 121 aa, Oryctolagus cuniculus
MDTRAPTQLLGLLLLWLPGATFAQVLTQTASSVSAAVGGTVTINCQSSQSVYNNYLSWYQQKPGQPPKLLIYTASSLASGVPSRFKGSGSGTQFTLTISEVQCDDAATYYCQGYYSGPIIT

SEQ ID NO: 523 - protein, 122 aa, Oryctolagus cuniculus
METGLRWLLLVAVLKGVQCQSLEESGGRLVTPGTPLTLTCTASGFSLNNYYIQWVRQAPGEGLEWIGIIYAGGSAYYATWANGRFTIAKTSSTTVDLKMTSLTTEDTATYFCARGTFDGYEL

SEQ ID NO: 524 - protein, 12 aa, Oryctolagus cuniculus
QSSQSVYNNYLS

SEQ ID NO: 525 - protein, 7 aa, Oryctolagus cuniculus
TASSLAS

SEQ ID NO: 526 - protein, 10 aa, Oryctolagus cuniculus
QGYYSGPIIT

SEQ ID NO: 527 - protein, 5 aa, Oryctolagus cuniculus
NYYIQ

SEQ ID NO: 528 - protein, 16 aa, Oryctolagus cuniculus
IIYAGGSAYYATWANG

SEQ ID NO: 529 - protein, 8 aa, Oryctolagus cuniculus
GTFDGYEL

SEQ ID NO: 530 - DNA, 363 bp, Oryctolagus cuniculus
  source 1..363: mol_type = genomic DNA
atggacacgagggcccccactcagctgctggggctcctgctgctctggctcccaggtgccacatttgcgcaagtgctgacccagactgcatcgtccgtgtctgcagctgtgggaggcacagtcaccatcaattgccagtccagtcagagtgtttataataactacttatcctggtatcagcagaaaccagggcagcctcccaagctcctgatctatactgcatccagcctggcatctggggtcccatcgcggttcaaaggcagtggatctgggacacagttcactctcaccatcagcgaagtgcagtgtgacgatgctgccacttactactgtcaaggctattatagtggtcctataattact

SEQ ID NO: 531 - DNA, 366 bp, Oryctolagus cuniculus
  source 1..366: mol_type = genomic DNA
atggagactgggctgcgctggcttctcctggtcgctgtgctcaaaggtgtccagtgtcagtcgctggaggagtccgggggtcgcctggtcacgcctgggacacccctgacactcacctgcacagcctctggattctccctcaataactactacatacaatgggtccgccaggctccaggggaggggctggaatggatcgggatcatttatgctggtggtagcgcatactacgcgacctgggcaaacggccgattcaccatcgccaaaacctcgtcgaccacggtggatctgaagatgaccagtctgacaaccgaggacacggccacctatttctgtgccagagggacatttgatggttatgagttg

SEQ ID NO: 532 - DNA, 36 bp, Oryctolagus cuniculus
  source 1..36: mol_type = genomic DNA
cagtccagtcagagtgtttataataactacttatcc

SEQ ID NO: 533 - DNA, 21 bp, Oryctolagus cuniculus
  source 1..21: mol_type = genomic DNA
actgcatccagcctggcatct

SEQ ID NO: 534 - DNA, 30 bp, Oryctolagus cuniculus
  source 1..30: mol_type = genomic DNA
caaggctattatagtggtcctataattact

SEQ ID NO: 535 - DNA, 15 bp, Oryctolagus cuniculus
  source 1..15: mol_type = genomic DNA
aactactacatacaa

SEQ ID NO: 536 - DNA, 48 bp, Oryctolagus cuniculus
  source 1..48: mol_type = genomic DNA
atcatttatgctggtggtagcgcatactacgcgacctgggcaaacggc

SEQ ID NO: 537 - DNA, 24 bp, Oryctolagus cuniculus
  source 1..24: mol_type = genomic DNA
gggacatttgatggttatgagttg

SEQ ID NO: 538 - protein, 122 aa, Oryctolagus cuniculus
MDTRAPTQLLGLLLLWLPGATFAQVLTQTPSPVSVPVGDTVTISCQSSESVYSNNLLSWYQQKPGQPPKLLIYRASNLASGVPSRFKGSGSGTQFTLTISGAQCDDAATYYCQGYYSGVINS

SEQ ID NO: 539 - protein, 124 aa, Oryctolagus cuniculus
METGLRWLLLVAVLKGVQCQSVEESGGRLVTPGTPLTLTCTVSGFSLSSYFMSWVRQAPGEGLEYIGFINPGGSAYYASWASGRLTISKTSTTVDLKITSPTTEDTATYFCARILIVSYGAFTI

SEQ ID NO: 540 - protein, 13 aa, Oryctolagus cuniculus
QSSESVYSNNLLS

SEQ ID NO: 541 - protein, 7 aa, Oryctolagus cuniculus
RASNLAS

SEQ ID NO: 542 - protein, 10 aa, Oryctolagus cuniculus
QGYYSGVINS

SEQ ID NO: 543 - protein, 5 aa, Oryctolagus cuniculus
SYFMS

SEQ ID NO: 544 - protein, 16 aa, Oryctolagus cuniculus
FINPGGSAYYASWASG

SEQ ID NO: 545 - protein, 11 aa, Oryctolagus cuniculus
ILIVSYGAFTI

SEQ ID NO: 546 - DNA, 366 bp, Oryctolagus cuniculus
  source 1..366: mol_type = genomic DNA
atggacacgagggcccccactcagctgctggggctcctgctgctctggctcccaggtgccacatttgcccaagtgctgacccagactccatcccctgtgtctgtccctgtgggagacacagtcaccatcagttgccagtccagtgagagcgtttatagtaataacctcttatcctggtatcagcagaaaccagggcagcctcccaagctcctgatctacagggcatccaatctggcatctggtgtcccatcgcggttcaaaggcagtggatctgggacacagttcactctcaccatcagcggcgcacagtgtgacgatgctgccacttactactgtcaaggctattatagtggtgtcattaatagt

SEQ ID NO: 547 - DNA, 372 bp, Oryctolagus cuniculus
  source 1..372: mol_type = genomic DNA
atggagactgggctgcgctggcttctcctggtcgctgtgctcaaaggtgtccagtgtcagtcggtggaggagtccgggggtcgcctggtcacgcctgggacacccctgacactcacctgcacagtgtctggattctccctcagtagctacttcatgagctgggtccgccaggctccaggggaggggctggaatacatcggattcattaatcctggtggtagcgcatactacgcgagctgggcgagtggccgactcaccatctccaaaacctcgaccacggtagatctgaaaatcaccagtccgacaaccgaggacacggccacctatttctgtgccaggattcttattgtttcttatggagcctttaccatc

SEQ ID NO: 548 - DNA, 39 bp, Oryctolagus cuniculus
  source 1..39: mol_type = genomic DNA
cagtccagtgagagcgtttatagtaataacctcttatcc

SEQ ID NO: 549 - DNA, 21 bp, Oryctolagus cuniculus
  source 1..21: mol_type = genomic DNA
agggcatccaatctggcatct

SEQ ID NO: 550 - DNA, 30 bp, Oryctolagus cuniculus
  source 1..30: mol_type = genomic DNA
caaggctattatagtggtgtcattaatagt

SEQ ID NO: 551 - DNA, 15 bp, Oryctolagus cuniculus
  source 1..15: mol_type = genomic DNA
agctacttcatgagc

SEQ ID NO: 552 - DNA, 48 bp, Oryctolagus cuniculus
  source 1..48: mol_type = genomic DNA
ttcattaatcctggtggtagcgcatactacgcgagctgggcgagtggc

SEQ ID NO: 553 - DNA, 33 bp, Oryctolagus cuniculus
  source 1..33: mol_type = genomic DNA
attcttattgtttcttatggagcctttaccatc

SEQ ID NO: 554 - protein, 122 aa, Oryctolagus cuniculus
MDTRAPTQLLGLLLLWLPGARCAYDMTQTPASVEVAVGGTVTIKCQATESIGNELSWYQQKPGQAPKLLIYSASTLASGVPSRFKGSGSGTQFTLTITGVECDDAATYYCQQGYSSANIDNA

SEQ ID NO: 555 - protein, 128 aa, Oryctolagus cuniculus
METGLRWLLLVAVLKGVQCQSLEESGGRLVTPGTPLTLTCTVSGFSLSKYYMSWVRQAPEKGLKYIGYIDSTTVNTYYATWARGRFTISKTSTTVDLKITSPTSEDTATYFCARGSTYFTDGGHRLDL

SEQ ID NO: 556 - protein, 11 aa, Oryctolagus cuniculus
QATESIGNELS

SEQ ID NO: 557 - protein, 7 aa, Oryctolagus cuniculus
SASTLAS

SEQ ID NO: 558 - protein, 12 aa, Oryctolagus cuniculus
QQGYSSANIDNA

SEQ ID NO: 559 - protein, 5 aa, Oryctolagus cuniculus
KYYMS

SEQ ID NO: 560 - protein, 17 aa, Oryctolagus cuniculus
YIDSTTVNTYYATWARG

SEQ ID NO: 561 - protein, 14 aa, Oryctolagus cuniculus
GSTYFTDGGHRLDL

SEQ ID NO: 562 - DNA, 366 bp, Oryctolagus cuniculus
  source 1..366: mol_type = genomic DNA
atggacacgagggcccccactcagctgctggggctcctgctgctctggctcccaggtgccagatgtgcctatgatatgacccagactccagcctctgtggaggtagctgtgggaggcacagtcaccatcaagtgccaggccactgagagcattggcaatgagttatcctggtatcagcagaaaccagggcaggctcccaagctcctgatctattctgcatccactctggcatctggggtcccatcgcggttcaaaggcagtggatctgggacacagttcactctcaccatcaccggcgtggagtgtgatgatgctgccacttactactgtcaacagggttatagtagtgctaatattgataatgct

SEQ ID NO: 563 - DNA, 384 bp, Oryctolagus cuniculus
  source 1..384: mol_type = genomic DNA
atggagactgggctgcgctggcttctcctggtcgctgtgctcaaaggtgtccagtgtcagtcgctggaggagtccgggggtcgcctggtcacgcctgggacacccctgacactcacctgcaccgtctctggattctccctcagtaagtactacatgagctgggtccgccaggctccagagaaggggctgaaatacatcggatacattgatagtactactgttaatacatactacgcgacctgggcgagaggccgattcaccatctccaaaacctcgaccacggtggatctgaagatcaccagtccgacaagtgaggacacggccacctatttctgtgccagaggaagtacttattttactgatggaggccatcggttggatctc

SEQ ID NO: 564 - DNA, 33 bp, Oryctolagus cuniculus
  source 1..33: mol_type = genomic DNA
caggccactgagagcattggcaatgagttatcc

SEQ ID NO: 565 - DNA, 21 bp, Oryctolagus cuniculus
  source 1..21: mol_type = genomic DNA
tctgcatccactctggcatct

SEQ ID NO: 566 - DNA, 36 bp, Oryctolagus cuniculus
  source 1..36: mol_type = genomic DNA
caacagggttatagtagtgctaatattgataatgct

SEQ ID NO: 567 - DNA, 15 bp, Oryctolagus cuniculus
  source 1..15: mol_type = genomic DNA
aagtactacatgagc

SEQ ID NO: 568 - DNA, 51 bp, Oryctolagus cuniculus
  source 1..51: mol_type = genomic DNA
tacattgatagtactactgttaatacatactacgcgacctgggcgagaggc

SEQ ID NO: 569 - DNA, 42 bp, Oryctolagus cuniculus
  source 1..42: mol_type = genomic DNA
ggaagtacttattttactgatggaggccatcggttggatctc

SEQ ID NO: 570 - protein, 122 aa, Oryctolagus cuniculus; identical to SEQ ID NO: 554

SEQ ID NO: 571 - protein, 124 aa, Oryctolagus cuniculus
METGLRWLLLVAVLKGVQCQSLEESGGRLVTPGTPLTLTCTVSGFSLSTYNMGWVRQAPGKGLEWIGSITIDGRTYYASWAKGRFTVSKSSTTVDLKMTSLTTGDTATYFCARILIVSYGAFTI

SEQ ID NO: 572 - protein, 11 aa, Oryctolagus cuniculus
QATESIGNELS

SEQ ID NO: 573 - protein, 7 aa, Oryctolagus cuniculus
SASTLAS

SEQ ID NO: 574 - protein, 12 aa, Oryctolagus cuniculus
QQGYSSANIDNA

SEQ ID NO: 575 - protein, 5 aa, Oryctolagus cuniculus
TYNMG

SEQ ID NO: 576 - protein, 16 aa, Oryctolagus cuniculus
SITIDGRTYYASWAKG

SEQ ID NO: 577 - protein, 11 aa, Oryctolagus cuniculus
ILIVSYGAFTI

SEQ ID NO: 578 - DNA, 366 bp, Oryctolagus cuniculus; identical to SEQ ID NO: 562
  source 1..366: mol_type = genomic DNA

SEQ ID NO: 579 - DNA, 372 bp, Oryctolagus cuniculus
  source 1..372: mol_type = genomic DNA
atggagactgggctgcgctggcttctcctggtcgctgtgctcaaaggtgtccagtgtcagtcgctggaggagtccgggggtcgcctggtaacgcctgggacacccctgacactcacctgcacagtctctggattctccctcagtacctacaacatgggctgggtccgccaggctccagggaaggggctggaatggatcggaagtattactattgatggtcgcacatactacgcgagctgggcgaaaggccgattcaccgtctccaaaagctcgaccacggtggatctgaaaatgaccagtctgacaaccggggacacggccacctatttctgtgccaggattcttattgtttcttatggggcctttaccatc

SEQ ID NO: 580 - DNA, 33 bp, Oryctolagus cuniculus; identical to SEQ ID NO: 564
  source 1..33: mol_type = genomic DNA

SEQ ID NO: 581 - DNA, 21 bp, Oryctolagus cuniculus
  source 1..21: mol_type = genomic DNA
tctgcatccactctggcatct

SEQ ID NO: 582 - DNA, 36 bp, Oryctolagus cuniculus; identical to SEQ ID NO: 566
  source 1..36: mol_type = genomic DNA

SEQ ID NO: 583 - DNA, 15 bp, Oryctolagus cuniculus
  source 1..15: mol_type = genomic DNA
acctacaacatgggc

SEQ ID NO: 584 - DNA, 48 bp, Oryctolagus cuniculus
  source 1..48: mol_type = genomic DNA
agtattactattgatggtcgcacatactacgcgagctgggcgaaaggc

SEQ ID NO: 585 - DNA, 33 bp, Oryctolagus cuniculus
  source 1..33: mol_type = genomic DNA
attcttattgtttcttatggggcctttaccatc

SEQ ID NO: 586 - protein, 105 aa, synthetic construct [sequence omitted]

SEQ ID NO: 587 - DNA, 315 bp, synthetic construct
  misc_feature 1..315: note = Kappa constant domain
  source 1..315: mol_type = other DNA
gtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 588 - protein, 330 aa, synthetic construct
  REGION 1..330: note = Gamma-1 constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 589 - DNA, 990 bp, synthetic construct
  misc_feature 1..990: note = Gamma-1 constant domain
  source 1..990: mol_type = other DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 590 - protein, 15 aa, Homo sapiens
VPPGEDSKDVAAPHR

SEQ ID NO: 591 - protein, 15 aa, Homo sapiens
GEDSKDVAAPHRQPL

SEQ ID NO: 592 - protein, 15 aa, Homo sapiens
SKDVAAPHRQPLTSS

SEQ ID NO: 593 - protein, 15 aa, Homo sapiens
VAAPHRQPLTSSERI

SEQ ID NO: 594 - protein, 15 aa, Homo sapiens
PHRQPLTSSERIDKQ

SEQ ID NO: 595 - protein, 15 aa, Homo sapiens
QPLTSSERIDKQIRY

SEQ ID NO: 596 - protein, 15 aa, Homo sapiens
TSSERIDKQIRYILD

SEQ ID NO: 597 - protein, 15 aa, Homo sapiens
ERIDKQIRYILDGIS

SEQ ID NO: 598 - protein, 15 aa, Homo sapiens
DKQIRYILDGISALR

SEQ ID NO: 599 - protein, 15 aa, Homo sapiens
IRYILDGISALRKET

SEQ ID NO: 600 - protein, 15 aa, Homo sapiens
ILDGISALRKETCNK

SEQ ID NO: 601 - protein, 15 aa, Homo sapiens
GISALRKETCNKSNM

SEQ ID NO: 602 - protein, 15 aa, Homo sapiens
ALRKETCNKSNMCES

SEQ ID NO: 603 - protein, 15 aa, Homo sapiens
KETCNKSNMCESSKE

SEQ ID NO: 604 - protein, 15 aa, Homo sapiens
CNKSNMCESSKEALA

SEQ ID NO: 605 - protein, 15 aa, Homo sapiens
SNMCESSKEALAENN

SEQ ID NO: 606 - protein, 15 aa, Homo sapiens
CESSKEALAENNLNL

SEQ ID NO: 607 - protein, 15 aa, Homo sapiens
SKEALAENNLNLPKM

SEQ ID NO: 608 - protein, 15 aa, Homo sapiens
ALAENNLNLPKMAEK

SEQ ID NO: 609 - protein, 15 aa, Homo sapiens
ENNLNLPKMAEKDGC

SEQ ID NO: 610 - protein, 15 aa, Homo sapiens
LNLPKMAEKDGCFQS

SEQ ID NO: 611 - protein, 15 aa, Homo sapiens
PKMAEKDGCFQSGFN

SEQ ID NO: 612 - protein, 15 aa, Homo sapiens
AEKDGCFQSGFNEET

SEQ ID NO: 613 - protein, 15 aa, Homo sapiens
DGCFQSGFNEETCLV

SEQ ID NO: 614 - protein, 15 aa, Homo sapiens
FQSGFNEETCLVKII

SEQ ID NO: 615 - protein, 15 aa, Homo sapiens
GFNEETCLVKIITGL

SEQ ID NO: 616 - protein, 15 aa, Homo sapiens
EETCLVKIITGLLEF

SEQ ID NO: 617 - protein, 15 aa, Homo sapiens
CLVKIITGLLEFEVY

SEQ ID NO: 618 - protein, 15 aa, Homo sapiens
KIITGLLEFEVYLEY

SEQ ID NO: 619 - protein, 15 aa, Homo sapiens
TGLLEFEVYLEYLQN

SEQ ID NO: 620 - protein, 15 aa, Homo sapiens
LEFEVYLEYLQNRFE

SEQ ID NO: 621 - protein, 15 aa, Homo sapiens
EVYLEYLQNRFESSE

SEQ ID NO: 622 - protein, 15 aa, Homo sapiens
LEYLQNRFESSEEQA

SEQ ID NO: 623 - protein, 15 aa, Homo sapiens
LQNRFESSEEQARAV

SEQ ID NO: 624 - protein, 15 aa, Homo sapiens
RFESSEEQARAVQMS

SEQ ID NO: 625 - protein, 15 aa, Homo sapiens
SSEEQARAVQMSTKV

SEQ ID NO: 626 - protein, 15 aa, Homo sapiens
EQARAVQMSTKVLIQ

SEQ ID NO: 627 - protein, 15 aa, Homo sapiens
RAVQMSTKVLIQFLQ

SEQ ID NO: 628 - protein, 15 aa, Homo sapiens
QMSTKVLIQFLQKKA

SEQ ID NO: 629 - protein, 15 aa, Homo sapiens
TKVLIQFLQKKAKNL

SEQ ID NO: 630 - protein, 15 aa, Homo sapiens
LIQFLQKKAKNLDAI

SEQ ID NO: 631 - protein, 15 aa, Homo sapiens
FLQKKAKNLDAITTP

SEQ ID NO: 632 - protein, 15 aa, Homo sapiens
KKAKNLDAITTPDPT

SEQ ID NO: 633 - protein, 15 aa, Homo sapiens
KNLDAITTPDPTTNA

SEQ ID NO: 634 - protein, 15 aa, Homo sapiens
DAITTPDPTTNASLL

SEQ ID NO: 635 - protein, 15 aa, Homo sapiens
TTPDPTTNASLLTKL

SEQ ID NO: 636 - protein, 15 aa, Homo sapiens
DPTTNASLLTKLQAQ

SEQ ID NO: 637 - protein, 15 aa, Homo sapiens
TNASLLTKLQAQNQW

SEQ ID NO: 638 - protein, 15 aa, Homo sapiens
SLLTKLQAQNQWLQD

SEQ ID NO: 639 - protein, 15 aa, Homo sapiens
TKLQAQNQWLQDMTT

SEQ ID NO: 640 - protein, 15 aa, Homo sapiens
QAQNQWLQDMTTHLI

SEQ ID NO: 641 - protein, 15 aa, Homo sapiens
NQWLQDMTTHLILRS

SEQ ID NO: 642 - protein, 15 aa, Homo sapiens
LQDMTTHLILRSFKE

SEQ ID NO: 643 - protein, 15 aa, Homo sapiens
MTTHLILRSFKEFLQ

SEQ ID NO: 644 - protein, 15 aa, Homo sapiens
HLILRSFKEFLQSSL

SEQ ID NO: 645 - protein, 15 aa, Homo sapiens
LRSFKEFLQSSLRAL

SEQ ID NO: 646 - protein, 15 aa, Homo sapiens
FKEFLQSSLRALRQM

SEQ ID NO: 647 - protein, 111 aa, Oryctolagus cuniculus
AYDMTQTPASVSAAVGGTVTIKCQASQSINNELSWYQQKPGQRPKLLIYRASTLASGVSSRFKGSGSGTEFTLTISDLECADAATYYCQQGYSLRNIDNAFGGGTEVVVKR

SEQ ID NO: 648 - protein, 88 aa, Homo sapiens
AIQMTQSPSSLSASVGDRVTITCRASQGIRNDLGWYQQKPGKAPKLLIYAASSLQSGVPSRFSGSGSGTDFTLTISSLQPEDFATYYC

SEQ ID NO: 649 - protein, 88 aa, Homo sapiens
DIQMTQSPSSLSASVGDRVTITCRASQGISNYLAWYQQKPGKVPKLLIYAASTLQSGVPSRFSGSGSGTDFTLTISSLQPEDVATYYC

SEQ ID NO: 650 - protein, 88 aa, Homo sapiens
DIQMTQSPSTLSASVGDRVTITCRASQSISSWLAWYQQKPGKAPKLLIYKASSLESGVPSRFSGSGSGTEFTLTISSLQPDDFATYYC

SEQ ID NO: 651 - protein, 111 aa, synthetic construct
  REGION 1..111: note = Humanized antibody
AIQMTQSPSSLSASVGDRVTITCQASQSINNELSWYQQKPGKAPKLLIYRASTLASGVPSRFSGSGSGTDFTLTISSLQPEDFATYYCQQGYSLRNIDNAFGGGTKVEIKR

SEQ ID NO: 652 - protein, 117 aa, Oryctolagus cuniculus
QSLEESGGRLVTPGTPLTLTCTASGFSLSNYYVTWVRQAPGKGLEWIGIIYGSDETAYATWAIGRFTISKTSTTVDLKMTSLTAADTATYFCARDDSSDWDAKFNLWGQGTLVTVSS

SEQ ID NO: 653 - protein, 97 aa, Homo sapiens
EVQLVESGGGLVQPGGSLRLSCAASGFTVSSNYMSWVRQAPGKGLEWVSVIYSGGSTYYADSVKGRFTISRDNSKNTLYLQMNSLRAEDTAVYYCAR

SEQ ID NO: 654 - protein, 97 aa, Homo sapiens
EVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCAR

SEQ ID NO: 655 - protein, 98 aa, Homo sapiens
EVQLLESGGGLVQPGGSLRLSCAASGFTFSSYAMSWVRQAPGKGLEWVSVIYSGGSSTYYADSVKGRFTISRDNSKNTLYLQMNSLRAEDTAVYYCAK

SEQ ID NO: 656 - protein, 120 aa, synthetic construct
  REGION 1..120: note = Humanized antibody
EVQLVESGGGLVQPGGSLRLSCAASGFSLSNYYVTWVRQAPGKGLEWVGIIYGSDETAYATWAIGRFTISRDNSKNTLYLQMNSLRAEDTAVYYCARDDSSDWDAKFNLWGQGTLVTVSS

SEQ ID NO: 657 - protein, 120 aa, synthetic construct
  REGION 1..120: note = Humanized antibody
EVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNLWGQGTLVTVSS

SEQ ID NO: 658 - protein, 166 aa, Oryctolagus cuniculus
METGLRWLLLVAVLKGVQCQSLEESGGRLVTPGTPLTLTCTASGFSLSNYYVTWVRQAPGKGLEWIGIIYGSDETAYATSAIGRFTISKTSTTVDLKMTSLTAADTATYFCARDDSSDWDAKFNLWGQGTLVTVSSASTKGPSVFPLAPSSKSTSGGTAALGCLVK

SEQ ID NO: 659 - protein, 16 aa, Oryctolagus cuniculus
IIYGSDETAYATSAIG

SEQ ID NO: 660 - protein, 122 aa, Oryctolagus cuniculus
MDTRAPTQLLGLLLLWLPGARCAYDMTQTPASVSAAVGGTVTIKCQASQSINNELSWYQQKPGQRPKLLIYRASTLASGVSSRFKGSGSGTEFTLTISDLECADAATYYCQQGYSLRNIDNA

SEQ ID NO: 661 - protein, 125 aa, Oryctolagus cuniculus
METGLRWLLLVAVLKGVQCQSLEESGGRLVTPGTPLTLTCTASGFSLSNYYVTWVRQAPGKGLEWIGIIYGSDETAYATWAIGRFTISKTSTTVDLKMTSLTAADTATYFCARDDSSDWDAKFNL

SEQ ID NO: 662 - DNA, 366 bp, Oryctolagus cuniculus
  source 1..366: mol_type = genomic DNA
atggacacgagggcccccactcagctgctggggctcctgctgctctggctcccaggtgccagatgtgcctatgatatgacccagactccagcctcggtgtctgcagctgtgggaggcacagtcaccatcaagtgccaggccagtcagagcattaacaatgaattatcctggtatcagcagaaaccagggcagcgtcccaagctcctgatctatagggcatccactctggcatctggggtctcatcgcggttcaaaggcagtggatctgggacagagttcactctcaccatcagcgacctggagtgtgccgatgctgccacttactactgtcaacagggttatagtctgaggaatattgataatgct

SEQ ID NO: 663 - DNA, 375 bp, Oryctolagus cuniculus
  source 1..375: mol_type = genomic DNA
atggagactgggctgcgctggcttctcctggtcgctgtgctcaaaggtgtccagtgtcagtcgctggaggagtccgggggtcgcctggtcacgcctgggacacccctgacactcacctgcacagcctctggattctccctcagtaactactacgtgacctgggtccgccaggctccagggaaggggctggaatggatcggaatcatttatggtagtgatgaaacggcctacgcgacctgggcgataggccgattcaccatctccaaaacctcgaccacggtggatctgaaaatgaccagtctgacagccgcggacacggccacctatttctgtgccagagatgatagtagtgactgggatgcaaaatttaacttg

SEQ ID NO: 664 - protein, 450 aa, Oryctolagus cuniculus
EVQLVESGGGLVQPGGSLRLSCAASGFSLSNYYVTWVRQAPGKGLEWVGIIYGSDETAYATWAIGRFTISRDNSKNTLYLQMNSLRAEDTAVYYCARDDSSDWDAKFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 665 - protein, 450 aa, Oryctolagus cuniculus
EVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 666 - protein, 216 aa, Oryctolagus cuniculus
IQMTQSPSSLSASVGDRVTITCQASQSINNELSWYQQKPGKAPKLLIYRASTLASGVPSRFSGSGSGTDFTLTISSLQPDDFATYYCQQGYSLRNIDNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 667 - protein, 122 aa, Oryctolagus cuniculus
MDTRAPTQLLGLLLLWLPGARCAYDMTQTPASVEVAVGGTVTINCQASETIYSWLSWYQQKPGQPPKLLIYQASDLASGVPSRFSGSGAGTEYTLTISGVQCDDAATYYCQQGYSGSNVDNV

SEQ ID NO: 668 - protein, 126 aa, Oryctolagus cuniculus
METGLRWLLLVAVLKGVQCQEQLKESGGRLVTPGTPLTLTCTASGFSLNDHAMGWVRQAPGKGLEYIGFINSGGSARYASWAEGRFTISRTSTTVDLKMTSLTTEDTATYFCVRGGAVWSIHSFDP

SEQ ID NO: 669 - DNA, 366 bp, Oryctolagus cuniculus
  source 1..366: mol_type = genomic DNA
atggacacgagggcccccactcagctgctggggctcctgctgctctggctcccaggtgccagatgtgcctatgatatgacccagactccagcctctgtggaggtagctgtgggaggcacagtcaccatcaattgccaggccagtgagaccatttacagttggttatcctggtatcagcagaagccagggcagcctcccaagctcctgatctaccaggcatccgatctggcatctggggtcccatcgcgattcagcggcagtggggctgggacagagtacactctcaccatcagcggcgtgcagtgtgacgatgctgccacttactactgtcaacagggttatagtggtagtaatgttgataatgtt

SEQ ID NO: 670 - DNA, 378 bp, Oryctolagus cuniculus
  source 1..378: mol_type = genomic DNA
atggagactgggctgcgctggcttctcctggtcgctgtgctcaaaggtgtccagtgtcaggagcagctgaaggagtccgggggtcgcctggtcacgcctgggacacccctgacacttacctgcacagcctctggattctccctcaatgaccatgcaatgggctgggtccgccaggctccagggaaggggctggaatacatcggattcattaatagtggtggtagcgcacgctacgcgagctgggcagaaggccgattcaccatctccagaacctcgaccacggtggatctgaaaatgaccagtctgacaaccgaggacacggccacctatttctgtgtcagagggggtgctgtttggagtattcatagttttgatccc

SEQ ID NO: 671 - protein, 123 aa, Oryctolagus cuniculus
MDTRAPTQLLGLLLLWLPGATFAAVLTQTPSPVSAAVGGTVSISCQASQSVYDNNYLSWFQQKPGQPPKLLIYGASTLASGVPSRFVGSGSGTQFTLTITDVQCDDAATYYCAGVYDDDSDNA

SEQ ID NO: 672 - protein, 125 aa, Oryctolagus cuniculus
METGLRWLLLVAVLKGVQCQSLEESGGRLVTPGTPLTLTCTASGFSLSVYYMNWVRQAPGKGLEWIGFITMSDNINYASWAKGRFTISKTSTTVDLKMTSPTTEDTATYFCARSRGWGTMGRLDL

SEQ ID NO: 673 - DNA, 369 bp, Oryctolagus cuniculus
  source 1..369: mol_type = genomic DNA
atggacacgagggcccccactcagctgctggggctcctgctgctctggctcccaggtgccacatttgccgccgtgctgacccagactccatctcccgtgtctgcagctgtgggaggcacagtcagcatcagttgccaggccagtcagagtgtttatgacaacaactacttatcctggtttcagcagaaaccagggcagcctcccaagctcctgatctatggtgcatccactctggcatctggggtcccatcgcggttcgtgggcagtggatctgggacacagttcactctcaccatcacagacgtgcagtgtgacgatgctgccacttactattgtgcaggcgtttatgatgatgatagtgataatgcc

SEQ ID NO: 674 - DNA, 375 bp, Oryctolagus cuniculus
  source 1..375: mol_type = genomic DNA
atggagactgggctgcgctggcttctcctggtggctgtgctcaaaggtgtccagtgtcagtcgctggaggagtccgggggtcgcctggtcacccctgggacacccctgacactcacctgcacagcctctggattctccctcagtgtctactacatgaactgggtccgccaggctccagggaaggggctggaatggatcggattcattacaatgagtgataatataaattacgcgagctgggcgaaaggccgattcaccatctccaaaacctcgaccacggtggatctgaaaatgaccagtccgacaaccgaggacacggccacctatttctgtgccaggagtcgtggctggggtacaatgggtcggttggatctc

SEQ ID NO: 675 - protein, 123 aa, Oryctolagus cuniculus
MDTRAPTQLLGLLLLWLPGAICDPVLTQTPSPVSAPVGGTVSISCQASQSVYENNYLSWFQQKPGQPPKLLIYGASTLDSGVPSRFKGSGSGTQFTLTITDVQCDDAATYYCAGVYDDDSDDA

SEQ ID NO: 676 - protein, 126 aa, Oryctolagus cuniculus
METGLRWLLLVAVLKGVQCQEQLKESGGGLVTPGGTLTLTCTASGFSLNAYYMNWVRQAPGKGLEWIGFITLNNNVAYANWAKGRFTFSKTSTTVDLKMTSPTPEDTATYFCARSRGWGAMGRLDL

SEQ ID NO: 677 - DNA, 369 bp, Oryctolagus cuniculus
  source 1..369: mol_type = genomic DNA
atggacacgagggcccccactcagctgctggggctcctgctgctctggctcccaggtgccatatgtgaccctgtgctgacccagactccatctcccgtatctgcacctgtgggaggcacagtcagcatcagttgccaggccagtcagagtgtttatgagaacaactatttatcctggtttcagcagaaaccagggcagcctcccaagctcctgatctatggtgcatccactctggattctggggtcccatcgcggttcaaaggcagtggatctgggacacagttcactctcaccattacagacgtgcagtgtgacgatgctgccacttactattgtgcaggcgtttatgatgatgatagtgatgatgcc

SEQ ID NO: 678 - DNA, 378 bp, Oryctolagus cuniculus
  source 1..378: mol_type = genomic DNA
atggagactgggctgcgctggcttctcctggtggctgtgctcaaaggtgtccagtgtcaggagcagctgaaggagtccggaggaggcctggtaacgcctggaggaaccctgacactcacctgcacagcctctggattctccctcaatgcctactacatgaactgggtccgccaggctccagggaaggggctggaatggatcggattcattactctgaataataatgtagcttacgcgaactgggcgaaaggccgattcaccttctccaaaacctcgaccacggtggatctgaaaatgaccagtccgacacccgaggacacggccacctatttctgtgccaggagtcgtggctggggtgcaatgggtcggttggatctc

SEQ ID NO: 679 - protein, 122 aa, Oryctolagus cuniculus
MDTRAPTQLLGLLLLWLPGATFAQVLTQTPSPVSAAVGGTVTINCQASQSVDDNNWLGWYQQKRGQPPKYLIYSASTLASGVPSRFKGSGSGTQFTLTISDLECDDAATYYCAGGFSGNIFA

SEQ ID NO: 680 - protein, 122 aa, Oryctolagus cuniculus
METGLRWLLLVAVLKGVQCQSVEESGGRLVTPGTPLTLTCTVSGFSLSSYAMSWVRQAPGKGLEWIGIIGGFGTTYYATWAKGRFTISKTSTTVDLRITSPTTEDTATYFCARGGPGNGGDI

SEQ ID NO: 681 - DNA, 366 bp, Oryctolagus cuniculus
  source 1..366: mol_type = genomic DNA
atggacacgagggcccccactcagctgctggggctcctgctgctctggctcccaggtgccacatttgcccaagtgctgacccagactccatcgcctgtgtctgcagctgtgggaggcacagtcaccatcaactgccaggccagtcagagtgttgatgataacaactggttaggctggtatcagcagaaacgagggcagcctcccaagtacctgatctattctgcatccactctggcatctggggtcccatcgcggttcaaaggcagtggatctgggacacagttcactctcaccatcagcgacctggagtgtgacgatgctgccacttactactgtgcaggcggttttagtggtaatatctttgct

SEQ ID NO: 682 - DNA, 366 bp, Oryctolagus cuniculus
  source 1..366: mol_type = genomic DNA
atggagactgggctgcgctggcttctcctggtcgctgtgctcaaaggtgtccagtgtcagtcggtggaggagtccgggggtcgcctggtcacgcctgggacacccctgacactcacctgcacagtctctggcttctccctcagtagctatgcaatgagctgggtccgccaggctccaggaaaggggctggagtggatcggaatcattggtggttttggtaccacatactacgcgacctgggcgaaaggccgattcaccatctccaaaacctcgaccacggtggatctgagaatcaccagtccgacaaccgaggacacggccacctatttctgtgccagaggtggtcctggtaatggtggtgacatc

SEQ ID NO: 683 - protein, 122 aa, Oryctolagus cuniculus
MDTRAPTQLLGLLLLWLPGATFAAVLTQTPSPVSVPVGGTVTIKCQSSQSVYNNFLSWYQQKPGQPPKLLIYQASKLASGVPDRFSGSGSGTQFTLTISGVQCDDAATYYCLGGYDDDADNA

SEQ ID NO: 684 - protein, 128 aa, Oryctolagus cuniculus
METGLRWLLLVAVLKGVQCQSVEESGGRLVTPGTPLTLTCTVSGIDLSDYAMSWVRQAPGKGLEWIGIIYAGSGSTWYASWAKGRFTISKTSTTVDLKITSPTTEDTATYFCARDGYDDYGDFDRLDL

SEQ ID NO: 685 - DNA, 366 bp, Oryctolagus cuniculus
  source 1..366: mol_type = genomic DNA
atggacacgagggcccccactcagctgctggggctcctgctgctctggctcccaggtgccacatttgcagccgtgctgacccagacaccatcgcccgtgtctgtacctgtgggaggcacagtcaccatcaagtgccagtccagtcagagtgtttataataatttcttatcgtggtatcagcagaaaccagggcagcctcccaagctcctgatctaccaggcatccaaactggcatctggggtcccagataggttcagcggcagtggatctgggacacagttcactctcaccatcagcggcgtgcagtgtgacgatgctgccacttactactgtctaggcggttatgatgatgatgctgataatgct

SEQ ID NO: 686 - DNA, 384 bp, Oryctolagus cuniculus
  source 1..384: mol_type = genomic DNA
atggagactgggctgcgctggcttctcctggtcgctgtgctcaaaggtgtccagtgtcagtcggtggaggagtccgggggtcgcctggtcacgcctgggacacccctgacgctcacctgcacagtctctggaatcgacctcagtgactatgcaatgagctgggtccgccaggctccagggaaggggctggaatggatcggaatcatttatgctggtagtggtagcacatggtacgcgagctgggcgaaaggccgattcaccatctccaaaacctcgaccacggtggatctgaaaatcaccagtccgacaaccgaggacacggccacctatttctgtgccagagatggatacgatgactatggtgatttcgatcgattggatctc

SEQ ID NO: 687 - protein, 122 aa, Oryctolagus cuniculus
MDTRAPTQLLGLLLLWLPGARCAYDMTQTPASVSAAVGGTVTIKCQASQSINNELSWYQQKSGQRPKLLIYRASTLASGVSSRFKGSGSGTEFTLTISDLECADAATYYCQQGYSLRNIDNA

SEQ ID NO: 688 - protein, 125 aa, Oryctolagus cuniculus
METGLRWLLLVAVLSGVQCQSLEESGGRLVTPGTPLTLTCTASGFSLSNYYMTWVRQAPGKGLEWIGMIYGSDETAYANWAIGRFTISKTSTTVDLKMTSLTAADTATYFCARDDSSDWDAKFNL

SEQ ID NO: 689 - DNA, 366 bp, Oryctolagus cuniculus
  source 1..366: mol_type = genomic DNA
atggacacgagggcccccactcagctgctggggctcctgctgctctggctcccaggtgccagatgtgcctatgatatgacccagactccagcctcggtgtctgcagctgtgggaggcacagtcaccatcaaatgccaggccagtcagagcattaacaatgaattatcctggtatcagcagaaatcagggcagcgtcccaagctcctgatctatagggcatccactctggcatctggggtctcatcgcggttcaaaggcagtggatctgggacagagttcactctcaccatcagcgacctggagtgtgccgatgctgccacttactactgtcaacagggttatagtctgaggaatattgataatgct

SEQ ID NO: 690 - DNA, 375 bp, Oryctolagus cuniculus
  source 1..375: mol_type = genomic DNA
atggagactgggctgcgctggcttctcctggtcgctgtgctctcaggtgtccagtgtcagtcgctggaggagtccgggggtcgcctggtcacgcctgggacacccctgacactcacctgcacagcctctggattctccctcagtaactactacatgacctgggtccgccaggctccagggaaggggctggaatggatcggaatgatttatggtagtgatgaaacagcctacgcgaactgggcgataggccgattcaccatctccaaaacctcgaccacggtggatctgaaaatgaccagtctgacagccgcggacacggccacctatttctgtgccagagatgatagtagtgactgggatgcaaaatttaacttg

SEQ ID NO: 691 - protein, 450 aa, Oryctolagus cuniculus
EVQLVESGGGLVQPGGSLRLSCAASGFSLSNYYMTWVRQAPGKGLEWVGMIYGSDETAYANWAIGRFTISRDNSKNTLYLQMNSLRAEDTAVYYCARDDSSDWDAKFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 692 - protein, 450 aa, Oryctolagus cuniculus
EVQLVESGGGLVQPGGSLRLSCAASGFSLSNYYMTWVRQAPGKGLEWVGMIYGSDETAYANSAIGRFTISRDNSKNTLYLQMNSLRAEDTAVYYCARDDSSDWDAKFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 693 - protein, 217 aa, Oryctolagus cuniculus
DIQMTQSPSTLSASVGDRVTITCQASQSINNELSWYQQKPGKAPKLLIYRASTLASGVPSRFSGSGSGTEFTLTISSLQPDDFATYYCQQGYSLRNIDNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 694 - DNA, 33 bp, Oryctolagus cuniculus
  source 1..33: mol_type = genomic DNA
caggccagtcagagcattaacaatgagttatcc

SEQ ID NO: 695 - DNA, 36 bp, Oryctolagus cuniculus
  source 1..36: mol_type = genomic DNA
caacagggttatagtctgaggaacattgataatgct

SEQ ID NO: 696 - DNA, 48 bp, Oryctolagus cuniculus
  source 1..48: mol_type = genomic DNA
atcatctatggtagtgatgaaaccgcctacgctacctccgctataggc

SEQ ID NO: 697 - DNA, 36 bp, Oryctolagus cuniculus
  source 1..36: mol_type = genomic DNA
gatgatagtagtgactgggatgcaaagttcaacttg

SEQ ID NO: 698 - DNA, 336 bp, Oryctolagus cuniculus
  source 1..336: mol_type = genomic DNA
gctatccagatgacccagtctccttcctccctgtctgcatctgtaggagacagagtcaccatcacttgccaggccagtcagagcattaacaatgagttatcctggtatcagcagaaaccagggaaagcccctaagctcctgatctatagggcatccactctggcatctggggtcccatcaaggttcagcggcagtggatctgggacagacttcactctcaccatcagcagcctgcagcctgatgattttgcaacttattactgccaacagggttatagtctgaggaacattgataatgctttcggcggagggaccaaggtggaaatcaaacgtacg

SEQ ID NO: 699 - protein, 112 aa, Oryctolagus cuniculus
AIQMTQSPSSLSASVGDRVTITCQASQSINNELSWYQQKPGKAPKLLIYRASTLASGVPSRFSGSGSGTDFTLTISSLQPDDFATYYCQQGYSLRNIDNAFGGGTKVEIKRT

SEQ ID NO: 700 - DNA, 360 bp, Oryctolagus cuniculus
  source 1..360: mol_type = genomic DNA
gaggtgcagctggtggagtctgggggaggcttggtccagcctggggggtccctgagactctcctgtgcagcctctggattctccctcagtaactactacgtgacctgggtccgtcaggctccagggaaggggctggagtgggtcggcatcatctatggtagtgatgaaaccgcctacgctacctccgctataggccgattcaccatctccagagacaattccaagaacaccctgtatcttcaaatgaacagcctgagagctgaggacactgctgtgtattactgtgctagagatgatagtagtgactgggatgcaaagttcaacttgtggggccaagggaccctcgtcaccgtctcgagc

SEQ ID NO: 701 - DNA, 651 bp, Oryctolagus cuniculus
  source 1..651: mol_type = genomic DNA
gctatccagatgacccagtctccttcctccctgtctgcatctgtaggagacagagtcaccatcacttgccaggccagtcagagcattaacaatgagttatcctggtatcagcagaaaccagggaaagcccctaagctcctgatctatagggcatccactctggcatctggggtcccatcaaggttcagcggcagtggatctgggacagacttcactctcaccatcagcagcctgcagcctgatgattttgcaacttattactgccaacagggttatagtctgaggaacattgataatgctttcggcggagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 702 - protein, 217 aa, Oryctolagus cuniculus
AIQMTQSPSSLSASVGDRVTITCQASQSINNELSWYQQKPGKAPKLLIYRASTLASGVPSRFSGSGSGTDFTLTISSLQPDDFATYYCQQGYSLRNIDNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 703 - DNA, 1350 bp, Oryctolagus cuniculus
  source 1..1350: mol_type = genomic DNA
gaggtgcagctggtggagtctgggggaggcttggtccagcctggggggtccctgagactctcctgtgcagcctctggattctccctcagtaactactacgtgacctgggtccgtcaggctccagggaaggggctggagtgggtcggcatcatctatggtagtgatgaaaccgcctacgctacctccgctataggccgattcaccatctccagagacaattccaagaacaccctgtatcttcaaatgaacagcctgagagctgaggacactgctgtgtattactgtgctagagatgatagtagtgactgggatgcaaagttcaacttgtggggccaagggaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 704 - protein, 450 aa, Oryctolagus cuniculus
EVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 705 - DNA, 705 bp, Oryctolagus cuniculus
  source 1..705: mol_type = genomic DNA
atgaagtgggtaacctttatttcccttctgtttctctttagcagcgcttattccgctatccagatgacccagtctccttcctccctgtctgcatctgtaggagacagagtcaccatcacttgccaggccagtcagagcattaacaatgagttatcctggtatcagcagaaaccagggaaagcccctaagctcctgatctatagggcatccactctggcatctggggtcccatcaaggttcagcggcagtggatctgggacagacttcactctcaccatcagcagcctgcagcctgatgattttgcaacttattactgccaacagggttatagtctgaggaacattgataatgctttcggcggagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 706 - protein, 235 aa, Oryctolagus cuniculus
MKWVTFISLLFLFSSAYSAIQMTQSPSSLSASVGDRVTITCQASQSINNELSWYQQKPGKAPKLLIYRASTLASGVPSRFSGSGSGTDFTLTISSLQPDDFATYYCQQGYSLRNIDNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 707 - DNA, 1404 bp, Oryctolagus cuniculus
  source 1..1404: mol_type = genomic DNA
atgaagtgggtaacctttatttcccttctgtttctctttagcagcgcttattccgaggtgcagctggtggagtctgggggaggcttggtccagcctggggggtccctgagactctcctgtgcagcctctggattctccctcagtaactactacgtgacctgggtccgtcaggctccagggaaggggctggagtgggtcggcatcatctatggtagtgatgaaaccgcctacgctacctccgctataggccgattcaccatctccagagacaattccaagaacaccctgtatcttcaaatgaacagcctgagagctgaggacactgctgtgtattactgtgctagagatgatagtagtgactgggatgcaaagttcaacttgtggggccaagggaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 708 - protein, 468 aa, Oryctolagus cuniculus
MKWVTFISLLFLFSSAYSEVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 709 - protein, 111 aa, Oryctolagus cuniculus
AIQMTQSPSSLSASVGDRVTITCQASQSINNELSWYQQKPGKAPKLLIYRASTLASGVPSRFSGSGSGTDFTLTISSLQPDDFATYYCQQGYSLRNIDNAFGGGTKVEIKR

SEQ ID NO: 710 - protein, 11 aa, Homo sapiens
RASQGIRNDLG

SEQ ID NO: 711 - protein, 11 aa, Homo sapiens
RASQGISNYLA

SEQ ID NO: 712 - protein, 11 aa, Homo sapiens
RASQSISSWLA

SEQ ID NO: 713 - protein, 7 aa, Homo sapiens
AASSLQS

SEQ ID NO: 714 - protein, 7 aa, Homo sapiens
AASTLQS

SEQ ID NO: 715 - protein, 7 aa, Homo sapiens
KASSLES

SEQ ID NO: 716 - protein, 5 aa, Homo sapiens
SNYMS

SEQ ID NO: 717 - protein, 16 aa, Homo sapiens
VIYSGGSTYYADSVKG

SEQ ID NO: 718 - protein, 17 aa, Homo sapiens
VIYSGGSSTYYADSVKG

SEQ ID NO: 719 - protein, 330 aa, synthetic construct
  REGION 1..330: note = Gamma-1 constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 720 - DNA, 297 bp, Oryctolagus cuniculus
  source 1..297: mol_type = genomic DNA
atccagatgacccagtctccttcctccctgtctgcatctgtaggagacagagtcaccatcacttgccaggccagtcagagcattaacaatgagttatcctggtatcagcagaaaccagggaaagcccctaagctcctgatctatagggcatccactctggcatctggggtcccatcaaggttcagcggcagtggatctgggacagacttcactctcaccatcagcagcctgcagcctgatgattttgcaacttattactgccaacagggttatagtctgaggaacattgataatgct

SEQ ID NO: 721 - DNA, 333 bp, Oryctolagus cuniculus
  source 1..333: mol_type = genomic DNA
gcctatgatatgacccagactccagcctcggtgtctgcagctgtgggaggcacagtcaccatcaagtgccaggccagtcagagcattaacaatgaattatcctggtatcagcagaaaccagggcagcgtcccaagctcctgatctatagggcatccactctggcatctggggtctcatcgcggttcaaaggcagtggatctgggacagagttcactctcaccatcagcgacctggagtgtgccgatgctgccacttactactgtcaacagggttatagtctgaggaatattgataatgctttcggcggagggaccgaggtggtggtcaaacgt

SEQ ID NO: 722 - DNA, 648 bp, Oryctolagus cuniculus
  source 1..648: mol_type = genomic DNA
atccagatgacccagtctccttcctccctgtctgcatctgtaggagacagagtcaccatcacttgccaggccagtcagagcattaacaatgagttatcctggtatcagcagaaaccagggaaagcccctaagctcctgatctatagggcatccactctggcatctggggtcccatcaaggttcagcggcagtggatctgggacagacttcactctcaccatcagcagcctgcagcctgatgattttgcaacttattactgccaacagggttatagtctgaggaacattgataatgctttcggcggagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 723 - DNA, 333 bp, Oryctolagus cuniculus
  source 1..333: mol_type = genomic DNA
gctatccagatgacccagtctccttcctccctgtctgcatctgtaggagacagagtcaccatcacttgccaggccagtcagagcattaacaatgagttatcctggtatcagcagaaaccagggaaagcccctaagctcctgatctatagggcatccactctggcatctggggtcccatcaaggttcagcggcagtggatctgggacagacttcactctcaccatcagcagcctgcagcctgatgattttgcaacttattactgccaacagggttatagtctgaggaacattgataatgctttcggcggagggaccaaggtggaaatcaaacgt

SEQ ID NO: 724 - DNA, 327 bp, Oryctolagus cuniculus
  source 1..327: mol_type = genomic DNA
gaggtgcagctggtggagtctgggggaggcttggtccagcctggggggtccctgagactctcctgtgcagcctctggattctccctcagtaactactacgtgacctgggtccgtcaggctccagggaaggggctggagtgggtcggcatcatctatggtagtgatgaaaccgcctacgctacctccgctataggccgattcaccatctccagagacaattccaagaacaccctgtatcttcaaatgaacagcctgagagctgaggacactgctgtgtattactgtgctagagatgatagtagtgactgggatgcaaagttcaacttg

SEQ ID NO: 725 - DNA, 351 bp, Oryctolagus cuniculus
  source 1..351: mol_type = genomic DNA
cagtcgctggaggagtccgggggtcgcctggtcacgcctgggacacccctgacactcacctgcacagcctctggattctccctcagtaactactacgtgacctgggtccgccaggctccagggaaggggctggaatggatcggaatcatttatggtagtgatgaaacggcctacgcgacctgggcgataggccgattcaccatctccaaaacctcgaccacggtggatctgaaaatgaccagtctgacagccgcggacacggccacctatttctgtgccagagatgatagtagtgactgggatgcaaaatttaacttgtggggccaaggcaccctggtcaccgtctcgagc

SEQ ID NO: 726 - protein, 224 aa, Homo sapiens
MEKLLCFLVLTSLSHAFGQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 727 - protein, 468 aa, Homo sapiens
MLAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQDSSSVPLPTFLVAGGSLAFGTLLCIAIVLRFKKTWKLRALKEGKTSMHPPYSLGQLVPERPRPTPVLVPLISPPVSPSSLGSDNTSSHNRPDARDPRSPYDISNTDYFFPR

SEQ ID NO: 728 - protein, 918 aa, Homo sapiens [sequence omitted]

SEQ ID NO: 729 - protein, 111 aa, Oryctolagus cuniculus
AYDMTQTPASVEVAVGGTVTINCQASETIYSWLSWYQQKPGQPPKLLIYQASDLASGVPSRFSGSGAGTEYTLTISGVQCDDAATYYCQQGYSGSNVDNVFGGGTEVVVKR

SEQ ID NO: 730 - protein, 88 aa, Homo sapiens; identical to SEQ ID NO: 650

SEQ ID NO: 731 - protein, 88 aa, Homo sapiens
DIQMTQSPSTLSASVGDRVTITCRASQSISSWLAWYQQKPGKAPKLLIYDASSLESGVPSRFSGSGSGTEFTLTISSLQPDDFATYYC

SEQ ID NO: 732 - protein, 88 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYC

SEQ ID NO: 733 - protein, 111 aa, synthetic construct
  REGION 1..111: note = Humanized antibody
DIQMTQSPSTLSASVGDRVTITCQASETIYSWLSWYQQKPGKAPKLLIYQASDLASGVPSRFSGSGSGTEFTLTISSLQPDDFATYYCQQGYSGSNVDNVFGGGTKVEIKR

SEQ ID NO: 734 - protein, 11 aa, Homo sapiens
FGGGTKVEIKR

SEQ ID NO: 735 - protein, 118 aa, Oryctolagus cuniculus
QEQLKESGGRLVTPGTPLTLTCTASGFSLNDHAMGWVRQAPGKGLEYIGFINSGGSARYASWAEGRFTISRTSTTVDLKMTSLTTEDTATYFCVRGGAVWSIHSFDPWGPGTLVTVSS

SEQ ID NO: 736 - protein, 98 aa, Homo sapiens
QVQLVESGGGLVQPGGSLRLSCSASGFTFSSYAMHWVRQAPGKGLEYVSAISSNGGSTYYADSVKGRFTISRDNSKNTLYLQMNSLRAEDTAVYYCAR

SEQ ID NO: 737 - protein, 97 aa, Homo sapiens; identical to SEQ ID NO: 653

SEQ ID NO: 738 - protein, 97 aa, Homo sapiens
EVQLVETGGGLIQPGGSLRLSCAASGFTVSSNYMSWVRQAPGKGLEWVSVIYSGGSTYYADSVKGRFTISRDNSKNTLYLQMNSLRAEDTAVYYCAR

SEQ ID NO: 739 - protein, 120 aa, synthetic construct
  REGION 1..120: note = Humanized antibody
QVQLVESGGGLVQPGGSLRLSCSASGFSLNDHAMGWVRQAPGKGLEYVGFINSGGSARYASSAEGRFTISRDNSKNTLYLQMNSLRAEDTAVYYCARGGAVWSIHSFDPWGQGTLVTVSS

SEQ ID NO: 740 - protein, 11 aa, Homo sapiens
WGQGTLVTVSS

SEQ ID NO: 741 - DNA, 32 bp, synthetic construct
  misc_feature 1..32: note = PCR Primer
  source 1..32: mol_type = other DNA
agcgcttattccgctatccagatgacccagtc

SEQ ID NO: 742 - DNA, 22 bp, synthetic construct
  misc_feature 1..22: note = PCR Primer
  source 1..22: mol_type = other DNA
cgtacgtttgatttccaccttg

SEQ ID NO: 743 - DNA, 32 bp, synthetic construct
  misc_feature 1..32: note = PCR Primer
  source 1..32: mol_type = other DNA
agcgcttattccgaggtgcagctggtggagtc

SEQ ID NO: 744 - DNA, 20 bp, synthetic construct
  misc_feature 1..20: note = PCR Primer
  source 1..20: mol_type = other DNA
ctcgagacggtgacgagggt

SEQ ID NO: 745 - protein, 450 aa, synthetic construct; identical to SEQ ID NO: 704
  REGION 1..450: note = Heavy Chain

SEQ ID NO: 746 - protein, 217 aa, synthetic construct; identical to SEQ ID NO: 702
  REGION 1..217: note = Light Chain